SEQUENCE LISTING (762 sequences)

SEQ ID NO: 1 - protein, 5 aa, synthetic construct
  REGION 1..5: note = Description of Artificial Sequence: Synthetic peptide
  REGION 1..5: note = LP7A5 HCDR1
DHYIN

SEQ ID NO: 2 - protein, 17 aa, synthetic construct
  REGION 1..17: note = Description of Artificial Sequence: Synthetic peptide
  REGION 1..17: note = LP7A5 HCDR2
QIYPGDGNTYYNQKFKG

SEQ ID NO: 3 - protein, 9 aa, synthetic construct
  REGION 1..9: note = Description of Artificial Sequence: Synthetic peptide
  REGION 1..9: note = LP7A5_1 HCDR3
NYGDYTIDF

SEQ ID NO: 4 - protein, 17 aa, synthetic construct
  REGION 1..17: note = Description of Artificial Sequence: Synthetic peptide
  REGION 1..17: note = LP7A5 LCDR1
KSSQSLLYSSNQKNYLA

SEQ ID NO: 5 - protein, 7 aa, synthetic construct
  REGION 1..7: note = Description of Artificial Sequence: Synthetic peptide
  REGION 1..7: note = LP7A5 LCDR2
WASTRES

SEQ ID NO: 6 - protein, 8 aa, synthetic construct
  REGION 1..8: note = Description of Artificial Sequence: Synthetic peptide
  REGION 1..8: note = LP7A5 LCDR3
QQYYRYHT

SEQ ID NO: 7 - protein, 118 aa, synthetic construct
  REGION 1..118: note = Description of Artificial Sequence: Synthetic polypeptide
  REGION 1..118: note = LP7A5 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSS

SEQ ID NO: 8 - protein, 112 aa, synthetic construct
  REGION 1..112: note = Description of Artificial Sequence: Synthetic polypeptide
  REGION 1..112: note = LP7A5 Light Chain Variable Region
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIK

SEQ ID NO: 9 - protein, 5 aa, synthetic construct
  REGION 1..5: note = Description of Artificial Sequence: Synthetic peptide
  REGION 1..5: note = I3RB217 HCDR1
SYWIS

SEQ ID NO: 10 - protein, 17 aa, synthetic construct
  REGION 1..17: note = Description of Artificial Sequence: Synthetic peptide
  REGION 1..17: note = I3RB217 HCDR2
IIDPSDSDTRYSPSFQG

SEQ ID NO: 11 - protein, 9 aa, synthetic construct
  REGION 1..9: note = Description of Artificial Sequence: Synthetic peptide
  REGION 1..9: note = I3RB217 HCDR3
GDGSTDLDY

SEQ ID NO: 12 - protein, 11 aa, synthetic construct
  REGION 1..11: note = Description of Artificial Sequence: Synthetic peptide
  REGION 1..11: note = I3RB217 LCDR1
RASQSVSSSYL

SEQ ID NO: 13 - protein, 7 aa, synthetic construct
  REGION 1..7: note = Description of Artificial Sequence: Synthetic peptide
  REGION 1..7: note = I3RB217 LCDR2
GASSRAT

SEQ ID NO: 14 - protein, 9 aa, synthetic construct
  REGION 1..9: note = Description of Artificial Sequence: Synthetic peptide
  REGION 1..9: note = I3RB217 LCDR3
QQDYGFPWT

SEQ ID NO: 15 - protein, 118 aa, synthetic construct
  REGION 1..118: note = Description of Artificial Sequence: Synthetic polypeptide
  REGION 1..118: note = I3RB217 Heavy Chain Variable Region
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSS

SEQ ID NO: 16 - protein, 108 aa, synthetic construct
  REGION 1..108: note = Description of Artificial Sequence: Synthetic polypeptide
  REGION 1..108: note = I3RB217 Light Chain Variable Region
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIK

SEQ ID NO: 17 - protein, 717 aa, synthetic construct
  REGION 1..717: note = Description of Artificial Sequence: Synthetic polypeptide
  REGION 1..717: note = Vgamma9 half antibody
MAWVWTLLFLMAAAQSIQADIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 18 - protein, 713 aa, synthetic construct
  REGION 1..713: note = Description of Artificial Sequence: Synthetic polypeptide
  REGION 1..713: note = CD123 half antibody
MAWVWTLLFLMAAAQSIQAEIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSEGKSSGSGSESKSTEGKSSGSGSESKSTGGSEVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 713 aa, synthetic construct; identical to SEQ ID NO: 18
  REGION 1..713: note = Description of Artificial Sequence: Synthetic polypeptide
  REGION 1..713: note = B23B49 half antibody

SEQ ID NO: 20 - DNA, 2151 bp, synthetic construct
  misc_feature 1..2151: note = Description of Artificial Sequence: Synthetic polynucleotide
  misc_feature 1..2151: note = Vgamma9 half antibody
  source 1..2151: mol_type = other DNA
atggcctgggtgtggaccctgctgttcctgatggccgccgcccagagcatccaggccgacatcgtgatgagccagagcccaagcagcctggccgtgagcgtgggcgagaaggtgaccatgagctgcaagagcagccagagcctgctgtacagcagcaaccagaagaactacctggcctggtaccagcagaagccaggccagagcccaaagctgctgatctactgggccagcacccgcgagagcggcgtgccagaccgcttcaccggcagcggcagcggcaccgacttcaccctgaccatcagcagcgtgaaggccgaggacctggccgtgtactactgccagcagtactaccgctaccacaccttcggcaccggcaccaagctggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagcgaggtgcagctgcagcagagcggcgccgagctggcccgcccaggcgccagcgtgaagctgagctgcaaggccagcggcttcaccttcaccgaccactacatcaactgggtgaagcagcgcaccggccagggcctggagtggatcggccagatctacccaggcgacggcaacacctactacaaccagaagttcaagggcaaggccaccctgaccgccgacaagagcagcagcaccgcctacatgcagctgagcagcctgaccagcgaggacagcgccgtgtacttctgcgccccaaactacggcgactacaccatcgacttctggggccagggcaccagcgtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 21 - DNA, 2139 bp, synthetic construct
  misc_feature 1..2139: note = Description of Artificial Sequence: Synthetic polynucleotide
  misc_feature 1..2139: note = CD123 half antibody
  source 1..2139: mol_type = other DNA
atggcctgggtgtggaccctgctgttcctgatggccgccgcccagagcatccaggccgagatcgtgctgacccagagcccaggcaccctgagcctgagcccaggcgagcgcgccaccctgagctgccgcgccagccagagcgtgagcagcagctacctggcctggtaccagcagaagccaggccaggccccacgcctgctgatctacggcgccagcagccgcgccaccggcatcccagaccgcttcagcggcagcggcagcggcaccgacttcaccctgaccatcagccgcctggagccagaggacttcgccgtgtactactgccagcaggactacggcttcccatggaccttcggccagggcaccaaggtggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagcgaggtgcagctggtgcagagcggcgccgaggtgaagaagccaggcgagagcctgaagatcagctgcaagggcagcggctacagcttcaccagctactggatcagctgggtgcgccagatgccaggcaagggcctggagtggatgggcatcatcgacccaagcgacagcgacacccgctacagcccaagcttccagggccaggtgaccatcagcgccgacaagagcatcagcaccgcctacctgcagtggagcagcctgaaggccagcgacaccgccatgtactactgcgcccgcggcgacggcagcaccgacctggactactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 22 - DNA, 2154 bp, synthetic construct
  misc_feature 1..2154: note = Description of Artificial Sequence: Synthetic polynucleotide
  misc_feature 1..2154: note = B23B49 half antibody
  source 1..2154: mol_type = other DNA
atggcctgggtgtggaccctgctgttcctgatggccgccgcccagagcatccaggccgacatcgtgatgacccagagcccagacagcctggccgtgagcctgggcgagcgcgccaccatcaactgccgcgccagccagagcgtggactacaacggcatcagctacatgcactggtaccagcagaagccaggccagccaccaaagctgctgatctacgccgccagcaacccagagagcggcgtgccagaccgcttcagcggcagcggcagcggcaccgacttcaccctgaccatcagcagcctgcaggccgaggacgtggccgtgtactactgccagcagatcatcgaggacccatggaccttcggccagggcaccaaggtggagatcaagcgcaccgtggccgccccaagcgtgttcatcttcccaccaagcgacgagcagctgaagagcggcaccgccagcgtggtgtgcctgctgaacaacttctacccacgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagcccagtgaccaagagcttcaaccgcggcgagtgcggcggcagcgagggcaagagcagcggcagcggcagcgagagcaagagcaccgagggcaagagcagcggcagcggcagcgagagcaagagcaccggcggcagccagatcaccctgaaggagagcggcccaaccctggtgaagccaacccagaccctgaccctgacctgcaccttcagcggcttcagcctgagcaccagcggcatgggcgtgagctggatccgccagccaccaggcaaggccctggagtggctggcccacatctactgggacgacgacaagcgctacaacccaagcctgaagagccgcctgaccatcaccaaggacaccagcaagaaccaggtggtgctgaccatgaccaacatggacccagtggacaccgccacctactactgcgcccgcctgtacggcttcacctacggcttcgcctactggggccagggcaccctggtgaccgtgagcagcgccagcaccaagggcccaagcgtgttcccactggccccatgcagccgcagcaccagcgagagcaccgccgccctgggctgcctggtgaaggactacttcccagagccagtgaccgtgagctggaacagcggcgccctgaccagcggcgtgcacaccttcccagccgtgctgcagagcagcggcctgtacagcctgagcagcgtggtgaccgtgccaagcagcagcctgggcaccaagacctacacctgcaacgtggaccacaagccaagcaacaccaaggtggacaagcgcgtggagagcaagtacggcccaccatgcccaccatgcccagccccagaggccgccggcggcccaagcgtgttcctgttcccaccaaagccaaaggacaccctgatgatcagccgcaccccagaggtgacctgcgtggtggtggacgtgagccaggaggacccagaggtgcagttcaactggtacgtggacggcgtggaggtgcacaacgccaagaccaagccacgcgaggagcagttcaacagcacctaccgcgtggtgagcgtgctgaccgtgctgcaccaggactggctgaacggcaaggagtacaagtgcaaggtgagcaacaagggcctgccaagcagcatcgagaagaccatcagcaaggccaagggccagccacgcgagccacaggtgtacaccctgccaccaagccaggaggagatgaccaagaaccaggtgagcctgtggtgcctggtgaagggcttctacccaagcgacatcgccgtggagtgggagagcaacggccagccagagaacaactacaagaccaccccaccagtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagcctgggcaag

SEQ ID NO: 23 - protein, 445 aa, synthetic construct
  REGION 1..445: note = Description of Artificial Sequence: Synthetic polypeptide
  REGION 1..445: note = VG1 heavy chain 1
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 24 - protein, 219 aa, synthetic construct
  REGION 1..219: note = Description of Artificial Sequence: Synthetic polypeptide
  REGION 1..219: note = VG1 light chain 1
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 445 aa, synthetic construct
  REGION 1..445: note = Description of Artificial Sequence: Synthetic polypeptide
  REGION 1..445: note = VG1 heavy chain 2
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 215 aa, synthetic construct
  REGION 1..215: note = Description of Artificial Sequence: Synthetic polypeptide
  REGION 1..215: note = VG1 light chain 2
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 447 aa, synthetic construct
  REGION 1..447: note = Description of Artificial Sequence: Synthetic polypeptide
  REGION 1..447: note = VG3 heavy chain 1
QVQLQESGPGLVKPSETLSLTCTVSGYSITSGYFWNWIRQPPGKGLEWIGYISYDGSNNYNPSLKSRVTISRDTSKNQFSLKLSSVTAADTAVYYCASPSPGTGYAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 28 - protein, 219 aa, synthetic construct
  REGION 1..219: note = Description of Artificial Sequence: Synthetic polypeptide
  REGION 1..219: note = VG3 light chain 1
DIQMTQSPSSLSASVGDRVTITCRSSQSLVHSNGNTYLHWYQQKPGKAPKFLIYKVSNRFSGVPSRFSGSGSGTDFTLTISSLQPEDFATYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 447 aa, synthetic construct
  REGION 1..447: note = Description of Artificial Sequence: Synthetic polypeptide
  REGION 1..447: note = VG3 heavy chain 2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 218 aa, synthetic construct
  REGION 1..218: note = Description of Artificial Sequence: Synthetic polypeptide
  REGION 1..218: note = VG3 light chain 2
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 9 aa, synthetic construct
  REGION 1..9: note = Description of Artificial Sequence: Synthetic peptide
  REGION 1..9: note = LP7A5_2 HCDR3
NMGMYTIDF

SEQ ID NO: 32 - protein, 9 aa, synthetic construct
  REGION 1..9: note = Description of Artificial Sequence: Synthetic peptide
  REGION 1..9: note = LP7A5_3 HCDR3
NMGMYTLDF

SEQ ID NO: 33 - protein, 9 aa, synthetic construct
  REGION 1..9: note = Description of Artificial Sequence: Synthetic peptide
  REGION 1..9: note = LP7A5_4 HCDR3
NYGDYTLDF

SEQ ID NO: 34 - protein, 118 aa, synthetic construct
  REGION 1..118: note = Description of Artificial Sequence: Synthetic polypeptide
  REGION 1..118: note = LP7A5_2 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNMGMYTIDFWGQGTSVTVSS

SEQ ID NO: 35 - protein, 118 aa, synthetic construct
  REGION 1..118: note = Description of Artificial Sequence: Synthetic polypeptide
  REGION 1..118: note = LP7A5_3 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNMGMYTLDFWGQGTSVTVSS

SEQ ID NO: 36 - protein, 118 aa, synthetic construct
  REGION 1..118: note = Description of Artificial Sequence: Synthetic polypeptide
  REGION 1..118: note = LP7A5_4 Heavy Chain Variable Region
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTLDFWGQGTSVTVSS

SEQ ID NO: 37-45 - skipped (9 entries)

SEQ ID NO: 46 - protein, 713 aa, synthetic construct; identical to SEQ ID NO: 18
  REGION 1..713: note = Description of Artificial Sequence: Synthetic polypeptide
  REGION 1..713: note = Anti-RSV ('knob'-arm) sequence expressed in CHO cells

SEQ ID NO: 47 - protein, 446 aa, synthetic construct
  REGION 1..446: note = Description of Artificial Sequence: Synthetic polypeptide
  REGION 1..446: note = C33B904 Heavy Chain
EVQLVESGGGLVQPGRSLRLSCAASGFTFDDYAMHWVRQAPGKGLEWVSGIGWSGGSIVYADSVKGRFTISRDNAKNSLYLQMNSLRAEDTALYYCAKDSPYGDFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 220 aa, synthetic construct
  REGION 1..220: note = Description of Artificial Sequence: Synthetic polypeptide
  REGION 1..220: note = C33B904 Light Chain
DIVMTQSPDSLAVSLGERATINCKSSQTVFYSSNNKNYLAWYQQKPGQPPKLLISWASTRKSGVPDRFSGSGSGTDFTLTVSSLQAEDVAVYYCQHYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49-57 - skipped (9 entries)

SEQ ID NO: 58 - protein, 454 aa, synthetic construct
  REGION 1..454: note = Description of Artificial Sequence: Synthetic polypeptide
  REGION 1..454: note = TRBC1 Heavy Chain
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 59 - protein, 219 aa, synthetic construct
  REGION 1..219: note = Description of Artificial Sequence: Synthetic polypeptide
  REGION 1..219: note = TRBC1 Light Chain
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 8 aa, synthetic construct
  REGION 1..8: note = Description of Artificial Sequence: Synthetic peptide
  REGION 1..8: note = TRGV9Ab HCDR1
GFTFTDHY

SEQ ID NO: 61 - protein, 8 aa, synthetic construct
  REGION 1..8: note = Description of Artificial Sequence: Synthetic peptide
  REGION 1..8: note = TRGV9Ab HCDR2
IYPGSGNT

SEQ ID NO: 62 - protein, 11 aa, synthetic construct
  REGION 1..11: note = Description of Artificial Sequence: Synthetic peptide
  REGION 1..11: note = TRGV9Ab HCDR3
ARNYGDYTIDF

SEQ ID NO: 63 - protein, 12 aa, synthetic construct
  REGION 1..12: note = Description of Artificial Sequence: Synthetic peptide
  REGION 1..12: note = TRGV9Ab LCDR1
QSVLYSSNNKNY

SEQ ID NO: 64 - skipped

SEQ ID NO: 65 - protein, 118 aa, synthetic construct
  REGION 1..118: note = Description of Artificial Sequence: Synthetic polypeptide
  REGION 1..118: note = TRGV9Ab_var17 Heavy Chain Variable Region
QVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSS

SEQ ID NO: 66 - protein, 112 aa, synthetic construct
  REGION 1..112: note = Description of Artificial Sequence: Synthetic polypeptide
  REGION 1..112: note = TRGV9Ab_var17 Light Chain Variable Region
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIK

SEQ ID NO: 67 - protein, 118 aa, synthetic construct
  REGION 1..118: note = Description of Artificial Sequence: Synthetic polypeptide
  REGION 1..118: note = TRGV9Ab_var29 Heavy Chain Variable Region
QVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSS

SEQ ID NO: 68 - protein, 112 aa, synthetic construct
  REGION 1..112: note = Description of Artificial Sequence: Synthetic polypeptide
  REGION 1..112: note = TRGV9Ab_var29 Light Chain Variable Region
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIK

SEQ ID NO: 69 - protein, 448 aa, synthetic construct
  REGION 1..448: note = Description of Artificial Sequence: Synthetic polypeptide
  REGION 1..448: note = TRGV9_7A5_1 Heavy Chain
EVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 70 - protein, 501 aa, synthetic construct
  REGION 1..501: note = Description of Artificial Sequence: Synthetic polypeptide
  REGION 1..501: note = TRGV9_var17 scFv
MAWVWTLLFLMAAAQSIQADIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIKGGSEGKSSGSGSESKSTGGSQVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 71 - protein, 448 aa, synthetic construct
  REGION 1..448: note = Description of Artificial Sequence: Synthetic polypeptide
  REGION 1..448: note = TRGV9_var17 Heavy Chain
QVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 72 - protein, 219 aa, synthetic construct
  REGION 1..219: note = Description of Artificial Sequence: Synthetic polypeptide
  REGION 1..219: note = TRGV9_var17 Light Chain
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 501 aa, synthetic construct
  REGION 1..501: note = Description of Artificial Sequence: Synthetic polypeptide
  REGION 1..501: note = TRGV9_var29 scFv
MAWVWTLLFLMAAAQSIQADIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIKGGSEGKSSGSGSESKSTGGSQVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 74 - protein, 448 aa, synthetic construct
  REGION 1..448: note = Description of Artificial Sequence: Synthetic polypeptide
  REGION 1..448: note = TRGV9_var29 Heavy Chain
QVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 75 - protein, 219 aa, synthetic construct
  REGION 1..219: note = Description of Artificial Sequence: Synthetic polypeptide
  REGION 1..219: note = TRGV9_var29 Light Chain
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 17 aa, synthetic construct
  REGION 1..17: note = Description of Artificial Sequence: Synthetic peptide
  REGION 1..17: note = TRGV9Ab HCDR2
QIYPGSGNTYYNQKFKG

SEQ ID NO: 77 - protein, 17 aa, synthetic construct
  REGION 1..17: note = Description of Artificial Sequence: Synthetic peptide
  REGION 1..17: note = TRGV9Ab LCDR1
KSSQSVLYSSNNKNYLA

SEQ ID NO: 78 - protein, 467 aa, synthetic construct
  REGION 1..467: note = Description of Artificial Sequence: Synthetic polypeptide
  REGION 1..467: note = Anti-TRGV9_7A5_1 (half-mAb) Heavy Chain A
MAWVWTLLFLMAAAQSIQAEVQLQQSGAELARPGASVKLSCKASGFTFTDHYINWVKQRTGQGLEWIGQIYPGDGNTYYNQKFKGKATLTADKSSSTAYMQLSSLTSEDSAVYFCAPNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 79 - protein, 238 aa, synthetic construct
  REGION 1..238: note = Description of Artificial Sequence: Synthetic polypeptide
  REGION 1..238: note = Anti-TRGV9_7A5_1 (half-mAb) Light Chain
MAWVWTLLFLMAAAQSIQADIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 467 aa, synthetic construct
  REGION 1..467: note = Description of Artificial Sequence: Synthetic polypeptide
  REGION 1..467: note = Anti- TRGV9_7A5_var17 Heavy Chain A
MAWVWTLLFLMAAAQSIQAQVQLVQSGAEVKKPGASVKVSCKASGFTFTDHYINWVRQATGQGLEWMGQIYPGSGNTYYNQKFKGRVTMTRDTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 81 - protein, 238 aa, synthetic construct
  REGION 1..238: note = Description of Artificial Sequence: Synthetic polypeptide
  REGION 1..238: note = Anti- TRGV9_7A5_var17 Light Chain
MAWVWTLLFLMAAAQSIQADIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 467 aa, synthetic construct
  REGION 1..467: note = Description of Artificial Sequence: Synthetic polypeptide
  REGION 1..467: note = Anti- TRGV9_7A5_var29 Heavy Chain A
MAWVWTLLFLMAAAQSIQAQVQLVQSGAEVKKPGASVKVSCKASGFTFTDHYINWVRQATGQGLEWMGQIYPGSGNTYYNQKFKGRVTMTRNTSISTAYMELSSLRSEDTAVYYCARNYGDYTIDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 83 - protein, 238 aa, synthetic construct
  REGION 1..238: note = Description of Artificial Sequence: Synthetic polypeptide
  REGION 1..238: note = Anti- TRGV9_7A5_var29 Light Chain
MAWVWTLLFLMAAAQSIQADIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 473 aa, synthetic construct
  REGION 1..473: note = Description of Artificial Sequence: Synthetic polypeptide
  REGION 1..473: note = Anti-TRBC1 (half-mAb) Heavy Chain B
MAWVWTLLFLMAAAQSIQAEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNRFTQKSLSLSPGK

SEQ ID NO: 85 - protein, 238 aa, synthetic construct
  REGION 1..238: note = Description of Artificial Sequence: Synthetic polypeptide
  REGION 1..238: note = Anti-TRBC1 (half-mAb) Light Chain
MAWVWTLLFLMAAAQSIQADVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 469 aa, synthetic construct
  REGION 1..469: note = Description of Artificial Sequence: Synthetic polypeptide
  REGION 1..469: note = Anti-RSV (half-mAb) Heavy Chain B
MAWVWTLLFLMAAAQSIQAQITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 87 - protein, 238 aa, synthetic construct
  REGION 1..238: note = Description of Artificial Sequence: Synthetic polypeptide
  REGION 1..238: note = Anti-RSV (half-mAb) Light Chain
METHSQVFVYMLLWLSGVEGDIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 495 aa, synthetic construct
  REGION 1..495: note = Description of Artificial Sequence: Synthetic polypeptide
  REGION 1..495: note = Anti-Null-scFv Heavy Chain B
MAWVWTLLFLMAAAQSIQADIQMTQSPSSLSASVGDRVTITCRASQSISSYLNWYQQKPGCAPKLLIYAASSLQSGVPSRFSGSGSGTDFTLTISSLQPEDFATYYCQQSYSTPLTFGQGTKVEIKGGGSGGSGGCPPCGGSGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 89 - protein, 8 aa, synthetic construct
  REGION 1..8: note = Description of Artificial Sequence: Synthetic peptide
GFTFSSNY

SEQ ID NO: 90 - protein, 8 aa, synthetic construct
  REGION 1..8: note = Description of Artificial Sequence: Synthetic peptide
IHGGTGGI

SEQ ID NO: 91 - protein, 11 aa, synthetic construct
  REGION 1..11: note = Description of Artificial Sequence: Synthetic peptide
ARRGYGAWFAY

SEQ ID NO: 92 - protein, 6 aa, synthetic construct
  REGION 1..6: note = Description of Artificial Sequence: Synthetic peptide
ENIHNY

SEQ ID NO: 93 - skipped

SEQ ID NO: 94 - protein, 9 aa, synthetic construct
  REGION 1..9: note = Description of Artificial Sequence: Synthetic peptide
QHFWSYPLT

SEQ ID NO: 95 - protein, 118 aa, synthetic construct
  REGION 1..118: note = Description of Artificial Sequence: Synthetic polypeptide
QGQMQQSGAELVKPGASVKLSCKTSGFTFSSNYISWLKQKPGQSLEWIAWIHGGTGGIGYNQKFTGKAQLTVDTSSTTAYMQFSSLTTEDSAIYYCARRGYGAWFAYWGQGTLVTVSA

SEQ ID NO: 96 - protein, 107 aa, synthetic construct
  REGION 1..107: note = Description of Artificial Sequence: Synthetic polypeptide
DIQMTQSPASLSASVGETVTITCRASENIHNYLAWYQQKQGKSPQLLVYNAKTLADGVPSRFSGSGSGTQYSLKINSLQPEDFGNYYCQHFWSYPLTFGAGTKLELK

SEQ ID NO: 97 - protein, 496 aa, synthetic construct
  REGION 1..496: note = Description of Artificial Sequence: Synthetic polypeptide
MAWVWTLLFLMAAAQSIQADIQMTQSPASLSASVGETVTITCRASENIHNYLAWYQQKQGKSPQLLVYNAKTLADGVPSRFSGSGSGTQYSLKINSLQPEDFGNYYCQHFWSYPLTFGAGTKLELKGGSEGKSSGSGSESKSTGGSQGQMQQSGAELVKPGASVKLSCKTSGFTFSSNYISWLKQKPGQSLEWIAWIHGGTGGIGYNQKFTGKAQLTVDTSSTTAYMQFSSLTTEDSAIYYCARRGYGAWFAYWGQGTLVTVSAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 98 - protein, 8 aa, synthetic construct
  REGION 1..8: note = Description of Artificial Sequence: Synthetic peptide
GFTFSNYD

SEQ ID NO: 99 - protein, 8 aa, synthetic construct
  REGION 1..8: note = Description of Artificial Sequence: Synthetic peptide
ISSSSSYI

SEQ ID NO: 100 - protein, 16 aa, synthetic construct
  REGION 1..16: note = Description of Artificial Sequence: Synthetic peptide
ARDVGVTDYYYYGMDV

SEQ ID NO: 101 - protein, 7 aa, synthetic construct
  REGION 1..7: note = Description of Artificial Sequence: Synthetic peptide
QSVASSY

SEQ ID NO: 102 - skipped

SEQ ID NO: 103 - protein, 10 aa, synthetic construct
  REGION 1..10: note = Description of Artificial Sequence: Synthetic peptide
QQYGSSPPYT

SEQ ID NO: 104 - protein, 124 aa, synthetic construct
  REGION 1..124: note = Description of Artificial Sequence: Synthetic polypeptide
EVQLVESGGGLVKPGGSLRLSCSASGFTFSNYDMNWVRQAPGKGLEWVSSISSSSSYIYYADSVKGRFTISRDNAKNSLYLQMNSLRAEDTAVYHCARDVGVTTDYYYYGMDVWGQGTMVTVSS

SEQ ID NO: 105 - protein, 109 aa, synthetic construct
  REGION 1..109: note = Description of Artificial Sequence: Synthetic polypeptide
EIVMTQSPGTLSLSPGDRATLSCRASQSVASSYLAWYQQKPGQSPRLLIYGASSRATGIPDRFSGSGSGTDFTLTISRLEPEDFAVYYCQQYGSSPPYTFGQGTRLEIK

SEQ ID NO: 106 - protein, 504 aa, synthetic construct
  REGION 1..504: note = Description of Artificial Sequence: Synthetic polypeptide
MAWVWTLLFLMAAAQSIQAEIVMTQSPGTLSLSPGDRATLSCRASQSVASSYLAWYQQKPGQSPRLLIYGASSRATGIPDRFSGSGSGTDFTLTISRLEPEDFAVYYCQQYGSSPPYTFGQGTRLEIKGGSEGKSSGSGSESKSTGGSEVQLVESGGGLVKPGGSLRLSCSASGFTFSNYDMNWVRQAPGKGLEWVSSISSSSSYIYYADSVKGRFTISRDNAKNSLYLQMNSLRAEDTAVYHCARDVGVTTDYYYYGMDVWGQGTMVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 107 - protein, 8 aa, synthetic construct
  REGION 1..8: note = Description of Artificial Sequence: Synthetic peptide
GDTFNNYA

SEQ ID NO: 108 - protein, 8 aa, synthetic construct
  REGION 1..8: note = Description of Artificial Sequence: Synthetic peptide
IIPFFGTP

SEQ ID NO: 109 - protein, 17 aa, synthetic construct
  REGION 1..17: note = Description of Artificial Sequence: Synthetic peptide
ARPGSGSPDYYYYDMDV

SEQ ID NO: 110 - protein, 11 aa, synthetic construct
  REGION 1..11: note = Description of Artificial Sequence: Synthetic peptide
QSLVHSDGNTY

SEQ ID NO: 111 - skipped

SEQ ID NO: 112 - protein, 9 aa, synthetic construct
  REGION 1..9: note = Description of Artificial Sequence: Synthetic peptide
MQATQFPLT

SEQ ID NO: 113 - protein, 124 aa, synthetic construct
  REGION 1..124: note = Description of Artificial Sequence: Synthetic polypeptide
EVQLVQSGAEVKKPGSSVKVSCKASGDTFNNYAISWVRQAPGQGLEWMGGIIPFFGTPDYAQKFQGRVTITADKSTSTAYMELSGLRSEDTAVYYCARPGSGSPDYYYYDMDVWGQGTTVTVSS

SEQ ID NO: 114 - protein, 112 aa, synthetic construct
  REGION 1..112: note = Description of Artificial Sequence: Synthetic polypeptide
DIVMTQTPLSSPVTLGQPASISCRSSQSLVHSDGNTYLSWLQQRPGQPPRLLIYKISNRFSGVPDRFSGSGAGTDFTLKINRVEAEDVGVYYCMQATQFPLTFGGGTKVEIK

SEQ ID NO: 115 - protein, 454 aa, synthetic construct
  REGION 1..454: note = Description of Artificial Sequence: Synthetic polypeptide
EVQLVQSGAEVKKPGSSVKVSCKASGDTFNNYAISWVRQAPGQGLEWMGGIIPFFGTPDYAQKFQGRVTITADKSTSTAYMELSGLRSEDTAVYYCARPGSGSPDYYYYDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 116 - protein, 219 aa, synthetic construct
  REGION 1..219: note = Description of Artificial Sequence: Synthetic polypeptide
DIVMTQTPLSSPVTLGQPASISCRSSQSLVHSDGNTYLSWLQQRPGQPPRLLIYKISNRFSGVPDRFSGSGAGTDFTLKINRVEAEDVGVYYCMQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 8 aa, synthetic construct
  REGION 1..8: note = Description of Artificial Sequence: Synthetic peptide
GGTFSSYA

SEQ ID NO: 118 - protein, 8 aa, synthetic construct
  REGION 1..8: note = Description of Artificial Sequence: Synthetic peptide
NIPIFNTA

SEQ ID NO: 119 - protein, 13 aa, synthetic construct
  REGION 1..13: note = Description of Artificial Sequence: Synthetic peptide
VREGTGYSYGLDY

SEQ ID NO: 120 - protein, 11 aa, synthetic construct
  REGION 1..11: note = Description of Artificial Sequence: Synthetic peptide
QSLIHSDGNTY

SEQ ID NO: 121 - skipped

SEQ ID NO: 122 - protein, 9 aa, synthetic construct
  REGION 1..9: note = Description of Artificial Sequence: Synthetic peptide
MQAKQFPIT

SEQ ID NO: 123 - protein, 120 aa, synthetic construct
  REGION 1..120: note = Description of Artificial Sequence: Synthetic polypeptide
EVQLVQSGAEVKKPGSSVKVSCKASGGTFSSYAISWVRQAPGQGLEWMGGNIPIFNTANYAQKFQDRVTITADKSTSTAYMELSSLRSEDTAVYYCVREGTGYSYGLDYWGQGTPVTVSS

SEQ ID NO: 124 - protein, 112 aa, synthetic construct
  REGION 1..112: note = Description of Artificial Sequence: Synthetic polypeptide
EIVMTQSPLSSPVTLGQPASISCRSSQSLIHSDGNTYLSWLQQRPGQPPRLLIYKISNRFSGVPDRFSGSGAGTDFTLKISRVEAEDVGIYYCMQAKQFPITFGQGTKVDIK

SEQ ID NO: 125 - protein, 441 aa, synthetic construct
  REGION 1..441: note = Description of Artificial Sequence: Synthetic polypeptide
EVQLVQSGAEVKKPGSSVKVSCKASGGTFSSYAISWVRQAPGQGLEWMGGNIPIFNTANYAQKFQDRVTITADKSTSTAYMELSSLRSEDTAVYYCVREGTGYSYGLDYWGQGTPVTVSSASTKGPSVTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 126 - protein, 219 aa, synthetic construct
  REGION 1..219: note = Description of Artificial Sequence: Synthetic polypeptide
EIVMTQSPLSSPVTLGQPASISCRSSQSLIHSDGNTYLSWLQQRPGQPPRLLIYKISNRFSGVPDRFSGSGAGTDFTLKISRVEAEDVGIYYCMQAKQFPITFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 10 aa, synthetic construct
  REGION 1..10: note = Description of Artificial Sequence: Synthetic peptide
GGSISSGGSY

SEQ ID NO: 128 - protein, 7 aa, synthetic construct
  REGION 1..7: note = Description of Artificial Sequence: Synthetic peptide
IYNSGST

SEQ ID NO: 129 - protein, 12 aa, synthetic construct
  REGION 1..12: note = Description of Artificial Sequence: Synthetic peptide
ARDSNYEWFFDL

SEQ ID NO: 130 - protein, 6 aa, synthetic construct
  REGION 1..6: note = Description of Artificial Sequence: Synthetic peptide
QSVSSY

SEQ ID NO: 131 - skipped

SEQ ID NO: 132 - protein, 9 aa, synthetic construct
  REGION 1..9: note = Description of Artificial Sequence: Synthetic peptide
QQRSNWPLT

SEQ ID NO: 133 - protein, 120 aa, synthetic construct
  REGION 1..120: note = Description of Artificial Sequence: Synthetic polypeptide
QVQLQESGPGLVKPSQTLSLTCTVSGGSISSGGSYWSWIRQHPGKGLEWIGYIYNSGSTYYNPSLKSRVSMSVDTSKNQFSLKLSSVTAADTAVYYCARDSNYEWFFDLWGPGTLVTVSS

SEQ ID NO: 134 - protein, 107 aa, synthetic construct
  REGION 1..107: note = Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 135 - protein, 450 aa, synthetic construct
  REGION 1..450: note = Description of Artificial Sequence: Synthetic polypeptide
QVQLQESGPGLVKPSQTLSLTCTVSGGSISSGGSYWSWIRQHPGKGLEWIGYIYNSGSTYYNPSLKSRVSMSVDTSKNQFSLKLSSVTAADTAVYYCARDSNYEWFFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 136 - protein, 214 aa, synthetic construct
  REGION 1..214: note = Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137-149 - skipped (13 entries)

SEQ ID NO: 150 - protein, 473 aa, synthetic construct
  REGION 1..473: note = Description of Artificial Sequence: Synthetic polypeptide
MAWVWTLLFLMAAAQSIQAEVQLVQSGAEVKKPGSSVKVSCKASGDTFNNYAISWVRQAPGQGLEWMGGIIPFFGTPDYAQKFQGRVTITADKSTSTAYMELSGLRSEDTAVYYCARPGSGSPDYYYYDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 151 - protein, 238 aa, synthetic construct
  REGION 1..238: note = Description of Artificial Sequence: Synthetic polypeptide
MAWVWTLLFLMAAAQSIQADIVMTQTPLSSPVTLGQPASISCRSSQSLVHSDGNTYLSWLQQRPGQPPRLLIYKISNRFSGVPDRFSGSGAGTDFTLKINRVEAEDVGVYYCMQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 469 aa, synthetic construct
  REGION 1..469: note = Description of Artificial Sequence: Synthetic polypeptide
MAWVWTLLFLMAAAQSIQAEVQLVQSGAEVKKPGSSVKVSCKASGGTFSSYAISWVRQAPGQGLEWMGGNIPIFNTANYAQKFQDRVTITADKSTSTAYMELSSLRSEDTAVYYCVREGTGYSYGLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 153 - protein, 238 aa, synthetic construct
  REGION 1..238: note = Description of Artificial Sequence: Synthetic polypeptide
MAWVWTLLFLMAAAQSIQAEIVMTQSPLSSPVTLGQPASISCRSSQSLIHSDGNTYLSWLQQRPGQPPRLLIYKISNRFSGVPDRFSGSGAGTDFTLKISRVEAEDVGIYYCMQAKQFPITFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 469 aa, synthetic construct
  REGION 1..469: note = Description of Artificial Sequence: Synthetic polypeptide
MAWVWTLLFLMAAAQSIQAQVQLQESGPGLVKPSQTLSLTCTVSGGSISSGGSYWSWIRQHPGKGLEWIGYIYNSGSTYYNPSLKSRVSMSVDTSKNQFSLKLSSVTAADTAVYYCARDSNYEWFFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPGK

SEQ ID NO: 155 - protein, 233 aa, synthetic construct
  REGION 1..233: note = Description of Artificial Sequence: Synthetic polypeptide
MAWVWTLLFLMAAAQSIQAEIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 493 aa, synthetic construct
  REGION 1..493: note = Description of Artificial Sequence: Synthetic polypeptide
MAWVWTLLFLMAAAQSIQAAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 157 - protein, 480 aa, synthetic construct
  REGION 1..480: note = Description of Artificial Sequence: Synthetic polypeptide
MAWVWTLLFLMAAAQSIQAAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 158-159 - skipped (2 entries)

SEQ ID NO: 160 - protein, 10 aa, synthetic construct
  REGION 1..10: note = Description of Artificial Sequence: Synthetic peptide
GFTFTDHYIN

SEQ ID NO: 161 - protein, 17 aa, synthetic construct
  REGION 1..17: note = Description of Artificial Sequence: Synthetic peptide
QIYPGDGNTYYNQKFKG

SEQ ID NO: 162 - protein, 9 aa, synthetic construct
  REGION 1..9: note = Description of Artificial Sequence: Synthetic peptide
NYGDYTIDF

SEQ ID NO: 163 - protein, 17 aa, synthetic construct
  REGION 1..17: note = Description of Artificial Sequence: Synthetic peptide
KSSQSLLYSSNQKNYLA

SEQ ID NO: 164 - protein, 7 aa, synthetic construct
  REGION 1..7: note = Description of Artificial Sequence: Synthetic peptide
WASTRES

SEQ ID NO: 165 - protein, 8 aa, synthetic construct
  REGION 1..8: note = Description of Artificial Sequence: Synthetic peptide
QQYYRYHT

SEQ ID NO: 166 - protein, 8 aa, synthetic construct
  REGION 1..8: note = Description of Artificial Sequence: Synthetic peptide
GFTFTDHY

SEQ ID NO: 167 - protein, 8 aa, synthetic construct
  REGION 1..8: note = Description of Artificial Sequence: Synthetic peptide
IYPGDGNT

SEQ ID NO: 168 - protein, 11 aa, synthetic construct
  REGION 1..11: note = Description of Artificial Sequence: Synthetic peptide
APNYGDYTIDF

SEQ ID NO: 169 - protein, 12 aa, synthetic construct
  REGION 1..12: note = Description of Artificial Sequence: Synthetic peptide
QSLLYSSNQKNY

SEQ ID NO: 170 - skipped

SEQ ID NO: 171 - protein, 8 aa, synthetic construct
  REGION 1..8: note = Description of Artificial Sequence: Synthetic peptide
QQYYRYHT

SEQ ID NO: 172 - protein, 5 aa, synthetic construct
  REGION 1..5: note = Description of Artificial Sequence: Synthetic peptide
DHYIN

SEQ ID NO: 173 - protein, 17 aa, synthetic construct
  REGION 1..17: note = Description of Artificial Sequence: Synthetic peptide
QIYPGDGNTYYNQKFKG

SEQ ID NO: 174 - protein, 9 aa, synthetic construct
  REGION 1..9: note = Description of Artificial Sequence: Synthetic peptide
NYGDYTIDF

SEQ ID NO: 175 - protein, 17 aa, synthetic construct
  REGION 1..17: note = Description of Artificial Sequence: Synthetic peptide
KSSQSLLYSSNQKNYLA

SEQ ID NO: 176 - protein, 7 aa, synthetic construct
  REGION 1..7: note = Description of Artificial Sequence: Synthetic peptide
WASTRES

SEQ ID NO: 177 - protein, 8 aa, synthetic construct
  REGION 1..8: note = Description of Artificial Sequence: Synthetic peptide
QQYYRYHT

SEQ ID NO: 178 - protein, 7 aa, synthetic construct
  REGION 1..7: note = Description of Artificial Sequence: Synthetic peptide
GFTFTDH

SEQ ID NO: 179 - protein, 4 aa, synthetic construct
  REGION 1..4: note = Description of Artificial Sequence: Synthetic peptide
PGDG

SEQ ID NO: 180 - protein, 7 aa, synthetic construct
  REGION 1..7: note = Description of Artificial Sequence: Synthetic peptide
YGDYTID

SEQ ID NO: 181 - protein, 13 aa, synthetic construct
  REGION 1..13: note = Description of Artificial Sequence: Synthetic peptide
SQSLLYSSNQKNY

SEQ ID NO: 182 - skipped

SEQ ID NO: 183 - protein, 5 aa, synthetic construct
  REGION 1..5: note = Description of Artificial Sequence: Synthetic peptide
YYRYH

SEQ ID NO: 184 - protein, 6 aa, synthetic construct
  REGION 1..6: note = Description of Artificial Sequence: Synthetic peptide
TDHYIN

SEQ ID NO: 185 - protein, 13 aa, synthetic construct
  REGION 1..13: note = Description of Artificial Sequence: Synthetic peptide
WIGQIYPGDGNTY

SEQ ID NO: 186 - protein, 10 aa, synthetic construct
  REGION 1..10: note = Description of Artificial Sequence: Synthetic peptide
APNYGDYTID

SEQ ID NO: 187 - protein, 13 aa, synthetic construct
  REGION 1..13: note = Description of Artificial Sequence: Synthetic peptide
LYSSNQKNYLAWY

SEQ ID NO: 188 - protein, 10 aa, synthetic construct
  REGION 1..10: note = Description of Artificial Sequence: Synthetic peptide
LLIYWASTRE

SEQ ID NO: 189 - protein, 7 aa, synthetic construct
  REGION 1..7: note = Description of Artificial Sequence: Synthetic peptide
QQYYRYH

SEQ ID NO: 190 - protein, 10 aa, synthetic construct
  REGION 1..10: note = Description of Artificial Sequence: Synthetic peptide
GFTFTDHYIN

SEQ ID NO: 191 - protein, 10 aa, synthetic construct
  REGION 1..10: note = Description of Artificial Sequence: Synthetic peptide
QIYPGDGNTY

SEQ ID NO: 192 - protein, 9 aa, synthetic construct
  REGION 1..9: note = Description of Artificial Sequence: Synthetic peptide
NYGDYTIDF

SEQ ID NO: 193 - protein, 17 aa, synthetic construct
  REGION 1..17: note = Description of Artificial Sequence: Synthetic peptide
KSSQSLLYSSNQKNYLA

SEQ ID NO: 194 - protein, 7 aa, synthetic construct
  REGION 1..7: note = Description of Artificial Sequence: Synthetic peptide
WASTRES

SEQ ID NO: 195 - protein, 8 aa, synthetic construct
  REGION 1..8: note = Description of Artificial Sequence: Synthetic peptide
QQYYRYHT

SEQ ID NO: 196 - protein, 10 aa, synthetic construct
  REGION 1..10: note = Description of Artificial Sequence: Synthetic peptide
GFTFTDHYIN

SEQ ID NO: 197 - protein, 17 aa, synthetic construct
  REGION 1..17: note = Description of Artificial Sequence: Synthetic peptide
QIYPGDGNTYYNQKFKG

SEQ ID NO: 198 - protein, 9 aa, synthetic construct
  REGION 1..9: note = Description of Artificial Sequence: Synthetic peptide
NMGMYTIDF

SEQ ID NO: 199 - protein, 17 aa, synthetic construct
  REGION 1..17: note = Description of Artificial Sequence: Synthetic peptide
KSSQSLLYSSNQKNYLA

SEQ ID NO: 200 - protein, 7 aa, synthetic construct
  REGION 1..7: note = Description of Artificial Sequence: Synthetic peptide
WASTRES

SEQ ID NO: 201 - protein, 8 aa, synthetic construct
  REGION 1..8: note = Description of Artificial Sequence: Synthetic peptide
QQYYRYHT

SEQ ID NO: 202 - protein, 8 aa, synthetic construct
  REGION 1..8: note = Description of Artificial Sequence: Synthetic peptide
GFTFTDHY

SEQ ID NO: 203 - protein, 8 aa, synthetic construct
  REGION 1..8: note = Description of Artificial Sequence: Synthetic peptide
IYPGDGNT

SEQ ID NO: 204 - protein, 11 aa, synthetic construct
  REGION 1..11: note = Description of Artificial Sequence: Synthetic peptide
APNMGMYTIDF

SEQ ID NO: 205 - protein, 12 aa, synthetic construct
  REGION 1..12: note = Description of Artificial Sequence: Synthetic peptide
QSLLYSSNQKNY

SEQ ID NO: 206 - skipped

SEQ ID NO: 207 - protein, 8 aa, synthetic construct
  REGION 1..8: note = Description of Artificial Sequence: Synthetic peptide
QQYYRYHT

SEQ ID NO: 208 - protein, 5 aa, synthetic construct
  REGION 1..5: note = Description of Artificial Sequence: Synthetic peptide
DHYIN

SEQ ID NO: 209 - protein, 17 aa, synthetic construct
  REGION 1..17: note = Description of Artificial Sequence: Synthetic peptide
QIYPGDGNTYYNQKFKG

SEQ ID NO: 210 - protein, 9 aa, synthetic construct
  REGION 1..9: note = Description of Artificial Sequence: Synthetic peptide
NMGMYTIDF

SEQ ID NO: 211 - protein, 17 aa, synthetic construct
  REGION 1..17: note = Description of Artificial Sequence: Synthetic peptide
KSSQSLLYSSNQKNYLA

SEQ ID NO: 212 - protein, 7 aa, synthetic construct
  REGION 1..7: note = Description of Artificial Sequence: Synthetic peptide
WASTRES

SEQ ID NO: 213 - protein, 8 aa, synthetic construct
  REGION 1..8: note = Description of Artificial Sequence: Synthetic peptide
QQYYRYHT

SEQ ID NO: 214 - protein, 7 aa, synthetic construct
  REGION 1..7: note = Description of Artificial Sequence: Synthetic peptide
GFTFTDH

SEQ ID NO: 215 - protein, 4 aa, synthetic construct
  REGION 1..4: note = Description of Artificial Sequence: Synthetic peptide
PGDG

SEQ ID NO: 216 - protein, 7 aa, synthetic construct
  REGION 1..7: note = Description of Artificial Sequence: Synthetic peptide
MGMYTID

SEQ ID NO: 217 - protein, 13 aa, synthetic construct
  REGION 1..13: note = Description of Artificial Sequence: Synthetic peptide
SQSLLYSSNQKNY

SEQ ID NO: 218 - skipped

SEQ ID NO: 219 - protein, 5 aa, synthetic construct
  REGION 1..5: note = Description of Artificial Sequence: Synthetic peptide
YYRYH

SEQ ID NO: 220 - protein, 6 aa, synthetic construct
  REGION 1..6: note = Description of Artificial Sequence: Synthetic peptide
TDHYIN

SEQ ID NO: 221 - protein, 13 aa, synthetic construct
  REGION 1..13: note = Description of Artificial Sequence: Synthetic peptide
WIGQIYPGDGNTY

SEQ ID NO: 222 - protein, 10 aa, synthetic construct
  REGION 1..10: note = Description of Artificial Sequence: Synthetic peptide
APNMGMYTID

SEQ ID NO: 223 - protein, 13 aa, synthetic construct
  REGION 1..13: note = Description of Artificial Sequence: Synthetic peptide
LYSSNQKNYLAWY

SEQ ID NO: 224 - protein, 10 aa, synthetic construct
  REGION 1..10: note = Description of Artificial Sequence: Synthetic peptide
LLIYWASTRE

SEQ ID NO: 225 - protein, 7 aa, synthetic construct
  REGION 1..7: note = Description of Artificial Sequence: Synthetic peptide
QQYYRYH

SEQ ID NO: 226 - protein, 10 aa, synthetic construct
  REGION 1..10: note = Description of Artificial Sequence: Synthetic peptide
GFTFTDHYIN

SEQ ID NO: 227 - protein, 10 aa, synthetic construct
  REGION 1..10: note = Description of Artificial Sequence: Synthetic peptide
QIYPGDGNTY

SEQ ID NO: 228 - protein, 9 aa, synthetic construct
  REGION 1..9: note = Description of Artificial Sequence: Synthetic peptide
NMGMYTIDF

SEQ ID NO: 229 - protein, 17 aa, synthetic construct
  REGION 1..17: note = Description of Artificial Sequence: Synthetic peptide
KSSQSLLYSSNQKNYLA

SEQ ID NO: 230 - protein, 7 aa, synthetic construct
  REGION 1..7: note = Description of Artificial Sequence: Synthetic peptide
WASTRES

SEQ ID NO: 231 - protein, 8 aa, synthetic construct
  REGION 1..8: note = Description of Artificial Sequence: Synthetic peptide
QQYYRYHT

SEQ ID NO: 232 - protein, 10 aa, synthetic construct
  REGION 1..10: note = Description of Artificial Sequence: Synthetic peptide
GFTFTDHYIN

SEQ ID NO: 233 - protein, 17 aa, synthetic construct
  REGION 1..17: note = Description of Artificial Sequence: Synthetic peptide
QIYPGDGNTYYNQKFKG

SEQ ID NO: 234 - protein, 9 aa, synthetic construct
  REGION 1..9: note = Description of Artificial Sequence: Synthetic peptide
NMGMYTLDF

SEQ ID NO: 235 - protein, 17 aa, synthetic construct
  REGION 1..17: note = Description of Artificial Sequence: Synthetic peptide
KSSQSLLYSSNQKNYLA

SEQ ID NO: 236 - protein, 7 aa, synthetic construct
  REGION 1..7: note = Description of Artificial Sequence: Synthetic peptide
WASTRES

SEQ ID NO: 237 - protein, 8 aa, synthetic construct
  REGION 1..8: note = Description of Artificial Sequence: Synthetic peptide
QQYYRYHT

SEQ ID NO: 238 - protein, 8 aa, synthetic construct
  REGION 1..8: note = Description of Artificial Sequence: Synthetic peptide
GFTFTDHY

SEQ ID NO: 239 - protein, 8 aa, synthetic construct
  REGION 1..8: note = Description of Artificial Sequence: Synthetic peptide
IYPGDGNT

SEQ ID NO: 240 - protein, 11 aa, synthetic construct
  REGION 1..11: note = Description of Artificial Sequence: Synthetic peptide
APNMGMYTLDF

SEQ ID NO: 241 - protein, 12 aa, synthetic construct
  REGION 1..12: note = Description of Artificial Sequence: Synthetic peptide
QSLLYSSNQKNY

SEQ ID NO: 242 - skipped

SEQ ID NO: 243 - protein, 8 aa, synthetic construct
  REGION 1..8: note = Description of Artificial Sequence: Synthetic peptide
QQYYRYHT

SEQ ID NO: 244 - protein, 5 aa, synthetic construct
  REGION 1..5: note = Description of Artificial Sequence: Synthetic peptide
DHYIN

SEQ ID NO: 245 - protein, 17 aa, synthetic construct
  REGION 1..17: note = Description of Artificial Sequence: Synthetic peptide
QIYPGDGNTYYNQKFKG

SEQ ID NO: 246 - protein, 9 aa, synthetic construct
  REGION 1..9: note = Description of Artificial Sequence: Synthetic peptide
NMGMYTLDF

SEQ ID NO: 247 - protein, 17 aa, synthetic construct
  REGION 1..17: note = Description of Artificial Sequence: Synthetic peptide
KSSQSLLYSSNQKNYLA

SEQ ID NO: 248 - protein, 7 aa, synthetic construct
  REGION 1..7: note = Description of Artificial Sequence: Synthetic peptide
WASTRES

SEQ ID NO: 249 - protein, 8 aa, synthetic construct
  REGION 1..8: note = Description of Artificial Sequence: Synthetic peptide
QQYYRYHT

SEQ ID NO: 250 - protein, 7 aa, synthetic construct
  REGION 1..7: note = Description of Artificial Sequence: Synthetic peptide
GFTFTDH

SEQ ID NO: 251 - protein, 4 aa, synthetic construct
  REGION 1..4: note = Description of Artificial Sequence: Synthetic peptide
PGDG

SEQ ID NO: 252 - protein, 7 aa, synthetic construct
  REGION 1..7: note = Description of Artificial Sequence: Synthetic peptide
MGMYTLD

SEQ ID NO: 253 - protein, 13 aa, synthetic construct
  REGION 1..13: note = Description of Artificial Sequence: Synthetic peptide
SQSLLYSSNQKNY

SEQ ID NO: 254 - skipped

SEQ ID NO: 255 - protein, 5 aa, synthetic construct
  REGION 1..5: note = Description of Artificial Sequence: Synthetic peptide
YYRYH

SEQ ID NO: 256 - protein, 6 aa, synthetic construct
  REGION 1..6: note = Description of Artificial Sequence: Synthetic peptide
TDHYIN

SEQ ID NO: 257 - protein, 13 aa, synthetic construct
  REGION 1..13: note = Description of Artificial Sequence: Synthetic peptide
WIGQIYPGDGNTY

SEQ ID NO: 258 - protein, 10 aa, synthetic construct
  REGION 1..10: note = Description of Artificial Sequence: Synthetic peptide
APNMGMYTLD

SEQ ID NO: 259 - protein, 13 aa, synthetic construct
  REGION 1..13: note = Description of Artificial Sequence: Synthetic peptide
LYSSNQKNYLAWY

SEQ ID NO: 260 - protein, 10 aa, synthetic construct
  REGION 1..10: note = Description of Artificial Sequence: Synthetic peptide
LLIYWASTRE

SEQ ID NO: 261 - protein, 7 aa, synthetic construct
  REGION 1..7: note = Description of Artificial Sequence: Synthetic peptide
QQYYRYH

SEQ ID NO: 262 - protein, 10 aa, synthetic construct
  REGION 1..10: note = Description of Artificial Sequence: Synthetic peptide
GFTFTDHYIN

SEQ ID NO: 263 - protein, 10 aa, synthetic construct
  REGION 1..10: note = Description of Artificial Sequence: Synthetic peptide
QIYPGDGNTY

SEQ ID NO: 264 - protein, 9 aa, synthetic construct
  REGION 1..9: note = Description of Artificial Sequence: Synthetic peptide
NMGMYTLDF

SEQ ID NO: 265 - protein, 17 aa, synthetic construct
  REGION 1..17: note = Description of Artificial Sequence: Synthetic peptide
KSSQSLLYSSNQKNYLA

SEQ ID NO: 266 - protein, 7 aa, synthetic construct
  REGION 1..7: note = Description of Artificial Sequence: Synthetic peptide
WASTRES

SEQ ID NO: 267 - protein, 8 aa, synthetic construct
  REGION 1..8: note = Description of Artificial Sequence: Synthetic peptide
QQYYRYHT

SEQ ID NO: 268 - protein, 10 aa, synthetic construct
  REGION 1..10: note = Description of Artificial Sequence: Synthetic peptide
GFTFTDHYIN

SEQ ID NO: 269 - protein, 17 aa, synthetic construct
  REGION 1..17: note = Description of Artificial Sequence: Synthetic peptide
QIYPGDGNTYYNQKFKG

SEQ ID NO: 270 - protein, 9 aa, synthetic construct
  REGION 1..9: note = Description of Artificial Sequence: Synthetic peptide
NYGDYTLDF

SEQ ID NO: 271 - protein, 17 aa, synthetic construct
  REGION 1..17: note = Description of Artificial Sequence: Synthetic peptide
KSSQSLLYSSNQKNYLA

SEQ ID NO: 272 - protein, 7 aa, synthetic construct
  REGION 1..7: note = Description of Artificial Sequence: Synthetic peptide
WASTRES

SEQ ID NO: 273 - protein, 8 aa, synthetic construct
  REGION 1..8: note = Description of Artificial Sequence: Synthetic peptide
QQYYRYHT

SEQ ID NO: 274 - protein, 8 aa, synthetic construct
  REGION 1..8: note = Description of Artificial Sequence: Synthetic peptide
GFTFTDHY

SEQ ID NO: 275 - protein, 8 aa, synthetic construct
  REGION 1..8: note = Description of Artificial Sequence: Synthetic peptide
IYPGDGNT

SEQ ID NO: 276 - protein, 11 aa, synthetic construct
  REGION 1..11: note = Description of Artificial Sequence: Synthetic peptide
APNYGDYTLDF

SEQ ID NO: 277 - protein, 12 aa, synthetic construct
  REGION 1..12: note = Description of Artificial Sequence: Synthetic peptide
QSLLYSSNQKNY

SEQ ID NO: 278 - skipped

SEQ ID NO: 279 - protein, 8 aa, synthetic construct
  REGION 1..8: note = Description of Artificial Sequence: Synthetic peptide
QQYYRYHT

SEQ ID NO: 280 - protein, 5 aa, synthetic construct
  REGION 1..5: note = Description of Artificial Sequence: Synthetic peptide
DHYIN

SEQ ID NO: 281 - protein, 17 aa, synthetic construct
  REGION 1..17: note = Description of Artificial Sequence: Synthetic peptide
QIYPGDGNTYYNQKFKG

SEQ ID NO: 282 - protein, 9 aa, synthetic construct
  REGION 1..9: note = Description of Artificial Sequence: Synthetic peptide
NYGDYTLDF

SEQ ID NO: 283 - protein, 17 aa, synthetic construct
  REGION 1..17: note = Description of Artificial Sequence: Synthetic peptide
KSSQSLLYSSNQKNYLA

SEQ ID NO: 284 - protein, 7 aa, synthetic construct
  REGION 1..7: note = Description of Artificial Sequence: Synthetic peptide
WASTRES

SEQ ID NO: 285 - protein, 8 aa, synthetic construct
  REGION 1..8: note = Description of Artificial Sequence: Synthetic peptide
QQYYRYHT

SEQ ID NO: 286 - protein, 7 aa, synthetic construct
  REGION 1..7: note = Description of Artificial Sequence: Synthetic peptide
GFTFTDH

SEQ ID NO: 287 - protein, 4 aa, synthetic construct
  REGION 1..4: note = Description of Artificial Sequence: Synthetic peptide
PGDG

SEQ ID NO: 288 - protein, 7 aa, synthetic construct
  REGION 1..7: note = Description of Artificial Sequence: Synthetic peptide
YGDYTLD

SEQ ID NO: 289 - protein, 13 aa, synthetic construct
  REGION 1..13: note = Description of Artificial Sequence: Synthetic peptide
SQSLLYSSNQKNY

SEQ ID NO: 290 - skipped

SEQ ID NO: 291 - protein, 5 aa, synthetic construct
  REGION 1..5: note = Description of Artificial Sequence: Synthetic peptide
YYRYH

SEQ ID NO: 292 - protein, 6 aa, synthetic construct
  REGION 1..6: note = Description of Artificial Sequence: Synthetic peptide
TDHYIN

SEQ ID NO: 293 - protein, 13 aa, synthetic construct
  REGION 1..13: note = Description of Artificial Sequence: Synthetic peptide
WIGQIYPGDGNTY

SEQ ID NO: 294 - protein, 10 aa, synthetic construct
  REGION 1..10: note = Description of Artificial Sequence: Synthetic peptide
APNYGDYTLD

SEQ ID NO: 295 - protein, 13 aa, synthetic construct
  REGION 1..13: note = Description of Artificial Sequence: Synthetic peptide
LYSSNQKNYLAWY

SEQ ID NO: 296 - protein, 10 aa, synthetic construct
  REGION 1..10: note = Description of Artificial Sequence: Synthetic peptide
LLIYWASTRE

SEQ ID NO: 297 - protein, 7 aa, synthetic construct
  REGION 1..7: note = Description of Artificial Sequence: Synthetic peptide
QQYYRYH

SEQ ID NO: 298 - protein, 10 aa, synthetic construct
  REGION 1..10: note = Description of Artificial Sequence: Synthetic peptide
GFTFTDHYIN

SEQ ID NO: 299 - protein, 10 aa, synthetic construct
  REGION 1..10: note = Description of Artificial Sequence: Synthetic peptide
QIYPGDGNTY

SEQ ID NO: 300 - protein, 9 aa, synthetic construct
  REGION 1..9: note = Description of Artificial Sequence: Synthetic peptide
NYGDYTLDF

SEQ ID NO: 301 - protein, 17 aa, synthetic construct
  REGION 1..17: note = Description of Artificial Sequence: Synthetic peptide
KSSQSLLYSSNQKNYLA

SEQ ID NO: 302 - protein, 7 aa, synthetic construct
  REGION 1..7: note = Description of Artificial Sequence: Synthetic peptide
WASTRES

SEQ ID NO: 303 - protein, 8 aa, synthetic construct
  REGION 1..8: note = Description of Artificial Sequence: Synthetic peptide
QQYYRYHT

SEQ ID NO: 304 - protein, 10 aa, synthetic construct
  REGION 1..10: note = Description of Artificial Sequence: Synthetic peptide
GFTFTDHYIN

SEQ ID NO: 305 - protein, 17 aa, synthetic construct
  REGION 1..17: note = Description of Artificial Sequence: Synthetic peptide
QIYPGSGNTYYNQKFKG

SEQ ID NO: 306 - protein, 9 aa, synthetic construct
  REGION 1..9: note = Description of Artificial Sequence: Synthetic peptide
NYGDYTIDF

SEQ ID NO: 307 - protein, 17 aa, synthetic construct
  REGION 1..17: note = Description of Artificial Sequence: Synthetic peptide
KSSQSVLYSSNNKNYLA

SEQ ID NO: 308 - protein, 7 aa, synthetic construct
  REGION 1..7: note = Description of Artificial Sequence: Synthetic peptide
WASTRES

SEQ ID NO: 309 - protein, 8 aa, synthetic construct
  REGION 1..8: note = Description of Artificial Sequence: Synthetic peptide
QQYYRYHT

SEQ ID NO: 310 - protein, 8 aa, synthetic construct
  REGION 1..8: note = Description of Artificial Sequence: Synthetic peptide
GFTFTDHY

SEQ ID NO: 311 - protein, 8 aa, synthetic construct
  REGION 1..8: note = Description of Artificial Sequence: Synthetic peptide
IYPGSGNT

SEQ ID NO: 312 - protein, 11 aa, synthetic construct
  REGION 1..11: note = Description of Artificial Sequence: Synthetic peptide
ARNYGDYTIDF

SEQ ID NO: 313 - protein, 12 aa, synthetic construct
  REGION 1..12: note = Description of Artificial Sequence: Synthetic peptide
QSVLYSSNNKNY

SEQ ID NO: 314 - skipped

SEQ ID NO: 315 - protein, 8 aa, synthetic construct
  REGION 1..8: note = Description of Artificial Sequence: Synthetic peptide
QQYYRYHT

SEQ ID NO: 316 - protein, 5 aa, synthetic construct
  REGION 1..5: note = Description of Artificial Sequence: Synthetic peptide
DHYIN

SEQ ID NO: 317 - protein, 17 aa, synthetic construct
  REGION 1..17: note = Description of Artificial Sequence: Synthetic peptide
QIYPGSGNTYYNQKFKG

SEQ ID NO: 318 - protein, 9 aa, synthetic construct
  REGION 1..9: note = Description of Artificial Sequence: Synthetic peptide
NYGDYTIDF

SEQ ID NO: 319 - protein, 17 aa, synthetic construct
  REGION 1..17: note = Description of Artificial Sequence: Synthetic peptide
KSSQSVLYSSNNKNYLA

SEQ ID NO: 320 - protein, 7 aa, synthetic construct
  REGION 1..7: note = Description of Artificial Sequence: Synthetic peptide
WASTRES

SEQ ID NO: 321 - protein, 8 aa, synthetic construct
  REGION 1..8: note = Description of Artificial Sequence: Synthetic peptide
QQYYRYHT

SEQ ID NO: 322 - protein, 7 aa, synthetic construct
  REGION 1..7: note = Description of Artificial Sequence: Synthetic peptide
GFTFTDH

SEQ ID NO: 323 - protein, 4 aa, synthetic construct
  REGION 1..4: note = Description of Artificial Sequence: Synthetic peptide
PGSG

SEQ ID NO: 324 - protein, 7 aa, synthetic construct
  REGION 1..7: note = Description of Artificial Sequence: Synthetic peptide
YGDYTID

SEQ ID NO: 325 - protein, 13 aa, synthetic construct
  REGION 1..13: note = Description of Artificial Sequence: Synthetic peptide
SQSVLYSSNNKNY

SEQ ID NO: 326 - skipped

SEQ ID NO: 327 - protein, 5 aa, synthetic construct
  REGION 1..5: note = Description of Artificial Sequence: Synthetic peptide
YYRYH

SEQ ID NO: 328 - protein, 6 aa, synthetic construct
  REGION 1..6: note = Description of Artificial Sequence: Synthetic peptide
TDHYIN

SEQ ID NO: 329 - protein, 13 aa, synthetic construct
  REGION 1..13: note = Description of Artificial Sequence: Synthetic peptide
WMGQIYPGSGNTY

SEQ ID NO: 330 - protein, 10 aa, synthetic construct
  REGION 1..10: note = Description of Artificial Sequence: Synthetic peptide
ARNYGDYTID

SEQ ID NO: 331 - protein, 13 aa, synthetic construct
  REGION 1..13: note = Description of Artificial Sequence: Synthetic peptide
LYSSNNKNYLAWY

SEQ ID NO: 332 - protein, 10 aa, synthetic construct
  REGION 1..10: note = Description of Artificial Sequence: Synthetic peptide
LLIYWASTRE

SEQ ID NO: 333 - protein, 7 aa, synthetic construct
  REGION 1..7: note = Description of Artificial Sequence: Synthetic peptide
QQYYRYH

SEQ ID NO: 334 - protein, 10 aa, synthetic construct
  REGION 1..10: note = Description of Artificial Sequence: Synthetic peptide
GFTFTDHYIN

SEQ ID NO: 335 - protein, 10 aa, synthetic construct
  REGION 1..10: note = Description of Artificial Sequence: Synthetic peptide
QIYPGSGNTY

SEQ ID NO: 336 - protein, 9 aa, synthetic construct
  REGION 1..9: note = Description of Artificial Sequence: Synthetic peptide
NYGDYTIDF

SEQ ID NO: 337 - protein, 17 aa, synthetic construct
  REGION 1..17: note = Description of Artificial Sequence: Synthetic peptide
KSSQSVLYSSNNKNYLA

SEQ ID NO: 338 - protein, 7 aa, synthetic construct
  REGION 1..7: note = Description of Artificial Sequence: Synthetic peptide
WASTRES

SEQ ID NO: 339 - protein, 8 aa, synthetic construct
  REGION 1..8: note = Description of Artificial Sequence: Synthetic peptide
QQYYRYHT

SEQ ID NO: 340 - protein, 10 aa, synthetic construct
  REGION 1..10: note = Description of Artificial Sequence: Synthetic peptide
GFTFTDHYIN

SEQ ID NO: 341 - protein, 17 aa, synthetic construct
  REGION 1..17: note = Description of Artificial Sequence: Synthetic peptide
QIYPGSGNTYYNQKFKG

SEQ ID NO: 342 - protein, 9 aa, synthetic construct
  REGION 1..9: note = Description of Artificial Sequence: Synthetic peptide
NYGDYTIDF

SEQ ID NO: 343 - protein, 17 aa, synthetic construct
  REGION 1..17: note = Description of Artificial Sequence: Synthetic peptide
KSSQSVLYSSNNKNYLA

SEQ ID NO: 344 - protein, 7 aa, synthetic construct
  REGION 1..7: note = Description of Artificial Sequence: Synthetic peptide
WASTRES

SEQ ID NO: 345 - protein, 8 aa, synthetic construct
  REGION 1..8: note = Description of Artificial Sequence: Synthetic peptide
QQYYRYHT

SEQ ID NO: 346 - protein, 8 aa, synthetic construct
  REGION 1..8: note = Description of Artificial Sequence: Synthetic peptide
GFTFTDHY

SEQ ID NO: 347 - protein, 8 aa, synthetic construct
  REGION 1..8: note = Description of Artificial Sequence: Synthetic peptide
IYPGSGNT

SEQ ID NO: 348 - protein, 11 aa, synthetic construct
  REGION 1..11: note = Description of Artificial Sequence: Synthetic peptide
ARNYGDYTIDF

SEQ ID NO: 349 - protein, 12 aa, synthetic construct
  REGION 1..12: note = Description of Artificial Sequence: Synthetic peptide
QSVLYSSNNKNY

SEQ ID NO: 350 - skipped

SEQ ID NO: 351 - protein, 8 aa, synthetic construct
  REGION 1..8: note = Description of Artificial Sequence: Synthetic peptide
QQYYRYHT

SEQ ID NO: 352 - protein, 5 aa, synthetic construct
  REGION 1..5: note = Description of Artificial Sequence: Synthetic peptide
DHYIN

SEQ ID NO: 353 - protein, 17 aa, synthetic construct
  REGION 1..17: note = Description of Artificial Sequence: Synthetic peptide
QIYPGSGNTYYNQKFKG

SEQ ID NO: 354 - protein, 9 aa, synthetic construct
  REGION 1..9: note = Description of Artificial Sequence: Synthetic peptide
NYGDYTIDF

SEQ ID NO: 355 - protein, 17 aa, synthetic construct
  REGION 1..17: note = Description of Artificial Sequence: Synthetic peptide
KSSQSVLYSSNNKNYLA

SEQ ID NO: 356 - protein, 7 aa, synthetic construct
  REGION 1..7: note = Description of Artificial Sequence: Synthetic peptide
WASTRES

SEQ ID NO: 357 - protein, 8 aa, synthetic construct
  REGION 1..8: note = Description of Artificial Sequence: Synthetic peptide
QQYYRYHT

SEQ ID NO: 358 - protein, 7 aa, synthetic construct
  REGION 1..7: note = Description of Artificial Sequence: Synthetic peptide
GFTFTDH

SEQ ID NO: 359 - protein, 4 aa, synthetic construct
  REGION 1..4: note = Description of Artificial Sequence: Synthetic peptide
PGSG

SEQ ID NO: 360 - protein, 7 aa, synthetic construct
  REGION 1..7: note = Description of Artificial Sequence: Synthetic peptide
YGDYTID

SEQ ID NO: 361 - protein, 13 aa, synthetic construct
  REGION 1..13: note = Description of Artificial Sequence: Synthetic peptide
SQSVLYSSNNKNY

SEQ ID NO: 362 - skipped

SEQ ID NO: 363 - protein, 5 aa, synthetic construct
  REGION 1..5: note = Description of Artificial Sequence: Synthetic peptide
YYRYH

SEQ ID NO: 364 - protein, 6 aa, synthetic construct
  REGION 1..6: note = Description of Artificial Sequence: Synthetic peptide
TDHYIN

SEQ ID NO: 365 - protein, 13 aa, synthetic construct
  REGION 1..13: note = Description of Artificial Sequence: Synthetic peptide
WMGQIYPGSGNTY

SEQ ID NO: 366 - protein, 10 aa, synthetic construct
  REGION 1..10: note = Description of Artificial Sequence: Synthetic peptide
ARNYGDYTID

SEQ ID NO: 367 - protein, 13 aa, synthetic construct
  REGION 1..13: note = Description of Artificial Sequence: Synthetic peptide
LYSSNNKNYLAWY

SEQ ID NO: 368 - protein, 10 aa, synthetic construct
  REGION 1..10: note = Description of Artificial Sequence: Synthetic peptide
LLIYWASTRE

SEQ ID NO: 369 - protein, 7 aa, synthetic construct
  REGION 1..7: note = Description of Artificial Sequence: Synthetic peptide
QQYYRYH

SEQ ID NO: 370 - protein, 10 aa, synthetic construct
  REGION 1..10: note = Description of Artificial Sequence: Synthetic peptide
GFTFTDHYIN

SEQ ID NO: 371 - protein, 10 aa, synthetic construct
  REGION 1..10: note = Description of Artificial Sequence: Synthetic peptide
QIYPGSGNTY

SEQ ID NO: 372 - protein, 9 aa, synthetic construct
  REGION 1..9: note = Description of Artificial Sequence: Synthetic peptide
NYGDYTIDF

SEQ ID NO: 373 - protein, 17 aa, synthetic construct
  REGION 1..17: note = Description of Artificial Sequence: Synthetic peptide
KSSQSVLYSSNNKNYLA

SEQ ID NO: 374 - protein, 7 aa, synthetic construct
  REGION 1..7: note = Description of Artificial Sequence: Synthetic peptide
WASTRES

SEQ ID NO: 375 - protein, 8 aa, synthetic construct
  REGION 1..8: note = Description of Artificial Sequence: Synthetic peptide
QQYYRYHT

SEQ ID NO: 376 - protein, 10 aa, synthetic construct
  REGION 1..10: note = Description of Artificial Sequence: Synthetic peptide
GFTFSSNYIS

SEQ ID NO: 377 - protein, 17 aa, synthetic construct
  REGION 1..17: note = Description of Artificial Sequence: Synthetic peptide
WIHGGTGGIGYNQKFTG

SEQ ID NO: 378 - protein, 9 aa, synthetic construct
  REGION 1..9: note = Description of Artificial Sequence: Synthetic peptide
RGYGAWFAY

SEQ ID NO: 379 - protein, 11 aa, synthetic construct
  REGION 1..11: note = Description of Artificial Sequence: Synthetic peptide
RASENIHNYLA

SEQ ID NO: 380 - protein, 7 aa, synthetic construct
  REGION 1..7: note = Description of Artificial Sequence: Synthetic peptide
NAKTLAD

SEQ ID NO: 381 - protein, 9 aa, synthetic construct
  REGION 1..9: note = Description of Artificial Sequence: Synthetic peptide
QHFWSYPLT

SEQ ID NO: 382 - protein, 8 aa, synthetic construct
  REGION 1..8: note = Description of Artificial Sequence: Synthetic peptide
GFTFSSNY

SEQ ID NO: 383 - protein, 8 aa, synthetic construct
  REGION 1..8: note = Description of Artificial Sequence: Synthetic peptide
IHGGTGGI

SEQ ID NO: 384 - protein, 11 aa, synthetic construct
  REGION 1..11: note = Description of Artificial Sequence: Synthetic peptide
ARRGYGAWFAY

SEQ ID NO: 385 - protein, 6 aa, synthetic construct
  REGION 1..6: note = Description of Artificial Sequence: Synthetic peptide
ENIHNY

SEQ ID NO: 386 - skipped

SEQ ID NO: 387 - protein, 9 aa, synthetic construct
  REGION 1..9: note = Description of Artificial Sequence: Synthetic peptide
QHFWSYPLT

SEQ ID NO: 388 - protein, 5 aa, synthetic construct
  REGION 1..5: note = Description of Artificial Sequence: Synthetic peptide
SNYIS

SEQ ID NO: 389 - protein, 17 aa, synthetic construct
  REGION 1..17: note = Description of Artificial Sequence: Synthetic peptide
WIHGGTGGIGYNQKFTG

SEQ ID NO: 390 - protein, 9 aa, synthetic construct
  REGION 1..9: note = Description of Artificial Sequence: Synthetic peptide
RGYGAWFAY

SEQ ID NO: 391 - protein, 11 aa, synthetic construct
  REGION 1..11: note = Description of Artificial Sequence: Synthetic peptide
RASENIHNYLA

SEQ ID NO: 392 - protein, 7 aa, synthetic construct
  REGION 1..7: note = Description of Artificial Sequence: Synthetic peptide
NAKTLAD

SEQ ID NO: 393 - protein, 9 aa, synthetic construct
  REGION 1..9: note = Description of Artificial Sequence: Synthetic peptide
QHFWSYPLT

SEQ ID NO: 394 - protein, 7 aa, synthetic construct
  REGION 1..7: note = Description of Artificial Sequence: Synthetic peptide
GFTFSSN

SEQ ID NO: 395 - protein, 4 aa, synthetic construct
  REGION 1..4: note = Description of Artificial Sequence: Synthetic peptide
GGTG

SEQ ID NO: 396 - protein, 7 aa, synthetic construct
  REGION 1..7: note = Description of Artificial Sequence: Synthetic peptide
GYGAWFA

SEQ ID NO: 397 - protein, 7 aa, synthetic construct
  REGION 1..7: note = Description of Artificial Sequence: Synthetic peptide
SENIHNY

SEQ ID NO: 398 - skipped

SEQ ID NO: 399 - protein, 6 aa, synthetic construct
  REGION 1..6: note = Description of Artificial Sequence: Synthetic peptide
FWSYPL

SEQ ID NO: 400 - protein, 6 aa, synthetic construct
  REGION 1..6: note = Description of Artificial Sequence: Synthetic peptide
SSNYIS

SEQ ID NO: 401 - protein, 13 aa, synthetic construct
  REGION 1..13: note = Description of Artificial Sequence: Synthetic peptide
WIAWIHGGTGGIG

SEQ ID NO: 402 - protein, 10 aa, synthetic construct
  REGION 1..10: note = Description of Artificial Sequence: Synthetic peptide
ARRGYGAWFA

SEQ ID NO: 403 - protein, 7 aa, synthetic construct
  REGION 1..7: note = Description of Artificial Sequence: Synthetic peptide
HNYLAWY

SEQ ID NO: 404 - protein, 10 aa, synthetic construct
  REGION 1..10: note = Description of Artificial Sequence: Synthetic peptide
LLVYNAKTLA

SEQ ID NO: 405 - protein, 8 aa, synthetic construct
  REGION 1..8: note = Description of Artificial Sequence: Synthetic peptide
QHFWSYPL

SEQ ID NO: 406 - protein, 10 aa, synthetic construct
  REGION 1..10: note = Description of Artificial Sequence: Synthetic peptide
GFTFSSNYIS

SEQ ID NO: 407 - protein, 10 aa, synthetic construct
  REGION 1..10: note = Description of Artificial Sequence: Synthetic peptide
WIHGGTGGIG

SEQ ID NO: 408 - protein, 9 aa, synthetic construct
  REGION 1..9: note = Description of Artificial Sequence: Synthetic peptide
RGYGAWFAY

SEQ ID NO: 409 - protein, 11 aa, synthetic construct
  REGION 1..11: note = Description of Artificial Sequence: Synthetic peptide
RASENIHNYLA

SEQ ID NO: 410 - protein, 7 aa, synthetic construct
  REGION 1..7: note = Description of Artificial Sequence: Synthetic peptide
NAKTLAD

SEQ ID NO: 411 - protein, 9 aa, synthetic construct
  REGION 1..9: note = Description of Artificial Sequence: Synthetic peptide
QHFWSYPLT

SEQ ID NO: 412 - protein, 10 aa, synthetic construct
  REGION 1..10: note = Description of Artificial Sequence: Synthetic peptide
GFTFSNYDMN

SEQ ID NO: 413 - protein, 17 aa, synthetic construct
  REGION 1..17: note = Description of Artificial Sequence: Synthetic peptide
SISSSSSYIYYADSVKG

SEQ ID NO: 414 - protein, 15 aa, synthetic construct
  REGION 1..15: note = Description of Artificial Sequence: Synthetic peptide
DVGVTTDYYYYGMDV

SEQ ID NO: 415 - protein, 12 aa, synthetic construct
  REGION 1..12: note = Description of Artificial Sequence: Synthetic peptide
RASQSVASSYLA

SEQ ID NO: 416 - protein, 7 aa, synthetic construct
  REGION 1..7: note = Description of Artificial Sequence: Synthetic peptide
GASSRAT

SEQ ID NO: 417 - protein, 10 aa, synthetic construct
  REGION 1..10: note = Description of Artificial Sequence: Synthetic peptide
QQYGSSPPYT

SEQ ID NO: 418 - protein, 8 aa, synthetic construct
  REGION 1..8: note = Description of Artificial Sequence: Synthetic peptide
GFTFSNYD

SEQ ID NO: 419 - protein, 8 aa, synthetic construct
  REGION 1..8: note = Description of Artificial Sequence: Synthetic peptide
ISSSSSYI

SEQ ID NO: 420 - protein, 17 aa, synthetic construct
  REGION 1..17: note = Description of Artificial Sequence: Synthetic peptide
ARDVGVTTDYYYYGMDV

SEQ ID NO: 421 - protein, 7 aa, synthetic construct
  REGION 1..7: note = Description of Artificial Sequence: Synthetic peptide
QSVASSY

SEQ ID NO: 422 - skipped

SEQ ID NO: 423 - protein, 10 aa, synthetic construct
  REGION 1..10: note = Description of Artificial Sequence: Synthetic peptide
QQYGSSPPYT

SEQ ID NO: 424 - protein, 5 aa, synthetic construct
  REGION 1..5: note = Description of Artificial Sequence: Synthetic peptide
NYDMN

SEQ ID NO: 425 - protein, 17 aa, synthetic construct
  REGION 1..17: note = Description of Artificial Sequence: Synthetic peptide
SISSSSSYIYYADSVKG

SEQ ID NO: 426 - protein, 15 aa, synthetic construct
  REGION 1..15: note = Description of Artificial Sequence: Synthetic peptide
DVGVTTDYYYYGMDV

SEQ ID NO: 427 - protein, 12 aa, synthetic construct
  REGION 1..12: note = Description of Artificial Sequence: Synthetic peptide
RASQSVASSYLA

SEQ ID NO: 428 - protein, 7 aa, synthetic construct
  REGION 1..7: note = Description of Artificial Sequence: Synthetic peptide
GASSRAT

SEQ ID NO: 429 - protein, 10 aa, synthetic construct
  REGION 1..10: note = Description of Artificial Sequence: Synthetic peptide
QQYGSSPPYT

SEQ ID NO: 430 - protein, 7 aa, synthetic construct
  REGION 1..7: note = Description of Artificial Sequence: Synthetic peptide
GFTFSNY

SEQ ID NO: 431 - protein, 4 aa, synthetic construct
  REGION 1..4: note = Description of Artificial Sequence: Synthetic peptide
SSSS

SEQ ID NO: 432 - protein, 13 aa, synthetic construct
  REGION 1..13: note = Description of Artificial Sequence: Synthetic peptide
VGVTTDYYYYGMD

SEQ ID NO: 433 - protein, 8 aa, synthetic construct
  REGION 1..8: note = Description of Artificial Sequence: Synthetic peptide
SQSVASSY

SEQ ID NO: 434 - skipped

SEQ ID NO: 435 - protein, 7 aa, synthetic construct
  REGION 1..7: note = Description of Artificial Sequence: Synthetic peptide
YGSSPPY

SEQ ID NO: 436 - protein, 6 aa, synthetic construct
  REGION 1..6: note = Description of Artificial Sequence: Synthetic peptide
SNYDMN

SEQ ID NO: 437 - protein, 13 aa, synthetic construct
  REGION 1..13: note = Description of Artificial Sequence: Synthetic peptide
WVSSISSSSSYIY

SEQ ID NO: 438 - protein, 16 aa, synthetic construct
  REGION 1..16: note = Description of Artificial Sequence: Synthetic peptide
ARDVGVTTDYYYYGMD

SEQ ID NO: 439 - protein, 8 aa, synthetic construct
  REGION 1..8: note = Description of Artificial Sequence: Synthetic peptide
ASSYLAWY

SEQ ID NO: 440 - protein, 10 aa, synthetic construct
  REGION 1..10: note = Description of Artificial Sequence: Synthetic peptide
LLIYGASSRA

SEQ ID NO: 441 - protein, 9 aa, synthetic construct
  REGION 1..9: note = Description of Artificial Sequence: Synthetic peptide
QQYGSSPPY

SEQ ID NO: 442 - protein, 10 aa, synthetic construct
  REGION 1..10: note = Description of Artificial Sequence: Synthetic peptide
GFTFSNYDMN

SEQ ID NO: 443 - protein, 10 aa, synthetic construct
  REGION 1..10: note = Description of Artificial Sequence: Synthetic peptide
SISSSSSYIY

SEQ ID NO: 444 - protein, 15 aa, synthetic construct
  REGION 1..15: note = Description of Artificial Sequence: Synthetic peptide
DVGVTTDYYYYGMDV

SEQ ID NO: 445 - protein, 12 aa, synthetic construct
  REGION 1..12: note = Description of Artificial Sequence: Synthetic peptide
RASQSVASSYLA

SEQ ID NO: 446 - protein, 7 aa, synthetic construct
  REGION 1..7: note = Description of Artificial Sequence: Synthetic peptide
GASSRAT

SEQ ID NO: 447 - protein, 10 aa, synthetic construct
  REGION 1..10: note = Description of Artificial Sequence: Synthetic peptide
QQYGSSPPYT

SEQ ID NO: 448 - protein, 10 aa, synthetic construct
  REGION 1..10: note = Description of Artificial Sequence: Synthetic peptide
GDTFNNYAIS

SEQ ID NO: 449 - protein, 17 aa, synthetic construct
  REGION 1..17: note = Description of Artificial Sequence: Synthetic peptide
GIIPFFGTPDYAQKFQG

SEQ ID NO: 450 - protein, 15 aa, synthetic construct
  REGION 1..15: note = Description of Artificial Sequence: Synthetic peptide
PGSGSPDYYYYDMDV

SEQ ID NO: 451 - protein, 16 aa, synthetic construct
  REGION 1..16: note = Description of Artificial Sequence: Synthetic peptide
RSSQSLVHSDGNTYLS

SEQ ID NO: 452 - protein, 7 aa, synthetic construct
  REGION 1..7: note = Description of Artificial Sequence: Synthetic peptide
KISNRFS

SEQ ID NO: 453 - protein, 9 aa, synthetic construct
  REGION 1..9: note = Description of Artificial Sequence: Synthetic peptide
MQATQFPLT

SEQ ID NO: 454 - protein, 8 aa, synthetic construct
  REGION 1..8: note = Description of Artificial Sequence: Synthetic peptide
GDTFNNYA

SEQ ID NO: 455 - protein, 8 aa, synthetic construct
  REGION 1..8: note = Description of Artificial Sequence: Synthetic peptide
IIPFFGTP

SEQ ID NO: 456 - protein, 17 aa, synthetic construct
  REGION 1..17: note = Description of Artificial Sequence: Synthetic peptide
ARPGSGSPDYYYYDMDV

SEQ ID NO: 457 - protein, 11 aa, synthetic construct
  REGION 1..11: note = Description of Artificial Sequence: Synthetic peptide
QSLVHSDGNTY

SEQ ID NO: 458 - skipped

SEQ ID NO: 459 - protein, 9 aa, synthetic construct
  REGION 1..9: note = Description of Artificial Sequence: Synthetic peptide
MQATQFPLT

SEQ ID NO: 460 - protein, 5 aa, synthetic construct
  REGION 1..5: note = Description of Artificial Sequence: Synthetic peptide
NYAIS

SEQ ID NO: 461 - protein, 17 aa, synthetic construct
  REGION 1..17: note = Description of Artificial Sequence: Synthetic peptide
GIIPFFGTPDYAQKFQG

SEQ ID NO: 462 - protein, 15 aa, synthetic construct
  REGION 1..15: note = Description of Artificial Sequence: Synthetic peptide
PGSGSPDYYYYDMDV

SEQ ID NO: 463 - protein, 16 aa, synthetic construct
  REGION 1..16: note = Description of Artificial Sequence: Synthetic peptide
RSSQSLVHSDGNTYLS

SEQ ID NO: 464 - protein, 7 aa, synthetic construct
  REGION 1..7: note = Description of Artificial Sequence: Synthetic peptide
KISNRFS

SEQ ID NO: 465 - protein, 9 aa, synthetic construct
  REGION 1..9: note = Description of Artificial Sequence: Synthetic peptide
MQATQFPLT

SEQ ID NO: 466 - protein, 7 aa, synthetic construct
  REGION 1..7: note = Description of Artificial Sequence: Synthetic peptide
GDTFNNY

SEQ ID NO: 467 - protein, 4 aa, synthetic construct
  REGION 1..4: note = Description of Artificial Sequence: Synthetic peptide
PFFG

SEQ ID NO: 468 - protein, 13 aa, synthetic construct
  REGION 1..13: note = Description of Artificial Sequence: Synthetic peptide
GSGSPDYYYYDMD

SEQ ID NO: 469 - protein, 12 aa, synthetic construct
  REGION 1..12: note = Description of Artificial Sequence: Synthetic peptide
SQSLVHSDGNTY

SEQ ID NO: 470 - skipped

SEQ ID NO: 471 - protein, 6 aa, synthetic construct
  REGION 1..6: note = Description of Artificial Sequence: Synthetic peptide
ATQFPL

SEQ ID NO: 472 - protein, 6 aa, synthetic construct
  REGION 1..6: note = Description of Artificial Sequence: Synthetic peptide
NNYAIS

SEQ ID NO: 473 - protein, 13 aa, synthetic construct
  REGION 1..13: note = Description of Artificial Sequence: Synthetic peptide
WMGGIIPFFGTPD

SEQ ID NO: 474 - protein, 16 aa, synthetic construct
  REGION 1..16: note = Description of Artificial Sequence: Synthetic peptide
ARPGSGSPDYYYYDMD

SEQ ID NO: 475 - protein, 12 aa, synthetic construct
  REGION 1..12: note = Description of Artificial Sequence: Synthetic peptide
VHSDGNTYLSWL

SEQ ID NO: 476 - protein, 10 aa, synthetic construct
  REGION 1..10: note = Description of Artificial Sequence: Synthetic peptide
LLIYKISNRF

SEQ ID NO: 477 - protein, 8 aa, synthetic construct
  REGION 1..8: note = Description of Artificial Sequence: Synthetic peptide
MQATQFPL

SEQ ID NO: 478 - protein, 10 aa, synthetic construct
  REGION 1..10: note = Description of Artificial Sequence: Synthetic peptide
GDTFNNYAIS

SEQ ID NO: 479 - protein, 10 aa, synthetic construct
  REGION 1..10: note = Description of Artificial Sequence: Synthetic peptide
GIIPFFGTPD

SEQ ID NO: 480 - protein, 15 aa, synthetic construct
  REGION 1..15: note = Description of Artificial Sequence: Synthetic peptide
PGSGSPDYYYYDMDV

SEQ ID NO: 481 - protein, 16 aa, synthetic construct
  REGION 1..16: note = Description of Artificial Sequence: Synthetic peptide
RSSQSLVHSDGNTYLS

SEQ ID NO: 482 - protein, 7 aa, synthetic construct
  REGION 1..7: note = Description of Artificial Sequence: Synthetic peptide
KISNRFS

SEQ ID NO: 483 - protein, 9 aa, synthetic construct
  REGION 1..9: note = Description of Artificial Sequence: Synthetic peptide
MQATQFPLT

SEQ ID NO: 484 - protein, 10 aa, synthetic construct
  REGION 1..10: note = Description of Artificial Sequence: Synthetic peptide
GGTFSSYAIS

SEQ ID NO: 485 - protein, 17 aa, synthetic construct
  REGION 1..17: note = Description of Artificial Sequence: Synthetic peptide
GNIPIFNTANYAQKFQD

SEQ ID NO: 486 - protein, 11 aa, synthetic construct
  REGION 1..11: note = Description of Artificial Sequence: Synthetic peptide
EGTGYSYGLDY

SEQ ID NO: 487 - protein, 16 aa, synthetic construct
  REGION 1..16: note = Description of Artificial Sequence: Synthetic peptide
RSSQSLIHSDGNTYLS

SEQ ID NO: 488 - protein, 7 aa, synthetic construct
  REGION 1..7: note = Description of Artificial Sequence: Synthetic peptide
KISNRFS

SEQ ID NO: 489 - protein, 9 aa, synthetic construct
  REGION 1..9: note = Description of Artificial Sequence: Synthetic peptide
MQAKQFPIT

SEQ ID NO: 490 - protein, 8 aa, synthetic construct
  REGION 1..8: note = Description of Artificial Sequence: Synthetic peptide
GGTFSSYA

SEQ ID NO: 491 - protein, 8 aa, synthetic construct
  REGION 1..8: note = Description of Artificial Sequence: Synthetic peptide
NIPIFNTA

SEQ ID NO: 492 - protein, 13 aa, synthetic construct
  REGION 1..13: note = Description of Artificial Sequence: Synthetic peptide
VREGTGYSYGLDY

SEQ ID NO: 493 - protein, 11 aa, synthetic construct
  REGION 1..11: note = Description of Artificial Sequence: Synthetic peptide
QSLIHSDGNTY

SEQ ID NO: 494 - skipped

SEQ ID NO: 495 - protein, 9 aa, synthetic construct
  REGION 1..9: note = Description of Artificial Sequence: Synthetic peptide
MQAKQFPIT

SEQ ID NO: 496 - protein, 5 aa, synthetic construct
  REGION 1..5: note = Description of Artificial Sequence: Synthetic peptide
SYAIS

SEQ ID NO: 497 - protein, 17 aa, synthetic construct
  REGION 1..17: note = Description of Artificial Sequence: Synthetic peptide
GNIPIFNTANYAQKFQD

SEQ ID NO: 498 - protein, 11 aa, synthetic construct
  REGION 1..11: note = Description of Artificial Sequence: Synthetic peptide
EGTGYSYGLDY

SEQ ID NO: 499 - protein, 16 aa, synthetic construct
  REGION 1..16: note = Description of Artificial Sequence: Synthetic peptide
RSSQSLIHSDGNTYLS

SEQ ID NO: 500 - protein, 7 aa, synthetic construct
  REGION 1..7: note = Description of Artificial Sequence: Synthetic peptide
KISNRFS

SEQ ID NO: 501 - protein, 9 aa, synthetic construct
  REGION 1..9: note = Description of Artificial Sequence: Synthetic peptide
MQAKQFPIT

SEQ ID NO: 502 - protein, 7 aa, synthetic construct
  REGION 1..7: note = Description of Artificial Sequence: Synthetic peptide
GGTFSSY

SEQ ID NO: 503 - protein, 4 aa, synthetic construct
  REGION 1..4: note = Description of Artificial Sequence: Synthetic peptide
PIFN

SEQ ID NO: 504 - protein, 9 aa, synthetic construct
  REGION 1..9: note = Description of Artificial Sequence: Synthetic peptide
GTGYSYGLD

SEQ ID NO: 505 - protein, 12 aa, synthetic construct
  REGION 1..12: note = Description of Artificial Sequence: Synthetic peptide
SQSLIHSDGNTY

SEQ ID NO: 506 - skipped

SEQ ID NO: 507 - protein, 6 aa, synthetic construct
  REGION 1..6: note = Description of Artificial Sequence: Synthetic peptide
AKQFPI

SEQ ID NO: 508 - protein, 6 aa, synthetic construct
  REGION 1..6: note = Description of Artificial Sequence: Synthetic peptide
SSYAIS

SEQ ID NO: 509 - protein, 13 aa, synthetic construct
  REGION 1..13: note = Description of Artificial Sequence: Synthetic peptide
WMGGNIPIFNTAN

SEQ ID NO: 510 - protein, 12 aa, synthetic construct
  REGION 1..12: note = Description of Artificial Sequence: Synthetic peptide
VREGTGYSYGLD

SEQ ID NO: 511 - protein, 12 aa, synthetic construct
  REGION 1..12: note = Description of Artificial Sequence: Synthetic peptide
IHSDGNTYLSWL

SEQ ID NO: 512 - protein, 10 aa, synthetic construct
  REGION 1..10: note = Description of Artificial Sequence: Synthetic peptide
LLIYKISNRF

SEQ ID NO: 513 - protein, 8 aa, synthetic construct
  REGION 1..8: note = Description of Artificial Sequence: Synthetic peptide
MQAKQFPI

SEQ ID NO: 514 - protein, 10 aa, synthetic construct
  REGION 1..10: note = Description of Artificial Sequence: Synthetic peptide
GGTFSSYAIS

SEQ ID NO: 515 - protein, 10 aa, synthetic construct
  REGION 1..10: note = Description of Artificial Sequence: Synthetic peptide
GNIPIFNTAN

SEQ ID NO: 516 - protein, 11 aa, synthetic construct
  REGION 1..11: note = Description of Artificial Sequence: Synthetic peptide
EGTGYSYGLDY

SEQ ID NO: 517 - protein, 16 aa, synthetic construct
  REGION 1..16: note = Description of Artificial Sequence: Synthetic peptide
RSSQSLIHSDGNTYLS

SEQ ID NO: 518 - protein, 7 aa, synthetic construct
  REGION 1..7: note = Description of Artificial Sequence: Synthetic peptide
KISNRFS

SEQ ID NO: 519 - protein, 9 aa, synthetic construct
  REGION 1..9: note = Description of Artificial Sequence: Synthetic peptide
MQAKQFPIT

SEQ ID NO: 520 - protein, 12 aa, synthetic construct
  REGION 1..12: note = Description of Artificial Sequence: Synthetic peptide
GGSISSGGSYWS

SEQ ID NO: 521 - protein, 16 aa, synthetic construct
  REGION 1..16: note = Description of Artificial Sequence: Synthetic peptide
YIYNSGSTYYNPSLKS

SEQ ID NO: 522 - protein, 10 aa, synthetic construct
  REGION 1..10: note = Description of Artificial Sequence: Synthetic peptide
DSNYEWFFDL

SEQ ID NO: 523 - protein, 11 aa, synthetic construct
  REGION 1..11: note = Description of Artificial Sequence: Synthetic peptide
RASQSVSSYLA

SEQ ID NO: 524 - protein, 7 aa, synthetic construct
  REGION 1..7: note = Description of Artificial Sequence: Synthetic peptide
DASNRAT

SEQ ID NO: 525 - protein, 9 aa, synthetic construct
  REGION 1..9: note = Description of Artificial Sequence: Synthetic peptide
QQRSNWPLT

SEQ ID NO: 526 - protein, 10 aa, synthetic construct
  REGION 1..10: note = Description of Artificial Sequence: Synthetic peptide
GGSISSGGSY

SEQ ID NO: 527 - protein, 7 aa, synthetic construct
  REGION 1..7: note = Description of Artificial Sequence: Synthetic peptide
IYNSGST

SEQ ID NO: 528 - protein, 12 aa, synthetic construct
  REGION 1..12: note = Description of Artificial Sequence: Synthetic peptide
ARDSNYEWFFDL

SEQ ID NO: 529 - protein, 6 aa, synthetic construct
  REGION 1..6: note = Description of Artificial Sequence: Synthetic peptide
QSVSSY

SEQ ID NO: 530 - skipped

SEQ ID NO: 531 - protein, 9 aa, synthetic construct
  REGION 1..9: note = Description of Artificial Sequence: Synthetic peptide
QQRSNWPLT

SEQ ID NO: 532 - protein, 7 aa, synthetic construct
  REGION 1..7: note = Description of Artificial Sequence: Synthetic peptide
SGGSYWS

SEQ ID NO: 533 - protein, 16 aa, synthetic construct
  REGION 1..16: note = Description of Artificial Sequence: Synthetic peptide
YIYNSGSTYYNPSLKS

SEQ ID NO: 534 - protein, 10 aa, synthetic construct
  REGION 1..10: note = Description of Artificial Sequence: Synthetic peptide
DSNYEWFFDL

SEQ ID NO: 535 - protein, 11 aa, synthetic construct
  REGION 1..11: note = Description of Artificial Sequence: Synthetic peptide
RASQSVSSYLA

SEQ ID NO: 536 - protein, 7 aa, synthetic construct
  REGION 1..7: note = Description of Artificial Sequence: Synthetic peptide
DASNRAT

SEQ ID NO: 537 - protein, 9 aa, synthetic construct
  REGION 1..9: note = Description of Artificial Sequence: Synthetic peptide
QQRSNWPLT

SEQ ID NO: 538 - protein, 9 aa, synthetic construct
  REGION 1..9: note = Description of Artificial Sequence: Synthetic peptide
GGSISSGGS

SEQ ID NO: 539 - skipped

SEQ ID NO: 540 - protein, 8 aa, synthetic construct
  REGION 1..8: note = Description of Artificial Sequence: Synthetic peptide
SNYEWFFD

SEQ ID NO: 541 - protein, 7 aa, synthetic construct
  REGION 1..7: note = Description of Artificial Sequence: Synthetic peptide
SQSVSSY

SEQ ID NO: 542 - skipped

SEQ ID NO: 543 - protein, 6 aa, synthetic construct
  REGION 1..6: note = Description of Artificial Sequence: Synthetic peptide
RSNWPL

SEQ ID NO: 544 - protein, 8 aa, synthetic construct
  REGION 1..8: note = Description of Artificial Sequence: Synthetic peptide
SSGGSYWS

SEQ ID NO: 545 - protein, 12 aa, synthetic construct
  REGION 1..12: note = Description of Artificial Sequence: Synthetic peptide
WIGYIYNSGSTY

SEQ ID NO: 546 - protein, 11 aa, synthetic construct
  REGION 1..11: note = Description of Artificial Sequence: Synthetic peptide
ARDSNYEWFFD

SEQ ID NO: 547 - protein, 7 aa, synthetic construct
  REGION 1..7: note = Description of Artificial Sequence: Synthetic peptide
SSYLAWY

SEQ ID NO: 548 - protein, 10 aa, synthetic construct
  REGION 1..10: note = Description of Artificial Sequence: Synthetic peptide
LLIYDASNRA

SEQ ID NO: 549 - protein, 8 aa, synthetic construct
  REGION 1..8: note = Description of Artificial Sequence: Synthetic peptide
QQRSNWPL

SEQ ID NO: 550 - protein, 12 aa, synthetic construct
  REGION 1..12: note = Description of Artificial Sequence: Synthetic peptide
GGSISSGGSYWS

SEQ ID NO: 551 - protein, 9 aa, synthetic construct
  REGION 1..9: note = Description of Artificial Sequence: Synthetic peptide
YIYNSGSTY

SEQ ID NO: 552 - protein, 10 aa, synthetic construct
  REGION 1..10: note = Description of Artificial Sequence: Synthetic peptide
DSNYEWFFDL

SEQ ID NO: 553 - protein, 11 aa, synthetic construct
  REGION 1..11: note = Description of Artificial Sequence: Synthetic peptide
RASQSVSSYLA

SEQ ID NO: 554 - protein, 7 aa, synthetic construct
  REGION 1..7: note = Description of Artificial Sequence: Synthetic peptide
DASNRAT

SEQ ID NO: 555 - protein, 9 aa, synthetic construct
  REGION 1..9: note = Description of Artificial Sequence: Synthetic peptide
QQRSNWPLT

SEQ ID NO: 556 - protein, 10 aa, synthetic construct
  REGION 1..10: note = Description of Artificial Sequence: Synthetic peptide
GYSFTSYWIS

SEQ ID NO: 557 - protein, 17 aa, synthetic construct
  REGION 1..17: note = Description of Artificial Sequence: Synthetic peptide
IIDPSDSDTRYSPSFQG

SEQ ID NO: 558 - protein, 9 aa, synthetic construct
  REGION 1..9: note = Description of Artificial Sequence: Synthetic peptide
GDGSTDLDY

SEQ ID NO: 559 - protein, 12 aa, synthetic construct
  REGION 1..12: note = Description of Artificial Sequence: Synthetic peptide
RASQSVSSSYLA

SEQ ID NO: 560 - protein, 7 aa, synthetic construct
  REGION 1..7: note = Description of Artificial Sequence: Synthetic peptide
GASSRAT

SEQ ID NO: 561 - protein, 9 aa, synthetic construct
  REGION 1..9: note = Description of Artificial Sequence: Synthetic peptide
QQDYGFPWT

SEQ ID NO: 562 - protein, 8 aa, synthetic construct
  REGION 1..8: note = Description of Artificial Sequence: Synthetic peptide
GYSFTSYW

SEQ ID NO: 563 - protein, 8 aa, synthetic construct
  REGION 1..8: note = Description of Artificial Sequence: Synthetic peptide
IDPSDSDT

SEQ ID NO: 564 - protein, 11 aa, synthetic construct
  REGION 1..11: note = Description of Artificial Sequence: Synthetic peptide
ARGDGSTDLDY

SEQ ID NO: 565 - protein, 7 aa, synthetic construct
  REGION 1..7: note = Description of Artificial Sequence: Synthetic peptide
QSVSSSY

SEQ ID NO: 566 - skipped

SEQ ID NO: 567 - protein, 9 aa, synthetic construct
  REGION 1..9: note = Description of Artificial Sequence: Synthetic peptide
QQDYGFPWT

SEQ ID NO: 568 - protein, 5 aa, synthetic construct
  REGION 1..5: note = Description of Artificial Sequence: Synthetic peptide
SYWIS

SEQ ID NO: 569 - protein, 17 aa, synthetic construct
  REGION 1..17: note = Description of Artificial Sequence: Synthetic peptide
IIDPSDSDTRYSPSFQG

SEQ ID NO: 570 - protein, 9 aa, synthetic construct
  REGION 1..9: note = Description of Artificial Sequence: Synthetic peptide
GDGSTDLDY

SEQ ID NO: 571 - protein, 12 aa, synthetic construct
  REGION 1..12: note = Description of Artificial Sequence: Synthetic peptide
RASQSVSSSYLA

SEQ ID NO: 572 - protein, 7 aa, synthetic construct
  REGION 1..7: note = Description of Artificial Sequence: Synthetic peptide
GASSRAT

SEQ ID NO: 573 - protein, 9 aa, synthetic construct
  REGION 1..9: note = Description of Artificial Sequence: Synthetic peptide
QQDYGFPWT

SEQ ID NO: 574 - protein, 7 aa, synthetic construct
  REGION 1..7: note = Description of Artificial Sequence: Synthetic peptide
GYSFTSY

SEQ ID NO: 575 - protein, 4 aa, synthetic construct
  REGION 1..4: note = Description of Artificial Sequence: Synthetic peptide
PSDS

SEQ ID NO: 576 - protein, 7 aa, synthetic construct
  REGION 1..7: note = Description of Artificial Sequence: Synthetic peptide
DGSTDLD

SEQ ID NO: 577 - protein, 8 aa, synthetic construct
  REGION 1..8: note = Description of Artificial Sequence: Synthetic peptide
SQSVSSSY

SEQ ID NO: 578 - skipped

SEQ ID NO: 579 - protein, 6 aa, synthetic construct
  REGION 1..6: note = Description of Artificial Sequence: Synthetic peptide
DYGFPW

SEQ ID NO: 580 - protein, 6 aa, synthetic construct
  REGION 1..6: note = Description of Artificial Sequence: Synthetic peptide
TSYWIS

SEQ ID NO: 581 - protein, 13 aa, synthetic construct
  REGION 1..13: note = Description of Artificial Sequence: Synthetic peptide
WMGIIDPSDSDTR

SEQ ID NO: 582 - protein, 10 aa, synthetic construct
  REGION 1..10: note = Description of Artificial Sequence: Synthetic peptide
ARGDGSTDLD

SEQ ID NO: 583 - protein, 8 aa, synthetic construct
  REGION 1..8: note = Description of Artificial Sequence: Synthetic peptide
SSSYLAWY

SEQ ID NO: 584 - protein, 10 aa, synthetic construct
  REGION 1..10: note = Description of Artificial Sequence: Synthetic peptide
LLIYGASSRA

SEQ ID NO: 585 - protein, 8 aa, synthetic construct
  REGION 1..8: note = Description of Artificial Sequence: Synthetic peptide
QQDYGFPW

SEQ ID NO: 586 - protein, 10 aa, synthetic construct
  REGION 1..10: note = Description of Artificial Sequence: Synthetic peptide
GYSFTSYWIS

SEQ ID NO: 587 - protein, 10 aa, synthetic construct
  REGION 1..10: note = Description of Artificial Sequence: Synthetic peptide
IIDPSDSDTR

SEQ ID NO: 588 - protein, 9 aa, synthetic construct
  REGION 1..9: note = Description of Artificial Sequence: Synthetic peptide
GDGSTDLDY

SEQ ID NO: 589 - protein, 12 aa, synthetic construct
  REGION 1..12: note = Description of Artificial Sequence: Synthetic peptide
RASQSVSSSYLA

SEQ ID NO: 590 - protein, 7 aa, synthetic construct
  REGION 1..7: note = Description of Artificial Sequence: Synthetic peptide
GASSRAT

SEQ ID NO: 591 - protein, 9 aa, synthetic construct
  REGION 1..9: note = Description of Artificial Sequence: Synthetic peptide
QQDYGFPWT

SEQ ID NO: 592-699 - skipped (108 entries)

SEQ ID NO: 700 - protein, 6 aa, synthetic construct
  REGION 1..6: note = Description of Artificial Sequence: Synthetic peptide
YPGDGN

SEQ ID NO: 701 - protein, 8 aa, synthetic construct
  REGION 1..8: note = Description of Artificial Sequence: Synthetic peptide
NYGDYTID

SEQ ID NO: 702 - protein, 6 aa, synthetic construct
  REGION 1..6: note = Description of Artificial Sequence: Synthetic peptide
YPGDGN

SEQ ID NO: 703 - protein, 8 aa, synthetic construct
  REGION 1..8: note = Description of Artificial Sequence: Synthetic peptide
NMGMYTID

SEQ ID NO: 704 - protein, 6 aa, synthetic construct
  REGION 1..6: note = Description of Artificial Sequence: Synthetic peptide
YPGDGN

SEQ ID NO: 705 - protein, 8 aa, synthetic construct
  REGION 1..8: note = Description of Artificial Sequence: Synthetic peptide
NMGMYTLD

SEQ ID NO: 706 - protein, 6 aa, synthetic construct
  REGION 1..6: note = Description of Artificial Sequence: Synthetic peptide
YPGDGN

SEQ ID NO: 707 - protein, 8 aa, synthetic construct
  REGION 1..8: note = Description of Artificial Sequence: Synthetic peptide
NYGDYTLD

SEQ ID NO: 708 - protein, 6 aa, synthetic construct
  REGION 1..6: note = Description of Artificial Sequence: Synthetic peptide
YPGSGN

SEQ ID NO: 709 - protein, 8 aa, synthetic construct
  REGION 1..8: note = Description of Artificial Sequence: Synthetic peptide
NYGDYTID

SEQ ID NO: 710 - protein, 6 aa, synthetic construct
  REGION 1..6: note = Description of Artificial Sequence: Synthetic peptide
YPGSGN

SEQ ID NO: 711 - protein, 8 aa, synthetic construct
  REGION 1..8: note = Description of Artificial Sequence: Synthetic peptide
NYGDYTID

SEQ ID NO: 712 - protein, 6 aa, synthetic construct
  REGION 1..6: note = Description of Artificial Sequence: Synthetic peptide
HGGTGG

SEQ ID NO: 713 - protein, 8 aa, synthetic construct
  REGION 1..8: note = Description of Artificial Sequence: Synthetic peptide
RGYGAWFA

SEQ ID NO: 714 - protein, 6 aa, synthetic construct
  REGION 1..6: note = Description of Artificial Sequence: Synthetic peptide
SSSSSY

SEQ ID NO: 715 - protein, 14 aa, synthetic construct
  REGION 1..14: note = Description of Artificial Sequence: Synthetic peptide
DVGVTTDYYYYGMD

SEQ ID NO: 716 - protein, 6 aa, synthetic construct
  REGION 1..6: note = Description of Artificial Sequence: Synthetic peptide
IPFFGT

SEQ ID NO: 717 - protein, 14 aa, synthetic construct
  REGION 1..14: note = Description of Artificial Sequence: Synthetic peptide
PGSGSPDYYYYDMD

SEQ ID NO: 718 - protein, 6 aa, synthetic construct
  REGION 1..6: note = Description of Artificial Sequence: Synthetic peptide
IPIFNT

SEQ ID NO: 719 - protein, 10 aa, synthetic construct
  REGION 1..10: note = Description of Artificial Sequence: Synthetic peptide
EGTGYSYGLD

SEQ ID NO: 720 - protein, 5 aa, synthetic construct
  REGION 1..5: note = Description of Artificial Sequence: Synthetic peptide
YNSGS

SEQ ID NO: 721 - protein, 9 aa, synthetic construct
  REGION 1..9: note = Description of Artificial Sequence: Synthetic peptide
DSNYEWFFD

SEQ ID NO: 722 - protein, 6 aa, synthetic construct
  REGION 1..6: note = Description of Artificial Sequence: Synthetic peptide
DPSDSD

SEQ ID NO: 723 - protein, 8 aa, synthetic construct
  REGION 1..8: note = Description of Artificial Sequence: Synthetic peptide
GDGSTDLD

SEQ ID NO: 724-729 - skipped (6 entries)

SEQ ID NO: 730 - protein, 10 aa, synthetic construct
  REGION 1..10: note = Description of Artificial Sequence: Synthetic peptide
GRTFSRYTMG

SEQ ID NO: 731 - protein, 17 aa, synthetic construct
  REGION 1..17: note = Description of Artificial Sequence: Synthetic peptide
AISWSGGRTNFAGSVKG

SEQ ID NO: 732 - protein, 13 aa, synthetic construct
  REGION 1..13: note = Description of Artificial Sequence: Synthetic peptide
DWLPVPGRESYDY

SEQ ID NO: 733 - protein, 10 aa, synthetic construct
  REGION 1..10: note = Description of Artificial Sequence: Synthetic peptide
GRTFSSYAMG

SEQ ID NO: 734 - protein, 17 aa, synthetic construct
  REGION 1..17: note = Description of Artificial Sequence: Synthetic peptide
AISWSGGTTYYADSVKG

SEQ ID NO: 735 - protein, 16 aa, synthetic construct
  REGION 1..16: note = Description of Artificial Sequence: Synthetic peptide
SLDCSGPGCHTAEYDY

SEQ ID NO: 736 - protein, 10 aa, synthetic construct
  REGION 1..10: note = Description of Artificial Sequence: Synthetic peptide
GRTFSEYAMG

SEQ ID NO: 737 - protein, 17 aa, synthetic construct
  REGION 1..17: note = Description of Artificial Sequence: Synthetic peptide
AISWTGSKTYYADSVKG

SEQ ID NO: 738 - protein, 16 aa, synthetic construct
  REGION 1..16: note = Description of Artificial Sequence: Synthetic peptide
SSDCSGPGCHTEEYDY

SEQ ID NO: 739 - protein, 10 aa, synthetic construct
  REGION 1..10: note = Description of Artificial Sequence: Synthetic peptide
GRTFSSYAMG

SEQ ID NO: 740 - protein, 17 aa, synthetic construct
  REGION 1..17: note = Description of Artificial Sequence: Synthetic peptide
AVSWSGGSTYYADSVKG

SEQ ID NO: 741 - protein, 16 aa, synthetic construct
  REGION 1..16: note = Description of Artificial Sequence: Synthetic peptide
SQDCSGPGCYTNEYDS

SEQ ID NO: 742 - protein, 10 aa, synthetic construct
  REGION 1..10: note = Description of Artificial Sequence: Synthetic peptide
GSIFSNYAMA

SEQ ID NO: 743 - protein, 17 aa, synthetic construct
  REGION 1..17: note = Description of Artificial Sequence: Synthetic peptide
AVSWSGGRTYYADSVKG

SEQ ID NO: 744 - protein, 16 aa, synthetic construct
  REGION 1..16: note = Description of Artificial Sequence: Synthetic peptide
SLSCSGPGCSLEEYDY

SEQ ID NO: 745 - protein, 10 aa, synthetic construct
  REGION 1..10: note = Description of Artificial Sequence: Synthetic peptide
GRPFSNYGMG

SEQ ID NO: 746 - protein, 17 aa, synthetic construct
  REGION 1..17: note = Description of Artificial Sequence: Synthetic peptide
GITWSGGSTHYADLVKG

SEQ ID NO: 747 - protein, 17 aa, synthetic construct
  REGION 1..17: note = Description of Artificial Sequence: Synthetic peptide
VFSGAETAYYPSTEYDY

SEQ ID NO: 748 - protein, 10 aa, synthetic construct
  REGION 1..10: note = Description of Artificial Sequence: Synthetic peptide
VRTFSDYRMG

SEQ ID NO: 749 - protein, 17 aa, synthetic construct
  REGION 1..17: note = Description of Artificial Sequence: Synthetic peptide
TISWSGGLTYYADSVKG

SEQ ID NO: 750 - protein, 14 aa, synthetic construct
  REGION 1..14: note = Description of Artificial Sequence: Synthetic peptide
GGGYAGGTYYHPEE

SEQ ID NO: 751 - protein, 122 aa, synthetic construct
  REGION 1..122: note = Description of Artificial Sequence: Synthetic polypeptide
EVQLVESGGGLVQAGDSLRLSCAASGRTFSRYTMGWFRQAPGKEREFVAAISWSGGRTNFAGSVKGRFTISRDNAKNTVYLQMNSLKPEDTAVYYCAADWLPVPGRESYDYWGQGTQVTVSS

SEQ ID NO: 752 - protein, 125 aa, synthetic construct
  REGION 1..125: note = Description of Artificial Sequence: Synthetic polypeptide
EVQLVESGGGLVQAGGSLRLSCTASGRTFSSYAMGWFRQAPGKEREFVAAISWSGGTTYYADSVKGRFTISRDNAKNTVSLQMNSLKPEDTAVYFCAASLDCSGPGCHTAEYDYWGQGTQVTVSS

SEQ ID NO: 753 - protein, 125 aa, synthetic construct
  REGION 1..125: note = Description of Artificial Sequence: Synthetic polypeptide
EVQLVESGGGLVQAGGSLRLSCAATGRTFSEYAMGWFRQAPGKEREFAAAISWIGSKTYYADSVKGRFTISRDNAKNTVYLQMNSLKPEDTAVYYCAASSDCSGPGCHTEEYDYWGQGTQVTVSS

SEQ ID NO: 754 - protein, 125 aa, synthetic construct
  REGION 1..125: note = Description of Artificial Sequence: Synthetic polypeptide
EVQLVESGGGLVQAGGSLRLSCAASGRTFSSYAMGWFRQAPGKEREFVAAVSWSGGSTYYADSVKGRFTISRDNARNTVYLQMNSLNPEDTAVYYCAASQDCSGPGCYTNEYDSWGQGTQVTVSS

SEQ ID NO: 755 - protein, 125 aa, synthetic construct
  REGION 1..125: note = Description of Artificial Sequence: Synthetic polypeptide
EVQLVESGGGLVQPGGSLRLSCAASGSIFSNYAMAWFRQAPEKERDFLAAVSWSGGRTYYADSVKGRFTISRDNAKNTVNLQMNSLKPEDTAVYYCAASLSCSGPGCSLEEYDYWGQGTQVTVSS

SEQ ID NO: 756 - protein, 126 aa, synthetic construct
  REGION 1..126: note = Description of Artificial Sequence: Synthetic polypeptide
EVQLVESGGGLVQAGGSLRLSCAVSGRPFSNYGMGWFRQAPGKEREFVAGITWSGGSTHYADLVKGRFTISRDNAKNTVHLQMNSLKPEDTAVYYCAAVFSGAETAYYPSTEYDYWGQGTQVTVSS

SEQ ID NO: 757 - protein, 123 aa, synthetic construct
  REGION 1..123: note = Description of Artificial Sequence: Synthetic polypeptide
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVSS

SEQ ID NO: 758 - protein, 11 aa, synthetic construct
  REGION 1..11: note = Description of Artificial Sequence: Synthetic peptide
  VARIANT 4: note = MOD_RES - Tyr or Met
  VARIANT 6: note = MOD_RES - Met or Asp
  VARIANT 9: note = MOD_RES - Ile or Leu
APNXGXYTXDF

SEQ ID NO: 759-761 - skipped (3 entries)

SEQ ID NO: 762 - protein, 6 aa, synthetic construct
  REGION 1..6: note = Description of Artificial Sequence: Synthetic peptide
  VARIANT 2: note = MOD_RES - Ala or Ser
  VARIANT 6: note = MOD_RES - Val or Leu
RXSQSX

(19) United States
(12) Patent Application Publication
(10) Pub. No.: US 2024/0150464 A1
(43) Pub. Date: May 9, 2024

(54) MATERIALS AND METHODS FOR MODULATING T CELL MEDIATED IMMUNITY

(71) Applicant: Janssen Biotech, Inc., Horsham, PA (US)

(72) Inventors: Rajkumar GANESAN, Blue Bell, PA (US); Iqbal S. GREWAL, Newtown, PA (US); Sanjaya SINGH, Blue Bell, PA (US)

(73) Assignee: Janssen Biotech, Inc., Horsham, PA (US)

(21) Appl. No.: 18/306,401

(22) Filed: Apr. 25, 2023

Related U.S. Application Data
(63) Continuation of application No. 16/869,401, filed on May 7, 2020, now Pat. No. 11,667,712.
(60) Provisional application No. 62/844,959, filed on May 8, 2019.
(60) Provisional application No. 62/844,966, filed on May 8, 2019.
(60) Provisional application No. 62/844,970, filed on May 8, 2019.
(60) Provisional application No. 62/844,976, filed on May 8, 2019.
(60) Provisional application No. 62/844,995, filed on May 8, 2019.

(51) Int. Cl. C07K 16/28; A61K 38/17

(52) U.S. Cl. CPC C07K 16/2809; A61K 38/1709; C07K 16/2866; C07K 2317/24; C07K 2317/31; C07K 2317/565

(57) ABSTRACT

Anti-TRGV9 antibodies or antigen binding fragments thereof are described. Also described are nucleic acids encoding the antibodies, compositions comprising the antibodies, methods of producing the antibodies, and methods of using the antibodies for treating or preventing diseases, such as cancer.

### CROSS-REFERENCE TO RELATED APPLICATIONS

This application claims the benefit of U.S. Ser. No. 62/844,959 filed May 8, 2019; U.S. Ser. No. 62/844,966 filed May 8, 2019; U.S. Ser. No. 62/844,970 filed May 8, 2019; U.S. Ser. No. 62/844,976 filed May 8, 2019; and U.S. Ser. No. 62/844,995 filed May 8, 2019, each of which is incorporated herein by reference in its entirety.

### FIELD

This invention relates to, among other things, anti-TRGV9 molecules, including anti-TRGV9 antibodies, anti-TRGV9/anti-cancer-associated antigen bispecific antibodies, as well as nucleic acids and expression vectors encoding the antibodies, recombinant cells containing the vectors, and compositions comprising the antibodies. Methods of making the antibodies, and methods of using the antibodies to kill cancer cells, are also provided.

### REFERENCE TO SEQUENCE LISTING SUBMITTED ELECTRONICALLY

This application contains a sequence listing, which is submitted electronically via EFS-Web as an XML file formatted sequence listing with a file name 253505_000335_SL.xml and a creation date of Jul. 28, 2023 and having a size of 838,153 bytes. The sequence listing submitted via EFS-Web is part of the specification and is herein incorporated by reference in its entirety.

### SUMMARY

In one aspect, provided herein is an antibody that binds to T Cell Receptor Gamma Variable 9 (TRGV9). In some embodiments, the antibody comprises a heavy chain variable region (VH) and a light chain variable region (VL).

In another aspect, provided herein is an antibody that binds to TRGV9, wherein the antibody comprises: (i) a VH comprising a VH complementarity determining region (CDR) 1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the antibody comprises a VH having an amino acid sequence of SEQ ID NO:34. In some embodiments, the antibody comprises a VL having an amino acid sequence of SEQ ID NO:8. In some embodiments, the antibody comprises a VH having an amino acid sequence of SEQ ID NO:34, and a VL having an amino acid sequence of SEQ ID NO:8. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the antibody comprises a VH having an amino acid sequence of SEQ ID NO:35. In some embodiments, the antibody comprises a VL having an amino acid sequence of SEQ ID NO:8. In some embodiments, the antibody comprises a VH having an amino acid sequence of SEQ ID NO:35, and a VL having an amino acid sequence of SEQ ID NO:8. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the antibody comprises a VH having an amino acid sequence of SEQ ID NO:36. In some embodiments, the antibody comprises a VL having an amino acid sequence of SEQ ID NO:8. In some embodiments, the antibody comprises a VH having an amino acid sequence of SEQ ID NO:36, and a VL having an amino acid sequence of SEQ ID NO:8. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6. In another aspect, provided herein is an antibody that binds to TRGV9, wherein the antibody comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6. In some embodiments, the antibody comprises a VH having an amino acid sequence of SEQ ID NO:65. In some embodiments, the antibody comprises a VL having an amino acid sequence of SEQ ID NO:66. In some embodiments, the antibody comprises a VH having an amino acid sequence of SEQ ID NO:65, and a VL having an amino acid sequence of SEQ ID NO:66. In some embodiments, the antibody comprises a VH having an amino acid sequence of SEQ ID NO:67. In some embodiments, the antibody comprises a VL having an amino acid sequence of SEQ ID NO:68. In some embodiments, the antibody comprises a VH having an amino acid sequence of SEQ ID NO:67, and a VL having an amino acid sequence of SEQ ID NO:68. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100; and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103. In some embodiments, the antibody comprises a VH having an amino acid sequence of SEQ ID NO:104. In some embodiments, the antibody comprises a VL having an amino acid sequence of SEQ ID NO:105. In some embodiments, the antibody comprises a VH having an amino acid sequence of SEQ ID NO:104, and a VL having an amino acid sequence of SEQ ID NO:105. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO: 109; and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In some embodiments, the antibody comprises a VH having an amino acid sequence of SEQ ID NO:113. In some embodiments, the antibody comprises a VL having an amino acid sequence of SEQ ID NO:114. In some embodiments, the antibody comprises a VH having an amino acid sequence of SEQ ID NO:113, and a VL having an amino acid sequence of SEQ ID NO:114. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119; and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In some embodiments, the antibody comprises a VH having an amino acid sequence of SEQ ID NO:123. In some embodiments, the antibody comprises a VL having an amino acid sequence of SEQ ID NO:124. In some embodiments, the antibody comprises a VH having an amino acid sequence of SEQ ID NO:123, and a VL having an amino acid sequence of SEQ ID NO:124. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In another aspect, provided herein is an antibody that binds to TRGV9, wherein the antibody comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO: 129; and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In some embodiments, the antibody comprises a VH having an amino acid sequence of SEQ ID NO:133. In some embodiments, the antibody comprises a VL having an amino acid sequence of SEQ ID NO:134. In some embodiments, the antibody comprises a VH having an amino acid sequence of SEQ ID NO:133, and a VL having an amino acid sequence of SEQ ID NO:134. In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

Also provided is a nucleic acid encoding an antibody that binds to a TRGV9 provided herein. Also provided is a vector comprising a nucleic acid encoding an antibody that binds to a TRGV9 provided herein. Also provided is a host cell comprising a vector comprising a nucleic acid encoding an antibody that binds to a TRGV9 provided herein. Also provided is a kit comprising the vector comprising a nucleic acid encoding an antibody that binds to a TRGV9 provided herein, and packaging for the same.

Also provided is a pharmaceutical composition comprising an antibody that binds to a TRGV9 provided herein, and a pharmaceutically acceptable carrier. Also provided is a method of producing the pharmaceutical composition, comprising combining the antibody with a pharmaceutically acceptable carrier to obtain the pharmaceutical composition.

Also provided is a method of activating a γδ T cell expressing TRGV9, comprising contacting the γδ T cell with an antibody that binds to a TRGV9 provided herein. In some embodiments, the contacting results in an increase in CD69, CD25, and/or Granzyme B expression, as compared to a control γδ T cell expressing TRGV9. Also provided is a method of inactivating a γδ T cell expressing TRGV9, comprising contacting the γδ T cell with an antibody that binds to a TRGV9 provided herein. Also provided is a method of blocking activation a γδ T cell expressing TRGV9, comprising contacting the γδ T cell with an antibody that binds to a TRGV9 provided herein. Also provided is a method of modulating the activity of a γδ T cell expressing TRGV9, comprising contacting the γδ T cell with an antibody that binds to a TRGV9 provided herein.

In another aspect, provided herein is a bispecific antibody comprising: (a) a first binding domain that binds to TRGV9, and (b) a second binding domain that binds to a second target that is not TRGV9. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain comprises a VH having an amino acid sequence of SEQ ID NO:7. In some embodiments, the first binding domain comprises a VL having an amino acid sequence of SEQ ID NO:8. In some embodiments, the first binding domain comprises a VH having an amino acid sequence of SEQ ID NO:7, and a VL having an amino acid sequence of SEQ ID NO:8. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain comprises a VH having an amino acid sequence of SEQ ID NO:34. In some embodiments, the first binding domain comprises a VL having an amino acid sequence of SEQ ID NO:8. In some embodiments, the first binding domain comprises a VH having an amino acid sequence of SEQ ID NO:34, and a VL having an amino acid sequence of SEQ ID NO:8. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain comprises a VH having an amino acid sequence of SEQ ID NO:35. In some embodiments, the first binding domain comprises a VL having an amino acid sequence of SEQ ID NO:8. In some embodiments, the first binding domain comprises a VH having an amino acid sequence of SEQ ID NO:35, and a VL having an amino acid sequence of SEQ ID NO:8. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In some embodiments, the first binding domain comprises a VH having an amino acid sequence of SEQ ID NO:36. In some embodiments, the first binding domain comprises a VL having an amino acid sequence of SEQ ID NO:8. In some embodiments, the first binding domain comprises a VH having an amino acid sequence of SEQ ID NO:36, and a VL having an amino acid sequence of SEQ ID NO:8. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6. In some embodiments, the first binding domain comprises a VH having an amino acid sequence of SEQ ID NO:65. In some embodiments, the first binding domain comprises a VL having an amino acid sequence of SEQ ID NO:66. In some embodiments, the first binding domain comprises a VH having an amino acid sequence of SEQ ID NO:65, and a VL having an amino acid sequence of SEQ ID NO:66. In some embodiments, the first binding domain comprises a VH having an amino acid sequence of SEQ ID NO:67. In some embodiments, the first binding domain comprises a VL having an amino acid sequence of SEQ ID NO:68. In some embodiments, the first binding domain a VH having an amino acid sequence of SEQ ID NO:67, and a VL having an amino acid sequence of SEQ ID NO:68. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94. In some embodiments, the first binding domain comprises a VH having an amino acid sequence of SEQ ID NO:95. In some embodiments, the first binding domain comprises a VL having an amino acid sequence of SEQ ID NO:96. In some embodiments, the first binding domain comprises a VH having an amino acid sequence of SEQ ID NO:95, and a VL having an amino acid sequence of SEQ ID NO:96. In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100, and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103. In some embodiments, the first binding domain comprises a VH having an amino acid sequence of SEQ ID NO:104. In some embodiments, the first binding domain comprises a VL having an amino acid sequence of SEQ ID NO:105. In some embodiments, the first binding domain comprises a VH having an amino acid sequence of SEQ ID NO:104, and a VL having an amino acid sequence of SEQ ID NO:105. In certain embodiments, the first binding domain of the bispecific antibody specifically binds TRGV9. In some embodiments, the TRGV9 is present on the surface of a 76 T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109, and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In some embodiments, the first binding domain comprises a VH having an amino acid sequence of SEQ ID NO:113. In some embodiments, the first binding domain comprises a VL having an amino acid sequence of SEQ ID NO:114. In some embodiments, the first binding domain comprises a VH having an amino acid sequence of SEQ ID NO:113, and a VL having an amino acid sequence of SEQ ID NO:114. In certain embodiments, the first binding domain of the bispecific antibody specifically binds TRGV9. In some embodiments, the TRGV9 is present on the surface of a 76 T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119, and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In some embodiments, the first binding domain comprises a VH having an amino acid sequence of SEQ ID NO:123. In some embodiments, the first binding domain comprises a VL having an amino acid sequence of SEQ ID NO:124. In some embodiments, the first binding domain comprises a VH having an amino acid sequence of SEQ ID NO:123, and a VL having an amino acid sequence of SEQ ID NO:124. In certain embodiments, the first binding domain of the bispecific antibody specifically binds TRGV9. In some embodiments, the TRGV9 is present on the surface of a 76 T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129, and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In some embodiments, the first binding domain comprises a VH having an amino acid sequence of SEQ ID NO:133. In some embodiments, the first binding domain comprises a VL having an amino acid sequence of SEQ ID NO:134. In some embodiments, the first binding domain comprises a VH having an amino acid sequence of SEQ ID NO:133, and a VL having an amino acid sequence of SEQ ID NO:134. In certain embodiments, the first binding domain of the bispecific antibody specifically binds TRGV9. In some embodiments, the TRGV9 is present on the surface of a 76 T cell. In some embodiments, the antibody is a humanized antibody. In certain embodiments, the antibody is an IgG antibody. In other embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to a cancer antigen present on the surface of a cancer cell. In some embodiments, the antigen on the surface of the cancer cell is a tumor-specific antigen, a tumor associated antigen, or a neoantigen. In certain embodiments, the first binding domain of the bispecific antibody specifically binds TRGV9. In some embodiments, the TRGV9 is present on the surface of a γδ T cell. In some embodiments, the cancer cell is killed when the bispecific antibody binds to the TRGV9 on the surface of the γδ T cell and the antigen on the surface of the cancer cell. In some embodiments, the first binding domain is humanized, the second binding domain is humanized, or both the first binding domain and the second binding domain are humanized. In some embodiments, the bispecific antibody is an IgG antibody. In some embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to CD123. In certain embodiments, the first binding domain of the bispecific antibody specifically binds TRGV9. In some embodiments, the TRGV9 is present on the surface of a γδ T cell. In some embodiments, the CD123 is on the surface of a cell. In certain embodiments, the TRGV9 is present on the surface of a γδ T cell, and the CD123 is on the surface of a cell. In some embodiments, the cell having the CD123 on the surface is killed when the bispecific antibody binds to the TRGV9 on the surface of the γδ T cell and the CD123 on the surface of the cell. In some embodiments, the CD123 is on the surface of a cancer cell. In certain embodiments, the TRGV9 is present on the surface of a γδ T cell, and the CD123 is on the surface of a cancer cell. In some embodiments, the cancer cell is killed when the bispecific antibody binds to the TRGV9 on the surface of the γδ T cell and the CD123 on the surface of the cell. In some embodiments, the first binding domain is humanized, the second binding domain is humanized, or both the first binding domain and the second binding domain are humanized. In some embodiments, the bispecific antibody is an IgG antibody. In some embodiments, the IgG antibody is an IgG1, IgG2, IgG3, or IgG4 antibody.

In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the CD123 is on the surface of a cell. In certain embodiments, the TRGV9 is present on the surface of a γδ T cell, and the CD123 is on the surface of a cell. In some embodiments, the cell having the CD123 on the surface is killed when the bispecific antibody binds to the TRGV9 on the surface of the γδ T cell and the CD123 on the surface of the cell. In some embodiments, the CD123 is on the surface of a cancer cell. In certain embodiments, the TRGV9 is present on the surface of a γδ T cell, and the CD123 is on the surface of a cancer cell. In some embodiments, the cancer cell is killed when the bispecific antibody binds to the TRGV9 on the surface of the γδ T cell and the CD123 on the surface of the cell. In some embodiments, the first binding domain is humanized, the second binding domain is humanized, or both the first binding domain and the second binding domain are humanized. In some embodiments, the bispecific antibody is an IgG antibody. In some embodiments, the IgG antibody is an IgG1, IgG2, IgG3, or IgG4 antibody.

In some embodiments of the various bispecific antibodies provided herein, the first binding domain that binds to TRGV9 comprises a VH CDR1, VH CDR2, VH CDR3, VL CDR1, VL CDR2 and VL CDR3 having amino acid sequences, as provided herein. In some embodiments, the first binding domain that binds to TRGV9 comprises a VH CDR1, VH CDR2 and VH CDR3 of a VH domain having an amino acid sequence, as provided herein. In some embodiments, the first binding domain that binds to TRGV9 comprises a VL CDR1, VL CDR2 and VL CDR3 of a VL domain having an amino acid sequence, as provided herein. In some embodiments, the first binding domain that binds to TRGV9 comprises a VH CDR1, VH CDR2 and VH CDR3 of a VH domain having an amino acid sequence, as provided herein; and a VL CDR1, VL CDR2 and VL CDR3 of a VL domain having an amino acid sequence, as provided herein. In some embodiments, the first binding domain that binds to TRGV9 comprises a VH domain having an amino acid sequence, as provided herein. In some embodiments, the first binding domain that binds to TRGV9 comprises a VL domain having an amino acid sequence, as provided herein. In some embodiments, the first binding domain that binds to TRGV9 comprises a VH domain having an amino acid sequence, as provided herein; and a VL domain having an amino acid sequence, as provided herein.

Also provided is a nucleic acid encoding a bispecific antibody comprising: (a) a first binding domain that binds to TRGV9, and (b) a second binding domain that binds to a second target that is not TRGV9, as provided herein. Also provided is a vector comprising a nucleic acid encoding a bispecific antibody that binds to a TRGV9 provided herein. Also provided is a host cell comprising a vector comprising a nucleic acid encoding a bispecific antibody that binds to a TRGV9 provided herein. Also provided is a kit comprising the vector comprising a nucleic acid encoding a bispecific antibody that binds to a TRGV9 provided herein, and packaging for the same.

In another aspect, provided herein is a pharmaceutical composition comprising a comprising: (a) a first binding domain that binds to TRGV9, and (b) a second binding domain that binds to a second target that is not TRGV9, and a pharmaceutically acceptable carrier. Any of the bispecific antibodies provided herein are contemplated in the pharmaceutical compositions. In certain embodiments, the second binding domain binds to CD123.

In another aspect, provided herein is a method of directing a γδ T cell expressing TRGV9 to a cancer cell, the method comprising contacting the γδ T cell with a bispecific antibody provided herein. In some embodiments, the contacting directs the γδ T cell to the cancer cell.

In another aspect, provided herein is a method of inhibiting growth or proliferation of cancer cells expressing a cancer antigen on the cell surface, the method comprising contacting the cancer cells with a bispecific antibody provided herein. In some embodiments, contacting the cancer cells with the pharmaceutical composition inhibits growth or proliferation of the cancer cells. In some embodiments, the cancer cells are in the presence of a γδ T cell expressing TRGV9 while in contact with the bispecific antibody.

In another aspect, provided herein is a method for eliminating cancer cells in a subject, comprising administering an effective amount of a bispecific antibody, as provided herein, to the subject. In some embodiments, the subject is a subject in need thereof. In some embodiments, the subject is a human.

In another aspect, provided herein is a method of activating a γδ T cell expressing TRGV9, comprising contacting the γδ T cell with the bispecific antibody, as provided herein.

Provided herein are isolated TRGV9 bispecific antibodies or antigen binding fragments thereof, the isolated TRGV9 bispecific antibody or antigen binding fragment thereof comprising:
a. a first heavy chain (HC1);
b. a second heavy chain (HC2);
c. a first light chain (LC1); and
d. a second light chain (LC2),
   wherein HC1 is associated with LC1 and HC2 is associated with LC2, and wherein HC1 comprises a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 comprises a light chain complementarity determining region 1 (LCDR1), LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8. In another embodiment, the isolated TRGV9 bispecific antibody or antigen binding fragment thereof comprises an HC1 comprising the amino acid sequence selected from SEQ ID NO:7, SEQ ID NO:34, SEQ ID NO:35, or SEQ ID NO:36, and LC1 comprises the amino acid sequence of SEQ ID NO:8.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of: SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:7, and LC1 comprises an amino acid sequence having at least 95% identity to the amino acid sequence of SEQ ID NO:8. In another embodiment, the isolated TRGV9 bispecific antibody or antigen binding fragment thereof comprises an HC1 comprising the amino acid sequence of SEQ ID NO:7, and LC1 comprises the amino acid sequence of SEQ ID NO:8.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of: SEQ ID NO:1, SEQ ID NO:2, and SEQ ID NO:31, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:34, and LC1 comprises an amino acid sequence having at least 95% identity to the amino acid sequence of SEQ ID NO:8. In another embodiment, the isolated TRGV9 bispecific antibody or antigen binding fragment thereof comprises an HC1 comprising the amino acid sequence of SEQ ID NO:34, and LC1 comprises the amino acid sequence of SEQ ID NO:8.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of: SEQ ID NO:1, SEQ ID NO:2, and SEQ ID NO:32, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:35, and LC1 comprises an amino acid sequence having at least 95% identity to the amino acid sequence of SEQ ID NO:8. In another embodiment, the isolated TRGV9 bispecific antibody or antigen binding fragment thereof comprises an HC1 comprising the amino acid sequence of SEQ ID NO:35, and LC1 comprises the amino acid sequence of SEQ ID NO:8.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of: SEQ ID NO:1, SEQ ID NO:2, and SEQ ID NO:33, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:36, and LC1 comprises an amino acid sequence having at least 95% identity to the amino acid sequence of SEQ ID NO:8. In another embodiment, the isolated TRGV9 bispecific antibody or antigen binding fragment thereof comprises an HC1 comprising the amino acid sequence of SEQ ID NO:36, and LC1 comprises the amino acid sequence of SEQ ID NO:8.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR3 comprising the amino acid sequences of SEQ ID NO:77, SEQ ID NO:5, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:65, and LC1 comprises an amino acid sequence having at least 95% identity to the amino acid sequence of SEQ ID NO:66. In another embodiment, the isolated TRGV9 bispecific antibody or antigen binding fragment thereof comprises an HC1 comprising the amino acid sequence of SEQ ID NO:65, and LC1 comprises the amino acid sequence of SEQ ID NO:66. In one embodiment, the isolated TRGV9 bispecific antibody or antigen binding fragment thereof comprises an HC1 comprising an amino acid sequence having at least 95% identity to an amino acid sequence of SEQ ID NO:67, and LC1 comprises an amino acid sequence having at least 95% identity to the amino acid sequence of SEQ ID NO:68. In another embodiment, the isolated TRGV9 bispecific antibody or antigen binding fragment thereof comprises an HC1 comprising the amino acid sequence of SEQ ID NO:67, and LC1 comprises the amino acid sequence of SEQ ID NO:68.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR3 comprising the amino acid sequences of SEQ ID NO:63, SEQ ID NO:64, and SEQ ID NO:6, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:65, and LC1 comprises an amino acid sequence having at least 95% identity to the amino acid sequence of SEQ ID NO:66. In another embodiment, the isolated TRGV9 bispecific antibody or antigen binding fragment thereof comprises an HC1 comprising the amino acid sequence of SEQ ID NO:65, and LC1 comprises the amino acid sequence of SEQ ID NO:66. In one embodiment, the isolated TRGV9 bispecific antibody or antigen binding fragment thereof comprises an HC1 comprising an amino acid sequence having at least 95% identity to an amino acid sequence of SEQ ID NO:67, and LC1 comprises an amino acid sequence having at least 95% identity to the amino acid sequence of SEQ ID NO:68. In another embodiment, the isolated TRGV9 bispecific antibody or antigen binding fragment thereof comprises an HC1 comprising the amino acid sequence of SEQ ID NO:67, and LC1 comprises the amino acid sequence of SEQ ID NO:68.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR3 comprising the amino acid sequences of SEQ ID NO:92, SEQ ID NO:93, and SEQ ID NO:94, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:95, and LC1 comprises an amino acid sequence having at least 95% identity to the amino acid sequence of SEQ ID NO:96. In another embodiment, the isolated TRGV9 bispecific antibody or antigen binding fragment thereof comprises an HC1 comprising the amino acid sequence of SEQ ID NO:95, and LC1 comprises the amino acid sequence of SEQ ID NO:96.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR3 comprising the amino acid sequences of SEQ ID NO:101, SEQ ID NO:102, and SEQ ID NO:103, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:104, and LC1 comprises an amino acid sequence having at least 95% identity to the amino acid sequence of SEQ ID NO:105. In another embodiment, the isolated TRGV9 bispecific antibody or antigen binding fragment thereof comprises an HC1 comprising the amino acid sequence of SEQ ID NO:104, and LC1 comprises the amino acid sequence of SEQ ID NO:105.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR3 comprising the amino acid sequences of SEQ ID NO:110, SEQ ID NO:111, and SEQ ID NO:112, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:113, and LC1 comprises an amino acid sequence having at least 95% identity to the amino acid sequence of SEQ ID NO:114. In another embodiment, the isolated TRGV9 bispecific antibody or antigen binding fragment thereof comprises an HC1 comprising the amino acid sequence of SEQ ID NO:113, and LC1 comprises the amino acid sequence of SEQ ID NO:114.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:117, SEQ ID NO:118, and SEQ ID NO:119, respectively, and LC1 comprises a LCDR1, LCDR2, and LCDR3 comprising the amino acid sequences of SEQ ID NO:120, SEQ ID NO:121, and SEQ ID NO:122, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:123, and LC1 comprises an amino acid sequence having at least 95% identity to the amino acid sequence of SEQ ID NO:124. In another embodiment, the isolated TRGV9 bispecific antibody or antigen binding fragment thereof comprises an HC1 comprising the amino acid sequence of SEQ ID NO:123, and LC1 comprises the amino acid sequence of SEQ ID NO:124.

Also provided herein are isolated TRGV9 bispecific antibodies or antigen binding fragments thereof, the isolated TRGV9 bispecific antibody or antigen binding fragment thereof comprising: (a) a HC1; (b) a HC2; (c) a LC1; and (d) a LC2, wherein HC1 is associated with LC1 and HC2 is associated with LC2, and wherein HC1 is associated with LC1 and HC2 is associated with LC2, and wherein HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR3 comprising the amino acid sequences of SEQ ID NO:130, SEQ ID NO:131, and SEQ ID NO:132, respectively, to form a binding site for a first antigen, and wherein HC2 and LC2 form a binding site for a second antigen. In one embodiment, the isolated TRGV9 bispecific antibody or antigen binding fragment thereof comprises an HC1 comprising an amino acid sequence having at least 95% identity to an amino acid sequence of SEQ ID NO:133, and LC1 comprises an amino acid sequence having at least 95% identity to the amino acid sequence of SEQ ID NO:134. In another embodiment, the isolated TRGV9 bispecific antibody or antigen binding fragment thereof comprises an HC1 comprising the amino acid sequence of SEQ ID NO:133, and LC1 comprises the amino acid sequence of SEQ ID NO:134.

In another embodiment, the binding site for a first antigen binds to TRGV9 on a γδ T cell.

In another embodiment, the binding site for a second antigen binds to a cancer antigen present on the surface of a cancer cell.

In another embodiment, the bispecific antibody binds to TRGV9 present on the surface of the γδ T cell and the binding of the cancer antigen present on the surface of the cancer cell results in the killing of the cancer cell.

In another embodiment, TRGV9 bispecific antibody comprises a humanized HC1 and a humanized LC1.

In another embodiment, the HC2 and LC2 of the TRGV9 antibody bind to CD123.

In another embodiment the bispecific antibody or antigen binding fragment thereof is an IgG1, an IgG2, an IgG3, or an IgG4 isotype.

In a specific embodiment, the bispecific antibody or antigen binding fragment thereof is an IgG4 isotype.

In another embodiment, the TRGV9 bispecific antibody or antigen binding fragment thereof induces γδ T cell dependent cytotoxicity of a cancer cell in vitro with an EC₅₀ of less than about 500 pM.

In another embodiment, the TRGV9 bispecific antibody or antigen binding fragment thereof induces γδ T cell dependent cytotoxicity of a cancer cell in vitro with an EC₅₀ of less than about 300 pM.

In another embodiment, the TRGV9 bispecific antibody or antigen binding fragment thereof induces γδ T cell dependent cytotoxicity of a cancer cell in vitro with an EC₅₀ of less than about 160 pM.

In one embodiment, the EC₅₀ is assessed with a mixture of γδ T effector cells and Kasumi3 AML target cells.

In another embodiment, effector cell to target cell ratio is about 0.01 to 1 to about 5 to 1.

In yet another embodiment, the effector cell to target cell ratio is about 0.1 to 1 to about 2 to 1.

In a specific embodiment, the effector cell to target cell ratio is about 1:1.

In another embodiment, the TRGV9 bispecific antibody or antigen binding fragment thereof is multivalent.

In another embodiment, the TRGV9 bispecific antibody or antigen binding fragment thereof is capable of binding at least three antigens.

In another embodiment, the TRGV9 bispecific antibody or antigen binding fragment thereof is capable of binding at least five antigens.

Also provided are isolated γδ T cell bispecific antibodies or antigen binding fragments thereof, the isolated γδ T cell bispecific antibody or antigen binding fragment thereof comprising:
a. a HC1;
b. a HC2;
c. a LC1; and
d. a LC2,
   wherein HC1 is associated with LC1 and HC2 is associated with LC2,
   wherein HC1 and LC1 form a binding site for a first antigen on a γδ T cell, and
   wherein HC2 and LC2 form a binding site for a second antigen.

Also provided herein are bispecific antibodies comprising: a first means capable of specifically binding a T cell receptor gamma chain; and a second means capable of specifically binding a target molecule that is not a T cell receptor gamma chain.

Also provided are processes for making a molecule capable of specifically binding to more than one target molecule, the molecule comprising: a step for performing a function of obtaining an oligopeptide or polypeptide capable of binding to a T cell receptor gamma chain; a step for performing a function of obtaining an oligopeptide or polypeptide capable of binding to a target; and a step for performing a function of providing a molecule capable of specifically binding to a T cell receptor gamma chain and a target molecule.

In one embodiment, the step in the process for performing a function of obtaining an oligopeptide or polypeptide capable of binding to a target is repeated n times and further comprising n steps for performing a function of providing a molecule capable of specifically binding to a T cell receptor gamma chain and n number of target molecules, wherein n is at least 2.

Provided herein are isolated anti-TRGV9/anti-CD123 bispecific antibodies or antigen binding fragments thereof comprising:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8. In another embodiment, the isolated anti-TRGV9/anti-CD123 bispecific antibody or antigen binding fragment comprises an HC1 comprising the amino acid sequence selected from SEQ ID NO:7, SEQ ID NO:34, SEQ ID NO:35, or SEQ ID NO:36, and LC1 comprises the amino acid sequence of SEQ ID NO:8. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:7, and LC1 comprises an amino acid sequence having at least 95% identity to the amino acid sequence of SEQ ID NO:8. In another embodiment, the isolated anti-TRGV9/anti-CD123 bispecific antibody or antigen binding fragment comprises an HC1 comprising the amino acid sequence of SEQ ID NO:7, and LC1 comprises the amino acid sequence of SEQ ID NO:8. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:31, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:34, and LC1 comprises an amino acid sequence having at least 95% identity to the amino acid sequence of SEQ ID NO:8. In another embodiment, the isolated anti-TRGV9/anti-CD123 bispecific antibody or antigen binding fragment comprises an HC1 comprising the amino acid sequence of SEQ ID NO:34, and LC1 comprises the amino acid sequence of SEQ ID NO:8. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:32, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:35, and LC1 comprises an amino acid sequence having at least 95% identity to the amino acid sequence of SEQ ID NO:8. In another embodiment, the isolated anti-TRGV9/anti-CD123 bispecific antibody or antigen binding fragment comprises an HC1 comprising the amino acid sequence of SEQ ID NO:35, and LC1 comprises the amino acid sequence of SEQ ID NO:8. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:33, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:36, and LC1 comprises an amino acid sequence having at least 95% identity to the amino acid sequence of SEQ ID NO:8. In another embodiment, the isolated anti-TRGV9/anti-CD123 bispecific antibody or antigen binding fragment comprises an HC1 comprising the amino acid sequence of SEQ ID NO:36, and LC1 comprises the amino acid sequence of SEQ ID NO:8. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR3 comprising the amino acid sequences of SEQ ID NO:77, SEQ ID NO:5, and SEQ ID NO:6, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:65, and LC1 comprises an amino acid sequence having at least 95% identity to the amino acid sequence of SEQ ID NO:66. In another embodiment, the isolated anti-TRGV9/anti-CD123 bispecific antibody or antigen binding fragment comprises an HC1 comprising the amino acid sequence of SEQ ID NO:65, and LC1 comprises the amino acid sequence of SEQ ID NO:66. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:67, and LC1 comprises an amino acid sequence having at least 95% identity to the amino acid sequence of SEQ ID NO:68. In another embodiment, the isolated anti-TRGV9/anti-CD123 bispecific antibody or antigen binding fragment comprises an HC1 comprising the amino acid sequence of SEQ ID NO:67, and LC1 comprises the amino acid sequence of SEQ ID NO:68. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR3 comprising the amino acid sequences of SEQ ID NO:63, SEQ ID NO:64, and SEQ ID NO:65, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO: 12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:65, and LC1 comprises an amino acid sequence having at least 95% identity to the amino acid sequence of SEQ ID NO:66. In another embodiment, the isolated anti-TRGV9/anti-CD123 bispecific antibody or antigen binding fragment comprises an HC1 comprising the amino acid sequence of SEQ ID NO:65, and LC1 comprises the amino acid sequence of SEQ ID NO:66. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:67, and LC1 comprises an amino acid sequence having at least 95% identity to the amino acid sequence of SEQ ID NO:68. In another embodiment, the isolated anti-TRGV9/anti-CD123 bispecific antibody or antigen binding fragment comprises an HC1 comprising the amino acid sequence of SEQ ID NO:67, and LC1 comprises the amino acid sequence of SEQ ID NO:68. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR3 comprising the amino acid sequences of SEQ ID NO:92, SEQ ID NO:93, and SEQ ID NO:94, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:95, and LC1 comprises an amino acid sequence having at least 95% identity to the amino acid sequence of SEQ ID NO:96. In another embodiment, the isolated anti-TRGV9/anti-CD123 bispecific antibody or antigen binding fragment comprises an HC1 comprising the amino acid sequence of SEQ ID NO:95, and LC1 comprises the amino acid sequence of SEQ ID NO:96. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR3 comprising the amino acid sequences of SEQ ID NO:101, SEQ ID NO:102, and SEQ ID NO:103, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:104, and LC1 comprises an amino acid sequence having at least 95% identity to the amino acid sequence of SEQ ID NO:105. In another embodiment, the isolated anti-TRGV9/anti-CD123 bispecific antibody or antigen binding fragment comprises an HC1 comprising the amino acid sequence of SEQ ID NO:104, and LC1 comprises the amino acid sequence of SEQ ID NO:105. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR3 comprising the amino acid sequences of SEQ ID NO:110, SEQ ID NO:111, and SEQ ID NO:112, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO: 12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:113, and LC1 comprises an amino acid sequence having at least 95% identity to the amino acid sequence of SEQ ID NO:114. In another embodiment, the isolated anti-TRGV9/anti-CD123 bispecific antibody or antigen binding fragment comprises an HC1 comprising the amino acid sequence of SEQ ID NO:113, and LC1 comprises the amino acid sequence of SEQ ID NO:114. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO: 117, SEQ ID NO: 118, and SEQ ID NO: 119, respectively, and LC1 comprises a LCDR1, LCDR2, and LCDR3 comprising the amino acid sequences of SEQ ID NO:120, SEQ ID NO:121, and SEQ ID NO:122, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:123, and LC1 comprises an amino acid sequence having at least 95% identity to the amino acid sequence of SEQ ID NO:124. In another embodiment, the isolated anti-TRGV9/anti-CD123 bispecific antibody or antigen binding fragment comprises an HC1 comprising the amino acid sequence of SEQ ID NO:123, and LC1 comprises the amino acid sequence of SEQ ID NO:124. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

Also provided herein are isolated anti-TRGV9/anti-CD123 bispecific antibodies or antigen binding fragments thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR3 comprising the amino acid sequences of SEQ ID NO:130, SEQ ID NO:131, and SEQ ID NO:132, respectively, to form a binding site for a first antigen that specifically binds Vγ9, and wherein HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In one embodiment, the isolated anti-TRGV9/anti-CD123 bispecific antibody or antigen binding fragment comprises an HC1 comprising an amino acid sequence having at least 95% identity to an amino acid sequence selected of SEQ ID NO:133, and LC1 comprises an amino acid sequence having at least 95% identity to the amino acid sequence of SEQ ID NO:134. In another embodiment, the isolated anti-TRGV9/anti-CD123 bispecific antibody or antigen binding fragment comprises an HC1 comprising the amino acid sequence of SEQ ID NO:133, and LC1 comprises the amino acid sequence of SEQ ID NO:134. In another embodiment, the isolated anti-TRGV9/anti-CD123 bispecific antibody or antigen binding fragment comprises an HC2 comprising an amino acid sequence having at least 95% identity to the amino acid sequence of SEQ ID NO:15 and LC2 comprises an amino acid sequence having at least 95% identity to the amino acid sequence of SEQ ID NO:16. In another embodiment, the isolated anti-TRGV9/anti-CD123 bispecific antibody or antigen binding fragment comprises an HC2 comprising the amino acid sequence of SEQ ID NO:15 and LC2 comprises the amino acid sequence of SEQ ID NO:16.

In another embodiment, the TRGV9 is on the surface of a γδ T cell.

In another embodiment, the CD123 is on the surface of a tumor cell or a CD34+ stem cell.

In another embodiment, the binding of the bispecific antibody to TRGV9 present on the surface of the γδ T cell and the binding of the CD123 on the surface of the cancer cell results in the killing of the cancer cell.

In another embodiment, the isolated anti-TRGV9/anti-CD123 bispecific antibody or antigen binding fragment thereof comprises a humanized HC1 and a humanized LC1.

In another embodiment, the isolated anti-TRGV9/anti-CD123 bispecific antibody or antigen binding fragment comprises a humanized HC2 and a humanized LC2.

In another embodiment, the isolated anti-TRGV9/anti-CD123 bispecific antibody or antigen binding fragment thereof is an IgG1, an IgG2, an IgG3, or an IgG4 isotype. In a specific embodiment, the bispecific antibody is an IgG4 isotype.

In another embodiment, the isolated anti-TRGV9/anti-CD123 bispecific antibody or antigen binding fragment thereof induces γδ T cell dependent cytotoxicity of a cancer cell in vitro with an EC₅₀ of less than about 500 pM.

In another embodiment, the isolated anti-TRGV9/anti-CD123 bispecific antibody or antigen binding fragment thereof induces γδ T cell dependent cytotoxicity of a cancer cell in vitro with an EC₅₀ of less than about 300 pM.

In another embodiment, the isolated anti-TRGV9/anti-CD123 bispecific antibody or antigen binding fragment thereof induces γδ T cell dependent cytotoxicity of a cancer cell in vitro with an EC₅₀ of less than about 160 pM.

In one embodiment, the EC₅₀ is assessed with a mixture of γδ T effector cells and Kasumi3 AML target cells.

In another embodiment, the effector cell to target cell ratio is about 0.01 to 1 to about 5 to 1.

In yet another embodiment, the effector cell to target cell ratio is about 0.1 to 1 to about 2 to 1.

In a specific embodiment, the effector cell to target cell ratio is about 1:1.

Also provided are methods of making the isolated anti-TRGV9/anti-CD123 bispecific antibody or antigen binding fragment provided herein, the method comprising culturing a cell comprising a nucleic acid encoding the anti-TRGV9/anti-CD123 bispecific antibody or antigen binding fragment thereof under conditions to produce the bispecific antibody or antigen binding fragment thereof and recovering the bispecific antibody or antigen binding fragment thereof.

In another aspect, provided herein are isolated TRGV9 bispecific antibodies or antigen epitope binding fragments thereof, wherein the isolated TRGV9 bispecific antibodies or antigen epitope binding fragments thereof comprise a binding site for a first antigen and a binding site for a second antigen, wherein the binding site for the first antigen binds a TRGV9 epitope on a γδ T cell and the binding site for the second antigen binds an epitope of the second antigen on a surface of a target cell, and the binding of the TRGV9 epitope on the γδ T cell and the binding of the second antigen epitope on the target cell results in the killing of the target cell.

In one embodiment, the TRGV9 bispecific antibodies or antigen binding fragments comprise:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 comprises a LCDR1, LCDR2, and LCDR2 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence selected from SEQ ID NO: 7, SEQ ID NO:34, SEQ ID NO:35, or SEQ ID NO:36, and LC1 comprises an amino acid sequence having at least 95% identity to the amino acid sequence of SEQ ID NO:8. In another embodiment, the TRGV9 bispecific antibodies or antigen binding fragments comprise an HC1 comprising the amino acid sequence selected from SEQ ID NO:7, SEQ ID NO:34, SEQ ID NO:35, or SEQ ID NO:36, and LC1 comprises the amino acid sequence of SEQ ID NO:8.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR2 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:7, and LC1 comprises an amino acid sequence having at least 95% identity to the amino acid sequence of SEQ ID NO:8. In another embodiment, the TRGV9 bispecific antibodies or antigen binding fragments comprise an HC1 comprising the amino acid sequence of SEQ ID NO:7, and LC1 comprises the amino acid sequence of SEQ ID NO:8.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:31, respectively, and LC1 comprises a LCDR1, LCDR2, and LCDR2 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:34, and LC1 comprises an amino acid sequence having at least 95% identity to the amino acid sequence of SEQ ID NO:8. In another embodiment, the TRGV9 bispecific antibodies or antigen binding fragments comprise an HC1 comprising the amino acid sequence of SEQ ID NO:34, and LC1 comprises the amino acid sequence of SEQ ID NO:8.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:32, respectively, and LC1 comprises a LCDR1, LCDR2, and LCDR2 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:35, and LC1 comprises an amino acid sequence having at least 95% identity to the amino acid sequence of SEQ ID NO:8. In another embodiment, the TRGV9 bispecific antibodies or antigen binding fragments comprise an HC1 comprising the amino acid sequence of SEQ ID NO:35, and LC1 comprises the amino acid sequence of SEQ ID NO:8.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:33, respectively, and LC1 comprises a LCDR1, LCDR2, and LCDR2 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:36, and LC1 comprises an amino acid sequence having at least 95% identity to the amino acid sequence of SEQ ID NO:8. In another embodiment, the TRGV9 bispecific antibodies or antigen binding fragments comprise an HC1 comprising the amino acid sequence of SEQ ID NO:36, and LC1 comprises the amino acid sequence of SEQ ID NO:8.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR2 comprising the amino acid sequences of SEQ ID NO:77, SEQ ID NO:5, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:65, and LC1 comprises an amino acid sequence having at least 95% identity to the amino acid sequence of SEQ ID NO:66. In another embodiment, the TRGV9 bispecific antibodies or antigen binding fragments comprise an HC1 comprising the amino acid sequence of SEQ ID NO:65, and LC1 comprises the amino acid sequence of SEQ ID NO:66. In another embodiment, the TRGV9 bispecific antibodies or antigen binding fragments comprise an HC1 comprising an amino acid sequence having at least 95% identity to an amino acid sequence of SEQ ID NO:67, and LC1 comprises an amino acid sequence having at least 95% identity to the amino acid sequence of SEQ ID NO:68. In another embodiment, the TRGV9 bispecific antibodies or antigen binding fragments comprise an HC1 comprising the amino acid sequence of SEQ ID NO:67, and LC1 comprises the amino acid sequence of SEQ ID NO:68.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR2 comprising the amino acid sequences of SEQ ID NO:63, SEQ ID NO:64, and SEQ ID NO:6, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:65, and LC1 comprises an amino acid sequence having at least 95% identity to the amino acid sequence of SEQ ID NO:66. In another embodiment, the TRGV9 bispecific antibodies or antigen binding fragments comprise an HC1 comprising the amino acid sequence of SEQ ID NO:65, and LC1 comprises the amino acid sequence of SEQ ID NO:66. In another embodiment, the TRGV9 bispecific antibodies or antigen binding fragments comprise an HC1 comprising an amino acid sequence having at least 95% identity to an amino acid sequence of SEQ ID NO:67, and LC1 comprises an amino acid sequence having at least 95% identity to the amino acid sequence of SEQ ID NO:68. In another embodiment, the TRGV9 bispecific antibodies or antigen binding fragments comprise an HC1 comprising the amino acid sequence of SEQ ID NO:67, and LC1 comprises the amino acid sequence of SEQ ID NO:68.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR2 comprising the amino acid sequences of SEQ ID NO:92, SEQ ID NO:93, and SEQ ID NO:94, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:95, and LC1 comprises an amino acid sequence having at least 95% identity to the amino acid sequence of SEQ ID NO:96. In another embodiment, the TRGV9 bispecific antibodies or antigen binding fragments comprise an HC1 comprising the amino acid sequence of SEQ ID NO:95, and LC1 comprises the amino acid sequence of SEQ ID NO:96.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR2 comprising the amino acid sequences of SEQ ID NO:101, SEQ ID NO:102, and SEQ ID NO:103, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:104, and LC1 comprises an amino acid sequence having at least 95% identity to the amino acid sequence of SEQ ID NO:105. In another embodiment, the TRGV9 bispecific antibodies or antigen binding fragments comprise an HC1 comprising the amino acid sequence of SEQ ID NO:104, and LC1 comprises the amino acid sequence of SEQ ID NO:105.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR2 comprising the amino acid sequences of SEQ ID NO:110, SEQ ID NO:111, and SEQ ID NO:112, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:113, and LC1 comprises an amino acid sequence having at least 95% identity to the amino acid sequence of SEQ ID NO:114. In another embodiment, the TRGV9 bispecific antibodies or antigen binding fragments comprise an HC1 comprising the amino acid sequence of SEQ ID NO:113, and LC1 comprises the amino acid sequence of SEQ ID NO:114.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:117, SEQ ID NO:118, and SEQ ID NO:119, respectively, and LC1 comprises a LCDR1, LCDR2, and LCDR2 comprising the amino acid sequences of SEQ ID NO:120, SEQ ID NO:121, and SEQ ID NO:122, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:123, and LC1 comprises an amino acid sequence having at least 95% identity to the amino acid sequence of SEQ ID NO:124. In another embodiment, the TRGV9 bispecific antibodies or antigen binding fragments comprise an HC1 comprising the amino acid sequence of SEQ ID NO:123, and LC1 comprises the amino acid sequence of SEQ ID NO:124.

In one embodiment, the TRGV9 bispecific antibodies or antigen binding fragments comprise: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR2 comprising the amino acid sequences of SEQ ID NO:130, SEQ ID NO:131, and SEQ ID NO:132, respectively, to form the binding site for the first antigen, and wherein HC2 and LC2 form the binding site for the second antigen epitope. In another embodiment, the TRGV9 bispecific antibodies or antigen binding fragments comprise an HC1 comprising an amino acid sequence having at least 95% identity to an amino acid sequence of SEQ ID NO:133, and LC1 comprises an amino acid sequence having at least 95% identity to the amino acid sequence of SEQ ID NO:134. In another embodiment, the TRGV9 bispecific antibodies or antigen binding fragments comprise an HC1 comprising the amino acid sequence of SEQ ID NO:133, and LC1 comprises the amino acid sequence of SEQ ID NO:134.

In another embodiment, the TRGV9 bispecific antibodies or antigen binding fragments comprise a humanized HC1 and a humanized LC.

In another embodiment, the TRGV9 bispecific antibodies or antigen binding fragments bind to a CD123 epitope.

In another embodiment, the TRGV9 bispecific antibodies or antigen binding fragments comprise and HC2 comprising an amino acid sequence having at least 95% identity to the amino acid sequence of SEQ ID NO:15 and LC2 comprises an amino acid sequence having at least 95% identity to the amino acid sequence of SEQ ID NO:16.

In another embodiment, the TRGV9 bispecific antibodies or antigen binding fragments comprise an HC2 comprising the amino acid sequence of SEQ ID NO:15 and LC2 comprises the amino acid sequence of SEQ ID NO:16.

In another embodiment, the TRGV9 bispecific antibodies or antigen binding fragments thereof are an IgG1, an IgG2, an IgG3, or an IgG4 isotype. In a specific embodiment, the bispecific antibodies or antigen binding fragments thereof fragment thereof are an IgG4 isotype.

In another embodiment, the TRGV9 bispecific antibodies or antigen binding fragments thereof induce γδ T cell dependent cytotoxicity of a cancer cell in vitro with an EC₅₀ of less than about 500 pM.

In another embodiment, the TRGV9 bispecific antibodies or antigen binding fragments thereof induce γδ T cell dependent cytotoxicity of a cancer cell in vitro with an EC₅₀ of less than about 300 pM.

In another embodiment, the TRGV9 bispecific antibodies or antigen binding fragments thereof induce γδ T cell dependent cytotoxicity of a cancer cell in vitro with an EC₅₀ of less than about 160 pM.

In one embodiment, the EC₅₀ is assessed with a mixture of γδ T effector cells and Kasumi3 AML target cells.

In another embodiment, the effector cell to target cell ratio is about 0.01 to 1 to about 5 to 1. In another embodiment, the effector cell to target cell ratio is about 0.1 to 1 to about 2 to 1. In a specific embodiment, the effector cell to target cell ratio is about 1:1.

Also provided are isolated γδ T cell bispecific antibodies or antigen binding fragments thereof, wherein the isolated γδ T cell bispecific antibody or antigen binding fragment thereof comprises a binding site for a first antigen epitope and a binding site for a second antigen epitope, wherein the binding site for the first antigen epitope binds a first antigen on a γδ T cell and the binding site for the second antigen epitope binds the second antigen epitope on a surface of a target cell, and the binding of the first antigen epitope on the γδ T cell and the binding of the second antigen epitope on the target cell results in the killing of the target cell.

In another aspect, provided herein are isolated nucleic acids encoding a TRGV9 bispecific antibody or antigen binding fragment thereof, the isolated TRGV9 bispecific antibody or antigen binding fragment thereof comprising:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8. In another embodiment, the isolated nucleic acid encodes a TRGV9 bispecific antibody comprising an HC1 comprising the amino acid sequence selected from SEQ ID NO:7, SEQ ID NO:34, SEQ ID NO:35, or SEQ ID NO:36, and LC1 comprises the amino acid sequence of SEQ ID NO: 8.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:7, and LC1 comprises an amino acid sequence having at least 95% identity to the amino acid sequence of SEQ ID NO:8. In another embodiment, the isolated nucleic acid encodes a TRGV9 bispecific antibody comprising an HC1 comprising the amino acid sequence of SEQ ID NO:7, and LC1 comprises the amino acid sequence of SEQ ID NO:8.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:31, respectively, and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:34, and LC1 comprises an amino acid sequence having at least 95% identity to the amino acid sequence of SEQ ID NO:8. In another embodiment, the isolated nucleic acid encodes a TRGV9 bispecific antibody comprising an HC1 comprising the amino acid sequence of SEQ ID NO:34, and LC1 comprises the amino acid sequence of SEQ ID NO:8.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:32, respectively, and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:35, and LC1 comprises an amino acid sequence having at least 95% identity to the amino acid sequence of SEQ ID NO:8. In another embodiment, the isolated nucleic acid encodes a TRGV9 bispecific antibody comprising an HC1 comprising the amino acid sequence of SEQ ID NO:35, and LC1 comprises the amino acid sequence of SEQ ID NO:8.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:33, respectively, and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:36, and LC1 comprises an amino acid sequence having at least 95% identity to the amino acid sequence of SEQ ID NO:8. In another embodiment, the isolated nucleic acid encodes a TRGV9 bispecific antibody comprising an HC1 comprising the amino acid sequence of SEQ ID NO:36, and LC1 comprises the amino acid sequence of SEQ ID NO:8.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR2 comprising the amino acid sequences of SEQ ID NO:77, SEQ ID NO:5, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:65, and LC1 comprises an amino acid sequence having at least 95% identity to the amino acid sequence of SEQ ID NO:66. In another embodiment, the isolated nucleic acid encodes a TRGV9 bispecific antibody comprising an HC1 comprising the amino acid sequence of SEQ ID NO:65, and LC1 comprises the amino acid sequence of SEQ ID NO:66. In one embodiment, the isolated nucleic acid encodes a TRGV9 bispecific antibody comprising an HC1 comprising an amino acid sequence having at least 95% identity to an amino acid sequence of SEQ ID NO:67, and LC1 comprises an amino acid sequence having at least 95% identity to the amino acid sequence of SEQ ID NO:68. In another embodiment, the isolated nucleic acid encodes a TRGV9 bispecific antibody comprising an HC1 comprising the amino acid sequence of SEQ ID NO:67, and LC1 comprises the amino acid sequence of SEQ ID NO:68.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR2 comprising the amino acid sequences of SEQ ID NO:63, SEQ ID NO:64, and SEQ ID NO:6, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:65, and LC1 comprises an amino acid sequence having at least 95% identity to the amino acid sequence of SEQ ID NO:66. In another embodiment, the isolated nucleic acid encodes a TRGV9 bispecific antibody comprising an HC1 comprising the amino acid sequence of SEQ ID NO:65, and LC1 comprises the amino acid sequence of SEQ ID NO:66. In one embodiment, the isolated nucleic acid encodes a TRGV9 bispecific antibody comprising an HC1 comprising an amino acid sequence having at least 95% identity to an amino acid sequence of SEQ ID NO:67, and LC1 comprises an amino acid sequence having at least 95% identity to the amino acid sequence of SEQ ID NO:68. In another embodiment, the isolated nucleic acid encodes a TRGV9 bispecific antibody comprising an HC1 comprising the amino acid sequence of SEQ ID NO:67, and LC1 comprises the amino acid sequence of SEQ ID NO:68.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR2 comprising the amino acid sequences of SEQ ID NO:92, SEQ ID NO:93, and SEQ ID NO:94, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:95, and LC1 comprises an amino acid sequence having at least 95% identity to the amino acid sequence of SEQ ID NO:96. In another embodiment, the isolated nucleic acid encodes a TRGV9 bispecific antibody comprising an HC1 comprising the amino acid sequence of SEQ ID NO:95, and LC1 comprises the amino acid sequence of SEQ ID NO:96.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR2 comprising the amino acid sequences of SEQ ID NO:101, SEQ ID NO: 102, and SEQ ID NO:103, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:104, and LC1 comprises an amino acid sequence having at least 95% identity to the amino acid sequence of SEQ ID NO:105. In another embodiment, the isolated nucleic acid encodes a TRGV9 bispecific antibody comprising an HC1 comprising the amino acid sequence of SEQ ID NO:104, and LC1 comprises the amino acid sequence of SEQ ID NO:105.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR2 comprising the amino acid sequences of SEQ ID NO:110, SEQ ID NO:111, and SEQ ID NO:112, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:113, and LC1 comprises an amino acid sequence having at least 95% identity to the amino acid sequence of SEQ ID NO:114. In another embodiment, the isolated nucleic acid encodes a TRGV9 bispecific antibody comprising an HC1 comprising the amino acid sequence of SEQ ID NO:113, and LC1 comprises the amino acid sequence of SEQ ID NO:114.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:117, SEQ ID NO:118, and SEQ ID NO:119, respectively, and LC1 comprises a LCDR1, LCDR2, and LCDR2 comprising the amino acid sequences of SEQ ID NO:120, SEQ ID NO:121, and SEQ ID NO:122, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:123, and LC1 comprises an amino acid sequence having at least 95% identity to the amino acid sequence of SEQ ID NO:124. In another embodiment, the isolated nucleic acid encodes a TRGV9 bispecific antibody comprising an HC1 comprising the amino acid sequence of SEQ ID NO:123, and LC1 comprises the amino acid sequence of SEQ ID NO:124.

In another aspect, provided herein are isolated nucleic acids encoding a TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR2 comprising the amino acid sequences of SEQ ID NO:130, SEQ ID NO:131, and SEQ ID NO:132, respectively, to form a binding site for a first antigen, and wherein HC2 and LC2 form a binding site for a second antigen. In one embodiment, the isolated nucleic acid encodes a TRGV9 bispecific antibody comprising an HC1 comprising an amino acid sequence having at least 95% identity to an amino acid sequence of SEQ ID NO:133, and LC1 comprises an amino acid sequence having at least 95% identity to the amino acid sequence of SEQ ID NO:134. In another embodiment, the isolated nucleic acid encodes a TRGV9 bispecific antibody comprising an HC1 comprising the amino acid sequence of SEQ ID NO:133, and LC1 comprises the amino acid sequence of SEQ ID NO:134.

In another embodiment, the isolated nucleic acid encodes a TRGV9 bispecific antibody comprising a binding site comprising a first antigen that binds to TRGV9 on a γδ T cell.

In another embodiment, the isolated nucleic acid encodes a TRGV9 bispecific antibody comprising a binding site for a second antigen that binds to a cancer antigen present on the surface of a cancer cell.

In another embodiment, the isolated nucleic acid encodes a TRGV9 bispecific antibody, wherein the binding of the bispecific antibody to TRGV9 present on the surface of the γδ T cell and the binding of the cancer antigen present on the surface of the cancer cell results in the killing of the cancer cell.

In another embodiment, the isolated nucleic acid encodes a TRGV9 bispecific antibody, wherein HC1 and LC1 are humanized.

In another embodiment, the isolated nucleic acid encodes a TRGV9 bispecific antibody, wherein HC2 and LC2 bind to CD123.

In another embodiment, the isolated nucleic acid encodes a TRGV9 bispecific antibody, wherein the bispecific antibody or antigen binding fragment thereof is an IgG1, an IgG2, an IgG3, or an IgG4 isotype.

In a specific embodiment, the bispecific antibody or antigen binding fragment thereof is an IgG4 isotype.

In another embodiment, the isolated nucleic acid encodes a TRGV9 bispecific antibody, wherein the bispecific antibody or antigen binding fragment thereof induces γδ T cell dependent cytotoxicity of a cancer cell in vitro with an EC₅₀ of less than about 500 pM.

In another embodiment, the isolated nucleic acid encodes a TRGV9 bispecific antibody, wherein the bispecific antibody or antigen binding fragment thereof induces γδ T cell dependent cytotoxicity of a cancer cell in vitro with an EC₅₀ of less than about 300 pM.

In another embodiment, the isolated nucleic acid encodes a TRGV9 bispecific antibody, wherein the bispecific antibody or antigen binding fragment thereof induces γδ T cell dependent cytotoxicity of a cancer cell in vitro with an EC₅₀ of less than about 160 pM.

In another embodiment, the isolated nucleic acid encodes a TRGV9 bispecific antibody, wherein the EC₅₀ is assessed with a mixture of γδ T effector cells and Kasumi3 AML target cells.

In another embodiment, the isolated nucleic acid encodes a TRGV9 bispecific antibody, wherein the effector cell to target cell ratio is about 0.01 to 1 to about 5 to 1. In one embodiment, the effector cell to target cell ratio is about 0.1 to 1 to about 2 to 1. In yet another embodiment, the effector cell to target cell ratio is about 1:1.

In another embodiment, the isolated nucleic acid encodes a TRGV9 bispecific antibody, wherein the bispecific antibody or antigen binding fragment thereof is multivalent.

In another embodiment, the isolated nucleic acid encodes a TRGV9 bispecific antibody, wherein the bispecific antibody or antigen binding fragment thereof is capable of binding at least three antigens.

In another embodiment, the isolated nucleic acid encodes a TRGV9 bispecific antibody, wherein the bispecific antibody or antigen binding fragment thereof is capable of binding at least five antigens.

Also provided are vectors comprising the isolated nucleic acids provided herein.

Also provided are host cells comprising the vectors provided herein.

Also provided are kits comprising the vectors provided herein and packaging for the same.

Provided herein are pharmaceutical compositions comprising: (A) an isolated TRGV9 bispecific antibody or antigen binding fragment thereof, the isolated TRGV9 bispecific antibody or antigen binding fragment thereof comprising:
a. a HC1;
b. a HC2;
c. a LC1; and
d. a LC2,
   wherein HC1 is associated with LC1 and HC2 is associated with LC2, and wherein HC1 comprises a HCDR1, HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen;
   and (B) a pharmaceutically acceptable carrier. In one embodiment, the pharmaceutical composition comprises a bispecific antibody comprising an HC1 comprising an amino acid sequence having at least 95% identity to an amino acid sequence selected from SEQ ID NO:7, SEQ ID NO:34, SEQ ID NO:35, or SEQ ID NO:36, and an LC1 comprising an amino acid sequence having at least 95% identity to the amino acid sequence of SEQ ID NO:8. In another embodiment, the pharmaceutical composition comprises a bispecific antibody comprising an HC1 comprising the amino acid sequence selected from SEQ ID NO:7, SEQ ID NO:34, SEQ ID NO:35, or SEQ ID NO:36, and an LC1 comprising the amino acid sequence of SEQ ID NO:8.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:3, respectively; and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:7, and an LC1 comprising an amino acid sequence having at least 95% identity to the amino acid sequence of SEQ ID NO:8. In another embodiment, the pharmaceutical composition comprises a bispecific antibody comprising an HC1 comprising the amino acid sequence of SEQ ID NO:7, and an LC1 comprising the amino acid sequence of SEQ ID NO:8.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:31, respectively; and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:34, and an LC1 comprising an amino acid sequence having at least 95% identity to the amino acid sequence of SEQ ID NO:8. In another embodiment, the pharmaceutical composition comprises a bispecific antibody comprising an HC1 comprising the amino acid sequence of SEQ ID NO:34, and an LC1 comprising the amino acid sequence of SEQ ID NO:8.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:32, respectively; and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:35, and an LC1 comprising an amino acid sequence having at least 95% identity to the amino acid sequence of SEQ ID NO:8. In another embodiment, the pharmaceutical composition comprises a bispecific antibody comprising an HC1 comprising the amino acid sequence of SEQ ID NO:35, and an LC1 comprising the amino acid sequence of SEQ ID NO:8.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:33, respectively; and LC1 comprises a LCDR1, LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:36, and an LC1 comprising an amino acid sequence having at least 95% identity to the amino acid sequence of SEQ ID NO:8. In another embodiment, the pharmaceutical composition comprises a bispecific antibody comprising an HC1 comprising the amino acid sequence of SEQ ID NO:36, and an LC1 comprising the amino acid sequence of SEQ ID NO:8.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:76, and SEQ ID NO:3, respectively; and LC1 comprises a LCDR1, LCDR2, and LCDR2 comprising the amino acid sequences of SEQ ID NO:77, SEQ ID NO:5, and SEQ ID NO:6, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:65, and an LC1 comprising an amino acid sequence having at least 95% identity to the amino acid sequence of SEQ ID NO:66. In another embodiment, the pharmaceutical composition comprises a bispecific antibody comprising an HC1 comprising the amino acid sequence of SEQ ID NO:65, and an LC1 comprising the amino acid sequence of SEQ ID NO:66. In one embodiment, the pharmaceutical composition comprises a bispecific antibody comprising an HC1 comprising an amino acid sequence having at least 95% identity to an amino acid sequence of SEQ ID NO:67, and an LC1 comprising an amino acid sequence having at least 95% identity to the amino acid sequence of SEQ ID NO:68. In another embodiment, the pharmaceutical composition comprises a bispecific antibody comprising an HC1 comprising the amino acid sequence of SEQ ID NO:67, and an LC1 comprising the amino acid sequence of SEQ ID NO:68.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:60, SEQ ID NO:61, and SEQ ID NO:62, respectively; and LC1 comprises a LCDR1, LCDR2, and LCDR2 comprising the amino acid sequences of SEQ ID NO:63, SEQ ID NO:64, and SEQ ID NO:6, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:65, and an LC1 comprising an amino acid sequence having at least 95% identity to the amino acid sequence of SEQ ID NO:66. In another embodiment, the pharmaceutical composition comprises a bispecific antibody comprising an HC1 comprising the amino acid sequence of SEQ ID NO:65, and an LC1 comprising the amino acid sequence of SEQ ID NO:66. In one embodiment, the pharmaceutical composition comprises a bispecific antibody comprising an HC1 comprising an amino acid sequence having at least 95% identity to an amino acid sequence of SEQ ID NO:67, and an LC1 comprising an amino acid sequence having at least 95% identity to the amino acid sequence of SEQ ID NO:68. In another embodiment, the pharmaceutical composition comprises a bispecific antibody comprising an HC1 comprising the amino acid sequence of SEQ ID NO:67, and an LC1 comprising the amino acid sequence of SEQ ID NO:68.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:89, SEQ ID NO:90, and SEQ ID NO:91, respectively; and LC1 comprises a LCDR1, LCDR2, and LCDR2 comprising the amino acid sequences of SEQ ID NO:92, SEQ ID NO:93, and SEQ ID NO:94, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:95, and an LC1 comprising an amino acid sequence having at least 95% identity to the amino acid sequence of SEQ ID NO:96. In another embodiment, the pharmaceutical composition comprises a bispecific antibody comprising an HC1 comprising the amino acid sequence of SEQ ID NO:95, and an LC1 comprising the amino acid sequence of SEQ ID NO:96.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:98, SEQ ID NO:99, and SEQ ID NO:100, respectively; and LC1 comprises a LCDR1, LCDR2, and LCDR2 comprising the amino acid sequences of SEQ ID NO:101, SEQ ID NO: 102, and SEQ ID NO:103, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:104, and an LC1 comprising an amino acid sequence having at least 95% identity to the amino acid sequence of SEQ ID NO:105. In another embodiment, the pharmaceutical composition comprises a bispecific antibody comprising an HC1 comprising the amino acid sequence of SEQ ID NO:104, and an LC1 comprising the amino acid sequence of SEQ ID NO:105.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:107, SEQ ID NO:108, and SEQ ID NO:109, respectively; and LC1 comprises a LCDR1, LCDR2, and LCDR2 comprising the amino acid sequences of SEQ ID NO:110, SEQ ID NO:111, and SEQ ID NO:112, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:113, and an LC1 comprising an amino acid sequence having at least 95% identity to the amino acid sequence of SEQ ID NO:114. In another embodiment, the pharmaceutical composition comprises a bispecific antibody comprising an HC1 comprising the amino acid sequence of SEQ ID NO:113, and an LC1 comprising the amino acid sequence of SEQ ID NO:114.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:117, SEQ ID NO:118, and SEQ ID NO:119, respectively; and LC1 comprises a LCDR1, LCDR2, and LCDR2 comprising the amino acid sequences of SEQ ID NO:120, SEQ ID NO:121, and SEQ ID NO:122, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:123, and an LC1 comprising an amino acid sequence having at least 95% identity to the amino acid sequence of SEQ ID NO:124. In another embodiment, the pharmaceutical composition comprises a bispecific antibody comprising an HC1 comprising the amino acid sequence of SEQ ID NO:123, and an LC1 comprising the amino acid sequence of SEQ ID NO:124.

Also provided herein are pharmaceutical compositions comprising: (A) an isolated TRGV9 bispecific antibody or antigen binding fragment thereof, the isolated TRGV9 bispecific antibody or antigen binding fragment thereof comprising: (a) a HC1; (b) a HC2; (c) a LC1; and (d) a LC2; wherein HC1 is associated with LC1 and HC2 is associated with LC2, and wherein HC1 comprises a HCDR1, HCDR2, and HCDR3 comprising the amino acid sequences of SEQ ID NO:127, SEQ ID NO:128, and SEQ ID NO:129, respectively; and LC1 comprises a LCDR1, LCDR2, and LCDR2 comprising the amino acid sequences of SEQ ID NO:130, SEQ ID NO:131, and SEQ ID NO:132, respectively, to form a binding site for a first antigen, and wherein HC2 and LC2 form a binding site for a second antigen; and (B) a pharmaceutically acceptable carrier. In one embodiment, the pharmaceutical composition comprises a bispecific antibody comprising an HC1 comprising an amino acid sequence having at least 95% identity to an amino acid sequence of SEQ ID NO:133, and an LC1 comprising an amino acid sequence having at least 95% identity to the amino acid sequence of SEQ ID NO:134. In another embodiment, the pharmaceutical composition comprises a bispecific antibody comprising an HC1 comprising the amino acid sequence of SEQ ID NO:133, and an LC1 comprising the amino acid sequence of SEQ ID NO:134.

In another embodiment, the pharmaceutical composition comprises a bispecific antibody comprising a binding site for a first antigen binds to TRGV9 on a γδ T cell.

In another embodiment, the pharmaceutical composition comprises a bispecific antibody, wherein the binding site for a second antigen binds to a cancer antigen present on the surface of a cancer cell.

In another embodiment, the pharmaceutical composition comprises a bispecific antibody, wherein the binding of the bispecific antibody to TRGV9 present on the surface of the γδ T cell and the binding of the cancer antigen present on the surface of the cancer cell results in the killing of the cancer cell.

In another embodiment, the pharmaceutical composition comprises a bispecific antibody, wherein HC1 and LC1 are humanized.

In another embodiment, the pharmaceutical composition comprises a bispecific antibody, wherein HC2 and LC2 bind to CD123.

In another embodiment, the pharmaceutical composition comprises a bispecific antibody, wherein the bispecific antibody or antigen binding fragment thereof is an IgG1, an IgG2, an IgG3, or an IgG4 isotype.

Also provided are methods of directing a Vγ9-expressing γδ T cell to a cancer cell, the method comprising contacting a Vγ9-expressing γδ T cell with the pharmaceutical compositions provided herein, wherein contacting the Vγ9-expressing γδ T cell with the pharmaceutical composition directs the Vγ9-expressing γδ T cell to a cancer cell.

Also provided are methods of inhibiting growth or proliferation of cancer cells expressing a cancer antigen on the cell surface, the method comprising contacting the cancer cells with the pharmaceutical compositions provided herein, wherein contacting the cancer cells with the pharmaceutical composition inhibits growth or proliferation of the cancer cells.

In one embodiment, the cancer cell is in the presence of a Vγ9-expressing γδ T cell while in contact with anti-TRGV9 bispecific antibody or antigen binding fragment thereof.

Also provided are methods for treating a cancer in a subject in need thereof, the method comprising:
a. identifying a subject in need of cancer treatment; and
b. administering to the subject in need thereof the pharmaceutical compositions provided herein,
   wherein administering the pharmaceutical composition to the subject in need thereof treats the cancer in the subject.

Also provided are methods of activating a Vγ9-expressing γδ T cell, the method comprising contacting the Vγ9-expressing γδ T cell with the pharmaceutical composition provided herein, wherein contacting the Vγ9-expressing γδ T cell with the pharmaceutical composition results in an increase in CD69, CD25, and/or Granzyme B expression as compared to a control Vγ9-expressing γδ T cell.

Also provided are methods of producing the pharmaceutical composition provided herein, the method comprising combining the bispecific antibody or antigen binding fragment thereof with a pharmaceutically acceptable carrier to obtain the pharmaceutical composition.

### BRIEF DESCRIPTION OF THE DRAWINGS

The foregoing summary, as well as the following detailed description of certain embodiments of the present application, will be better understood when read in conjunction with the appended drawings. It should be understood, however, that the application is not limited to the precise embodiments shown in the drawings.

FIG. 1 shows a schematic demonstrating the binding of an anti-TRGV9/anti-tumor associated antigen (TAA) bispecific antibody to recruit γδ T cells to a cancer cell and to induce cancer cell death.

FIG. 2 shows a graph demonstrating that Zoledronic acid selectively expands Vγ9Vδ2 T cells from whole peripheral blood mononuclear cells (PBMCs).

FIGS. 3A-3E show phenotypic characterization of Vγ9+γδ T cells. FIG. 3A shows a schematic depiction of gates used to describe the differentiation of γδ T cells (left). Representative FACS-dot plots show the differentiation profile of Vγ9⁺γδ T cells from fresh PBMCs (left) and PBMCs cultured ex vivo with Zoledronic acid+IL-2+IL-15 for 14 days (right). Numbers in quadrants mirror the frequency (mean±SEM) of the respective population among fresh and activated Vγ9⁺γδ T cells. Represented data is mean (±SEM) of five donors (n=5) from a single experiment. FIG. 3B shows numbers in representative dot plots mirroring the frequency (mean±SEM) of Vγ9⁺γδ T cells positive for respective activation marker either from fresh PBMCs (upper row) or PBMCs cultured with Zoledronic acid+IL-2+IL-15 for 14 days (lower row). Represented data is mean (±SEM) of seven donors (n=7) for CD62L, CD69, CD44 expression data from two independent experiments. n=5 donors for NKG2D and 2 donors for CD45RO and CD71 expression data respectively from a single experiment. FIG. 3C shows numbers above gates in dot plots depicting the frequency (mean±SEM) of Vγ9⁺γδ T cells positive for respective inhibitory receptor surface expression either from fresh PBMCs (upper row) or PBMCs cultured with Zoledronic acid+TL-2+IL-15 for day 14 days (lower row). Data shown here is mean (±SEM) of five donors (n=5) for PD1, CTLA4, TIGIT and LAG3 surface expression and seven donors (n=7) for 2B4 and TIM3 surface expression data from two independent experiments. FIG. 3D shows representative FACS dot plots demonstrating the frequency (mean±SEM) of Vγ9⁺γ6 T cells expressing intracellular Granzyme B (left column) and Perforin (right column) from fresh PBMCs (upper row) and PBMCs cultured ex vivo with Zoledronic acid+IL-2+IL-15 for 14 days (lower row). Depicted data is mean (±SEM) of four (n=4) and seven (n=7) donors for Granzyme B and Perforin data respectively from two independent experiments. FIG. 3E shows bars representing the mean (±SEM) concentration (pg/mL) of cytokine in the cell culture supernatant on day 0 and day 14 of PBMCs culture with Zoledronic acid+TL-2+IL-15. Represented data is mean (±SEM) of four wells (n=4) from a single donor.

FIG. 4 shows a histogram demonstrating that VG1 (an anti-TRGV9/anti-CD123 bispecific antibody) recruits Vγ9⁺ T cells as demonstrated by conjugate formation between γδ T cells and Kasumi-3 cells.

FIGS. 5A-5C show graphs demonstrating VG1 (anti-TRGV9/anti-CD123 bispecific antibody) bispecific mediated γδ T cell cytotoxicity against Kasumi-3 cells at different effector to target cell ratios (1:1 for FIG. 5A; 5:1 for FIG. 5B; and 10:1 for FIG. 5C).

FIGS. 6A-6C show graphs demonstrating CD69 (FIG. 6A), CD25 (FIG. 6B), or Granzyme B (FIG. 6C) expression on Vγ9+γδ T cells, non-Vγ9+γδ T cells, and Pan-T cells (non γδ T cells) co-cultured with Kasumi-3 cells and VG1, VG3, or no bispecific antibody.

FIG. 7 shows selective cell binding of anti-TAA1 (TAA mIgG2a, TAA1B1) to transfected Jurkat cells. The EC₅₀ for binding was ˜1 to 2 nM. TAA1B1 did not show any significant binding to a control ALL cell line that endogenously expresses an unrelated protein (TAA1-NULL), but does not express TAA1.

FIG. 8 shows selective protein binding of anti-TAA1 (TAA1 mIgG2a, TAA1B1) to a recombinant TAA1 protein (TAA1W16). TAA1B1 did not show any significant binding to the unrelated protein (TAA1-NULL-W16).

FIG. 9 shows phenotyping of Vg9+ cells used for cytotoxicity studies of a TAA1×Vγ9 bispecific (TAA1B50) from a healthy donor.

FIG. 10 shows that the anti-TRGV9/anti-TAA1 bispecific antibody mediates γδ T cell cytotoxicity against TAA1 expressing Jurkat cells in vitro. Cytotoxicity values represented here were subtracted of basal cytotoxicity value observed in the absence of 15 bispecific antibody. EC₅₀ values were calculated as described in methods. Representative data shown here are from a single experiment.

FIG. 11 shows bispecific antibody mediated cytotoxicity. Expanded and enriched Vγ9Vδ2 T cells from various donors were used to induce cytotoxicity to Jurkat cell line (E:T ratio 1:1) in presence of VT9xTAA1 at indicated concentrations. Assay was conducted for 16 hrs. Percent dead target cells for various conditions are given in the figure.

FIG. 12 shows that the anti-TRGV9/anti-TAA2 bispecific antibody (TAA2V9B106 (B3)) binds γδ T cells (left panel) and mediates γδ T cell cytotoxicity against TAA2 expressing H929 cells in vitro (right panel). EC₅₀ values were calculated as described in methods. Representative data shown here are from a single experiment.

FIG. 13 shows that the anti-TRGV9/anti-TAA2 bispecific antibody (HC1: VG9B420-LH-scFv; HC2: TAA2-Fab (TAA2V9B71.001)) binds γδ T cells (left panel) and mediates γδ T cell cytotoxicity against TAA2 expressing H929 cells in vitro (right panel). EC₅₀ values were calculated as described in methods. Representative data shown here are from a single experiment.

FIG. 14 shows that the anti-TRGV9/anti-TAA2 bispecific antibody (VG9SB10SC1087_P18_D08-Fab RF, TAA2-scFv (TAA2V9B100.001)) binds γδ T cells (left panel) and mediates γδ T cell cytotoxicity against TAA2 expressing H929 cells in vitro (right panel). EC₅₀ values were calculated as described in methods. Representative data shown here are from a single experiment.

FIG. 15 shows that the anti-TRGV9/anti-TAA2 bispecific antibody (VG9SB10SC1087_P18_C12-Fab RF, TAA2-scFv (TAA2V9B101.001)) binds γδ T cells (left panel) and mediates γδ T cell cytotoxicity against TAA2 expressing H929 cells in vitro (right panel). EC₅₀ values were calculated as described in methods. Representative data shown here are from a single experiment.

FIG. 16 shows that the anti-TRGV9/anti-TAA2 bispecific antibody (VG9SB10SC1087_P19_C03-Fab RF, TAA2-scFv (TAA2V9B103.001)) binds γδ T cells (left panel) and mediates γδ T cell cytotoxicity against TAA2 expressing H929 cells in vitro (right panel). EC₅₀ values were calculated as described in methods. Representative data shown here are from a single experiment.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, such as a mammal, such as a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., or such as a human.

It should also be understood that the terms “about,” “approximately,” “generally,” “substantially,” and like terms, used herein when referring to a dimension or characteristic of a component, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-TRGV9/anti-cancer-associated antigen bispecific antibodies and polynucleotides that encode them, anti-TRGV9/anti-CD123 bispecific antibodies and polynucleotides that encode them, TRGV9 polypeptides and TRGV9 polynucleotides that encode them, CD123 polypeptides and CD123 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule provided herein. The “host cell” can be any type of cell, e.g., a primary cell, a cell in culture, or a cell from a cell line. In one embodiment, a “host cell” is a cell transfected with a nucleic acid molecule provided herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed bispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

Antibodies

Provided herein are anti-TRGV9 antibodies or antigen-binding fragments thereof, nucleic acids and expression vectors encoding the antibodies, recombinant cells containing the vectors, and compositions comprising the antibodies. Also provided herein are anti-TRGV9 bispecific antibodies or antigen-binding fragments thereof, nucleic acids and expression vectors encoding the bispecific antibodies, recombinant cells containing the vectors, and compositions comprising the bispecific antibodies. Also provided herein are anti-TRGV9/anti-CD123 bispecific antibodies or antigen-binding fragments thereof, nucleic acids and expression vectors encoding the bispecific antibodies, recombinant cells containing the vectors, and compositions comprising the bispecific antibodies. Methods of making the antibodies, and methods of using the antibodies to treat diseases, including cancer, are also provided. The antibodies disclosed herein possess one or more desirable functional properties, including but not limited to high-affinity binding to TRGV9 and/or high affinity binding to CD123, high specificity to TRGV9 and/or high specificity to CD123, and the ability to treat or prevent cancer when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In certain embodiments, the antibodies provided herein are IgG1. In some embodiments, the antibodies provided herein are IgG2. In some embodiments, the antibodies provided herein are IgG3. In some embodiments, the antibodies provided herein are IgG4. Antibody light chains of vertebrate species can be assigned to one of two clearly distinct types, namely kappa and lambda, based on the amino acid sequences of their constant domains. Accordingly, the antibodies provided herein can contain a kappa or lambda light chain constant domain. According to particular embodiments, the antibodies provided herein include heavy and/or light chain constant regions from rat or human antibodies.

In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). A “CDR” refers to one of three hypervariable regions (HCDR1, HCDR2 or HCDR3) within the non-framework region of the immunoglobulin (Ig or antibody) VH 3-sheet framework, or one of three hypervariable regions (LCDR1, LCDR2 or LCDR3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the Sequence Listing, and tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, MD. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System© (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the Sequence Listing.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term an “isolated antibody” refers to an antibody which is substantially free of other antibodies having different antigenic specificities (e.g., an isolated antibody that specifically binds to TRGV9 is substantially free of antibodies that do not bind to Vγ9; an isolated antibody that specifically binds to CD123 is substantially free of antibodies that do not bind to CD123). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies provided herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a TRGV9 antigen) and a second immunoglobulin variable domain sequence that has binding specificity for a second epitope (e.g., an epitope on a tumor-associated antigen (e.g., a CD123 antigen)). In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on TRGV9 and the second epitope is located on CD123. In an embodiment, the first epitope is located on TRGV9 and the second epitope is located on PD-1, PD-L1, CTLA-4, EGFR, HER-2, CD19, CD20, CD3 and/or other cancer associated immune suppressors or surface antigens.

The term “half antibody” as used herein refers to one immunoglobulin heavy chain associated with one immunoglobulin light chain. An exemplary half-antibody is depicted in SEQ ID NO:17. One skilled in the art will readily appreciate that a half-antibody can encompass a fragment thereof and can also have an antigen binding domain consisting of a single variable domain, e.g., originating from a camelidae.

As used herein, the term “TRGV9” refers to a polypeptide capable of forming a T cell receptor when expressed on the surface of γδ T cells. TRGV9-expressing γδ T cells are among the first T cells to develop in the human fetus and are the predominant γδ T cell subset in healthy adult peripheral blood cells. The term “TRGV9” includes any TRGV9 variant, isoform, and species homolog, which is naturally expressed by cells (including T cells) or can be expressed on cells transfected with genes or cDNA encoding the polypeptide. Unless noted, the TRGV9, in specific embodiments, is a human TRGV9. A human TRGV9 amino acid sequence is provided by GenBank Accession Number NG_001336.2.

The term “CD123” refers to a molecule that is found on cells which helps transmit the signal of interleukin-3, a soluble cytokine that is important in the immune system. CD123 can also be referred to as the “interleukin-3 receptor.” The receptor belongs to the type I cytokine receptor family and is a heterodimer with a unique alpha chain paired with the common beta subunit (beta c or CD131). The CD123 receptor can be found on pluripotent progenitor cells and can induce tyrosine phosphorylation within the cell and promote proliferation and differentiation within hematopoietic cell lines. CD123 can also be expressed in acute myeloid leukemia (AML) subtypes. The term “CD123” includes any CD123 variant, isoform, and species homolog, which is naturally expressed by cells (including T cells) or can be expressed on cells transfected with genes or cDNA encoding those polypeptides, unless noted, in specific embodiments the “CD123” is a human CD123. A human CD123 amino acid sequence is provided by GenBank Accession Number AY789109.1.

As used herein, an antibody that “specifically binds to TRGV9” refers to an antibody that binds to a TRGV9, such as a human TRGV9, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

As used herein, an antigen binding domain or antigen binding fragment that “specifically binds to a tumor-associated antigen” refers to an antigen binding domain or antigen binding fragment that binds a tumor-associated antigen, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system.

As used herein, an antibody that “specifically binds to CD123” refers to an antibody that binds to a CD123, such as a human CD123, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

In one aspect, provided herein is an antibody that binds to TRGV9. In some embodiments, the antibody comprises a heavy chain variable region and a light chain variable region. In a some embodiments, the TRGV9 antibody is not a single domain antibody or nanobody. In some embodiments, the TRGV1 antibody is a humanized antibody.

In certain embodiments, provided herein is an anti-TRGV9 antibody comprising a VH region, VL region, VH CDR1, VH CDR2, VH CDR3, VL CDR1, VL CDR2, and/or VL CDR3 of any one of the antibodies described herein. In some embodiments, provided herein is an anti-TRGV9 antibody comprising a VH region of any one of the antibodies described herein. In some embodiments, provided herein is an anti-TRGV9 antibody comprising a VL region of any one of the antibodies described herein. In some embodiments, provided herein is an anti-TRGV9 antibody comprising a VH region of any one of the antibodies described herein, and a VL region of any one of the antibodies described herein. In some embodiments, provided herein is an anti-TRGV9 antibody comprising a VH CDR1, VH CDR2, and VH CDR3 of any one of the antibodies described herein. In some embodiments, provided herein is an anti-TRGV9 antibody comprising a VL CDR1, VL CDR2, and VL CDR3 of any one of the antibodies described herein. In some embodiments, provided herein is an anti-TRGV9 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of TRGV9 antibodies provided herein are provided in the Sequence Listing, as well as Tables 1-31.

In certain embodiments, provided herein is an anti-TRGV9 bispecific antibody comprising a binding domain that binds to TRGV9 having a VH region, VL region, VH CDR1, VH CDR2, VH CDR3, VL CDR1, VL CDR2, and/or VL CDR3 of any one of the antibodies described herein. In some embodiments, provided herein is an anti-TRGV9 bispecific antibody comprising a binding domain that binds to TRGV9 having a VH region of any one of the antibodies described herein. In some embodiments, provided herein is an anti-TRGV9 bispecific antibody comprising a binding domain that binds to TRGV9 having a VL region of any one of the antibodies described herein. In some embodiments, provided herein is an anti-TRGV9 bispecific antibody comprising a binding domain that binds to TRGV9 having a VH region of any one of the antibodies described herein, and a VL region of any one of the antibodies described herein. In some embodiments, provided herein is an anti-TRGV9 bispecific antibody comprising a binding domain that binds to TRGV9 having a VH CDR1, VH CDR2, and VH CDR3 of any one of the antibodies described. In some embodiments, provided herein is an anti-TRGV9 bispecific antibody comprising a binding domain that binds to TRGV9 having a VL CDR1, VL CDR2, and VL CDR3 of any one of the antibodies described herein. In some embodiments, provided herein is an anti-TRGV9 bispecific antibody comprising a binding domain that binds to TRGV9 having a VH CDR1, VH CDR2, and VH CDR3 of any one of the antibodies described herein; and a VL CDR1, VL CDR2, and VL CDR3 of any one of the antibodies described herein.

In certain embodiments, the anti-TRGV9 antibody is a bispecific antibody. In some embodiments, the anti-TRGV9 bispecific antibody further comprises a second binding domain that binds to CD123 having a VH region, VL region, VH CDR1, VH CDR2, VH CDR3, VL CDR1, VL CDR2, and/or VL CDR3 of an anti-CD123 antibody provided herein. In some embodiments, the anti-TRGV9 bispecific antibody further comprises a second binding domain that binds to CD123 having a VH region of an anti-CD123 antibody provided herein. In some embodiments, the anti-TRGV9 bispecific antibody further comprises a second binding domain that binds to CD123 having a VL region of an anti-CD123 antibody provided herein. In some embodiments, the anti-TRGV9 bispecific antibody further comprises a second binding domain that binds to CD123 having a VH region of an anti-CD123 antibody provided herein, and a VL region of an anti-CD123 antibody provided herein. In some embodiments, the anti-TRGV9 bispecific antibody further comprises a second binding domain that binds to CD123 having a VH CDR1, VH CDR2, and VH CDR3 of an anti-CD123 antibody provided herein. In some embodiments, the anti-TRGV9 bispecific antibody further comprises a second binding domain that binds to CD123 having a VL CDR1, VL CDR2, and VL CDR3 of an anti-CD123 antibody provided herein. In some embodiments, the anti-TRGV9 bispecific antibody further comprises a second binding domain that binds to CD123 having a VH CDR1, VH CDR2, and VH CDR3 of an anti-CD123 antibody provided herein, and a VL CDR1, VL CDR2, and VL CDR3 of an anti-CD123 antibody provided herein.

In certain embodiments, provided is an anti-TRGV9 antibody that is an intact antibody. In other embodiments, provided is an anti-TRGV9 antibody is an antigen binding fragment of the anti-TRGV9 antibody. In some embodiments, the antigen binding fragment of the anti-TRGV9 antibody is a functional fragment. In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the anti-TRGV9 antibody comprises a VH region and a VL region. In some embodiments, the anti-TRGV9 antibody is not a single chain antibody. In some embodiments, the anti-TRGV9 antibody is not a single domain antibody. In some embodiments, the anti-TRGV9 antibody is not a nanobody. In certain embodiments, the anti-TRGV9 antibody is not a VHH antibody. In certain embodiments, the anti-TRGV9 antibody is not a llama antibody. In some embodiments, the anti-TRGV9 bispecific antibody does not comprise a single chain antibody. In some embodiments, the anti-TRGV9 bispecific antibody does not comprise a single domain antibody. In certain embodiments, the anti-TRGV9 bispecific antibody does not comprise a nanobody. In certain embodiments, the anti-TRGV9 bispecific antibody does not comprise a VHH antibody. In certain embodiments, the anti-TRGV9 bispecific antibody does not comprise a llama antibody. In some embodiments, the anti-TRGV9 antibody is a multispecific antibody. In other embodiments, the anti-TRGV9 antibody is a bispecific antibody. In certain embodiments, the multispecific antibody comprises an antigen binding fragment of an anti-TRGV9 antibody provided herein. In other embodiments, the bispecific antibody comprises an antigen binding fragment of an anti-TRGV9 antibody provided herein. In some embodiments, the anti-TRGV9 antibody is an agonistic antibody. In certain embodiments, the anti-TRGV9 antibody activates γδ T cells. In other embodiments, the anti-TRGV9 antibody is an antagonistic antibody. In certain embodiments, the anti-TRGV9 antibody inactivates γδ T cells. In some embodiments, the anti-TRGV9 antibody blocks activation of γδ T cells. In some embodiments, the anti-TRGV9 antibody modulates the activity of γδ T cells. In some embodiments, the anti-TRGV9 antibody neither activates or inactivates the activity of γδ T cells. In specific embodiments, the γδ T cells are human γδ T cells. In specific embodiments, provided is a bispecific antibody comprising a TRGV9 antibody provided herein in a knob-in-hole format. In some embodiments, an anti-TRGV9 antibody provided herein may be comprised in a bispecific antibody. In some embodiments, an anti-TRGV9 bispecific antibody provided herein may be comprised in a multispecific antibody. In certain embodiments, a bispecific antibody provided herein comprises a first binding domain comprising an anti-TRGV9 antibody provided herein that binds to a first TRGV9 epitope, and a second binding domain comprising an anti-TRGV9 antibody provided herein that binds to a second TRGV9 epitope, wherein the first TRGV9 epitope and the second TRGV9 epitope are not the same. In a specific embodiment, a TRGV9 antibody, or antigen binding fragment thereof, provided herein specifically binds to TRGV9. In certain embodiments, a TRGV9 antibody, or antigen binding fragment thereof, provided herein does not bind to an epitope of V62.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:34; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is an antibody that binds to TRGV9, wherein the antibody comprises: (i) a VH comprising a VH CDR1 having an amino acid sequence of SEQ ID NO:196, a VH CDR2 having an amino acid sequence of SEQ ID NO:197, and a VH CDR3 having an amino acid sequence of SEQ ID NO:198; and (ii) a VL comprising a VL CDR1 having an amino acid sequence of SEQ ID NO:199, a VL CDR2 having an amino acid sequence of SEQ ID NO:200, and a VL CDR3 having an amino acid sequence of SEQ ID NO:201. In another aspect, provided herein is an antibody that binds to TRGV9, wherein the antibody comprises: (i) a VH comprising a VH CDR1 having an amino acid sequence of SEQ ID NO:202, a VH CDR2 having an amino acid sequence of SEQ ID NO:203, and a VH CDR3 having an amino acid sequence of SEQ ID NO:204; and (ii) a VL comprising a VL CDR1 having an amino acid sequence of SEQ ID NO:205, a VL CDR2 having an amino acid sequence of SEQ ID NO:206, and a VL CDR3 having an amino acid sequence of SEQ ID NO:207. In another aspect, provided herein is an antibody that binds to TRGV9, wherein the antibody comprises: (i) a VH comprising a VH CDR1 having an amino acid sequence of SEQ ID NO:208, a VH CDR2 having an amino acid sequence of SEQ ID NO:209, and a VH CDR3 having an amino acid sequence of SEQ ID NO:210; and (ii) a VL comprising a VL CDR1 having an amino acid sequence of SEQ ID NO:211, a VL CDR2 having an amino acid sequence of SEQ ID NO:212, and a VL CDR3 having an amino acid sequence of SEQ ID NO:213. In another aspect, provided herein is an antibody that binds to TRGV9, wherein the antibody comprises: (i) a VH comprising a VH CDR1 having an amino acid sequence of SEQ ID NO:214, a VH CDR2 having an amino acid sequence of SEQ ID NO:215, and a VH CDR3 having an amino acid sequence of SEQ ID NO:216; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In another aspect, provided herein is an antibody that binds to TRGV9, wherein the antibody comprises: (i) a VH comprising a VH CDR1 having an amino acid sequence of SEQ ID NO:214, a VH CDR2 having an amino acid sequence of SEQ ID NO:702, and a VH CDR3 having an amino acid sequence of SEQ ID NO:703; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In another aspect, provided herein is an antibody that binds to TRGV9, wherein the antibody comprises: (i) a VH comprising a VH CDR1 having an amino acid sequence of SEQ ID NO:220, a VH CDR2 having an amino acid sequence of SEQ ID NO:221, and a VH CDR3 having an amino acid sequence of SEQ ID NO:222; and (ii) a VL comprising a VL CDR1 having an amino acid sequence of SEQ ID NO:223, a VL CDR2 having an amino acid sequence of SEQ ID NO:224, and a VL CDR3 having an amino acid sequence of SEQ ID NO:225. In another aspect, provided herein is an antibody that binds to TRGV9, wherein the antibody comprises: (i) a VH comprising a VH CDR1 having an amino acid sequence of SEQ ID NO:226, a VH CDR2 having an amino acid sequence of SEQ ID NO:227, and a VH CDR3 having an amino acid sequence of SEQ ID NO:228; and (ii) a VL comprising a VL CDR1 having an amino acid sequence of SEQ ID NO:229, a VL CDR2 having an amino acid sequence of SEQ ID NO:230, and a VL CDR3 having an amino acid sequence of SEQ ID NO:231. In some embodiments, the antibody comprises a VH having an amino acid sequence of SEQ ID NO:34. In some embodiments, the antibody comprises a VL having an amino acid sequence of SEQ ID NO:8. In some embodiments, the antibody comprises a VH having an amino acid sequence of SEQ ID NO:34, and a VL having an amino acid sequence of SEQ ID NO:8. In some embodiments, the antibody comprises a VH comprising an amino acid sequence having at least 95% identity to the amino acid sequence of SEQ ID NO:34. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:34, and a VL comprising an amino acid sequence having at least 95% identity to the amino acid sequence of SEQ ID NO:8.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:35; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is an antibody that binds to TRGV9, wherein the antibody comprises: (i) a VH comprising a VH CDR1 having an amino acid sequence of SEQ ID NO:232, a VH CDR2 having an amino acid sequence of SEQ ID NO:233, and a VH CDR3 having an amino acid sequence of SEQ ID NO:234; and (ii) a VL comprising a VL CDR1 having an amino acid sequence of SEQ ID NO:235, a VL CDR2 having an amino acid sequence of SEQ ID NO:236, and a VL CDR3 having an amino acid sequence of SEQ ID NO:237. In another aspect, provided herein is an antibody that binds to TRGV9, wherein the antibody comprises: (i) a VH comprising a VH CDR1 having an amino acid sequence of SEQ ID NO:238, a VH CDR2 having an amino acid sequence of SEQ ID NO:239, and a VH CDR3 having an amino acid sequence of SEQ ID NO:240; and (ii) a VL comprising a VL CDR1 having an amino acid sequence of SEQ ID NO:241, a VL CDR2 having an amino acid sequence of SEQ ID NO:242, and a VL CDR3 having an amino acid sequence of SEQ ID NO:243. In another aspect, provided herein is an antibody that binds to TRGV9, wherein the antibody comprises: (i) a VH comprising a VH CDR1 having an amino acid sequence of SEQ ID NO:244, a VH CDR2 having an amino acid sequence of SEQ ID NO:245, and a VH CDR3 having an amino acid sequence of SEQ ID NO:246; and (ii) a VL comprising a VL CDR1 having an amino acid sequence of SEQ ID NO:247, a VL CDR2 having an amino acid sequence of SEQ ID NO:248, and a VL CDR3 having an amino acid sequence of SEQ ID NO:249. In another aspect, provided herein is an antibody that binds to TRGV9, wherein the antibody comprises: (i) a VH comprising a VH CDR1 having an amino acid sequence of SEQ ID NO:250, a VH CDR2 having an amino acid sequence of SEQ ID NO:251, and a VH CDR3 having an amino acid sequence of SEQ ID NO:252; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In another aspect, provided herein is an antibody that binds to TRGV9, wherein the antibody comprises: (i) a VH comprising a VH CDR1 having an amino acid sequence of SEQ ID NO:250, a VH CDR2 having an amino acid sequence of SEQ ID NO:704, and a VH CDR3 having an amino acid sequence of SEQ ID NO:705; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In another aspect, provided herein is an antibody that binds to TRGV9, wherein the antibody comprises: (i) a VH comprising a VH CDR1 having an amino acid sequence of SEQ ID NO:256, a VH CDR2 having an amino acid sequence of SEQ ID NO:257, and a VH CDR3 having an amino acid sequence of SEQ ID NO:258; and (ii) a VL comprising a VL CDR1 having an amino acid sequence of SEQ ID NO:259, a VL CDR2 having an amino acid sequence of SEQ ID NO:260, and a VL CDR3 having an amino acid sequence of SEQ ID NO:261. In another aspect, provided herein is an antibody that binds to TRGV9, wherein the antibody comprises: (i) a VH comprising a VH CDR1 having an amino acid sequence of SEQ ID NO:262, a VH CDR2 having an amino acid sequence of SEQ ID NO:263, and a VH CDR3 having an amino acid sequence of SEQ ID NO:264; and (ii) a VL comprising a VL CDR1 having an amino acid sequence of SEQ ID NO:265, a VL CDR2 having an amino acid sequence of SEQ ID NO:266, and a VL CDR3 having an amino acid sequence of SEQ ID NO:267. In some embodiments, the antibody comprises a VH having an amino acid sequence of SEQ ID NO:35. In some embodiments, the antibody comprises a VL having an amino acid sequence of SEQ ID NO:8. In some embodiments, the antibody comprises a VH having an amino acid sequence of SEQ ID NO:35, and a VL having an amino acid sequence of SEQ ID NO:8. In some embodiments, the antibody comprises a VH comprising an amino acid sequence having at least 95% identity to the amino acid sequence of SEQ ID NO:35. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:35, and a VL comprising an amino acid sequence having at least 95% identity to the amino acid sequence of SEQ ID NO:8.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:36; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is an antibody that binds to TRGV9, wherein the antibody comprises: (i) a VH comprising a VH CDR1 having an amino acid sequence of SEQ ID NO:268, a VH CDR2 having an amino acid sequence of SEQ ID NO:269, and a VH CDR3 having an amino acid sequence of SEQ ID NO:270; and (ii) a VL comprising a VL CDR1 having an amino acid sequence of SEQ ID NO:271, a VL CDR2 having an amino acid sequence of SEQ ID NO:272, and a VL CDR3 having an amino acid sequence of SEQ ID NO:273. In another aspect, provided herein is an antibody that binds to TRGV9, wherein the antibody comprises: (i) a VH comprising a VH CDR1 having an amino acid sequence of SEQ ID NO:274, a VH CDR2 having an amino acid sequence of SEQ ID NO:275, and a VH CDR3 having an amino acid sequence of SEQ ID NO:276; and (ii) a VL comprising a VL CDR1 having an amino acid sequence of SEQ ID NO:277, a VL CDR2 having an amino acid sequence of SEQ ID NO:278, and a VL CDR3 having an amino acid sequence of SEQ ID NO:279. In another aspect, provided herein is an antibody that binds to TRGV9, wherein the antibody comprises: (i) a VH comprising a VH CDR1 having an amino acid sequence of SEQ ID NO:280, a VH CDR2 having an amino acid sequence of SEQ ID NO:281, and a VH CDR3 having an amino acid sequence of SEQ ID NO:282; and (ii) a VL comprising a VL CDR1 having an amino acid sequence of SEQ ID NO:283, a VL CDR2 having an amino acid sequence of SEQ ID NO:284, and a VL CDR3 having an amino acid sequence of SEQ ID NO:285. In another aspect, provided herein is an antibody that binds to TRGV9, wherein the antibody comprises: (i) a VH comprising a VH CDR1 having an amino acid sequence of SEQ ID NO:286, a VH CDR2 having an amino acid sequence of SEQ ID NO:287, and a VH CDR3 having an amino acid sequence of SEQ ID NO:288; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In another aspect, provided herein is an antibody that binds to TRGV9, wherein the antibody comprises: (i) a VH comprising a VH CDR1 having an amino acid sequence of SEQ ID NO:286, a VH CDR2 having an amino acid sequence of SEQ ID NO:706, and a VH CDR3 having an amino acid sequence of SEQ ID NO:707; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In another aspect, provided herein is an antibody that binds to TRGV9, wherein the antibody comprises: (i) a VH comprising a VH CDR1 having an amino acid sequence of SEQ ID NO:292, a VH CDR2 having an amino acid sequence of SEQ ID NO:293, and a VH CDR3 having an amino acid sequence of SEQ ID NO:294; and (ii) a VL comprising a VL CDR1 having an amino acid sequence of SEQ ID NO:295, a VL CDR2 having an amino acid sequence of SEQ ID NO:296, and a VL CDR3 having an amino acid sequence of SEQ ID NO:297. In another aspect, provided herein is an antibody that binds to TRGV9, wherein the antibody comprises: (i) a VH comprising a VH CDR1 having an amino acid sequence of SEQ ID NO:298, a VH CDR2 having an amino acid sequence of SEQ ID NO:299, and a VH CDR3 having an amino acid sequence of SEQ ID NO:300; and (ii) a VL comprising a VL CDR1 having an amino acid sequence of SEQ ID NO:301, a VL CDR2 having an amino acid sequence of SEQ ID NO:302, and a VL CDR3 having an amino acid sequence of SEQ ID NO:303. In some embodiments, the antibody comprises a VH having an amino acid sequence of SEQ ID NO:36. In some embodiments, the antibody comprises a VL having an amino acid sequence of SEQ ID NO:8. In some embodiments, the antibody comprises a VH having an amino acid sequence of SEQ ID NO:36, and a VL having an amino acid sequence of SEQ ID NO:8. In some embodiments, the antibody comprises a VH comprising an amino acid sequence having at least 95% identity to the amino acid sequence of SEQ ID NO:36. In some embodiments, the antibody comprises a VL comprising an amino acid sequence having at least 95% identity to the amino acid sequence of SEQ ID NO:8. In some embodiments, the antibody comprises a VH comprising an amino acid sequence having at least 95% identity to the amino acid sequence of SEQ ID NO:36, and a VL comprising an amino acid sequence having at least 95% identity to the amino acid sequence of SEQ ID NO:8.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:65; and (ii) a VL comprising a VL CDR1, a VL CDR2, and a VL CDR3 having amino acid sequences of the VL CDR1, VL CDR2, and VL CDR3, respectively, of SEQ ID NO:66. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:67; and (ii) a VL comprising a VL CDR1, a VL CDR2, and a VL CDR3 having amino acid sequences of the VL CDR1, VL CDR2, and VL CDR3, respectively, of SEQ ID NO:68. In another aspect, provided herein is an antibody that binds to TRGV9, wherein the antibody comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6. In another aspect, provided herein is an antibody that binds to TRGV9, wherein the antibody comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6. In another aspect, provided herein is an antibody that binds to TRGV9, wherein the antibody comprises: (i) a VH comprising a VH CDR1 having an amino acid sequence of SEQ ID NO:304, a VH CDR2 having an amino acid sequence of SEQ ID NO:305, and a VH CDR3 having an amino acid sequence of SEQ ID NO:306; and (ii) a VL comprising a VL CDR1 having an amino acid sequence of SEQ ID NO:307, a VL CDR2 having an amino acid sequence of SEQ ID NO:308, and a VL CDR3 having an amino acid sequence of SEQ ID NO:309. In another aspect, provided herein is an antibody that binds to TRGV9, wherein the antibody comprises: (i) a VH comprising a VH CDR1 having an amino acid sequence of SEQ ID NO:310, a VH CDR2 having an amino acid sequence of SEQ ID NO:311, and a VH CDR3 having an amino acid sequence of SEQ ID NO:312; and (ii) a VL comprising a VL CDR1 having an amino acid sequence of SEQ ID NO:313, a VL CDR2 having an amino acid sequence of SEQ ID NO:314, and a VL CDR3 having an amino acid sequence of SEQ ID NO:315. In another aspect, provided herein is an antibody that binds to TRGV9, wherein the antibody comprises: (i) a VH comprising a VH CDR1 having an amino acid sequence of SEQ ID NO:316, a VH CDR2 having an amino acid sequence of SEQ ID NO:317, and a VH CDR3 having an amino acid sequence of SEQ ID NO:318; and (ii) a VL comprising a VL CDR1 having an amino acid sequence of SEQ ID NO:319, a VL CDR2 having an amino acid sequence of SEQ ID NO:320, and a VL CDR3 having an amino acid sequence of SEQ ID NO:321. In another aspect, provided herein is an antibody that binds to TRGV9, wherein the antibody comprises: (i) a VH comprising a VH CDR1 having an amino acid sequence of SEQ ID NO:322, a VH CDR2 having an amino acid sequence of SEQ ID NO:323, and a VH CDR3 having an amino acid sequence of SEQ ID NO:324; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In another aspect, provided herein is an antibody that binds to TRGV9, wherein the antibody comprises: (i) a VH comprising a VH CDR1 having an amino acid sequence of SEQ ID NO:322, a VH CDR2 having an amino acid sequence of SEQ ID NO:708, and a VH CDR3 having an amino acid sequence of SEQ ID NO:709; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In another aspect, provided herein is an antibody that binds to TRGV9, wherein the antibody comprises: (i) a VH comprising a VH CDR1 having an amino acid sequence of SEQ ID NO:328, a VH CDR2 having an amino acid sequence of SEQ ID NO:329, and a VH CDR3 having an amino acid sequence of SEQ ID NO:330; and (ii) a VL comprising a VL CDR1 having an amino acid sequence of SEQ ID NO:331, a VL CDR2 having an amino acid sequence of SEQ ID NO:332, and a VL CDR3 having an amino acid sequence of SEQ ID NO:333. In another aspect, provided herein is an antibody that binds to TRGV9, wherein the antibody comprises: (i) a VH comprising a VH CDR1 having an amino acid sequence of SEQ ID NO:334, a VH CDR2 having an amino acid sequence of SEQ ID NO:335, and a VH CDR3 having an amino acid sequence of SEQ ID NO:336; and (ii) a VL comprising a VL CDR1 having an amino acid sequence of SEQ ID NO:337, a VL CDR2 having an amino acid sequence of SEQ ID NO:338, and a VL CDR3 having an amino acid sequence of SEQ ID NO:339. In another aspect, provided herein is an antibody that binds to TRGV9, wherein the antibody comprises: (i) a VH comprising a VH CDR1 having an amino acid sequence of SEQ ID NO:340, a VH CDR2 having an amino acid sequence of SEQ ID NO:341, and a VH CDR3 having an amino acid sequence of SEQ ID NO:342; and (ii) a VL comprising a VL CDR1 having an amino acid sequence of SEQ ID NO:343, a VL CDR2 having an amino acid sequence of SEQ ID NO:344, and a VL CDR3 having an amino acid sequence of SEQ ID NO:345. In another aspect, provided herein is an antibody that binds to TRGV9, wherein the antibody comprises: (i) a VH comprising a VH CDR1 having an amino acid sequence of SEQ ID NO:346, a VH CDR2 having an amino acid sequence of SEQ ID NO:347, and a VH CDR3 having an amino acid sequence of SEQ ID NO:348; and (ii) a VL comprising a VL CDR1 having an amino acid sequence of SEQ ID NO:349, a VL CDR2 having an amino acid sequence of SEQ ID NO:350, and a VL CDR3 having an amino acid sequence of SEQ ID NO:351. In another aspect, provided herein is an antibody that binds to TRGV9, wherein the antibody comprises: (i) a VH comprising a VH CDR1 having an amino acid sequence of SEQ ID NO:352, a VH CDR2 having an amino acid sequence of SEQ ID NO:353, and a VH CDR3 having an amino acid sequence of SEQ ID NO:354; and (ii) a VL comprising a VL CDR1 having an amino acid sequence of SEQ ID NO:355, a VL CDR2 having an amino acid sequence of SEQ ID NO:356, and a VL CDR3 having an amino acid sequence of SEQ ID NO:357. In another aspect, provided herein is an antibody that binds to TRGV9, wherein the antibody comprises: (i) a VH comprising a VH CDR1 having an amino acid sequence of SEQ ID NO:358, a VH CDR2 having an amino acid sequence of SEQ ID NO:359, and a VH CDR3 having an amino acid sequence of SEQ ID NO:360; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In another aspect, provided herein is an antibody that binds to TRGV9, wherein the antibody comprises: (i) a VH comprising a VH CDR1 having an amino acid sequence of SEQ ID NO:358, a VH CDR2 having an amino acid sequence of SEQ ID NO:710, and a VH CDR3 having an amino acid sequence of SEQ ID NO:711; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In another aspect, provided herein is an antibody that binds to TRGV9, wherein the antibody comprises: (i) a VH comprising a VH CDR1 having an amino acid sequence of SEQ ID NO:364, a VH CDR2 having an amino acid sequence of SEQ ID NO:365, and a VH CDR3 having an amino acid sequence of SEQ ID NO:366; and (ii) a VL comprising a VL CDR1 having an amino acid sequence of SEQ ID NO:367, a VL CDR2 having an amino acid sequence of SEQ ID NO:368, and a VL CDR3 having an amino acid sequence of SEQ ID NO:369. In another aspect, provided herein is an antibody that binds to TRGV9, wherein the antibody comprises: (i) a VH comprising a VH CDR1 having an amino acid sequence of SEQ ID NO:370, a VH CDR2 having an amino acid sequence of SEQ ID NO:371, and a VH CDR3 having an amino acid sequence of SEQ ID NO:372; and (ii) a VL comprising a VL CDR1 having an amino acid sequence of SEQ ID NO:373, a VL CDR2 having an amino acid sequence of SEQ ID NO:374, and a VL CDR3 having an amino acid sequence of SEQ ID NO:375. In some embodiments, the antibody comprises a VH having an amino acid sequence of SEQ ID NO:65. In some embodiments, the antibody comprises a VL having an amino acid sequence of SEQ ID NO:66. In some embodiments, the antibody comprises a VH having an amino acid sequence of SEQ ID NO:65, and a VL having an amino acid sequence of SEQ ID NO:66. In some embodiments, the antibody comprises a VH having an amino acid sequence of SEQ ID NO:67. In some embodiments, the antibody comprises a VL having an amino acid sequence of SEQ ID NO:68. In some embodiments, the antibody comprises a VH having an amino acid sequence of SEQ ID NO:67, and a VL having an amino acid sequence of SEQ ID NO:68. In some embodiments, the antibody comprises a heavy chain having an amino acid sequence of SEQ ID NO:71. In some embodiments, the antibody comprises a light chain having an amino acid sequence of SEQ ID NO:72. In some embodiments, the antibody comprises a heavy chain having an amino acid sequence of SEQ ID NO:71, and a light chain having an amino acid sequence of SEQ ID NO:72. In some embodiments, the antibody comprises an amino acid sequence of SEQ ID NO:70. In some embodiments, the antibody comprises a heavy chain having an amino acid sequence of SEQ ID NO:74. In some embodiments, the antibody comprises a light chain having an amino acid sequence of SEQ ID NO:75. In some embodiments, the antibody comprises a heavy chain having an amino acid sequence of SEQ ID NO:74, and a light chain having an amino acid sequence of SEQ ID NO:75. In some embodiments, the antibody comprises an amino acid sequence of SEQ ID NO:73. In some embodiments, the antibody comprises a VH comprising an amino acid sequence having at least 95% identity to the amino acid sequence of SEQ ID NO:65. In some embodiments, the antibody comprises a VL comprising an amino acid sequence having at least 95% identity to the amino acid sequence of SEQ ID NO:66. In some embodiments, the antibody comprises a VH comprising an amino acid sequence having at least 95% identity to the amino acid sequence of SEQ ID NO:65, and a VL comprising an amino acid sequence having at least 95% identity to the amino acid sequence of SEQ ID NO:66. In some embodiments, the antibody comprises a VH comprising an amino acid sequence having at least 95% identity to the amino acid sequence of SEQ ID NO:67. In some embodiments, the antibody comprises a VL comprising an amino acid sequence having at least 95% identity to the amino acid sequence of SEQ ID NO:68. In some embodiments, the antibody comprises a VH comprising an amino acid sequence having at least 95% identity to the amino acid sequence of SEQ ID NO:67, and a VL comprising an amino acid sequence having at least 95% identity to the amino acid sequence of SEQ ID NO:68. In some embodiments, the antibody comprises a heavy chain comprising an amino acid sequence having at least 95% identity to the amino acid sequence of SEQ ID NO:71. In some embodiments, the antibody comprises a light chain comprising an amino acid sequence having at least 95% identity to the amino acid sequence of SEQ ID NO:72. In some embodiments, the antibody comprises a heavy chain comprising an amino acid sequence having at least 95% identity to the amino acid sequence of SEQ ID NO:71, and a light chain comprising an amino acid sequence having at least 95% identity to the amino acid sequence of SEQ ID NO:72. In some embodiments, the antibody comprises an amino acid sequence of SEQ ID NO:70. In some embodiments, the antibody comprises a heavy chain comprising an amino acid sequence having at least 95% identity to the amino acid sequence of SEQ ID NO:74. In some embodiments, the antibody comprises a light chain comprising an amino acid sequence having at least 95% identity to the amino acid sequence of SEQ ID NO:75. In some embodiments, the antibody comprises a heavy chain comprising an amino acid sequence having at least 95% identity to the amino acid sequence of SEQ ID NO:74, and a light chain comprising an amino acid sequence having at least 95% identity to the amino acid sequence of SEQ ID NO:75. In some embodiments, the antibody comprises an amino acid sequence of SEQ ID NO:73.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:104; and (ii) a VL comprising a VL CDR1, a VL CDR2, and a VL CDR3 having amino acid sequences of the VL CDR1, VL CDR2, and VL CDR3, respectively, of SEQ ID NO:105. In another aspect, provided herein is an antibody that binds to TRGV9, wherein the antibody comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100; and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103. In another aspect, provided herein is an antibody that binds to TRGV9, wherein the antibody comprises: (i) a VH comprising a VH CDR1 having an amino acid sequence of SEQ ID NO:412, a VH CDR2 having an amino acid sequence of SEQ ID NO:413, and a VH CDR3 having an amino acid sequence of SEQ ID NO:414; and (ii) a VL comprising a VL CDR1 having an amino acid sequence of SEQ ID NO:415, a VL CDR2 having an amino acid sequence of SEQ ID NO:416, and a VL CDR3 having an amino acid sequence of SEQ ID NO:417. In another aspect, provided herein is an antibody that binds to TRGV9, wherein the antibody comprises: (i) a VH comprising a VH CDR1 having an amino acid sequence of SEQ ID NO:418, a VH CDR2 having an amino acid sequence of SEQ ID NO:419, and a VH CDR3 having an amino acid sequence of SEQ ID NO:420; and (ii) a VL comprising a VL CDR1 having an amino acid sequence of SEQ ID NO:421, a VL CDR2 having an amino acid sequence of SEQ ID NO:422, and a VL CDR3 having an amino acid sequence of SEQ ID NO:423. In another aspect, provided herein is an antibody that binds to TRGV9, wherein the antibody comprises: (i) a VH comprising a VH CDR1 having an amino acid sequence of SEQ ID NO:424, a VH CDR2 having an amino acid sequence of SEQ ID NO:425, and a VH CDR3 having an amino acid sequence of SEQ ID NO:426; and (ii) a VL comprising a VL CDR1 having an amino acid sequence of SEQ ID NO:427, a VL CDR2 having an amino acid sequence of SEQ ID NO:428, and a VL CDR3 having an amino acid sequence of SEQ ID NO:429. In another aspect, provided herein is an antibody that binds to TRGV9, wherein the antibody comprises: (i) a VH comprising a VH CDR1 having an amino acid sequence of SEQ ID NO:430, a VH CDR2 having an amino acid sequence of SEQ ID NO:431, and a VH CDR3 having an amino acid sequence of SEQ ID NO:432; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In another aspect, provided herein is an antibody that binds to TRGV9, wherein the antibody comprises: (i) a VH comprising a VH CDR1 having an amino acid sequence of SEQ ID NO:430, a VH CDR2 having an amino acid sequence of SEQ ID NO:714, and a VH CDR3 having an amino acid sequence of SEQ ID NO:715; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In another aspect, provided herein is an antibody that binds to TRGV9, wherein the antibody comprises: (i) a VH comprising a VH CDR1 having an amino acid sequence of SEQ ID NO:436, a VH CDR2 having an amino acid sequence of SEQ ID NO:437, and a VH CDR3 having an amino acid sequence of SEQ ID NO:438; and (ii) a VL comprising a VL CDR1 having an amino acid sequence of SEQ ID NO:439, a VL CDR2 having an amino acid sequence of SEQ ID NO:440, and a VL CDR3 having an amino acid sequence of SEQ ID NO:441. In another aspect, provided herein is an antibody that binds to TRGV9, wherein the antibody comprises: (i) a VH comprising a VH CDR1 having an amino acid sequence of SEQ ID NO:442, a VH CDR2 having an amino acid sequence of SEQ ID NO:443, and a VH CDR3 having an amino acid sequence of SEQ ID NO:444; and (ii) a VL comprising a VL CDR1 having an amino acid sequence of SEQ ID NO:445, a VL CDR2 having an amino acid sequence of SEQ ID NO:446, and a VL CDR3 having an amino acid sequence of SEQ ID NO:447. In some embodiments, the antibody comprises a VH having an amino acid sequence of SEQ ID NO:104. In some embodiments, the antibody comprises a VL having an amino acid sequence of SEQ ID NO:105. In some embodiments, the antibody comprises a VH having an amino acid sequence of SEQ ID NO:104, and a VL having an amino acid sequence of SEQ ID NO:105. In some embodiments, the antibody comprises an amino acid sequence of SEQ ID NO:106. In some embodiments, the antibody comprises a VH comprising an amino acid sequence having at least 95% identity to the amino acid sequence of SEQ ID NO:104. In some embodiments, the antibody comprises a VL comprising an amino acid sequence having at least 95% identity to the amino acid sequence of SEQ ID NO:105. In some embodiments, the antibody comprises a VH comprising an amino acid sequence having at least 95% identity to the amino acid sequence of SEQ ID NO:104, and a VL comprising an amino acid sequence having at least 95% identity to the amino acid sequence of SEQ ID NO:105. In some embodiments, the antibody comprises an amino acid sequence of SEQ ID NO:106.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:113; and (ii) a VL comprising a VL CDR1, a VL CDR2, and a VL CDR3 having amino acid sequences of the VL CDR1, VL CDR2, and VL CDR3, respectively, of SEQ ID NO:114. In another aspect, provided herein is an antibody that binds to TRGV9, wherein the antibody comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109; and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In another aspect, provided herein is an antibody that binds to TRGV9, wherein the antibody comprises: (i) a VH comprising a VH CDR1 having an amino acid sequence of SEQ ID NO:448, a VH CDR2 having an amino acid sequence of SEQ ID NO:449, and a VH CDR3 having an amino acid sequence of SEQ ID NO:450; and (ii) a VL comprising a VL CDR1 having an amino acid sequence of SEQ ID NO:451, a VL CDR2 having an amino acid sequence of SEQ ID NO:452, and a VL CDR3 having an amino acid sequence of SEQ ID NO:453. In another aspect, provided herein is an antibody that binds to TRGV9, wherein the antibody comprises: (i) a VH comprising a VH CDR1 having an amino acid sequence of SEQ ID NO:454, a VH CDR2 having an amino acid sequence of SEQ ID NO:455, and a VH CDR3 having an amino acid sequence of SEQ ID NO:456; and (ii) a VL comprising a VL CDR1 having an amino acid sequence of SEQ ID NO:457, a VL CDR2 having an amino acid sequence of SEQ ID NO:458, and a VL CDR3 having an amino acid sequence of SEQ ID NO:459. In another aspect, provided herein is an antibody that binds to TRGV9, wherein the antibody comprises: (i) a VH comprising a VH CDR1 having an amino acid sequence of SEQ ID NO:460, a VH CDR2 having an amino acid sequence of SEQ ID NO:461, and a VH CDR3 having an amino acid sequence of SEQ ID NO:462; and (ii) a VL comprising a VL CDR1 having an amino acid sequence of SEQ ID NO:463, a VL CDR2 having an amino acid sequence of SEQ ID NO:464, and a VL CDR3 having an amino acid sequence of SEQ ID NO:465. In another aspect, provided herein is an antibody that binds to TRGV9, wherein the antibody comprises: (i) a VH comprising a VH CDR1 having an amino acid sequence of SEQ ID NO:466, a VH CDR2 having an amino acid sequence of SEQ ID NO:467, and a VH CDR3 having an amino acid sequence of SEQ ID NO:468; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In another aspect, provided herein is an antibody that binds to TRGV9, wherein the antibody comprises: (i) a VH comprising a VH CDR1 having an amino acid sequence of SEQ ID NO:466, a VH CDR2 having an amino acid sequence of SEQ ID NO:716, and a VH CDR3 having an amino acid sequence of SEQ ID NO:717; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In another aspect, provided herein is an antibody that binds to TRGV9, wherein the antibody comprises: (i) a VH comprising a VH CDR1 having an amino acid sequence of SEQ ID NO:472, a VH CDR2 having an amino acid sequence of SEQ ID NO:473, and a VH CDR3 having an amino acid sequence of SEQ ID NO:474; and (ii) a VL comprising a VL CDR1 having an amino acid sequence of SEQ ID NO:475, a VL CDR2 having an amino acid sequence of SEQ ID NO:476, and a VL CDR3 having an amino acid sequence of SEQ ID NO:477. In another aspect, provided herein is an antibody that binds to TRGV9, wherein the antibody comprises: (i) a VH comprising a VH CDR1 having an amino acid sequence of SEQ ID NO:478, a VH CDR2 having an amino acid sequence of SEQ ID NO:479, and a VH CDR3 having an amino acid sequence of SEQ ID NO:480; and (ii) a VL comprising a VL CDR1 having an amino acid sequence of SEQ ID NO:481, a VL CDR2 having an amino acid sequence of SEQ ID NO:482, and a VL CDR3 having an amino acid sequence of SEQ ID NO:483. In some embodiments, the antibody comprises a VH having an amino acid sequence of SEQ ID NO:113. In some embodiments, the antibody comprises a VL having an amino acid sequence of SEQ ID NO:114. In some embodiments, the antibody comprises a VH having an amino acid sequence of SEQ ID NO:113, and a VL having an amino acid sequence of SEQ ID NO:114. In some embodiments, the antibody comprises a heavy chain having an amino acid sequence of SEQ ID NO:115. In some embodiments, the antibody comprises a light chain having an amino acid sequence of SEQ ID NO:116. In some embodiments, the antibody comprises a heavy chain having an amino acid sequence of SEQ ID NO:115, and a light chain having an amino acid sequence of SEQ ID NO:116. In some embodiments, the antibody comprises a VH comprising an amino acid sequence having at least 95% identity to the amino acid sequence of SEQ ID NO:113. In some embodiments, the antibody comprises a VL comprising an amino acid sequence having at least 95% identity to the amino acid sequence of SEQ ID NO:114. In some embodiments, the antibody comprises a VH comprising an amino acid sequence having at least 95% identity to the amino acid sequence of SEQ ID NO:113, and a VL comprising an amino acid sequence having at least 95% identity to the amino acid sequence of SEQ ID NO:114. In some embodiments, the antibody comprises a heavy chain comprising an amino acid sequence having at least 95% identity to the amino acid sequence of SEQ ID NO:115. In some embodiments, the antibody comprises a light chain comprising an amino acid sequence having at least 95% identity to the amino acid sequence of SEQ ID NO:116. In some embodiments, the antibody comprises a heavy chain comprising an amino acid sequence having at least 95% identity to the amino acid sequence of SEQ ID NO:115, and a light chain comprising an amino acid sequence having at least 95% identity to the amino acid sequence of SEQ ID NO:116.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:123; and (ii) a VL comprising a VL CDR1, a VL CDR2, and a VL CDR3 having amino acid sequences of the VL CDR1, VL CDR2, and VL CDR3, respectively, of SEQ ID NO:124. In another aspect, provided herein is an antibody that binds to TRGV9, wherein the antibody comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119; and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In another aspect, provided herein is an antibody that binds to TRGV9, wherein the antibody comprises: (i) a VH comprising a VH CDR1 having an amino acid sequence of SEQ ID NO:484, a VH CDR2 having an amino acid sequence of SEQ ID NO:485, and a VH CDR3 having an amino acid sequence of SEQ ID NO:486; and (ii) a VL comprising a VL CDR1 having an amino acid sequence of SEQ ID NO:487, a VL CDR2 having an amino acid sequence of SEQ ID NO:488, and a VL CDR3 having an amino acid sequence of SEQ ID NO:489. In another aspect, provided herein is an antibody that binds to TRGV9, wherein the antibody comprises: (i) a VH comprising a VH CDR1 having an amino acid sequence of SEQ ID NO:490, a VH CDR2 having an amino acid sequence of SEQ ID NO:491, and a VH CDR3 having an amino acid sequence of SEQ ID NO:492; and (ii) a VL comprising a VL CDR1 having an amino acid sequence of SEQ ID NO:493, a VL CDR2 having an amino acid sequence of SEQ ID NO:494, and a VL CDR3 having an amino acid sequence of SEQ ID NO:495. In another aspect, provided herein is an antibody that binds to TRGV9, wherein the antibody comprises: (i) a VH comprising a VH CDR1 having an amino acid sequence of SEQ ID NO:496, a VH CDR2 having an amino acid sequence of SEQ ID NO:497, and a VH CDR3 having an amino acid sequence of SEQ ID NO:498; and (ii) a VL comprising a VL CDR1 having an amino acid sequence of SEQ ID NO:499, a VL CDR2 having an amino acid sequence of SEQ ID NO:500, and a VL CDR3 having an amino acid sequence of SEQ ID NO:501. In another aspect, provided herein is an antibody that binds to TRGV9, wherein the antibody comprises: (i) a VH comprising a VH CDR1 having an amino acid sequence of SEQ ID NO:502, a VH CDR2 having an amino acid sequence of SEQ ID NO:503, and a VH CDR3 having an amino acid sequence of SEQ ID NO:504; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In another aspect, provided herein is an antibody that binds to TRGV9, wherein the antibody comprises: (i) a VH comprising a VH CDR1 having an amino acid sequence of SEQ ID NO:502, a VH CDR2 having an amino acid sequence of SEQ ID NO:718, and a VH CDR3 having an amino acid sequence of SEQ ID NO:719; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In another aspect, provided herein is an antibody that binds to TRGV9, wherein the antibody comprises: (i) a VH comprising a VH CDR1 having an amino acid sequence of SEQ ID NO:508, a VH CDR2 having an amino acid sequence of SEQ ID NO:509, and a VH CDR3 having an amino acid sequence of SEQ ID NO:510; and (ii) a VL comprising a VL CDR1 having an amino acid sequence of SEQ ID NO:511, a VL CDR2 having an amino acid sequence of SEQ ID NO:512, and a VL CDR3 having an amino acid sequence of SEQ ID NO:513. In another aspect, provided herein is an antibody that binds to TRGV9, wherein the antibody comprises: (i) a VH comprising a VH CDR1 having an amino acid sequence of SEQ ID NO:514, a VH CDR2 having an amino acid sequence of SEQ ID NO:515, and a VH CDR3 having an amino acid sequence of SEQ ID NO:516; and (ii) a VL comprising a VL CDR1 having an amino acid sequence of SEQ ID NO:517, a VL CDR2 having an amino acid sequence of SEQ ID NO:518, and a VL CDR3 having an amino acid sequence of SEQ ID NO:519. In some embodiments, the antibody comprises a VH having an amino acid sequence of SEQ ID NO:123. In some embodiments, the antibody comprises a VL having an amino acid sequence of SEQ ID NO:124. In some embodiments, the antibody comprises a VH having an amino acid sequence of SEQ ID NO:123, and a VL having an amino acid sequence of SEQ ID NO:124. In some embodiments, the antibody comprises a heavy chain having an amino acid sequence of SEQ ID NO:125. In some embodiments, the antibody comprises a light chain having an amino acid sequence of SEQ ID NO:126. In some embodiments, the antibody comprises a heavy chain having an amino acid sequence of SEQ ID NO:125, and a light chain having an amino acid sequence of SEQ ID NO:126. In some embodiments, the antibody comprises a VH comprising an amino acid sequence having at least 95% identity to the amino acid sequence of SEQ ID NO:123. In some embodiments, the antibody comprises a VL comprising an amino acid sequence having at least 95% identity to the amino acid sequence of SEQ ID NO:124. In some embodiments, the antibody comprises a VH comprising an amino acid sequence having at least 95% identity to the amino acid sequence of SEQ ID NO:123, and a VL comprising an amino acid sequence having at least 95% identity to the amino acid sequence of SEQ ID NO:124. In some embodiments, the antibody comprises a heavy chain comprising an amino acid sequence having at least 95% identity to the amino acid sequence of SEQ ID NO:125. In some embodiments, the antibody comprises a light chain comprising an amino acid sequence having at least 95% identity to the amino acid sequence of SEQ ID NO:126. In some embodiments, the antibody comprises a heavy chain comprising an amino acid sequence having at least 95% identity to the amino acid sequence of SEQ ID NO:125, and a light chain comprising an amino acid sequence having at least 95% identity to the amino acid sequence of SEQ ID NO:126.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:133; and (ii) a VL comprising a VL CDR1, a VL CDR2, and a VL CDR3 having amino acid sequences of the VL CDR1, VL CDR2, and VL CDR3, respectively, of SEQ ID NO:134. In another aspect, provided herein is an antibody that binds to TRGV9, wherein the antibody comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129; and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In another aspect, provided herein is an antibody that binds to TRGV9, wherein the antibody comprises: (i) a VH comprising a VH CDR1 having an amino acid sequence of SEQ ID NO:520, a VH CDR2 having an amino acid sequence of SEQ ID NO:521, and a VH CDR3 having an amino acid sequence of SEQ ID NO:522; and (ii) a VL comprising a VL CDR1 having an amino acid sequence of SEQ ID NO:523, a VL CDR2 having an amino acid sequence of SEQ ID NO:524, and a VL CDR3 having an amino acid sequence of SEQ ID NO:525. In another aspect, provided herein is an antibody that binds to TRGV9, wherein the antibody comprises: (i) a VH comprising a VH CDR1 having an amino acid sequence of SEQ ID NO:526, a VH CDR2 having an amino acid sequence of SEQ ID NO:527, and a VH CDR3 having an amino acid sequence of SEQ ID NO:528; and (ii) a VL comprising a VL CDR1 having an amino acid sequence of SEQ ID NO:529, a VL CDR2 having an amino acid sequence of SEQ ID NO:530, and a VL CDR3 having an amino acid sequence of SEQ ID NO:531. In another aspect, provided herein is an antibody that binds to TRGV9, wherein the antibody comprises: (i) a VH comprising a VH CDR1 having an amino acid sequence of SEQ ID NO:532, a VH CDR2 having an amino acid sequence of SEQ ID NO:533, and a VH CDR3 having an amino acid sequence of SEQ ID NO:534; and (ii) a VL comprising a VL CDR1 having an amino acid sequence of SEQ ID NO:535, a VL CDR2 having an amino acid sequence of SEQ ID NO:536, and a VL CDR3 having an amino acid sequence of SEQ ID NO:537. In another aspect, provided herein is an antibody that binds to TRGV9, wherein the antibody comprises: (i) a VH comprising a VH CDR1 having an amino acid sequence of SEQ ID NO:538, a VH CDR2 having an amino acid sequence of SEQ ID NO:539, and a VH CDR3 having an amino acid sequence of SEQ ID NO:540; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In another aspect, provided herein is an antibody that binds to TRGV9, wherein the antibody comprises: (i) a VH comprising a VH CDR1 having an amino acid sequence of SEQ ID NO:538, a VH CDR2 having an amino acid sequence of SEQ ID NO:720, and a VH CDR3 having an amino acid sequence of SEQ ID NO:721; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In another aspect, provided herein is an antibody that binds to TRGV9, wherein the antibody comprises: (i) a VH comprising a VH CDR1 having an amino acid sequence of SEQ ID NO:544, a VH CDR2 having an amino acid sequence of SEQ ID NO:545, and a VH CDR3 having an amino acid sequence of SEQ ID NO:546; and (ii) a VL comprising a VL CDR1 having an amino acid sequence of SEQ ID NO:547, a VL CDR2 having an amino acid sequence of SEQ ID NO:548, and a VL CDR3 having an amino acid sequence of SEQ ID NO:549. In another aspect, provided herein is an antibody that binds to TRGV9, wherein the antibody comprises: (i) a VH comprising a VH CDR1 having an amino acid sequence of SEQ ID NO:550, a VH CDR2 having an amino acid sequence of SEQ ID NO:551, and a VH CDR3 having an amino acid sequence of SEQ ID NO:552; and (ii) a VL comprising a VL CDR1 having an amino acid sequence of SEQ ID NO:553, a VL CDR2 having an amino acid sequence of SEQ ID NO:554, and a VL CDR3 having an amino acid sequence of SEQ ID NO:555. In some embodiments, the antibody comprises a VH having an amino acid sequence of SEQ ID NO:133. In some embodiments, the antibody comprises a VL having an amino acid sequence of SEQ ID NO:134. In some embodiments, the antibody comprises a VH having an amino acid sequence of SEQ ID NO:133, and a VL having an amino acid sequence of SEQ ID NO:134. In some embodiments, the antibody comprises a heavy chain having an amino acid sequence of SEQ ID NO:135. In some embodiments, the antibody comprises a light chain having an amino acid sequence of SEQ ID NO:136. In some embodiments, the antibody comprises a heavy chain having an amino acid sequence of SEQ ID NO:135, and a light chain having an amino acid sequence of SEQ ID NO:136. In some embodiments, the antibody comprises a VH comprising an amino acid sequence having at least 95% identity to the amino acid sequence of SEQ ID NO:133. In some embodiments, the antibody comprises a VL comprising an amino acid sequence having at least 95% identity to the amino acid sequence of SEQ ID NO:134. In some embodiments, the antibody comprises a VH comprising an amino acid sequence having at least 95% identity to the amino acid sequence of SEQ ID NO:133, and a VL comprising an amino acid sequence having at least 95% identity to the amino acid sequence of SEQ ID NO:134. In some embodiments, the antibody comprises a heavy chain comprising an amino acid sequence having at least 95% identity to the amino acid sequence of SEQ ID NO:135. In some embodiments, the antibody comprises a light chain comprising an amino acid sequence having at least 95% identity to the amino acid sequence of SEQ ID NO:136. In some embodiments, the antibody comprises a heavy chain comprising an amino acid sequence having at least 95% identity to the amino acid sequence of SEQ ID NO:135, and a light chain comprising an amino acid sequence having at least 95% identity to the amino acid sequence of SEQ ID NO:136.

In some embodiments, the anti-TRGV9 antibody is not a single chain antibody. In some embodiments, the anti-TRGV9 antibody is not a single domain antibody. In some embodiments, the anti-TRGV9 antibody is not a nanobody. In certain embodiments, the anti-TRGV9 antibody is not a VHH antibody. In certain embodiments, the anti-TRGV9 antibody is not a llama antibody.

In some embodiments, an anti-TRGV9 antibody provided herein does not comprise a VH CDR1, VH CDR2, and VH CDR3 having the amino acid sequence of SEQ ID NOs:730, 731, and 732, respectively. In some embodiments, an anti-TRGV9 antibody provided herein does not comprise a VH CDR1, VH CDR2, and VH CDR3 having the amino acid sequence of SEQ ID NOs:733, 734, and 735, respectively. In some embodiments, an anti-TRGV9 antibody provided herein does not comprise a VH CDR1, VH CDR2, and VH CDR3 having the amino acid sequence of SEQ ID NOs:736, 737, and 738, respectively. In some embodiments, an anti-TRGV9 antibody provided herein does not comprise a VH CDR1, VH CDR2, and VH CDR3 having the amino acid sequence of SEQ ID NOs:739, 740, and 741, respectively. In some embodiments, an anti-TRGV9 antibody provided herein does not comprise a VH CDR1, VH CDR2, and VH CDR3 having the amino acid sequence of SEQ ID NOs:742, 743, and 744, respectively. In some embodiments, an anti-TRGV9 antibody provided herein does not comprise a VH CDR1, VH CDR2, and VH CDR3 having the amino acid sequence of SEQ ID NOs:745, 746, and 747, respectively. In some embodiments, an anti-TRGV9 antibody provided herein does not comprise a VH CDR1, VH CDR2, and VH CDR3 having the amino acid sequence of SEQ ID NOs:748, 749, and 750, respectively. In some embodiments, an anti-TRGV9 antibody provided herein does not comprise a VH domain having the amino acid sequence of SEQ ID NO:751. In some embodiments, an anti-TRGV9 antibody provided herein does not comprise a VH domain having the amino acid sequence of SEQ ID NO:752. In some embodiments, an anti-TRGV9 antibody provided herein does not comprise a VH domain having the amino acid sequence of SEQ ID NO:753. In some embodiments, an anti-TRGV9 antibody provided herein does not comprise a VH domain having the amino acid sequence of SEQ ID NO:754. In some embodiments, an anti-TRGV9 antibody provided herein does not comprise a VH domain having the amino acid sequence of SEQ ID NO:755. In some embodiments, an anti-TRGV9 antibody provided herein does not comprise a VH domain having the amino acid sequence of SEQ ID NO:756. In some embodiments, an anti-TRGV9 antibody provided herein does not comprise a VH domain having the amino acid sequence of SEQ ID NO:757.

In another aspect, provided herein is an anti-TRGV9 antibody, comprising a VH domain comprising a VH CDR3 having the amino acid sequence of APNxGzYTbDF (SEQ ID NO:758), wherein x is Y or M, z is M or D, and b is I or L. In another aspect, provided herein is an anti-TRGV9 antibody, comprising a VH domain comprising the amino acid sequence of SEQ ID NO:758. In another aspect, provided herein is an anti-TRGV9 antibody, comprising a VH domain comprising a VH CDR1 having the amino acid sequence of GxTFzz, wherein x is F, D or G, and z is S or N. In another aspect, provided herein is an anti-TRGV9 antibody, comprising a VH domain comprising the amino acid sequence of SEQ ID NO:761. In another aspect, provided herein is an anti-TRGV9 antibody, comprising a VL domain comprising a VL CDR1 having the amino acid sequence of RxSQSz (SEQ ID NO:762), wherein x is A or S, and z is V or L. In another aspect, provided herein is an anti-TRGV9 antibody, comprising a VL domain comprising the amino acid sequence of SEQ ID NO:761.

In another aspect, provided herein is an antibody that binds to TRGV9, wherein the antibody comprises: (i) a VH comprising a VH CDR1, a VH CDR2, and a VH CDR3; and (ii) a VL comprising a VL CDR1, a VL CDR2, and a VL CDR3. In some embodiments, the VH CDR1 comprises a first polar amino acid. In some embodiments, the VH CDR1 comprises a last polar uncharged amino acid. In some embodiments, the VH CDR1 comprises at least one tyrosine. In some embodiments, the VH CDR1 comprises at least 20% hydrophobic amino acids. In some embodiments, the VH CDR1 comprises at least two hydrophobic amino acids. In some embodiments, the VH CDR1 comprises at least about 40% hydrophobic amino acids. In some embodiments, the VH CDR1 comprises the VH CDR1 comprises a first polar amino acid, a last polar uncharged amino acid, at least one tyrosine, at least 20% hydrophobic amino acids, at least two hydrophobic amino acids, and at least about 40% hydrophobic amino acids. Any combination of two or more of the above-mentioned VH CDR1 structural features are also contemplated. In some embodiments, the VH CDR2 comprises a polar amino acid at residue 13. In some embodiments, the VH CDR2 comprises a hydrophobic at amino acid position 15. In some embodiments, the VH CDR2 comprises a phenylalanine (F) or leucine (L) at position 15. In some embodiments, the VH CDR2 comprises a polar amino acid at position 14. In some embodiments, the VH CDR2 comprises a lysine (K) or serine (S) at position 14. In some embodiments, the VH CDR2 comprises a hydrophobic amino acid at position 2. In some embodiments, the VH CDR2 comprises a hydrophobic amino acid at position 3. In some embodiments, the VH CDR2 comprises and a polar penultimate amino acid. In some embodiments, the VH CDR2 comprises a polar amino acid at residue 13, a hydrophobic at amino acid position 15, a phenylalanine (F) or leucine (L) at position 15, a polar amino acid at position 14, a lysine (K) or serine (S) at position 14, a hydrophobic amino acid at position 2 or 3, and a polar penultimate amino acid. Any combination of two or more of the above-mentioned VH CDR2 structural features are also contemplated. In some embodiments, the VH CDR3 does not comprise a polar charged amino acid at position 3. In some embodiments, the VH CDR3 comprises a hydrophobic or polar charged amino acid at position 7. In some embodiments, the VH CDR3 comprises a polar uncharged or hydrophobic amino acid at position 6. In some embodiments, the VH CDR3 comprises no polar charged amino acid at position 3, a hydrophobic or polar charged amino acid at position 7, and a polar uncharged or hydrophobic amino acid at position 6. Any combination of two or more of the above-mentioned VH CDR3 structural features are also contemplated. In some embodiments, the VL CDR1 comprises a polar amino acid at position 4. In some embodiments, the VL CDR1 comprises a first amino acid that is polar charged. In some embodiments, the VL CDR1 comprises a polar uncharged or hydrophobic amino acid at position 2. In some embodiments, the VL CDR1 comprises a serine at position 3. In some embodiments, the VL CDR1 comprises a polar amino acid at position 5. In some embodiments, the VL CDR1 comprises a hydrophobic amino acid at position 6. In some embodiments, the VL CDR1 comprises a polar amino acid at position 4, a first amino acid that is polar charged, a polar uncharged or hydrophobic amino acid at position 2, a serine at position 3, a polar amino acid at position 5, and a hydrophobic amino acid at position 6. Any combination of two or more of the above-mentioned VL CDR1 structural features are also contemplated. In some embodiments, the VL CDR2 comprises a polar amino acid at position 7. In some embodiments, the VL CDR2 comprises a polar charged or hydrophobic amino acid at position 6. In some embodiments, the VL CDR2 comprises a polar charged amino acid at position 3. In some embodiments, the VL CDR2 comprises a polar uncharged amino acid at position 4. In some embodiments, the VL CDR2 comprises a hydrophobic amino acid at position 2. In some embodiments, the VL CDR2 comprises a polar amino acid at position 7, a polar charged or hydrophobic amino acid at position 6, a polar charged amino acid at position 3, a polar uncharged amino acid at position 4, and a hydrophobic amino acid at position 2. Any combination of two or more of the above-mentioned VL CDR2 structural features are also contemplated. In some embodiments, the VL CDR3 comprises a hydrophobic terminal amino acid. In some embodiments, the VL CDR3 comprises a terminal tyrosine. In some embodiments, the VL CDR3 comprises a polar uncharged amino acid at position 5. In some embodiments, the VL CDR3 comprises a polar amino acid at position 2. In some embodiments, the VL CDR3 comprises a polar uncharged or hydrophobic amino acid at position 1. In some embodiments, the VL CDR3 comprises a hydrophobic amino acid at position 3. In some embodiments, the VL CDR3 comprises a hydrophylic or polar uncharged amino acid at position 6. In some embodiments, the VL CDR3 comprises no polar or hydrophobic amino acid at position 7. In some embodiments, the VL CDR3 comprises a hydrophobic terminal amino acid, a terminal tyrosine, a polar uncharged amino acid at position 5, a polar amino acid at position 2, a polar uncharged or hydrophobic amino acid at position 1, a hydrophobic amino acid at position 3, a hydrophylic or polar uncharged amino acid at position 6, and no polar or hydrophobic amino acid at position 7. Any combination of two or more of the above-mentioned VL CDR3 structural features are also contemplated. In specific embodiments, residue position numbering is according to Exemplary numbering.

In some embodiments, the anti-TRGV9 antibody is a multispecific antibody. In other embodiments, the anti-TRGV9 antibody is a bispecific antibody. In certain embodiments, the multispecific antibody comprises an antigen binding fragment of an anti-TRGV9 antibody provided herein. In some embodiments, the multispecific antibody comprises a first binding domain that binds to a first TRGV9 epitope and a second domain that binds to a second TRGV9 epitope, wherein the first TRGV9 epitope and the second TRGV9 epitope are different. In certain embodiments, the multispecific antibody further comprises a third binding domain that binds to a target that is not TRGV9.

In another aspect, provided herein is a bispecific antibody comprising: (a) a first binding domain that binds to TRGV9, and (b) a second binding domain that binds to a second target that is not TRGV9.

In some embodiments, the bispecific antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In a some embodiments, the TRGV9 antibody is not a single domain antibody or nanobody.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:7; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:160, a VH CDR2 having an amino acid sequence of SEQ ID NO:161, and a VH CDR3 having an amino acid sequence of SEQ ID NO:162; and (ii) a VL comprising a VL CDR1 having an amino acid sequence of SEQ ID NO:163, a VL CDR2 having an amino acid sequence of SEQ ID NO:164, and a VL CDR3 having an amino acid sequence of SEQ ID NO:165. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:166, a VH CDR2 having an amino acid sequence of SEQ ID NO:167, and a VH CDR3 having an amino acid sequence of SEQ ID NO:168; and (ii) a VL comprising a VL CDR1 having an amino acid sequence of SEQ ID NO:169, a VL CDR2 having an amino acid sequence of SEQ ID NO:170, and a VL CDR3 having an amino acid sequence of SEQ ID NO:171. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:172, a VH CDR2 having an amino acid sequence of SEQ ID NO:173, and a VH CDR3 having an amino acid sequence of SEQ ID NO:174; and (ii) a VL comprising a VL CDR1 having an amino acid sequence of SEQ ID NO:175, a VL CDR2 having an amino acid sequence of SEQ ID NO:176, and a VL CDR3 having an amino acid sequence of SEQ ID NO:177. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:178, a VH CDR2 having an amino acid sequence of SEQ ID NO:179, and a VH CDR3 having an amino acid sequence of SEQ ID NO:180; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:178, a VH CDR2 having an amino acid sequence of SEQ ID NO:700, and a VH CDR3 having an amino acid sequence of SEQ ID NO:701; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:184, a VH CDR2 having an amino acid sequence of SEQ ID NO:185, and a VH CDR3 having an amino acid sequence of SEQ ID NO:186; and (ii) a VL comprising a VL CDR1 having an amino acid sequence of SEQ ID NO:187, a VL CDR2 having an amino acid sequence of SEQ ID NO:188, and a VL CDR3 having an amino acid sequence of SEQ ID NO:189. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:190, a VH CDR2 having an amino acid sequence of SEQ ID NO:191, and a VH CDR3 having an amino acid sequence of SEQ ID NO:192; and (ii) a VL comprising a VL CDR1 having an amino acid sequence of SEQ ID NO:193, a VL CDR2 having an amino acid sequence of SEQ ID NO:194, and a VL CDR3 having an amino acid sequence of SEQ ID NO:195. In some embodiments, the first binding domain comprises a VH having an amino acid sequence of SEQ ID NO:7. In some embodiments, the first binding domain comprises a VL having an amino acid sequence of SEQ ID NO:8. In some embodiments, the first binding domain comprises a VH having an amino acid sequence of SEQ ID NO:7, and a VL having an amino acid sequence of SEQ ID NO:8. In some embodiments, the first binding domain comprises a heavy chain having an amino acid sequence of SEQ ID NO:23. In some embodiments, the first binding domain comprises a light chain having an amino acid sequence of SEQ ID NO:24. In some embodiments, the first binding domain comprises a heavy chain having an amino acid sequence of SEQ ID NO:23, and a light chain having an amino acid sequence of SEQ ID NO:24. In some embodiments, the first binding domain comprises an amino acid sequence of SEQ ID NO:17. In some embodiments, the first binding domain comprises a heavy chain having an amino acid sequence of SEQ ID NO:69. In some embodiments, the first binding domain comprises a light chain having an amino acid sequence of SEQ ID NO:24. In some embodiments, the first binding domain comprises a heavy chain having an amino acid sequence of SEQ ID NO:69, and a light chain having an amino acid sequence of SEQ ID NO:24. In some embodiments, the first binding domain comprises a VH comprising an amino acid sequence having at least 95% identity to the amino acid sequence of SEQ ID NO:7. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:7, and a VL comprising an amino acid sequence having at least 95% identity to the amino acid sequence of SEQ ID NO:8. In some embodiments, the first binding domain comprises a heavy chain comprising an amino acid sequence having at least 95% identity to the amino acid sequence of SEQ ID NO:23. In some embodiments, the first binding domain comprises a light chain comprising an amino acid sequence having at least 95% identity to the amino acid sequence of SEQ ID NO:24. In some embodiments, the first binding domain comprises a heavy chain comprising an amino acid sequence having at least 95% identity to the amino acid sequence of SEQ ID NO:23, and a light chain comprising an amino acid sequence having at least 95% identity to the amino acid sequence of SEQ ID NO:24. In some embodiments, the first binding domain comprises an amino acid sequence of SEQ ID NO:17. In some embodiments, the first binding domain comprises a heavy chain comprising an amino acid sequence having at least 95% identity to the amino acid sequence of SEQ ID NO:69. In some embodiments, the first binding domain comprises a light chain comprising an amino acid sequence having at least 95% identity to the amino acid sequence of SEQ ID NO:24. In some embodiments, the first binding domain comprises a heavy chain comprising an amino acid sequence having at least 95% identity to the amino acid sequence of SEQ ID NO:69, and a light chain comprising an amino acid sequence having at least 95% identity to the amino acid sequence of SEQ ID NO:24.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:34; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:196, a VH CDR2 having an amino acid sequence of SEQ ID NO:197, and a VH CDR3 having an amino acid sequence of SEQ ID NO:198; and (ii) a VL comprising a VL CDR1 having an amino acid sequence of SEQ ID NO:199, a VL CDR2 having an amino acid sequence of SEQ ID NO:200, and a VL CDR3 having an amino acid sequence of SEQ ID NO:201. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:202, a VH CDR2 having an amino acid sequence of SEQ ID NO:203, and a VH CDR3 having an amino acid sequence of SEQ ID NO:204; and (ii) a VL comprising a VL CDR1 having an amino acid sequence of SEQ ID NO:205, a VL CDR2 having an amino acid sequence of SEQ ID NO:206, and a VL CDR3 having an amino acid sequence of SEQ ID NO:207. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:208, a VH CDR2 having an amino acid sequence of SEQ ID NO:209, and a VH CDR3 having an amino acid sequence of SEQ ID NO:210; and (ii) a VL comprising a VL CDR1 having an amino acid sequence of SEQ ID NO:211, a VL CDR2 having an amino acid sequence of SEQ ID NO:212, and a VL CDR3 having an amino acid sequence of SEQ ID NO:213. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:214, a VH CDR2 having an amino acid sequence of SEQ ID NO:215, and a VH CDR3 having an amino acid sequence of SEQ ID NO:216; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:214, a VH CDR2 having an amino acid sequence of SEQ ID NO:702, and a VH CDR3 having an amino acid sequence of SEQ ID NO:703; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:220, a VH CDR2 having an amino acid sequence of SEQ ID NO:221, and a VH CDR3 having an amino acid sequence of SEQ ID NO:222; and (ii) a VL comprising a VL CDR1 having an amino acid sequence of SEQ ID NO:223, a VL CDR2 having an amino acid sequence of SEQ ID NO:224, and a VL CDR3 having an amino acid sequence of SEQ ID NO:225. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:226, a VH CDR2 having an amino acid sequence of SEQ ID NO:227, and a VH CDR3 having an amino acid sequence of SEQ ID NO:228; and (ii) a VL comprising a VL CDR1 having an amino acid sequence of SEQ ID NO:229, a VL CDR2 having an amino acid sequence of SEQ ID NO:230, and a VL CDR3 having an amino acid sequence of SEQ ID NO:231. In some embodiments, the first binding domain comprises a VH having an amino acid sequence of SEQ ID NO:34. In some embodiments, the first binding domain comprises a VL having an amino acid sequence of SEQ ID NO:8. In some embodiments, the first binding domain comprises a VH having an amino acid sequence of SEQ ID NO:34, and a VL having an amino acid sequence of SEQ ID NO:8. In some embodiments, the first binding domain comprises a VH comprising an amino acid sequence having at least 95% identity to the amino acid sequence of SEQ ID NO:34. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:34, and a VL comprising an amino acid sequence having at least 95% identity to the amino acid sequence of SEQ ID NO:8.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:35; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:232, a VH CDR2 having an amino acid sequence of SEQ ID NO:233, and a VH CDR3 having an amino acid sequence of SEQ ID NO:234; and (ii) a VL comprising a VL CDR1 having an amino acid sequence of SEQ ID NO:235, a VL CDR2 having an amino acid sequence of SEQ ID NO:236, and a VL CDR3 having an amino acid sequence of SEQ ID NO:237. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:238, a VH CDR2 having an amino acid sequence of SEQ ID NO:239, and a VH CDR3 having an amino acid sequence of SEQ ID NO:240; and (ii) a VL comprising a VL CDR1 having an amino acid sequence of SEQ ID NO:241, a VL CDR2 having an amino acid sequence of SEQ ID NO:242, and a VL CDR3 having an amino acid sequence of SEQ ID NO:243. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:244, a VH CDR2 having an amino acid sequence of SEQ ID NO:245, and a VH CDR3 having an amino acid sequence of SEQ ID NO:246; and (ii) a VL comprising a VL CDR1 having an amino acid sequence of SEQ ID NO:247, a VL CDR2 having an amino acid sequence of SEQ ID NO:248, and a VL CDR3 having an amino acid sequence of SEQ ID NO:249. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:250, a VH CDR2 having an amino acid sequence of SEQ ID NO:251, and a VH CDR3 having an amino acid sequence of SEQ ID NO:252; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:250, a VH CDR2 having an amino acid sequence of SEQ ID NO:704, and a VH CDR3 having an amino acid sequence of SEQ ID NO:705; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:256, a VH CDR2 having an amino acid sequence of SEQ ID NO:257, and a VH CDR3 having an amino acid sequence of SEQ ID NO:258; and (ii) a VL comprising a VL CDR1 having an amino acid sequence of SEQ ID NO:259, a VL CDR2 having an amino acid sequence of SEQ ID NO:260, and a VL CDR3 having an amino acid sequence of SEQ ID NO:261. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:262, a VH CDR2 having an amino acid sequence of SEQ ID NO:263, and a VH CDR3 having an amino acid sequence of SEQ ID NO:264; and (ii) a VL comprising a VL CDR1 having an amino acid sequence of SEQ ID NO:265, a VL CDR2 having an amino acid sequence of SEQ ID NO:266, and a VL CDR3 having an amino acid sequence of SEQ ID NO:267. In some embodiments, the first binding domain comprises a VH having an amino acid sequence of SEQ ID NO:35. In some embodiments, the first binding domain comprises a VL having an amino acid sequence of SEQ ID NO:8. In some embodiments, the first binding domain comprises a VH having an amino acid sequence of SEQ ID NO:35, and a VL having an amino acid sequence of SEQ ID NO:8. In some embodiments, the first binding domain comprises a VH comprising an amino acid sequence having at least 95% identity to the amino acid sequence of SEQ ID NO:35. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:35, and a VL comprising an amino acid sequence having at least 95% identity to the amino acid sequence of SEQ ID NO:8.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:36; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:268, a VH CDR2 having an amino acid sequence of SEQ ID NO:269, and a VH CDR3 having an amino acid sequence of SEQ ID NO:270; and (ii) a VL comprising a VL CDR1 having an amino acid sequence of SEQ ID NO:271, a VL CDR2 having an amino acid sequence of SEQ ID NO:272, and a VL CDR3 having an amino acid sequence of SEQ ID NO:273. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:274, a VH CDR2 having an amino acid sequence of SEQ ID NO:275, and a VH CDR3 having an amino acid sequence of SEQ ID NO:276; and (ii) a VL comprising a VL CDR1 having an amino acid sequence of SEQ ID NO:277, a VL CDR2 having an amino acid sequence of SEQ ID NO:278, and a VL CDR3 having an amino acid sequence of SEQ ID NO:279. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:280, a VH CDR2 having an amino acid sequence of SEQ ID NO:281, and a VH CDR3 having an amino acid sequence of SEQ ID NO:282; and (ii) a VL comprising a VL CDR1 having an amino acid sequence of SEQ ID NO:283, a VL CDR2 having an amino acid sequence of SEQ ID NO:284, and a VL CDR3 having an amino acid sequence of SEQ ID NO:285. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:286, a VH CDR2 having an amino acid sequence of SEQ ID NO:287, and a VH CDR3 having an amino acid sequence of SEQ ID NO:288; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:286, a VH CDR2 having an amino acid sequence of SEQ ID NO:706, and a VH CDR3 having an amino acid sequence of SEQ ID NO:707; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:292, a VH CDR2 having an amino acid sequence of SEQ ID NO:293, and a VH CDR3 having an amino acid sequence of SEQ ID NO:294; and (ii) a VL comprising a VL CDR1 having an amino acid sequence of SEQ ID NO:295, a VL CDR2 having an amino acid sequence of SEQ ID NO:296, and a VL CDR3 having an amino acid sequence of SEQ ID NO:297. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:298, a VH CDR2 having an amino acid sequence of SEQ ID NO:299, and a VH CDR3 having an amino acid sequence of SEQ ID NO:300; and (ii) a VL comprising a VL CDR1 having an amino acid sequence of SEQ ID NO:301, a VL CDR2 having an amino acid sequence of SEQ ID NO:302, and a VL CDR3 having an amino acid sequence of SEQ ID NO:303. In some embodiments, the first binding domain comprises a VH having an amino acid sequence of SEQ ID NO:36. In some embodiments, the first binding domain comprises a VL having an amino acid sequence of SEQ ID NO:8. In some embodiments, the first binding domain comprises a VH having an amino acid sequence of SEQ ID NO:36, and a VL having an amino acid sequence of SEQ ID NO:8. In some embodiments, the first binding domain comprises a VH comprising an amino acid sequence having at least 95% identity to the amino acid sequence of SEQ ID NO:36. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:36, and a VL comprising an amino acid sequence having at least 95% identity to the amino acid sequence of SEQ ID NO:8.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:65; and (ii) a VL comprising a VL CDR1, a VL CDR2, and a VL CDR3 having amino acid sequences of the VL CDR1, VL CDR2, and VL CDR3, respectively, of SEQ ID NO:66. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:67; and (ii) a VL comprising a VL CDR1, a VL CDR2, and a VL CDR3 having amino acid sequences of the VL CDR1, VL CDR2, and VL CDR3, respectively, of SEQ ID NO:68. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:304, a VH CDR2 having an amino acid sequence of SEQ ID NO:305, and a VH CDR3 having an amino acid sequence of SEQ ID NO:306; and (ii) a VL comprising a VL CDR1 having an amino acid sequence of SEQ ID NO:307, a VL CDR2 having an amino acid sequence of SEQ ID NO:308, and a VL CDR3 having an amino acid sequence of SEQ ID NO:309. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:310, a VH CDR2 having an amino acid sequence of SEQ ID NO:311, and a VH CDR3 having an amino acid sequence of SEQ ID NO:312; and (ii) a VL comprising a VL CDR1 having an amino acid sequence of SEQ ID NO:313, a VL CDR2 having an amino acid sequence of SEQ ID NO:314, and a VL CDR3 having an amino acid sequence of SEQ ID NO:315. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:316, a VH CDR2 having an amino acid sequence of SEQ ID NO:317, and a VH CDR3 having an amino acid sequence of SEQ ID NO:318; and (ii) a VL comprising a VL CDR1 having an amino acid sequence of SEQ ID NO:319, a VL CDR2 having an amino acid sequence of SEQ ID NO:320, and a VL CDR3 having an amino acid sequence of SEQ ID NO:321. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:322, a VH CDR2 having an amino acid sequence of SEQ ID NO:323, and a VH CDR3 having an amino acid sequence of SEQ ID NO:324; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:322, a VH CDR2 having an amino acid sequence of SEQ ID NO:708, and a VH CDR3 having an amino acid sequence of SEQ ID NO:709; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:328, a VH CDR2 having an amino acid sequence of SEQ ID NO:329, and a VH CDR3 having an amino acid sequence of SEQ ID NO:330; and (ii) a VL comprising a VL CDR1 having an amino acid sequence of SEQ ID NO:331, a VL CDR2 having an amino acid sequence of SEQ ID NO:332, and a VL CDR3 having an amino acid sequence of SEQ ID NO:333. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:334, a VH CDR2 having an amino acid sequence of SEQ ID NO:335, and a VH CDR3 having an amino acid sequence of SEQ ID NO:336; and (ii) a VL comprising a VL CDR1 having an amino acid sequence of SEQ ID NO:337, a VL CDR2 having an amino acid sequence of SEQ ID NO:338, and a VL CDR3 having an amino acid sequence of SEQ ID NO:339. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:340, a VH CDR2 having an amino acid sequence of SEQ ID NO:341, and a VH CDR3 having an amino acid sequence of SEQ ID NO:342; and (ii) a VL comprising a VL CDR1 having an amino acid sequence of SEQ ID NO:343, a VL CDR2 having an amino acid sequence of SEQ ID NO:344, and a VL CDR3 having an amino acid sequence of SEQ ID NO:345. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:346, a VH CDR2 having an amino acid sequence of SEQ ID NO:347, and a VH CDR3 having an amino acid sequence of SEQ ID NO:348; and (ii) a VL comprising a VL CDR1 having an amino acid sequence of SEQ ID NO:349, a VL CDR2 having an amino acid sequence of SEQ ID NO:350, and a VL CDR3 having an amino acid sequence of SEQ ID NO:351. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:352, a VH CDR2 having an amino acid sequence of SEQ ID NO:353, and a VH CDR3 having an amino acid sequence of SEQ ID NO:354; and (ii) a VL comprising a VL CDR1 having an amino acid sequence of SEQ ID NO:355, a VL CDR2 having an amino acid sequence of SEQ ID NO:356, and a VL CDR3 having an amino acid sequence of SEQ ID NO:357. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:358, a VH CDR2 having an amino acid sequence of SEQ ID NO:359, and a VH CDR3 having an amino acid sequence of SEQ ID NO:360; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:358, a VH CDR2 having an amino acid sequence of SEQ ID NO:710, and a VH CDR3 having an amino acid sequence of SEQ ID NO:711; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:364, a VH CDR2 having an amino acid sequence of SEQ ID NO:365, and a VH CDR3 having an amino acid sequence of SEQ ID NO:366; and (ii) a VL comprising a VL CDR1 having an amino acid sequence of SEQ ID NO:367, a VL CDR2 having an amino acid sequence of SEQ ID NO:368, and a VL CDR3 having an amino acid sequence of SEQ ID NO:369. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:370, a VH CDR2 having an amino acid sequence of SEQ ID NO:371, and a VH CDR3 having an amino acid sequence of SEQ ID NO:372; and (ii) a VL comprising a VL CDR1 having an amino acid sequence of SEQ ID NO:373, a VL CDR2 having an amino acid sequence of SEQ ID NO:374, and a VL CDR3 having an amino acid sequence of SEQ ID NO:375. In some embodiments, the first binding domain comprises a VH having an amino acid sequence of SEQ ID NO:65. In some embodiments, the first binding domain comprises a VL having an amino acid sequence of SEQ ID NO:66. In some embodiments, the first binding domain comprises a VH having an amino acid sequence of SEQ ID NO:65, and a VL having an amino acid sequence of SEQ ID NO:66. In some embodiments, the first binding domain comprises a VH having an amino acid sequence of SEQ ID NO:67. In some embodiments, the first binding domain comprises a VL having an amino acid sequence of SEQ ID NO:68. In some embodiments, the first binding domain a VH having an amino acid sequence of SEQ ID NO:67, and a VL having an amino acid sequence of SEQ ID NO:68. In some embodiments, the first binding domain comprises a heavy chain having an amino acid sequence of SEQ ID NO:71. In some embodiments, the first binding domain comprises a light chain having an amino acid sequence of SEQ ID NO:72. In some embodiments, the first binding domain comprises a heavy chain having an amino acid sequence of SEQ ID NO:71, and a light chain having an amino acid sequence of SEQ ID NO:72. In some embodiments, the first binding domain comprises an amino acid sequence of SEQ ID NO:70. In some embodiments, the first binding domain comprises a heavy chain having an amino acid sequence of SEQ ID NO:74. In some embodiments, the first binding domain comprises a light chain having an amino acid sequence of SEQ ID NO:75. In some embodiments, the first binding domain comprises a heavy chain having an amino acid sequence of SEQ ID NO:74, and a light chain having an amino acid sequence of SEQ ID NO:75. In some embodiments, the first binding domain comprises an amino acid sequence of SEQ ID NO:73. In some embodiments, the first binding domain comprises a VH comprising an amino acid sequence having at least 95% identity to the amino acid sequence of SEQ ID NO:65. In some embodiments, the first binding domain comprises a VL comprising an amino acid sequence having at least 95% identity to the amino acid sequence of SEQ ID NO:66. In some embodiments, the first binding domain comprises a VH comprising an amino acid sequence having at least 95% identity to the amino acid sequence of SEQ ID NO:65, and a VL comprising an amino acid sequence having at least 95% identity to the amino acid sequence of SEQ ID NO:66. In some embodiments, the first binding domain comprises a VH comprising an amino acid sequence having at least 95% identity to the amino acid sequence of SEQ ID NO:67. In some embodiments, the first binding domain comprises a VL comprising an amino acid sequence having at least 95% identity to the amino acid sequence of SEQ ID NO:68. In some embodiments, the first binding domain a VH comprising an amino acid sequence having at least 95% identity to the amino acid sequence of SEQ ID NO:67, and a VL comprising an amino acid sequence having at least 95% identity to the amino acid sequence of SEQ ID NO:68. In some embodiments, the first binding domain comprises a heavy chain comprising an amino acid sequence having at least 95% identity to the amino acid sequence of SEQ ID NO:71. In some embodiments, the first binding domain comprises a light chain comprising an amino acid sequence having at least 95% identity to the amino acid sequence of SEQ ID NO:72. In some embodiments, the first binding domain comprises a heavy chain comprising an amino acid sequence having at least 95% identity to the amino acid sequence of SEQ ID NO:71, and a light chain comprising an amino acid sequence having at least 95% identity to the amino acid sequence of SEQ ID NO:72. In some embodiments, the first binding domain comprises an amino acid sequence having at least 95% identity to an amino acid sequence of SEQ ID NO:70. In some embodiments, the first binding domain comprises a heavy chain comprising an amino acid sequence having at least 95% identity to the amino acid sequence of SEQ ID NO:74. In some embodiments, the first binding domain comprises a light chain comprising an amino acid sequence having at least 95% identity to the amino acid sequence of SEQ ID NO:75. In some embodiments, the first binding domain comprises a heavy chain comprising an amino acid sequence having at least 95% identity to the amino acid sequence of SEQ ID NO:74, and a light chain comprising an amino acid sequence having at least 95% identity to the amino acid sequence of SEQ ID NO:75. In some embodiments, the first binding domain comprises an amino acid sequence having at least 95% identity to an amino acid sequence of SEQ ID NO:73.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:95; and (ii) a VL comprising a VL CDR1, a VL CDR2, and a VL CDR3 having amino acid sequences of the VL CDR1, VL CDR2, and VL CDR3, respectively, of SEQ ID NO:96. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:376, a VH CDR2 having an amino acid sequence of SEQ ID NO:377, and a VH CDR3 having an amino acid sequence of SEQ ID NO:378; and (ii) a VL comprising a VL CDR1 having an amino acid sequence of SEQ ID NO:379, a VL CDR2 having an amino acid sequence of SEQ ID NO:380, and a VL CDR3 having an amino acid sequence of SEQ ID NO:381. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:382, a VH CDR2 having an amino acid sequence of SEQ ID NO:383, and a VH CDR3 having an amino acid sequence of SEQ ID NO:384; and (ii) a VL comprising a VL CDR1 having an amino acid sequence of SEQ ID NO:385, a VL CDR2 having an amino acid sequence of SEQ ID NO:386, and a VL CDR3 having an amino acid sequence of SEQ ID NO:387. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:388, a VH CDR2 having an amino acid sequence of SEQ ID NO:389, and a VH CDR3 having an amino acid sequence of SEQ ID NO:390; and (ii) a VL comprising a VL CDR1 having an amino acid sequence of SEQ ID NO:391, a VL CDR2 having an amino acid sequence of SEQ ID NO:392, and a VL CDR3 having an amino acid sequence of SEQ ID NO:393. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:394, a VH CDR2 having an amino acid sequence of SEQ ID NO:395, and a VH CDR3 having an amino acid sequence of SEQ ID NO:396; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:394, a VH CDR2 having an amino acid sequence of SEQ ID NO:712, and a VH CDR3 having an amino acid sequence of SEQ ID NO:713; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:400, a VH CDR2 having an amino acid sequence of SEQ ID NO:401, and a VH CDR3 having an amino acid sequence of SEQ ID NO:402; and (ii) a VL comprising a VL CDR1 having an amino acid sequence of SEQ ID NO:403, a VL CDR2 having an amino acid sequence of SEQ ID NO:404, and a VL CDR3 having an amino acid sequence of SEQ ID NO:405. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:406, a VH CDR2 having an amino acid sequence of SEQ ID NO:407, and a VH CDR3 having an amino acid sequence of SEQ ID NO:408; and (ii) a VL comprising a VL CDR1 having an amino acid sequence of SEQ ID NO:409, a VL CDR2 having an amino acid sequence of SEQ ID NO:410, and a VL CDR3 having an amino acid sequence of SEQ ID NO:411. In some embodiments, the first binding domain comprises a VH having an amino acid sequence of SEQ ID NO:95. In some embodiments, the first binding domain comprises a VL having an amino acid sequence of SEQ ID NO:96. In some embodiments, the first binding domain comprises a VH having an amino acid sequence of SEQ ID NO:95, and a VL having an amino acid sequence of SEQ ID NO:96. In some embodiments, the first binding domain comprises an amino acid sequence of SEQ ID NO:97. In some embodiments, the first binding domain comprises a VH comprising an amino acid sequence having at least 95% identity to the amino acid sequence of SEQ ID NO:95. In some embodiments, the first binding domain comprises a VL comprising an amino acid sequence having at least 95% identity to the amino acid sequence of SEQ ID NO:96. In some embodiments, the first binding domain comprises a VH comprising an amino acid sequence having at least 95% identity to the amino acid sequence of SEQ ID NO:95, and a VL comprising an amino acid sequence having at least 95% identity to the amino acid sequence of SEQ ID NO:96. In some embodiments, the first binding domain comprises an amino acid sequence having at least 95% identity to an amino acid sequence of SEQ ID NO:97.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:104; and (ii) a VL comprising a VL CDR1, a VL CDR2, and a VL CDR3 having amino acid sequences of the VL CDR1, VL CDR2, and VL CDR3, respectively, of SEQ ID NO:105. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100, and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:412, a VH CDR2 having an amino acid sequence of SEQ ID NO:413, and a VH CDR3 having an amino acid sequence of SEQ ID NO:414; and (ii) a VL comprising a VL CDR1 having an amino acid sequence of SEQ ID NO:415, a VL CDR2 having an amino acid sequence of SEQ ID NO:416, and a VL CDR3 having an amino acid sequence of SEQ ID NO:417. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:418, a VH CDR2 having an amino acid sequence of SEQ ID NO:419, and a VH CDR3 having an amino acid sequence of SEQ ID NO:420; and (ii) a VL comprising a VL CDR1 having an amino acid sequence of SEQ ID NO:421, a VL CDR2 having an amino acid sequence of SEQ ID NO:422, and a VL CDR3 having an amino acid sequence of SEQ ID NO:423. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:424, a VH CDR2 having an amino acid sequence of SEQ ID NO:425, and a VH CDR3 having an amino acid sequence of SEQ ID NO:426; and (ii) a VL comprising a VL CDR1 having an amino acid sequence of SEQ ID NO:427, a VL CDR2 having an amino acid sequence of SEQ ID NO:428, and a VL CDR3 having an amino acid sequence of SEQ ID NO:429. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:430, a VH CDR2 having an amino acid sequence of SEQ ID NO:431, and a VH CDR3 having an amino acid sequence of SEQ ID NO:432; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:430, a VH CDR2 having an amino acid sequence of SEQ ID NO:714, and a VH CDR3 having an amino acid sequence of SEQ ID NO:715; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:436, a VH CDR2 having an amino acid sequence of SEQ ID NO:437, and a VH CDR3 having an amino acid sequence of SEQ ID NO:438; and (ii) a VL comprising a VL CDR1 having an amino acid sequence of SEQ ID NO:439, a VL CDR2 having an amino acid sequence of SEQ ID NO:440, and a VL CDR3 having an amino acid sequence of SEQ ID NO:441. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:442, a VH CDR2 having an amino acid sequence of SEQ ID NO:443, and a VH CDR3 having an amino acid sequence of SEQ ID NO:444; and (ii) a VL comprising a VL CDR1 having an amino acid sequence of SEQ ID NO:445, a VL CDR2 having an amino acid sequence of SEQ ID NO:446, and a VL CDR3 having an amino acid sequence of SEQ ID NO:447. In some embodiments, the first binding domain comprises a VH having an amino acid sequence of SEQ ID NO:104. In some embodiments, the first binding domain comprises a VL having an amino acid sequence of SEQ ID NO:105. In some embodiments, the first binding domain comprises a VH having an amino acid sequence of SEQ ID NO:104, and a VL having an amino acid sequence of SEQ ID NO:105. In some embodiments, the first binding domain comprises an amino acid sequence of SEQ ID NO:106. In some embodiments, the first binding domain comprises a VH comprising an amino acid sequence having at least 95% identity to the amino acid sequence of SEQ ID NO:104. In some embodiments, the first binding domain comprises a VL comprising an amino acid sequence having at least 95% identity to the amino acid sequence of SEQ ID NO:105. In some embodiments, the first binding domain comprises a VH comprising an amino acid sequence having at least 95% identity to the amino acid sequence of SEQ ID NO:104, and a VL comprising an amino acid sequence having at least 95% identity to the amino acid sequence of SEQ ID NO:105. In some embodiments, the first binding domain comprises an amino acid sequence having at least 95% identity to an amino acid sequence of SEQ ID NO:106.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:113; and (ii) a VL comprising a VL CDR1, a VL CDR2, and a VL CDR3 having amino acid sequences of the VL CDR1, VL CDR2, and VL CDR3, respectively, of SEQ ID NO:114. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109, and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:448, a VH CDR2 having an amino acid sequence of SEQ ID NO:449, and a VH CDR3 having an amino acid sequence of SEQ ID NO:450; and (ii) a VL comprising a VL CDR1 having an amino acid sequence of SEQ ID NO:451, a VL CDR2 having an amino acid sequence of SEQ ID NO:452, and a VL CDR3 having an amino acid sequence of SEQ ID NO:453. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:454, a VH CDR2 having an amino acid sequence of SEQ ID NO:455, and a VH CDR3 having an amino acid sequence of SEQ ID NO:456; and (ii) a VL comprising a VL CDR1 having an amino acid sequence of SEQ ID NO:457, a VL CDR2 having an amino acid sequence of SEQ ID NO:458, and a VL CDR3 having an amino acid sequence of SEQ ID NO:459. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:460, a VH CDR2 having an amino acid sequence of SEQ ID NO:461, and a VH CDR3 having an amino acid sequence of SEQ ID NO:462; and (ii) a VL comprising a VL CDR1 having an amino acid sequence of SEQ ID NO:463, a VL CDR2 having an amino acid sequence of SEQ ID NO:464, and a VL CDR3 having an amino acid sequence of SEQ ID NO:465. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:466, a VH CDR2 having an amino acid sequence of SEQ ID NO:467, and a VH CDR3 having an amino acid sequence of SEQ ID NO:468; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:466, a VH CDR2 having an amino acid sequence of SEQ ID NO:716, and a VH CDR3 having an amino acid sequence of SEQ ID NO:717; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:472, a VH CDR2 having an amino acid sequence of SEQ ID NO:473, and a VH CDR3 having an amino acid sequence of SEQ ID NO:474; and (ii) a VL comprising a VL CDR1 having an amino acid sequence of SEQ ID NO:475, a VL CDR2 having an amino acid sequence of SEQ ID NO:476, and a VL CDR3 having an amino acid sequence of SEQ ID NO:477. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:478, a VH CDR2 having an amino acid sequence of SEQ ID NO:479, and a VH CDR3 having an amino acid sequence of SEQ ID NO:480; and (ii) a VL comprising a VL CDR1 having an amino acid sequence of SEQ ID NO:481, a VL CDR2 having an amino acid sequence of SEQ ID NO:482, and a VL CDR3 having an amino acid sequence of SEQ ID NO:483. In some embodiments, the first binding domain comprises a VH having an amino acid sequence of SEQ ID NO:113. In some embodiments, the first binding domain comprises a VL having an amino acid sequence of SEQ ID NO:114. In some embodiments, the first binding domain comprises a VH having an amino acid sequence of SEQ ID NO:113, and a VL having an amino acid sequence of SEQ ID NO:114. In some embodiments, the first binding domain comprises a heavy chain having an amino acid sequence of SEQ ID NO:115. In some embodiments, the first binding domain comprises a light chain having an amino acid sequence of SEQ ID NO:116. In some embodiments, the first binding domain comprises a heavy chain having an amino acid sequence of SEQ ID NO:115, and a light chain having an amino acid sequence of SEQ ID NO:116. In some embodiments, the first binding domain comprises a VH comprising an amino acid sequence having at least 95% identity to the amino acid sequence of SEQ ID NO:113. In some embodiments, the first binding domain comprises a VL comprising an amino acid sequence having at least 95% identity to the amino acid sequence of SEQ ID NO:114. In some embodiments, the first binding domain comprises a VH comprising an amino acid sequence having at least 95% identity to the amino acid sequence of SEQ ID NO:113, and a VL comprising an amino acid sequence having at least 95% identity to the amino acid sequence of SEQ ID NO:114. In some embodiments, the first binding domain comprises a heavy chain comprising an amino acid sequence having at least 95% identity to the amino acid sequence of SEQ ID NO:115. In some embodiments, the first binding domain comprises a light chain comprising an amino acid sequence having at least 95% identity to the amino acid sequence of SEQ ID NO:116. In some embodiments, the first binding domain comprises a heavy chain comprising an amino acid sequence having at least 95% identity to the amino acid sequence of SEQ ID NO:115, and a light chain comprising an amino acid sequence having at least 95% identity to the amino acid sequence of SEQ ID NO:116.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:123; and (ii) a VL comprising a VL CDR1, a VL CDR2, and a VL CDR3 having amino acid sequences of the VL CDR1, VL CDR2, and VL CDR3, respectively, of SEQ ID NO:124. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119, and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:484, a VH CDR2 having an amino acid sequence of SEQ ID NO:485, and a VH CDR3 having an amino acid sequence of SEQ ID NO:486; and (ii) a VL comprising a VL CDR1 having an amino acid sequence of SEQ ID NO:487, a VL CDR2 having an amino acid sequence of SEQ ID NO:488, and a VL CDR3 having an amino acid sequence of SEQ ID NO:489. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:490, a VH CDR2 having an amino acid sequence of SEQ ID NO:491, and a VH CDR3 having an amino acid sequence of SEQ ID NO:492; and (ii) a VL comprising a VL CDR1 having an amino acid sequence of SEQ ID NO:493, a VL CDR2 having an amino acid sequence of SEQ ID NO:494, and a VL CDR3 having an amino acid sequence of SEQ ID NO:495. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:496, a VH CDR2 having an amino acid sequence of SEQ ID NO:497, and a VH CDR3 having an amino acid sequence of SEQ ID NO:498; and (ii) a VL comprising a VL CDR1 having an amino acid sequence of SEQ ID NO:499, a VL CDR2 having an amino acid sequence of SEQ ID NO:500, and a VL CDR3 having an amino acid sequence of SEQ ID NO:501. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:502, a VH CDR2 having an amino acid sequence of SEQ ID NO:503, and a VH CDR3 having an amino acid sequence of SEQ ID NO:504; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:502, a VH CDR2 having an amino acid sequence of SEQ ID NO:718, and a VH CDR3 having an amino acid sequence of SEQ ID NO:719; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:508, a VH CDR2 having an amino acid sequence of SEQ ID NO:509, and a VH CDR3 having an amino acid sequence of SEQ ID NO:510; and (ii) a VL comprising a VL CDR1 having an amino acid sequence of SEQ ID NO:511, a VL CDR2 having an amino acid sequence of SEQ ID NO:512, and a VL CDR3 having an amino acid sequence of SEQ ID NO:513. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:514, a VH CDR2 having an amino acid sequence of SEQ ID NO:515, and a VH CDR3 having an amino acid sequence of SEQ ID NO:516; and (ii) a VL comprising a VL CDR1 having an amino acid sequence of SEQ ID NO:517, a VL CDR2 having an amino acid sequence of SEQ ID NO:518, and a VL CDR3 having an amino acid sequence of SEQ ID NO:519. In some embodiments, the first binding domain comprises a VH having an amino acid sequence of SEQ ID NO:123. In some embodiments, the first binding domain comprises a VL having an amino acid sequence of SEQ ID NO:124. In some embodiments, the first binding domain comprises a VH having an amino acid sequence of SEQ ID NO:123, and a VL having an amino acid sequence of SEQ ID NO:124. In some embodiments, the first binding domain comprises a heavy chain having an amino acid sequence of SEQ ID NO:125. In some embodiments, the first binding domain comprises a light chain having an amino acid sequence of SEQ ID NO:126. In some embodiments, the first binding domain comprises a heavy chain having an amino acid sequence of SEQ ID NO:125, and a light chain having an amino acid sequence of SEQ ID NO:126. In some embodiments, the first binding domain comprises a VH comprising an amino acid sequence having at least 95% identity to the amino acid sequence of SEQ ID NO:123. In some embodiments, the first binding domain comprises a VL comprising an amino acid sequence having at least 95% identity to the amino acid sequence of SEQ ID NO:124. In some embodiments, the first binding domain comprises a VH comprising an amino acid sequence having at least 95% identity to the amino acid sequence of SEQ ID NO:123, and a VL comprising an amino acid sequence having at least 95% identity to the amino acid sequence of SEQ ID NO:124. In some embodiments, the first binding domain comprises a heavy chain comprising an amino acid sequence having at least 95% identity to the amino acid sequence of SEQ ID NO:125. In some embodiments, the first binding domain comprises a light chain comprising an amino acid sequence having at least 95% identity to the amino acid sequence of SEQ ID NO:126. In some embodiments, the first binding domain comprises a heavy chain comprising an amino acid sequence having at least 95% identity to the amino acid sequence of SEQ ID NO:125, and a light chain comprising an amino acid sequence having at least 95% identity to the amino acid sequence of SEQ ID NO:126.

In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:133; and (ii) a VL comprising a VL CDR1, a VL CDR2, and a VL CDR3 having amino acid sequences of the VL CDR1, VL CDR2, and VL CDR3, respectively, of SEQ ID NO:134. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129, and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:520, a VH CDR2 having an amino acid sequence of SEQ ID NO:521, and a VH CDR3 having an amino acid sequence of SEQ ID NO:522; and (ii) a VL comprising a VL CDR1 having an amino acid sequence of SEQ ID NO:523, a VL CDR2 having an amino acid sequence of SEQ ID NO:524, and a VL CDR3 having an amino acid sequence of SEQ ID NO:525. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:526, a VH CDR2 having an amino acid sequence of SEQ ID NO:527, and a VH CDR3 having an amino acid sequence of SEQ ID NO:528; and (ii) a VL comprising a VL CDR1 having an amino acid sequence of SEQ ID NO:529, a VL CDR2 having an amino acid sequence of SEQ ID NO:530, and a VL CDR3 having an amino acid sequence of SEQ ID NO:531. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:532, a VH CDR2 having an amino acid sequence of SEQ ID NO:533, and a VH CDR3 having an amino acid sequence of SEQ ID NO:534; and (ii) a VL comprising a VL CDR1 having an amino acid sequence of SEQ ID NO:535, a VL CDR2 having an amino acid sequence of SEQ ID NO:536, and a VL CDR3 having an amino acid sequence of SEQ ID NO:537. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:538, a VH CDR2 having an amino acid sequence of SEQ ID NO:539, and a VH CDR3 having an amino acid sequence of SEQ ID NO:540; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:538, a VH CDR2 having an amino acid sequence of SEQ ID NO:720, and a VH CDR3 having an amino acid sequence of SEQ ID NO:721; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:544, a VH CDR2 having an amino acid sequence of SEQ ID NO:545, and a VH CDR3 having an amino acid sequence of SEQ ID NO:546; and (ii) a VL comprising a VL CDR1 having an amino acid sequence of SEQ ID NO:547, a VL CDR2 having an amino acid sequence of SEQ ID NO:548, and a VL CDR3 having an amino acid sequence of SEQ ID NO:549. In another aspect, provided herein is a bispecific antibody comprising: (a) a first binding domain that binds to TRGV9, and (b) a second binding domain that binds to a second target that is not TRGV9, wherein the first binding domain comprises: (i) a VH comprising a VH CDR1 having an amino acid sequence of SEQ ID NO:550, a VH CDR2 having an amino acid sequence of SEQ ID NO:551, and a VH CDR3 having an amino acid sequence of SEQ ID NO:552; and (ii) a VL comprising a VL CDR1 having an amino acid sequence of SEQ ID NO:553, a VL CDR2 having an amino acid sequence of SEQ ID NO:554, and a VL CDR3 having an amino acid sequence of SEQ ID NO:555. In some embodiments, the first binding domain comprises a VH having an amino acid sequence of SEQ ID NO:133. In some embodiments, the first binding domain comprises a VL having an amino acid sequence of SEQ ID NO:134. In some embodiments, the first binding domain comprises a VH having an amino acid sequence of SEQ ID NO:133, and a VL having an amino acid sequence of SEQ ID NO:134. In some embodiments, the first binding domain comprises a heavy chain having an amino acid sequence of SEQ ID NO:135. In some embodiments, the first binding domain comprises a light chain having an amino acid sequence of SEQ ID NO:136. In some embodiments, the first binding domain comprises a heavy chain having an amino acid sequence of SEQ ID NO:135, and a light chain having an amino acid sequence of SEQ ID NO:136. In some embodiments, the first binding domain comprises a VH comprising an amino acid sequence having at least 95% identity to the amino acid sequence of SEQ ID NO:133. In some embodiments, the first binding domain comprises a VL comprising an amino acid sequence having at least 95% identity to the amino acid sequence of SEQ ID NO:134. In some embodiments, the first binding domain comprises a VH comprising an amino acid sequence having at least 95% identity to the amino acid sequence of SEQ ID NO:133, and a VL comprising an amino acid sequence having at least 95% identity to the amino acid sequence of SEQ ID NO:134. In some embodiments, the first binding domain comprises a heavy chain comprising an amino acid sequence having at least 95% identity to the amino acid sequence of SEQ ID NO:135. In some embodiments, the first binding domain comprises a light chain comprising an amino acid sequence having at least 95% identity to the amino acid sequence of SEQ ID NO:136. In some embodiments, the first binding domain comprises a heavy chain comprising an amino acid sequence having at least 95% identity to the amino acid sequence of SEQ ID NO:135, and a light chain comprising an amino acid sequence having at least 95% identity to the amino acid sequence of SEQ ID NO:136.

In another aspect, provided herein is a bispecific antibody comprising: (a) a first binding domain that binds to TRGV9, and (b) a second binding domain that binds to a cancer antigen present on the surface of a cancer cell. In some embodiments, the antigen on the surface of the cancer cell is a tumor-specific antigen. In some embodiments, the antigen on the surface of the cancer cell is a tumor associated antigen. In some embodiments, the antigen on the surface of the cancer cell is a neoantigen. In certain embodiments, the first binding domain of the bispecific antibody specifically binds TRGV9. In some embodiments, the TRGV9 is present on the surface of a 76 T cell. In some embodiments, the cancer cell is killed when the bispecific antibody binds to the TRGV9 on the surface of the γδ T cell and the antigen on the surface of the cancer cell. Bispecific antibodies comprising any of the TRGV9 antibodies provided herein as the first binding domain are contemplated, in certain embodiments.

In another aspect, provided herein is a bispecific antibody comprising: (a) a first binding domain that binds to TRGV9, and (b) a second binding domain that binds to CD123. In certain embodiments, the first binding domain of the bispecific antibody specifically binds TRGV9. In some embodiments, the TRGV9 is present on the surface of a γδ T cell. In some embodiments, the CD123 is on the surface of a cell. In certain embodiments, the TRGV9 is present on the surface of a γδ T cell, and the CD123 is on the surface of a cell. In some embodiments, the cell having the CD123 on the surface is killed when the bispecific antibody binds to the TRGV9 on the surface of the γδ T cell and the CD123 on the surface of the cell. In some embodiments, the CD123 is on the surface of a cancer cell. In certain embodiments, the TRGV9 is present on the surface of a γδ T cell, and the CD123 is on the surface of a cancer cell. In some embodiments, the cancer cell is killed when the bispecific antibody binds to the TRGV9 on the surface of the γδ T cell and the CD123 on the surface of the cell. Bispecific antibodies comprising any of the TRGV9 antibodies provided herein as the first binding domain are contemplated, in certain embodiments. In addition, bispecific antibodies comprising any of the TRGV9 antibodies provided herein as the first binding domain, and a second binding domain that binds to CD123 are also contemplated in certain embodiments.

In another aspect, provided herein is a bispecific antibody comprising: (a) a first binding domain that binds to TRGV9, and (b) a second binding domain that binds to CD123, wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:556, a VH CDR2 having an amino acid sequence of SEQ ID NO:557, and a VH CDR3 having an amino acid sequence of SEQ ID NO:558; and (ii) a VL comprising a VL CDR1 having an amino acid sequence of SEQ ID NO:559, a VL CDR2 having an amino acid sequence of SEQ ID NO:560, and a VL CDR3 having an amino acid sequence of SEQ ID NO:561. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:562, a VH CDR2 having an amino acid sequence of SEQ ID NO:563, and a VH CDR3 having an amino acid sequence of SEQ ID NO:564; and (ii) a VL comprising a VL CDR1 having an amino acid sequence of SEQ ID NO:565, a VL CDR2 having an amino acid sequence of SEQ ID NO:566, and a VL CDR3 having an amino acid sequence of SEQ ID NO:567. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:568, a VH CDR2 having an amino acid sequence of SEQ ID NO:569, and a VH CDR3 having an amino acid sequence of SEQ ID NO:570; and (ii) a VL comprising a VL CDR1 having an amino acid sequence of SEQ ID NO:571, a VL CDR2 having an amino acid sequence of SEQ ID NO:572, and a VL CDR3 having an amino acid sequence of SEQ ID NO:573. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:574, a VH CDR2 having an amino acid sequence of SEQ ID NO:575, and a VH CDR3 having an amino acid sequence of SEQ ID NO:576; and (ii) a VL comprising a VL CDR1 having an amino acid sequence of SEQ ID NO:577, a VL CDR2 having an amino acid sequence of SEQ ID NO:578, and a VL CDR3 having an amino acid sequence of SEQ ID NO:579. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:574, a VH CDR2 having an amino acid sequence of SEQ ID NO:722, and a VH CDR3 having an amino acid sequence of SEQ ID NO:723; and (ii) a VL comprising a VL CDR1 having an amino acid sequence of SEQ ID NO:577, a VL CDR2 having an amino acid sequence of SEQ ID NO:578, and a VL CDR3 having an amino acid sequence of SEQ ID NO:579. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:580, a VH CDR2 having an amino acid sequence of SEQ ID NO:581, and a VH CDR3 having an amino acid sequence of SEQ ID NO:582; and (ii) a VL comprising a VL CDR1 having an amino acid sequence of SEQ ID NO:583, a VL CDR2 having an amino acid sequence of SEQ ID NO:584, and a VL CDR3 having an amino acid sequence of SEQ ID NO:585. In another aspect, provided herein is a bispecific antibody comprising: (a) a first binding domain that binds to TRGV9, and (b) a second binding domain that binds to CD123, wherein the second binding domain comprises: (i) a VH comprising a VH CDR1 having an amino acid sequence of SEQ ID NO:586, a VH CDR2 having an amino acid sequence of SEQ ID NO:587, and a VH CDR3 having an amino acid sequence of SEQ ID NO:588; and (ii) a VL comprising a VL CDR1 having an amino acid sequence of SEQ ID NO:589, a VL CDR2 having an amino acid sequence of SEQ ID NO:590, and a VL CDR3 having an amino acid sequence of SEQ ID NO:591. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence having at least 95% identity to an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:7; and (ii) a VL comprising a VL CDR1, a VL CDR2, and a VL CDR3 having amino acid sequences of the VL CDR1, VL CDR2, and VL CDR3, respectively, of SEQ ID NO:8; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:7. In some embodiments, the first binding domain comprises a VL having an amino acid sequence of SEQ ID NO:8. In some embodiments, the first binding domain comprises a VH having an amino acid sequence of SEQ ID NO:7, and a VL having an amino acid sequence of SEQ ID NO:8. In some embodiments, the first binding domain comprises a heavy chain having an amino acid sequence of SEQ ID NO:23. In some embodiments, the first binding domain comprises a light chain having an amino acid sequence of SEQ ID NO:24. In some embodiments, the first binding domain comprises a heavy chain having an amino acid sequence of SEQ ID NO:23, and a light chain having an amino acid sequence of SEQ ID NO:24. In some embodiments, the first binding domain comprises an amino acid sequence of SEQ ID NO:17. In some embodiments, the first binding domain comprises a heavy chain having an amino acid sequence of SEQ ID NO:69. In some embodiments, the first binding domain comprises a light chain having an amino acid sequence of SEQ ID NO:24. In some embodiments, the first binding domain comprises a heavy chain having an amino acid sequence of SEQ ID NO:69, and a light chain having an amino acid sequence of SEQ ID NO:24.

In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:7. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:7, and a VL comprising an amino acid sequence having at least 95% identity to the amino acid sequence of SEQ ID NO:8. In some embodiments, the first binding domain comprises a heavy chain comprising an amino acid sequence having at least 95% identity to the amino acid sequence of SEQ ID NO:23. In some embodiments, the first binding domain comprises a light chain comprising an amino acid sequence having at least 95% identity to the amino acid sequence of SEQ ID NO:24. In some embodiments, the first binding domain comprises a heavy chain comprising an amino acid sequence having at least 95% identity to the amino acid sequence of SEQ ID NO:23, and a light chain comprising an amino acid sequence having at least 95% identity to the amino acid sequence of SEQ ID NO:24. In some embodiments, the first binding domain comprises an amino acid sequence of SEQ ID NO:17. In some embodiments, the first binding domain comprises a heavy chain comprising an amino acid sequence having at least 95% identity to the amino acid sequence of SEQ ID NO:69. In some embodiments, the first binding domain comprises a light chain comprising an amino acid sequence having at least 95% identity to the amino acid sequence of SEQ ID NO:24. In some embodiments, the first binding domain comprises a heavy chain comprising an amino acid sequence having at least 95% identity to the amino acid sequence of SEQ ID NO:69, and a light chain comprising an amino acid sequence having at least 95% identity to the amino acid sequence of SEQ ID NO:24. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:34; and (ii) a VL comprising a VL CDR1, a VL CDR2, and a VL CDR3 having amino acid sequences of the VL CDR1, VL CDR2, and VL CDR3, respectively, of SEQ ID NO:8; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:34. In some embodiments, the first binding domain comprises a VL having an amino acid sequence of SEQ ID NO:8. In some embodiments, the first binding domain comprises a VH having an amino acid sequence of SEQ ID NO:34, and a VL having an amino acid sequence of SEQ ID NO:8. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:34. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:34, and a VL comprising an amino acid sequence having at least 95% identity to the amino acid sequence of SEQ ID NO:8. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:35; and (ii) a VL comprising a VL CDR1, a VL CDR2, and a VL CDR3 having amino acid sequences of the VL CDR1, VL CDR2, and VL CDR3, respectively, of SEQ ID NO:8; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:35. In some embodiments, the first binding domain comprises a VL having an amino acid sequence of SEQ ID NO:8. In some embodiments, the first binding domain comprises a VH having an amino acid sequence of SEQ ID NO:35, and a VL having an amino acid sequence of SEQ ID NO:8. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:35. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:35, and a VL comprising an amino acid sequence having at least 95% identity to the amino acid sequence of SEQ ID NO:8. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:36; and (ii) a VL comprising a VL CDR1, a VL CDR2, and a VL CDR3 having amino acid sequences of the VL CDR1, VL CDR2, and VL CDR3, respectively, of SEQ ID NO:8; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and (ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:36. In some embodiments, the first binding domain comprises a VL having an amino acid sequence of SEQ ID NO:8. In some embodiments, the first binding domain comprises a VH having an amino acid sequence of SEQ ID NO:36, and a VL having an amino acid sequence of SEQ ID NO:8. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:36. In some embodiments, the first binding domain comprises a VL comprising an amino acid sequence having at least 95% identity to the amino acid sequence of SEQ ID NO:8. In some embodiments, the first binding domain comprises a VH comprising an amino acid sequence having at least 95% identity to the amino acid sequence of SEQ ID NO:36, and a VL comprising an amino acid sequence having at least 95% identity to the amino acid sequence of SEQ ID NO:8. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:65; and (ii) a VL comprising a VL CDR1, a VL CDR2, and a VL CDR3 having amino acid sequences of the VL CDR1, VL CDR2, and VL CDR3, respectively, of SEQ ID NO:66; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:67; and (ii) a VL comprising a VL CDR1, a VL CDR2, and a VL CDR3 having amino acid sequences of the VL CDR1, VL CDR2, and VL CDR3, respectively, of SEQ ID NO:68; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:1, a VH CDR2 having an amino acid sequence of SEQ ID NO:76, and a VH CDR3 having an amino acid sequence of SEQ ID NO:3; and (ii) a VL comprising a VL CDR1 having an amino acid sequence of SEQ ID NO:77, a VL CDR2 having an amino acid sequence of SEQ ID NO:5, and a VL CDR3 having an amino acid sequence of SEQ ID NO:6; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and (ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:65. In some embodiments, the first binding domain comprises a VL having an amino acid sequence of SEQ ID NO:66. In some embodiments, the first binding domain comprises a VH having an amino acid sequence of SEQ ID NO:65, and a VL having an amino acid sequence of SEQ ID NO:66. In some embodiments, the first binding domain comprises a VH having an amino acid sequence of SEQ ID NO:67. In some embodiments, the first binding domain comprises a VL having an amino acid sequence of SEQ ID NO:68. In some embodiments, the first binding domain a VH having an amino acid sequence of SEQ ID NO:67, and a VL having an amino acid sequence of SEQ ID NO:68. In some embodiments, the first binding domain comprises a heavy chain having an amino acid sequence of SEQ ID NO:71. In some embodiments, the first binding domain comprises a light chain having an amino acid sequence of SEQ ID NO:72. In some embodiments, the first binding domain comprises a heavy chain having an amino acid sequence of SEQ ID NO:71, and a light chain having an amino acid sequence of SEQ ID NO:72. In some embodiments, the first binding domain comprises an amino acid sequence of SEQ ID NO:70. In some embodiments, the first binding domain comprises a heavy chain having an amino acid sequence of SEQ ID NO:74. In some embodiments, the first binding domain comprises a light chain having an amino acid sequence of SEQ ID NO:75. In some embodiments, the first binding domain comprises a heavy chain having an amino acid sequence of SEQ ID NO:74, and a light chain having an amino acid sequence of SEQ ID NO:75. In some embodiments, the first binding domain comprises an amino acid sequence of SEQ ID NO:73. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:65. In some embodiments, the first binding domain comprises a VL comprising an amino acid sequence having at least 95% identity to the amino acid sequence of SEQ ID NO:66. In some embodiments, the first binding domain comprises a VH comprising an amino acid sequence having at least 95% identity to the amino acid sequence of SEQ ID NO:65, and a VL comprising an amino acid sequence having at least 95% identity to the amino acid sequence of SEQ ID NO:66. In some embodiments, the first binding domain comprises a VH comprising an amino acid sequence having at least 95% identity to the amino acid sequence of SEQ ID NO:67. In some embodiments, the first binding domain comprises a VL comprising an amino acid sequence having at least 95% identity to the amino acid sequence of SEQ ID NO:68. In some embodiments, the first binding domain a VH comprising an amino acid sequence having at least 95% identity to the amino acid sequence of SEQ ID NO:67, and a VL comprising an amino acid sequence having at least 95% identity to the amino acid sequence of SEQ ID NO:68. In some embodiments, the first binding domain comprises a heavy chain comprising an amino acid sequence having at least 95% identity to the amino acid sequence of SEQ ID NO:71. In some embodiments, the first binding domain comprises a light chain comprising an amino acid sequence having at least 95% identity to the amino acid sequence of SEQ ID NO:72. In some embodiments, the first binding domain comprises a heavy chain comprising an amino acid sequence having at least 95% identity to the amino acid sequence of SEQ ID NO:71, and a light chain comprising an amino acid sequence having at least 95% identity to the amino acid sequence of SEQ ID NO:72. In some embodiments, the first binding domain comprises an amino acid sequence of SEQ ID NO:70. In some embodiments, the first binding domain comprises a heavy chain comprising an amino acid sequence having at least 95% identity to the amino acid sequence of SEQ ID NO:74. In some embodiments, the first binding domain comprises a light chain comprising an amino acid sequence having at least 95% identity to the amino acid sequence of SEQ ID NO:75. In some embodiments, the first binding domain comprises a heavy chain comprising an amino acid sequence having at least 95% identity to the amino acid sequence of SEQ ID NO:74, and a light chain comprising an amino acid sequence having at least 95% identity to the amino acid sequence of SEQ ID NO:75. In some embodiments, the first binding domain comprises an amino acid sequence of SEQ ID NO:73. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:95; and (ii) a VL comprising a VL CDR1, a VL CDR2, and a VL CDR3 having amino acid sequences of the VL CDR1, VL CDR2, and VL CDR3, respectively, of SEQ ID NO:96; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and (ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:95. In some embodiments, the first binding domain comprises a VL having an amino acid sequence of SEQ ID NO:96. In some embodiments, the first binding domain comprises a VH having an amino acid sequence of SEQ ID NO:95, and a VL having an amino acid sequence of SEQ ID NO:96. In some embodiments, the first binding domain comprises an amino acid sequence of SEQ ID NO:97. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:95. In some embodiments, the first binding domain comprises a VL comprising an amino acid sequence having at least 95% identity to the amino acid sequence of SEQ ID NO:96. In some embodiments, the first binding domain comprises a VH comprising an amino acid sequence having at least 95% identity to the amino acid sequence of SEQ ID NO:95, and a VL comprising an amino acid sequence having at least 95% identity to the amino acid sequence of SEQ ID NO:96. In some embodiments, the first binding domain comprises an amino acid sequence of SEQ ID NO:97. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:104; and (ii) a VL comprising a VL CDR1, a VL CDR2, and a VL CDR3 having amino acid sequences of the VL CDR1, VL CDR2, and VL CDR3, respectively, of SEQ ID NO:105; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100, and (ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:104. In some embodiments, the first binding domain comprises a VL having an amino acid sequence of SEQ ID NO:105. In some embodiments, the first binding domain comprises a VH having an amino acid sequence of SEQ ID NO:104, and a VL having an amino acid sequence of SEQ ID NO:105. In some embodiments, the first binding domain comprises an amino acid sequence of SEQ ID NO:106. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:104. In some embodiments, the first binding domain comprises a VL comprising an amino acid sequence having at least 95% identity to the amino acid sequence of SEQ ID NO:105. In some embodiments, the first binding domain comprises a VH comprising an amino acid sequence having at least 95% identity to the amino acid sequence of SEQ ID NO:104, and a VL comprising an amino acid sequence having at least 95% identity to the amino acid sequence of SEQ ID NO:105. In some embodiments, the first binding domain comprises an amino acid sequence of SEQ ID NO:106. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:113; and (ii) a VL comprising a VL CDR1, a VL CDR2, and a VL CDR3 having amino acid sequences of the VL CDR1, VL CDR2, and VL CDR3, respectively, of SEQ ID NO:114; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109, and (ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VH having an amino acid sequence of SEQ ID NO:113. In some embodiments, the first binding domain comprises a VL having an amino acid sequence of SEQ ID NO:114. In some embodiments, the first binding domain comprises a VH having an amino acid sequence of SEQ ID NO:113, and a VL having an amino acid sequence of SEQ ID NO:114. In some embodiments, the first binding domain comprises a heavy chain having an amino acid sequence of SEQ ID NO:115. In some embodiments, the first binding domain comprises a light chain having an amino acid sequence of SEQ ID NO:116. In some embodiments, the first binding domain comprises a heavy chain having an amino acid sequence of SEQ ID NO:115, and a light chain having an amino acid sequence of SEQ ID NO:116. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VH comprising an amino acid sequence having at least 95% identity to the amino acid sequence of SEQ ID NO:113. In some embodiments, the first binding domain comprises a VL comprising an amino acid sequence having at least 95% identity to the amino acid sequence of SEQ ID NO:114. In some embodiments, the first binding domain comprises a VH comprising an amino acid sequence having at least 95% identity to the amino acid sequence of SEQ ID NO:113, and a VL comprising an amino acid sequence having at least 95% identity to the amino acid sequence of SEQ ID NO:114. In some embodiments, the first binding domain comprises a heavy chain comprising an amino acid sequence having at least 95% identity to the amino acid sequence of SEQ ID NO:115. In some embodiments, the first binding domain comprises a light chain comprising an amino acid sequence having at least 95% identity to the amino acid sequence of SEQ ID NO:116. In some embodiments, the first binding domain comprises a heavy chain comprising an amino acid sequence having at least 95% identity to the amino acid sequence of SEQ ID NO:115, and a light chain comprising an amino acid sequence having at least 95% identity to the amino acid sequence of SEQ ID NO:116. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:123; and (ii) a VL comprising a VL CDR1, a VL CDR2, and a VL CDR3 having amino acid sequences of the VL CDR1, VL CDR2, and VL CDR3, respectively, of SEQ ID NO:124; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119, and (ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122. In some embodiments, the first binding domain comprises a VH having an amino acid sequence of SEQ ID NO:123; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VL having an amino acid sequence of SEQ ID NO:124. In some embodiments, the first binding domain comprises a VH having an amino acid sequence of SEQ ID NO:123, and a VL having an amino acid sequence of SEQ ID NO:124. In some embodiments, the first binding domain comprises a heavy chain having an amino acid sequence of SEQ ID NO:125. In some embodiments, the first binding domain comprises a light chain having an amino acid sequence of SEQ ID NO:126. In some embodiments, the first binding domain comprises a heavy chain having an amino acid sequence of SEQ ID NO:125, and a light chain having an amino acid sequence of SEQ ID NO:126. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VL comprising an amino acid sequence having at least 95% identity to the amino acid sequence of SEQ ID NO:124. In some embodiments, the first binding domain comprises a VH comprising an amino acid sequence having at least 95% identity to the amino acid sequence of SEQ ID NO:123, and a VL comprising an amino acid sequence having at least 95% identity to the amino acid sequence of SEQ ID NO:124. In some embodiments, the first binding domain comprises a heavy chain comprising an amino acid sequence having at least 95% identity to the amino acid sequence of SEQ ID NO:125. In some embodiments, the first binding domain comprises a light chain comprising an amino acid sequence having at least 95% identity to the amino acid sequence of SEQ ID NO:126. In some embodiments, the first binding domain comprises a heavy chain comprising an amino acid sequence having at least 95% identity to the amino acid sequence of SEQ ID NO:125, and a light chain comprising an amino acid sequence having at least 95% identity to the amino acid sequence of SEQ ID NO:126. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:133; and (ii) a VL comprising a VL CDR1, a VL CDR2, and a VL CDR3 having amino acid sequences of the VL CDR1, VL CDR2, and VL CDR3, respectively, of SEQ ID NO:134; and wherein the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In another aspect, provided herein is a bispecific antibody comprising: (a) a first binding domain that binds to TRGV9, and (b) a second binding domain that binds to CD123, wherein the first binding domain comprises: (i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129, and (ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO:132. In some embodiments, the first binding domain comprises a VH having an amino acid sequence of SEQ ID NO:133; and wherein the second binding domain comprises: (i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and (ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO:14. In some embodiments, the first binding domain comprises a VL having an amino acid sequence of SEQ ID NO:134. In some embodiments, the first binding domain comprises a VH having an amino acid sequence of SEQ ID NO:133, and a VL having an amino acid sequence of SEQ ID NO:134. In some embodiments, the first binding domain comprises a heavy chain having an amino acid sequence of SEQ ID NO:135. In some embodiments, the first binding domain comprises a light chain having an amino acid sequence of SEQ ID NO:136. In some embodiments, the first binding domain comprises a heavy chain having an amino acid sequence of SEQ ID NO:135, and a light chain having an amino acid sequence of SEQ ID NO:136. In some embodiments, the second binding domain comprises a VH having an amino acid sequence of SEQ ID NO:15. In some embodiments, the second binding domain comprises a VL having an amino acid sequence of SEQ ID NO:16. In some embodiments, the second binding domain comprises a VH having an amino acid sequence of SEQ ID NO:15, and a VL having an amino acid sequence of SEQ ID NO:16. In some embodiments, the second binding domain comprises a heavy chain having an amino acid sequence of SEQ ID NO:25. In some embodiments, the second binding domain comprises a light chain having an amino acid sequence of SEQ ID NO:26. In some embodiments, the second binding domain comprises a heavy chain having an amino acid sequence of SEQ ID NO:25, and a light chain having an amino acid sequence of SEQ ID NO:26. In some embodiments, the second binding domain comprises an amino acid sequence of SEQ ID NO:18. In some embodiments, the first binding domain comprises a VL comprising an amino acid sequence having at least 95% identity to the amino acid sequence of SEQ ID NO:134. In some embodiments, the first binding domain comprises a VH comprising an amino acid sequence having at least 95% identity to the amino acid sequence of SEQ ID NO:133, and a VL comprising an amino acid sequence having at least 95% identity to the amino acid sequence of SEQ ID NO:134. In some embodiments, the first binding domain comprises a heavy chain comprising an amino acid sequence having at least 95% identity to the amino acid sequence of SEQ ID NO:135. In some embodiments, the first binding domain comprises a light chain comprising an amino acid sequence having at least 95% identity to the amino acid sequence of SEQ ID NO:136. In some embodiments, the first binding domain comprises a heavy chain comprising an amino acid sequence having at least 95% identity to the amino acid sequence of SEQ ID NO:135, and a light chain comprising an amino acid sequence having at least 95% identity to the amino acid sequence of SEQ ID NO:136. In some embodiments, the second binding domain comprises a VH comprising an amino acid sequence having at least 95% identity to the amino acid sequence of SEQ ID NO:15. In some embodiments, the second binding domain comprises a VL comprising an amino acid sequence having at least 95% identity to the amino acid sequence of SEQ ID NO:16. In some embodiments, the second binding domain comprises a VH comprising an amino acid sequence having at least 95% identity to the amino acid sequence of SEQ ID NO:15, and a VL comprising an amino acid sequence having at least 95% identity to the amino acid sequence of SEQ ID NO:16. In some embodiments, the second binding domain comprises a heavy chain comprising an amino acid sequence having at least 95% identity to the amino acid sequence of SEQ ID NO:25. In some embodiments, the second binding domain comprises a light chain comprising an amino acid sequence having at least 95% identity to the amino acid sequence of SEQ ID NO:26. In some embodiments, the second binding domain comprises a heavy chain comprising an amino acid sequence having at least 95% identity to the amino acid sequence of SEQ ID NO:25, and a light chain comprising an amino acid sequence having at least 95% identity to the amino acid sequence of SEQ ID NO:26. In some embodiments, the second binding domain comprises an amino acid sequence of SEQ ID NO:18.

In another aspect, provided herein is a bispecific antibody comprising: (a) a first binding domain that binds to TRGV9, and (b) a second binding domain that binds to CD123. In some embodiments, the first binding domain that binds to TRGV9 comprises: (i) a VH comprising a VH CDR1 having an amino acid sequence of SEQ ID NO:160, a VH CDR2 having an amino acid sequence of SEQ ID NO:161, and a VH CDR3 having an amino acid sequence of SEQ ID NO:162; and (ii) a VL comprising a VL CDR1 having an amino acid sequence of SEQ ID NO:163, a VL CDR2 having an amino acid sequence of SEQ ID NO:164, and a VL CDR3 having an amino acid sequence of SEQ ID NO:165. In some embodiments, the first binding domain that binds to TRGV9 comprises: (i) a VH comprising a VH CDR1 having an amino acid sequence of SEQ ID NO:166, a VH CDR2 having an amino acid sequence of SEQ ID NO:167, and a VH CDR3 having an amino acid sequence of SEQ ID NO:168; and (ii) a VL comprising a VL CDR1 having an amino acid sequence of SEQ ID NO:169, a VL CDR2 having an amino acid sequence of SEQ ID NO:170, and a VL CDR3 having an amino acid sequence of SEQ ID NO:171. In some embodiments, the first binding domain that binds to TRGV9 comprises: (i) a VH comprising a VH CDR1 having an amino acid sequence of SEQ ID NO:172, a VH CDR2 having an amino acid sequence of SEQ ID NO:173, and a VH CDR3 having an amino acid sequence of SEQ ID NO:174; and (ii) a VL comprising a VL CDR1 having an amino acid sequence of SEQ ID NO:175, a VL CDR2 having an amino acid sequence of SEQ ID NO:176, and a VL CDR3 having an amino acid sequence of SEQ ID NO:177. In some embodiments, the first binding domain that binds to TRGV9 comprises: (i) a VH comprising a VH CDR1 having an amino acid sequence of SEQ ID NO:178, a VH CDR2 having an amino acid sequence of SEQ ID NO:179, and a VH CDR3 having an amino acid sequence of SEQ ID NO:180; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In some embodiments, the first binding domain that binds to TRGV9 comprises: (i) a VH comprising a VH CDR1 having an amino acid sequence of SEQ ID NO:178, a VH CDR2 having an amino acid sequence of SEQ ID NO:700, and a VH CDR3 having an amino acid sequence of SEQ ID NO:701; and (ii) a VL comprising a VL CDR1 having an amino acid sequence of SEQ ID NO:181, a VL CDR2 having an amino acid sequence of SEQ ID NO:182, and a VL CDR3 having an amino acid sequence of SEQ ID NO:183. In some embodiments, the first binding domain that binds to TRGV9 comprises: (i) a VH comprising a VH CDR1 having an amino acid sequence of SEQ ID NO:184, a VH CDR2 having an amino acid sequence of SEQ ID NO:185, and a VH CDR3 having an amino acid sequence of SEQ ID NO:186; and (ii) a VL comprising a VL CDR1 having an amino acid sequence of SEQ ID NO:187, a VL CDR2 having an amino acid sequence of SEQ ID NO:188, and a VL CDR3 having an amino acid sequence of SEQ ID NO:189. In some embodiments, the first binding domain that binds to TRGV9 comprises: (i) a VH comprising a VH CDR1 having an amino acid sequence of SEQ ID NO:190, a VH CDR2 having an amino acid sequence of SEQ ID NO:191, and a VH CDR3 having an amino acid sequence of SEQ ID NO:192; and (ii) a VL comprising a VL CDR1 having an amino acid sequence of SEQ ID NO:193, a VL CDR2 having an amino acid sequence of SEQ ID NO:194, and a VL CDR3 having an amino acid sequence of SEQ ID NO:195. In some embodiments, the first binding domain that binds to TRGV9 comprises: (i) a VH comprising a VH CDR1 having an amino acid sequence of SEQ ID NO:196, a VH CDR2 having an amino acid sequence of SEQ ID NO:197, and a VH CDR3 having an amino acid sequence of SEQ ID NO:198; and (ii) a VL comprising a VL CDR1 having an amino acid sequence of SEQ ID NO:199, a VL CDR2 having an amino acid sequence of SEQ ID NO:200, and a VL CDR3 having an amino acid sequence of SEQ ID NO:201. In some embodiments, the first binding domain that binds to TRGV9 comprises: (i) a VH comprising a VH CDR1 having an amino acid sequence of SEQ ID NO:202, a VH CDR2 having an amino acid sequence of SEQ ID NO:203, and a VH CDR3 having an amino acid sequence of SEQ ID NO:204; and (ii) a VL comprising a VL CDR1 having an amino acid sequence of SEQ ID NO:205, a VL CDR2 having an amino acid sequence of SEQ ID NO:206, and a VL CDR3 having an amino acid sequence of SEQ ID NO:207. In some embodiments, the first binding domain that binds to TRGV9 comprises: (i) a VH comprising a VH CDR1 having an amino acid sequence of SEQ ID NO:208, a VH CDR2 having an amino acid sequence of SEQ ID NO:209, and a VH CDR3 having an amino acid sequence of SEQ ID NO:210; and (ii) a VL comprising a VL CDR1 having an amino acid sequence of SEQ ID NO:211, a VL CDR2 having an amino acid sequence of SEQ ID NO:212, and a VL CDR3 having an amino acid sequence of SEQ ID NO:213. In some embodiments, the first binding domain that binds to TRGV9 comprises: (i) a VH comprising a VH CDR1 having an amino acid sequence of SEQ ID NO:214, a VH CDR2 having an amino acid sequence of SEQ ID NO:215, and a VH CDR3 having an amino acid sequence of SEQ ID NO:216; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In some embodiments, the first binding domain that binds to TRGV9 comprises: (i) a VH comprising a VH CDR1 having an amino acid sequence of SEQ ID NO:214, a VH CDR2 having an amino acid sequence of SEQ ID NO:702, and a VH CDR3 having an amino acid sequence of SEQ ID NO:703; and (ii) a VL comprising a VL CDR1 having an amino acid sequence of SEQ ID NO:217, a VL CDR2 having an amino acid sequence of SEQ ID NO:218, and a VL CDR3 having an amino acid sequence of SEQ ID NO:219. In some embodiments, the first binding domain that binds to TRGV9 comprises: (i) a VH comprising a VH CDR1 having an amino acid sequence of SEQ ID NO:220, a VH CDR2 having an amino acid sequence of SEQ ID NO:221, and a VH CDR3 having an amino acid sequence of SEQ ID NO:222; and (ii) a VL comprising a VL CDR1 having an amino acid sequence of SEQ ID NO:223, a VL CDR2 having an amino acid sequence of SEQ ID NO:224, and a VL CDR3 having an amino acid sequence of SEQ ID NO:225. In some embodiments, the first binding domain that binds to TRGV9 comprises: (i) a VH comprising a VH CDR1 having an amino acid sequence of SEQ ID NO:226, a VH CDR2 having an amino acid sequence of SEQ ID NO:227, and a VH CDR3 having an amino acid sequence of SEQ ID NO:228; and (ii) a VL comprising a VL CDR1 having an amino acid sequence of SEQ ID NO:229, a VL CDR2 having an amino acid sequence of SEQ ID NO:230, and a VL CDR3 having an amino acid sequence of SEQ ID NO:231. In some embodiments, the first binding domain that binds to TRGV9 comprises: (i) a VH comprising a VH CDR1 having an amino acid sequence of SEQ ID NO:232, a VH CDR2 having an amino acid sequence of SEQ ID NO:233, and a VH CDR3 having an amino acid sequence of SEQ ID NO:234; and (ii) a VL comprising a VL CDR1 having an amino acid sequence of SEQ ID NO:235, a VL CDR2 having an amino acid sequence of SEQ ID NO:236, and a VL CDR3 having an amino acid sequence of SEQ ID NO:237. In some embodiments, the first binding domain that binds to TRGV9 comprises: (i) a VH comprising a VH CDR1 having an amino acid sequence of SEQ ID NO:238, a VH CDR2 having an amino acid sequence of SEQ ID NO:239, and a VH CDR3 having an amino acid sequence of SEQ ID NO:240; and (ii) a VL comprising a VL CDR1 having an amino acid sequence of SEQ ID NO:241, a VL CDR2 having an amino acid sequence of SEQ ID NO:242, and a VL CDR3 having an amino acid sequence of SEQ ID NO:243. In some embodiments, the first binding domain that binds to TRGV9 comprises: (i) a VH comprising a VH CDR1 having an amino acid sequence of SEQ ID NO:244, a VH CDR2 having an amino acid sequence of SEQ ID NO:245, and a VH CDR3 having an amino acid sequence of SEQ ID NO:246; and (ii) a VL comprising a VL CDR1 having an amino acid sequence of SEQ ID NO:247, a VL CDR2 having an amino acid sequence of SEQ ID NO:248, and a VL CDR3 having an amino acid sequence of SEQ ID NO:249. In some embodiments, the first binding domain that binds to TRGV9 comprises: (i) a VH comprising a VH CDR1 having an amino acid sequence of SEQ ID NO:250, a VH CDR2 having an amino acid sequence of SEQ ID NO:251, and a VH CDR3 having an amino acid sequence of SEQ ID NO:252; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In some embodiments, the first binding domain that binds to TRGV9 comprises: (i) a VH comprising a VH CDR1 having an amino acid sequence of SEQ ID NO:250, a VH CDR2 having an amino acid sequence of SEQ ID NO:704, and a VH CDR3 having an amino acid sequence of SEQ ID NO:705; and (ii) a VL comprising a VL CDR1 having an amino acid sequence of SEQ ID NO:253, a VL CDR2 having an amino acid sequence of SEQ ID NO:254, and a VL CDR3 having an amino acid sequence of SEQ ID NO:255. In some embodiments, the first binding domain that binds to TRGV9 comprises: (i) a VH comprising a VH CDR1 having an amino acid sequence of SEQ ID NO:256, a VH CDR2 having an amino acid sequence of SEQ ID NO:257, and a VH CDR3 having an amino acid sequence of SEQ ID NO:258; and (ii) a VL comprising a VL CDR1 having an amino acid sequence of SEQ ID NO:259, a VL CDR2 having an amino acid sequence of SEQ ID NO:260, and a VL CDR3 having an amino acid sequence of SEQ ID NO:261. In some embodiments, the first binding domain that binds to TRGV9 comprises: (i) a VH comprising a VH CDR1 having an amino acid sequence of SEQ ID NO:262, a VH CDR2 having an amino acid sequence of SEQ ID NO:263, and a VH CDR3 having an amino acid sequence of SEQ ID NO:264; and (ii) a VL comprising a VL CDR1 having an amino acid sequence of SEQ ID NO:265, a VL CDR2 having an amino acid sequence of SEQ ID NO:266, and a VL CDR3 having an amino acid sequence of SEQ ID NO:267. In some embodiments, the first binding domain that binds to TRGV9 comprises: (i) a VH comprising a VH CDR1 having an amino acid sequence of SEQ ID NO:268, a VH CDR2 having an amino acid sequence of SEQ ID NO:269, and a VH CDR3 having an amino acid sequence of SEQ ID NO:270; and (ii) a VL comprising a VL CDR1 having an amino acid sequence of SEQ ID NO:271, a VL CDR2 having an amino acid sequence of SEQ ID NO:272, and a VL CDR3 having an amino acid sequence of SEQ ID NO:273. In some embodiments, the first binding domain that binds to TRGV9 comprises: (i) a VH comprising a VH CDR1 having an amino acid sequence of SEQ ID NO:274, a VH CDR2 having an amino acid sequence of SEQ ID NO:275, and a VH CDR3 having an amino acid sequence of SEQ ID NO:276; and (ii) a VL comprising a VL CDR1 having an amino acid sequence of SEQ ID NO:277, a VL CDR2 having an amino acid sequence of SEQ ID NO:278, and a VL CDR3 having an amino acid sequence of SEQ ID NO:279. In some embodiments, the first binding domain that binds to TRGV9 comprises: (i) a VH comprising a VH CDR1 having an amino acid sequence of SEQ ID NO:280, a VH CDR2 having an amino acid sequence of SEQ ID NO:281, and a VH CDR3 having an amino acid sequence of SEQ ID NO:282; and (ii) a VL comprising a VL CDR1 having an amino acid sequence of SEQ ID NO:283, a VL CDR2 having an amino acid sequence of SEQ ID NO:284, and a VL CDR3 having an amino acid sequence of SEQ ID NO:285. In some embodiments, the first binding domain that binds to TRGV9 comprises: (i) a VH comprising a VH CDR1 having an amino acid sequence of SEQ ID NO:286, a VH CDR2 having an amino acid sequence of SEQ ID NO:287, and a VH CDR3 having an amino acid sequence of SEQ ID NO:288; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In some embodiments, the first binding domain that binds to TRGV9 comprises: (i) a VH comprising a VH CDR1 having an amino acid sequence of SEQ ID NO:286, a VH CDR2 having an amino acid sequence of SEQ ID NO:706, and a VH CDR3 having an amino acid sequence of SEQ ID NO:707; and (ii) a VL comprising a VL CDR1 having an amino acid sequence of SEQ ID NO:289, a VL CDR2 having an amino acid sequence of SEQ ID NO:290, and a VL CDR3 having an amino acid sequence of SEQ ID NO:291. In some embodiments, the first binding domain that binds to TRGV9 comprises: (i) a VH comprising a VH CDR1 having an amino acid sequence of SEQ ID NO:292, a VH CDR2 having an amino acid sequence of SEQ ID NO:293, and a VH CDR3 having an amino acid sequence of SEQ ID NO:294; and (ii) a VL comprising a VL CDR1 having an amino acid sequence of SEQ ID NO:295, a VL CDR2 having an amino acid sequence of SEQ ID NO:296, and a VL CDR3 having an amino acid sequence of SEQ ID NO:297. In some embodiments, the first binding domain that binds to TRGV9 comprises: (i) a VH comprising a VH CDR1 having an amino acid sequence of SEQ ID NO:298, a VH CDR2 having an amino acid sequence of SEQ ID NO:299, and a VH CDR3 having an amino acid sequence of SEQ ID NO:300; and (ii) a VL comprising a VL CDR1 having an amino acid sequence of SEQ ID NO:301, a VL CDR2 having an amino acid sequence of SEQ ID NO:302, and a VL CDR3 having an amino acid sequence of SEQ ID NO:303. In some embodiments, the first binding domain that binds to TRGV9 comprises: (i) a VH comprising a VH CDR1 having an amino acid sequence of SEQ ID NO:304, a VH CDR2 having an amino acid sequence of SEQ ID NO:305, and a VH CDR3 having an amino acid sequence of SEQ ID NO:306; and (ii) a VL comprising a VL CDR1 having an amino acid sequence of SEQ ID NO:307, a VL CDR2 having an amino acid sequence of SEQ ID NO:308, and a VL CDR3 having an amino acid sequence of SEQ ID NO:309. In some embodiments, the first binding domain that binds to TRGV9 comprises: (i) a VH comprising a VH CDR1 having an amino acid sequence of SEQ ID NO:310, a VH CDR2 having an amino acid sequence of SEQ ID NO:311, and a VH CDR3 having an amino acid sequence of SEQ ID NO:312; and (ii) a VL comprising a VL CDR1 having an amino acid sequence of SEQ ID NO:313, a VL CDR2 having an amino acid sequence of SEQ ID NO:314, and a VL CDR3 having an amino acid sequence of SEQ ID NO:315. In some embodiments, the first binding domain that binds to TRGV9 comprises: (i) a VH comprising a VH CDR1 having an amino acid sequence of SEQ ID NO:316, a VH CDR2 having an amino acid sequence of SEQ ID NO:317, and a VH CDR3 having an amino acid sequence of SEQ ID NO:318; and (ii) a VL comprising a VL CDR1 having an amino acid sequence of SEQ ID NO:319, a VL CDR2 having an amino acid sequence of SEQ ID NO:320, and a VL CDR3 having an amino acid sequence of SEQ ID NO:321. In some embodiments, the first binding domain that binds to TRGV9 comprises: (i) a VH comprising a VH CDR1 having an amino acid sequence of SEQ ID NO:322, a VH CDR2 having an amino acid sequence of SEQ ID NO:323, and a VH CDR3 having an amino acid sequence of SEQ ID NO:324; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In some embodiments, the first binding domain that binds to TRGV9 comprises: (i) a VH comprising a VH CDR1 having an amino acid sequence of SEQ ID NO:322, a VH CDR2 having an amino acid sequence of SEQ ID NO:708, and a VH CDR3 having an amino acid sequence of SEQ ID NO:709; and (ii) a VL comprising a VL CDR1 having an amino acid sequence of SEQ ID NO:325, a VL CDR2 having an amino acid sequence of SEQ ID NO:326, and a VL CDR3 having an amino acid sequence of SEQ ID NO:327. In some embodiments, the first binding domain that binds to TRGV9 comprises: (i) a VH comprising a VH CDR1 having an amino acid sequence of SEQ ID NO:328, a VH CDR2 having an amino acid sequence of SEQ ID NO:329, and a VH CDR3 having an amino acid sequence of SEQ ID NO:330; and (ii) a VL comprising a VL CDR1 having an amino acid sequence of SEQ ID NO:331, a VL CDR2 having an amino acid sequence of SEQ ID NO:332, and a VL CDR3 having an amino acid sequence of SEQ ID NO:333. In some embodiments, the first binding domain that binds to TRGV9 comprises: (i) a VH comprising a VH CDR1 having an amino acid sequence of SEQ ID NO:334, a VH CDR2 having an amino acid sequence of SEQ ID NO:335, and a VH CDR3 having an amino acid sequence of SEQ ID NO:336; and (ii) a VL comprising a VL CDR1 having an amino acid sequence of SEQ ID NO:337, a VL CDR2 having an amino acid sequence of SEQ ID NO:338, and a VL CDR3 having an amino acid sequence of SEQ ID NO:339. In some embodiments, the first binding domain that binds to TRGV9 comprises: (i) a VH comprising a VH CDR1 having an amino acid sequence of SEQ ID NO:340, a VH CDR2 having an amino acid sequence of SEQ ID NO:341, and a VH CDR3 having an amino acid sequence of SEQ ID NO:342; and (ii) a VL comprising a VL CDR1 having an amino acid sequence of SEQ ID NO:343, a VL CDR2 having an amino acid sequence of SEQ ID NO:344, and a VL CDR3 having an amino acid sequence of SEQ ID NO:345. In some embodiments, the first binding domain that binds to TRGV9 comprises: (i) a VH comprising a VH CDR1 having an amino acid sequence of SEQ ID NO:346, a VH CDR2 having an amino acid sequence of SEQ ID NO:347, and a VH CDR3 having an amino acid sequence of SEQ ID NO:348; and (ii) a VL comprising a VL CDR1 having an amino acid sequence of SEQ ID NO:349, a VL CDR2 having an amino acid sequence of SEQ ID NO:350, and a VL CDR3 having an amino acid sequence of SEQ ID NO:351. In some embodiments, the first binding domain that binds to TRGV9 comprises: (i) a VH comprising a VH CDR1 having an amino acid sequence of SEQ ID NO:352, a VH CDR2 having an amino acid sequence of SEQ ID NO:353, and a VH CDR3 having an amino acid sequence of SEQ ID NO:354; and (ii) a VL comprising a VL CDR1 having an amino acid sequence of SEQ ID NO:355, a VL CDR2 having an amino acid sequence of SEQ ID NO:356, and a VL CDR3 having an amino acid sequence of SEQ ID NO:357. In some embodiments, the first binding domain that binds to TRGV9 comprises: (i) a VH comprising a VH CDR1 having an amino acid sequence of SEQ ID NO:358, a VH CDR2 having an amino acid sequence of SEQ ID NO:359, and a VH CDR3 having an amino acid sequence of SEQ ID NO:360; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In some embodiments, the first binding domain that binds to TRGV9 comprises: (i) a VH comprising a VH CDR1 having an amino acid sequence of SEQ ID NO:358, a VH CDR2 having an amino acid sequence of SEQ ID NO:710, and a VH CDR3 having an amino acid sequence of SEQ ID NO:711; and (ii) a VL comprising a VL CDR1 having an amino acid sequence of SEQ ID NO:361, a VL CDR2 having an amino acid sequence of SEQ ID NO:362, and a VL CDR3 having an amino acid sequence of SEQ ID NO:363. In some embodiments, the first binding domain that binds to TRGV9 comprises: (i) a VH comprising a VH CDR1 having an amino acid sequence of SEQ ID NO:364, a VH CDR2 having an amino acid sequence of SEQ ID NO:365, and a VH CDR3 having an amino acid sequence of SEQ ID NO:366; and (ii) a VL comprising a VL CDR1 having an amino acid sequence of SEQ ID NO:367, a VL CDR2 having an amino acid sequence of SEQ ID NO:368, and a VL CDR3 having an amino acid sequence of SEQ ID NO:369. In some embodiments, the first binding domain that binds to TRGV9 comprises: (i) a VH comprising a VH CDR1 having an amino acid sequence of SEQ ID NO:370, a VH CDR2 having an amino acid sequence of SEQ ID NO:371, and a VH CDR3 having an amino acid sequence of SEQ ID NO:372; and (ii) a VL comprising a VL CDR1 having an amino acid sequence of SEQ ID NO:373, a VL CDR2 having an amino acid sequence of SEQ ID NO:374, and a VL CDR3 having an amino acid sequence of SEQ ID NO:375. In some embodiments, the first binding domain that binds to TRGV9 comprises: (i) a VH comprising a VH CDR1 having an amino acid sequence of SEQ ID NO:376, a VH CDR2 having an amino acid sequence of SEQ ID NO:377, and a VH CDR3 having an amino acid sequence of SEQ ID NO:378; and (ii) a VL comprising a VL CDR1 having an amino acid sequence of SEQ ID NO:379, a VL CDR2 having an amino acid sequence of SEQ ID NO:380, and a VL CDR3 having an amino acid sequence of SEQ ID NO:381. In some embodiments, the first binding domain that binds to TRGV9 comprises: (i) a VH comprising a VH CDR1 having an amino acid sequence of SEQ ID NO:382, a VH CDR2 having an amino acid sequence of SEQ ID NO:383, and a VH CDR3 having an amino acid sequence of SEQ ID NO:384; and (ii) a VL comprising a VL CDR1 having an amino acid sequence of SEQ ID NO:385, a VL CDR2 having an amino acid sequence of SEQ ID NO:386, and a VL CDR3 having an amino acid sequence of SEQ ID NO:387. In some embodiments, the first binding domain that binds to TRGV9 comprises: (i) a VH comprising a VH CDR1 having an amino acid sequence of SEQ ID NO:388, a VH CDR2 having an amino acid sequence of SEQ ID NO:389, and a VH CDR3 having an amino acid sequence of SEQ ID NO:390; and (ii) a VL comprising a VL CDR1 having an amino acid sequence of SEQ ID NO:391, a VL CDR2 having an amino acid sequence of SEQ ID NO:392, and a VL CDR3 having an amino acid sequence of SEQ ID NO:393. In some embodiments, the first binding domain that binds to TRGV9 comprises: (i) a VH comprising a VH CDR1 having an amino acid sequence of SEQ ID NO:394, a VH CDR2 having an amino acid sequence of SEQ ID NO:395, and a VH CDR3 having an amino acid sequence of SEQ ID NO:396; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In some embodiments, the first binding domain that binds to TRGV9 comprises: (i) a VH comprising a VH CDR1 having an amino acid sequence of SEQ ID NO:394, a VH CDR2 having an amino acid sequence of SEQ ID NO:712, and a VH CDR3 having an amino acid sequence of SEQ ID NO:713; and (ii) a VL comprising a VL CDR1 having an amino acid sequence of SEQ ID NO:397, a VL CDR2 having an amino acid sequence of SEQ ID NO:398, and a VL CDR3 having an amino acid sequence of SEQ ID NO:399. In some embodiments, the first binding domain that binds to TRGV9 comprises: (i) a VH comprising a VH CDR1 having an amino acid sequence of SEQ ID NO:400, a VH CDR2 having an amino acid sequence of SEQ ID NO:401, and a VH CDR3 having an amino acid sequence of SEQ ID NO:402; and (ii) a VL comprising a VL CDR1 having an amino acid sequence of SEQ ID NO:403, a VL CDR2 having an amino acid sequence of SEQ ID NO:404, and a VL CDR3 having an amino acid sequence of SEQ ID NO:405. In some embodiments, the first binding domain that binds to TRGV9 comprises: (i) a VH comprising a VH CDR1 having an amino acid sequence of SEQ ID NO:406, a VH CDR2 having an amino acid sequence of SEQ ID NO:407, and a VH CDR3 having an amino acid sequence of SEQ ID NO:408; and (ii) a VL comprising a VL CDR1 having an amino acid sequence of SEQ ID NO:409, a VL CDR2 having an amino acid sequence of SEQ ID NO:410, and a VL CDR3 having an amino acid sequence of SEQ ID NO:411. In some embodiments, the first binding domain that binds to TRGV9 comprises: (i) a VH comprising a VH CDR1 having an amino acid sequence of SEQ ID NO:412, a VH CDR2 having an amino acid sequence of SEQ ID NO:413, and a VH CDR3 having an amino acid sequence of SEQ ID NO:414; and (ii) a VL comprising a VL CDR1 having an amino acid sequence of SEQ ID NO:415, a VL CDR2 having an amino acid sequence of SEQ ID NO:416, and a VL CDR3 having an amino acid sequence of SEQ ID NO:417. In some embodiments, the first binding domain that binds to TRGV9 comprises: (i) a VH comprising a VH CDR1 having an amino acid sequence of SEQ ID NO:418, a VH CDR2 having an amino acid sequence of SEQ ID NO:419, and a VH CDR3 having an amino acid sequence of SEQ ID NO:420; and (ii) a VL comprising a VL CDR1 having an amino acid sequence of SEQ ID NO:421, a VL CDR2 having an amino acid sequence of SEQ ID NO:422, and a VL CDR3 having an amino acid sequence of SEQ ID NO:423. In some embodiments, the first binding domain that binds to TRGV9 comprises: (i) a VH comprising a VH CDR1 having an amino acid sequence of SEQ ID NO:424, a VH CDR2 having an amino acid sequence of SEQ ID NO:425, and a VH CDR3 having an amino acid sequence of SEQ ID NO:426; and (ii) a VL comprising a VL CDR1 having an amino acid sequence of SEQ ID NO:427, a VL CDR2 having an amino acid sequence of SEQ ID NO:428, and a VL CDR3 having an amino acid sequence of SEQ ID NO:429. In some embodiments, the first binding domain that binds to TRGV9 comprises: (i) a VH comprising a VH CDR1 having an amino acid sequence of SEQ ID NO:430, a VH CDR2 having an amino acid sequence of SEQ ID NO:431, and a VH CDR3 having an amino acid sequence of SEQ ID NO:432; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In some embodiments, the first binding domain that binds to TRGV9 comprises: (i) a VH comprising a VH CDR1 having an amino acid sequence of SEQ ID NO:430, a VH CDR2 having an amino acid sequence of SEQ ID NO:714, and a VH CDR3 having an amino acid sequence of SEQ ID NO:715; and (ii) a VL comprising a VL CDR1 having an amino acid sequence of SEQ ID NO:433, a VL CDR2 having an amino acid sequence of SEQ ID NO:434, and a VL CDR3 having an amino acid sequence of SEQ ID NO:435. In some embodiments, the first binding domain that binds to TRGV9 comprises: (i) a VH comprising a VH CDR1 having an amino acid sequence of SEQ ID NO:436, a VH CDR2 having an amino acid sequence of SEQ ID NO:437, and a VH CDR3 having an amino acid sequence of SEQ ID NO:438; and (ii) a VL comprising a VL CDR1 having an amino acid sequence of SEQ ID NO:439, a VL CDR2 having an amino acid sequence of SEQ ID NO:440, and a VL CDR3 having an amino acid sequence of SEQ ID NO:441. In some embodiments, the first binding domain that binds to TRGV9 comprises: (i) a VH comprising a VH CDR1 having an amino acid sequence of SEQ ID NO:442, a VH CDR2 having an amino acid sequence of SEQ ID NO:443, and a VH CDR3 having an amino acid sequence of SEQ ID NO:444; and (ii) a VL comprising a VL CDR1 having an amino acid sequence of SEQ ID NO:445, a VL CDR2 having an amino acid sequence of SEQ ID NO:446, and a VL CDR3 having an amino acid sequence of SEQ ID NO:447. In some embodiments, the first binding domain that binds to TRGV9 comprises: (i) a VH comprising a VH CDR1 having an amino acid sequence of SEQ ID NO:448, a VH CDR2 having an amino acid sequence of SEQ ID NO:449, and a VH CDR3 having an amino acid sequence of SEQ ID NO:450; and (ii) a VL comprising a VL CDR1 having an amino acid sequence of SEQ ID NO:451, a VL CDR2 having an amino acid sequence of SEQ ID NO:452, and a VL CDR3 having an amino acid sequence of SEQ ID NO:453. In some embodiments, the first binding domain that binds to TRGV9 comprises: (i) a VH comprising a VH CDR1 having an amino acid sequence of SEQ ID NO:454, a VH CDR2 having an amino acid sequence of SEQ ID NO:455, and a VH CDR3 having an amino acid sequence of SEQ ID NO:456; and (ii) a VL comprising a VL CDR1 having an amino acid sequence of SEQ ID NO:457, a VL CDR2 having an amino acid sequence of SEQ ID NO:458, and a VL CDR3 having an amino acid sequence of SEQ ID NO:459. In some embodiments, the first binding domain that binds to TRGV9 comprises: (i) a VH comprising a VH CDR1 having an amino acid sequence of SEQ ID NO:460, a VH CDR2 having an amino acid sequence of SEQ ID NO:461, and a VH CDR3 having an amino acid sequence of SEQ ID NO:462; and (ii) a VL comprising a VL CDR1 having an amino acid sequence of SEQ ID NO:463, a VL CDR2 having an amino acid sequence of SEQ ID NO:464, and a VL CDR3 having an amino acid sequence of SEQ ID NO:465. In some embodiments, the first binding domain that binds to TRGV9 comprises: (i) a VH comprising a VH CDR1 having an amino acid sequence of SEQ ID NO:466, a VH CDR2 having an amino acid sequence of SEQ ID NO:467, and a VH CDR3 having an amino acid sequence of SEQ ID NO:468; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In some embodiments, the first binding domain that binds to TRGV9 comprises: (i) a VH comprising a VH CDR1 having an amino acid sequence of SEQ ID NO:466, a VH CDR2 having an amino acid sequence of SEQ ID NO:716, and a VH CDR3 having an amino acid sequence of SEQ ID NO:717; and (ii) a VL comprising a VL CDR1 having an amino acid sequence of SEQ ID NO:469, a VL CDR2 having an amino acid sequence of SEQ ID NO:470, and a VL CDR3 having an amino acid sequence of SEQ ID NO:471. In some embodiments, the first binding domain that binds to TRGV9 comprises: (i) a VH comprising a VH CDR1 having an amino acid sequence of SEQ ID NO:472, a VH CDR2 having an amino acid sequence of SEQ ID NO:473, and a VH CDR3 having an amino acid sequence of SEQ ID NO:474; and (ii) a VL comprising a VL CDR1 having an amino acid sequence of SEQ ID NO:475, a VL CDR2 having an amino acid sequence of SEQ ID NO:476, and a VL CDR3 having an amino acid sequence of SEQ ID NO:477. In some embodiments, the first binding domain that binds to TRGV9 comprises: (i) a VH comprising a VH CDR1 having an amino acid sequence of SEQ ID NO:478, a VH CDR2 having an amino acid sequence of SEQ ID NO:479, and a VH CDR3 having an amino acid sequence of SEQ ID NO:480; and (ii) a VL comprising a VL CDR1 having an amino acid sequence of SEQ ID NO:481, a VL CDR2 having an amino acid sequence of SEQ ID NO:482, and a VL CDR3 having an amino acid sequence of SEQ ID NO:483. In some embodiments, the first binding domain that binds to TRGV9 comprises: (i) a VH comprising a VH CDR1 having an amino acid sequence of SEQ ID NO:484, a VH CDR2 having an amino acid sequence of SEQ ID NO:485, and a VH CDR3 having an amino acid sequence of SEQ ID NO:486; and (ii) a VL comprising a VL CDR1 having an amino acid sequence of SEQ ID NO:487, a VL CDR2 having an amino acid sequence of SEQ ID NO:488, and a VL CDR3 having an amino acid sequence of SEQ ID NO:489. In some embodiments, the first binding domain that binds to TRGV9 comprises: (i) a VH comprising a VH CDR1 having an amino acid sequence of SEQ ID NO:490, a VH CDR2 having an amino acid sequence of SEQ ID NO:491, and a VH CDR3 having an amino acid sequence of SEQ ID NO:492; and (ii) a VL comprising a VL CDR1 having an amino acid sequence of SEQ ID NO:493, a VL CDR2 having an amino acid sequence of SEQ ID NO:494, and a VL CDR3 having an amino acid sequence of SEQ ID NO:495. In some embodiments, the first binding domain that binds to TRGV9 comprises: (i) a VH comprising a VH CDR1 having an amino acid sequence of SEQ ID NO:496, a VH CDR2 having an amino acid sequence of SEQ ID NO:497, and a VH CDR3 having an amino acid sequence of SEQ ID NO:498; and (ii) a VL comprising a VL CDR1 having an amino acid sequence of SEQ ID NO:499, a VL CDR2 having an amino acid sequence of SEQ ID NO:500, and a VL CDR3 having an amino acid sequence of SEQ ID NO:501. In some embodiments, the first binding domain that binds to TRGV9 comprises: (i) a VH comprising a VH CDR1 having an amino acid sequence of SEQ ID NO:502, a VH CDR2 having an amino acid sequence of SEQ ID NO:503, and a VH CDR3 having an amino acid sequence of SEQ ID NO:504; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In some embodiments, the first binding domain that binds to TRGV9 comprises: (i) a VH comprising a VH CDR1 having an amino acid sequence of SEQ ID NO:502, a VH CDR2 having an amino acid sequence of SEQ ID NO:718, and a VH CDR3 having an amino acid sequence of SEQ ID NO:719; and (ii) a VL comprising a VL CDR1 having an amino acid sequence of SEQ ID NO:505, a VL CDR2 having an amino acid sequence of SEQ ID NO:506, and a VL CDR3 having an amino acid sequence of SEQ ID NO:507. In some embodiments, the first binding domain that binds to TRGV9 comprises: (i) a VH comprising a VH CDR1 having an amino acid sequence of SEQ ID NO:508, a VH CDR2 having an amino acid sequence of SEQ ID NO:509, and a VH CDR3 having an amino acid sequence of SEQ ID NO:510; and (ii) a VL comprising a VL CDR1 having an amino acid sequence of SEQ ID NO:511, a VL CDR2 having an amino acid sequence of SEQ ID NO:512, and a VL CDR3 having an amino acid sequence of SEQ ID NO:513. In some embodiments, the first binding domain that binds to TRGV9 comprises: (i) a VH comprising a VH CDR1 having an amino acid sequence of SEQ ID NO:514, a VH CDR2 having an amino acid sequence of SEQ ID NO:515, and a VH CDR3 having an amino acid sequence of SEQ ID NO:516; and (ii) a VL comprising a VL CDR1 having an amino acid sequence of SEQ ID NO:517, a VL CDR2 having an amino acid sequence of SEQ ID NO:518, and a VL CDR3 having an amino acid sequence of SEQ ID NO:519. In some embodiments, the first binding domain that binds to TRGV9 comprises: (i) a VH comprising a VH CDR1 having an amino acid sequence of SEQ ID NO:520, a VH CDR2 having an amino acid sequence of SEQ ID NO:521, and a VH CDR3 having an amino acid sequence of SEQ ID NO:522; and (ii) a VL comprising a VL CDR1 having an amino acid sequence of SEQ ID NO:523, a VL CDR2 having an amino acid sequence of SEQ ID NO:524, and a VL CDR3 having an amino acid sequence of SEQ ID NO:525. In some embodiments, the first binding domain that binds to TRGV9 comprises: (i) a VH comprising a VH CDR1 having an amino acid sequence of SEQ ID NO:526, a VH CDR2 having an amino acid sequence of SEQ ID NO:527, and a VH CDR3 having an amino acid sequence of SEQ ID NO:528; and (ii) a VL comprising a VL CDR1 having an amino acid sequence of SEQ ID NO:529, a VL CDR2 having an amino acid sequence of SEQ ID NO:530, and a VL CDR3 having an amino acid sequence of SEQ ID NO:531. In some embodiments, the first binding domain that binds to TRGV9 comprises: (i) a VH comprising a VH CDR1 having an amino acid sequence of SEQ ID NO:532, a VH CDR2 having an amino acid sequence of SEQ ID NO:533, and a VH CDR3 having an amino acid sequence of SEQ ID NO:534; and (ii) a VL comprising a VL CDR1 having an amino acid sequence of SEQ ID NO:535, a VL CDR2 having an amino acid sequence of SEQ ID NO:536, and a VL CDR3 having an amino acid sequence of SEQ ID NO:537. In some embodiments, the first binding domain that binds to TRGV9 comprises: (i) a VH comprising a VH CDR1 having an amino acid sequence of SEQ ID NO:538, a VH CDR2 having an amino acid sequence of SEQ ID NO:539, and a VH CDR3 having an amino acid sequence of SEQ ID NO:540; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In some embodiments, the first binding domain that binds to TRGV9 comprises: (i) a VH comprising a VH CDR1 having an amino acid sequence of SEQ ID NO:538, a VH CDR2 having an amino acid sequence of SEQ ID NO:720, and a VH CDR3 having an amino acid sequence of SEQ ID NO:721; and (ii) a VL comprising a VL CDR1 having an amino acid sequence of SEQ ID NO:541, a VL CDR2 having an amino acid sequence of SEQ ID NO:542, and a VL CDR3 having an amino acid sequence of SEQ ID NO:543. In some embodiments, the first binding domain that binds to TRGV9 comprises: (i) a VH comprising a VH CDR1 having an amino acid sequence of SEQ ID NO:544, a VH CDR2 having an amino acid sequence of SEQ ID NO:545, and a VH CDR3 having an amino acid sequence of SEQ ID NO:546; and (ii) a VL comprising a VL CDR1 having an amino acid sequence of SEQ ID NO:547, a VL CDR2 having an amino acid sequence of SEQ ID NO:548, and a VL CDR3 having an amino acid sequence of SEQ ID NO:549. In some embodiments, the first binding domain that binds to TRGV9 comprises: (i) a VH comprising a VH CDR1 having an amino acid sequence of SEQ ID NO:550, a VH CDR2 having an amino acid sequence of SEQ ID NO:551, and a VH CDR3 having an amino acid sequence of SEQ ID NO:552; and (ii) a VL comprising a VL CDR1 having an amino acid sequence of SEQ ID NO:553, a VL CDR2 having an amino acid sequence of SEQ ID NO:554, and a VL CDR3 having an amino acid sequence of SEQ ID NO:555. In some embodiments, the second binding domain that binds CD123 comprises: (i) a VH comprising a VH CDR1 having an amino acid sequence of SEQ ID NO:556, a VH CDR2 having an amino acid sequence of SEQ ID NO:557, and a VH CDR3 having an amino acid sequence of SEQ ID NO:558; and (ii) a VL comprising a VL CDR1 having an amino acid sequence of SEQ ID NO:559, a VL CDR2 having an amino acid sequence of SEQ ID NO:560, and a VL CDR3 having an amino acid sequence of SEQ ID NO:561. In some embodiments, the second binding domain that binds CD123 comprises: (i) a VH comprising a VH CDR1 having an amino acid sequence of SEQ ID NO:562, a VH CDR2 having an amino acid sequence of SEQ ID NO:563, and a VH CDR3 having an amino acid sequence of SEQ ID NO:564; and (ii) a VL comprising a VL CDR1 having an amino acid sequence of SEQ ID NO:565, a VL CDR2 having an amino acid sequence of SEQ ID NO:566, and a VL CDR3 having an amino acid sequence of SEQ ID NO:567. In some embodiments, the second binding domain that binds CD123 comprises: (i) a VH comprising a VH CDR1 having an amino acid sequence of SEQ ID NO:568, a VH CDR2 having an amino acid sequence of SEQ ID NO:569, and a VH CDR3 having an amino acid sequence of SEQ ID NO:570; and (ii) a VL comprising a VL CDR1 having an amino acid sequence of SEQ ID NO:571, a VL CDR2 having an amino acid sequence of SEQ ID NO:572, and a VL CDR3 having an amino acid sequence of SEQ ID NO:573. In some embodiments, the second binding domain that binds CD123 comprises: (i) a VH comprising a VH CDR1 having an amino acid sequence of SEQ ID NO:574, a VH CDR2 having an amino acid sequence of SEQ ID NO:575, and a VH CDR3 having an amino acid sequence of SEQ ID NO:576; and (ii) a VL comprising a VL CDR1 having an amino acid sequence of SEQ ID NO:577, a VL CDR2 having an amino acid sequence of SEQ ID NO:578, and a VL CDR3 having an amino acid sequence of SEQ ID NO:579. In some embodiments, the second binding domain that binds CD123 comprises: (i) a VH comprising a VH CDR1 having an amino acid sequence of SEQ ID NO:574, a VH CDR2 having an amino acid sequence of SEQ ID NO:722, and a VH CDR3 having an amino acid sequence of SEQ ID NO:723; and (ii) a VL comprising a VL CDR1 having an amino acid sequence of SEQ ID NO:577, a VL CDR2 having an amino acid sequence of SEQ ID NO:578, and a VL CDR3 having an amino acid sequence of SEQ ID NO:579. In some embodiments, the second binding domain that binds CD123 comprises: (i) a VH comprising a VH CDR1 having an amino acid sequence of SEQ ID NO:580, a VH CDR2 having an amino acid sequence of SEQ ID NO:581, and a VH CDR3 having an amino acid sequence of SEQ ID NO:582; and (ii) a VL comprising a VL CDR1 having an amino acid sequence of SEQ ID NO:583, a VL CDR2 having an amino acid sequence of SEQ ID NO:584, and a VL CDR3 having an amino acid sequence of SEQ ID NO:585. In some embodiments, the second binding domain that binds CD123 comprises: (i) a VH comprising a VH CDR1 having an amino acid sequence of SEQ ID NO:586, a VH CDR2 having an amino acid sequence of SEQ ID NO:587, and a VH CDR3 having an amino acid sequence of SEQ ID NO:588; and (ii) a VL comprising a VL CDR1 having an amino acid sequence of SEQ ID NO:589, a VL CDR2 having an amino acid sequence of SEQ ID NO:590, and a VL CDR3 having an amino acid sequence of SEQ ID NO:591.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:7; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:34; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:35; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:36; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:65; and (ii) a VL comprising a VL CDR1, a VL CDR2, and a VL CDR3 having amino acid sequences of the VL CDR1, VL CDR2, and VL CDR3, respectively, of SEQ ID NO:66. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:67; and (ii) a VL comprising a VL CDR1, a VL CDR2, and a VL CDR3 having amino acid sequences of the VL CDR1, VL CDR2, and VL CDR3, respectively, of SEQ ID NO:68. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:95; and (ii) a VL comprising a VL CDR1, a VL CDR2, and a VL CDR3 having amino acid sequences of the VL CDR1, VL CDR2, and VL CDR3, respectively, of SEQ ID NO:96. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:104; and (ii) a VL comprising a VL CDR1, a VL CDR2, and a VL CDR3 having amino acid sequences of the VL CDR1, VL CDR2, and VL CDR3, respectively, of SEQ ID NO:105. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:113; and (ii) a VL comprising a VL CDR1, a VL CDR2, and a VL CDR3 having amino acid sequences of the VL CDR1, VL CDR2, and VL CDR3, respectively, of SEQ ID NO:114. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:123; and (ii) a VL comprising a VL CDR1, a VL CDR2, and a VL CDR3 having amino acid sequences of the VL CDR1, VL CDR2, and VL CDR3, respectively, of SEQ ID NO:124. In another aspect, provided herein is an antibody that binds to TRGV9, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO:133; and (ii) a VL comprising a VL CDR1, a VL CDR2, and a VL CDR3 having amino acid sequences of the VL CDR1, VL CDR2, and VL CDR3, respectively, of SEQ ID NO:134. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:7; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:34; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:35; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:36; and (ii) a VL comprising a VL CDR1, a VL CDR2, and a VL CDR3 having amino acid sequences of the VL CDR1, VL CDR2, and VL CDR3, respectively, of SEQ ID NO:8. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:65; and (ii) a VL comprising a VL CDR1, a VL CDR2, and a VL CDR3 having amino acid sequences of the VL CDR1, VL CDR2, and VL CDR3, respectively, of SEQ ID NO:66. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:67; and (ii) a VL comprising a VL CDR1, a VL CDR2, and a VL CDR3 having amino acid sequences of the VL CDR1, VL CDR2, and VL CDR3, respectively, of SEQ ID NO:68. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:95; and (ii) a VL comprising a VL CDR1, a VL CDR2, and a VL CDR3 having amino acid sequences of the VL CDR1, VL CDR2, and VL CDR3, respectively, of SEQ ID NO:96. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:104; and (ii) a VL comprising a VL CDR1, a VL CDR2, and a VL CDR3 having amino acid sequences of the VL CDR1, VL CDR2, and VL CDR3, respectively, of SEQ ID NO:105. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:113; and (ii) a VL comprising a VL CDR1, a VL CDR2, and a VL CDR3 having amino acid sequences of the VL CDR1, VL CDR2, and VL CDR3, respectively, of SEQ ID NO:114. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:123; and (ii) a VL comprising a VL CDR1, a VL CDR2, and a VL CDR3 having amino acid sequences of the VL CDR1, VL CDR2, and VL CDR3, respectively, of SEQ ID NO:124. In another aspect, provided herein is a bispecific antibody having a first binding domain that binds to TRGV9, wherein the first binding domain comprises: (i) a VH comprising a VH CDR1, a VH CDR2, and a VH CDR3 having amino acid sequences of the VH CDR1, VH CDR2, and VH CDR3, respectively, of SEQ ID NO:133; and (ii) a VL comprising a VL CDR1, a VL CDR2, and a VL CDR3 having amino acid sequences of the VL CDR1, VL CDR2, and VL CDR3, respectively, of SEQ ID NO:134. In certain embodiments, the bispecific antibody comprises a second binding domain that binds to tumor associated antigen. In certain embodiments, the bispecific antibody comprises a second binding domain that binds to CD123. In some embodiments, the second binding domain comprises: (i) a VH comprising a VH CDR1, a VH CDR2, and a VH CDR3 having amino acid sequences of the VH CDR1, VH CDR2, and VH CDR3, respectively, of SEQ ID NO:15; and (ii) a VL comprising a VL CDR1, a VL CDR2, and a VL CDR3 having amino acid sequences of the VL CDR1, VL CDR2, and VL CDR3, respectively, of SEQ ID NO:16. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

According to a particular aspect, provided herein is an isolated TRGV9 antibody or antigen-binding fragment thereof comprising (a) a first heavy chain (HC1); (b) a second heavy chain (HC2); (c) a first light chain (LC1); and (d) a second light chain (LC2). The HC1 can be associated with the LC1 and the HC2 can be associated with LC2. The HC1 can comprise a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 can comprise a light chain complementarity determining region 1 (LCDR1), LCDR2, and LCDR3 comprising the amino acid sequences of SEQ ID NO:4, SEQ ID NO:5, and SEQ ID NO:6, respectively. The HC1 and LC1 form a binding site for a first antigen, and the HC2 and LC2 form a binding site for a second antigen. The binding site for a first antigen can, for example, bind a TRGV9 on a γδ T cell. The binding site for a second antigen can, for example, bind a cancer antigen present on the surface of a cancer cell. The binding of the TRGV9 bispecific antibody to TRGV9 present on the surface of the γδ T cell, and the binding of the tumor associated antigen present on the surface of the cancer cell can, for example, result in the killing of the cancer cell.

According to another particular aspect, provided herein is an isolated TRGV9 antibody or antigen-binding fragment thereof comprising (a) a HC1; (b) a HC2; (c) a LC1; and (d) a LC2. The HC1 can be associated with the LC1 and the HC2 can be associated with LC2. In some embodiments, the HC1 comprises a HCDR1, HCDR2, and HCDR3 comprising the amino acid sequences of SEQ ID NO:1, SEQ ID NO:2, and SEQ ID NO:3, and LC1 comprises a LCDR1, LCDR2, and LCDR3 comprising the amino acid sequences of SEQ ID NO:4, SEQ ID NO:5, and SEQ ID NO:6, respectively. In some embodiments, the HC1 comprises a HCDR1, HCDR2, and HCDR3 comprising the amino acid sequences of SEQ ID NO:1, SEQ ID NO:2, and SEQ ID NO:31, and LC1 comprises a LCDR1, LCDR2, and LCDR3 comprising the amino acid sequences of SEQ ID NO:4, SEQ ID NO:5, and SEQ ID NO:6, respectively. In some embodiments, the HC1 comprises a HCDR1, HCDR2, and HCDR3 comprising the amino acid sequences of SEQ ID NO:1, SEQ ID NO:2, and SEQ ID NO:32, and LC1 comprises a LCDR1, LCDR2, and LCDR3 comprising the amino acid sequences of SEQ ID NO:4, SEQ ID NO:5, and SEQ ID NO:6, respectively. In some embodiments, the HC1 comprises a HCDR1, HCDR2, and HCDR3 comprising the amino acid sequences of SEQ ID NO:1, SEQ ID NO:2, and SEQ ID NO:33, and LC1 comprises a LCDR1, LCDR2, and LCDR3 comprising the amino acid sequences of SEQ ID NO:4, SEQ ID NO:5, and SEQ ID NO:6, respectively. In some embodiments, the HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR3 comprising the amino acid sequences of SEQ ID NO:77, SEQ ID NO:5, and SEQ ID NO:6, respectively. In some embodiments, the HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR3 comprising the amino acid sequences of SEQ ID NO:63, SEQ ID NO:64, and SEQ ID NO:6, respectively. In some embodiments, the HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR3 comprising the amino acid sequences of SEQ ID NO:92, SEQ ID NO:93, and SEQ ID NO:94, respectively. In some embodiments, the HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR3 comprising the amino acid sequences of SEQ ID NO:101, SEQ ID NO:102, and SEQ ID NO:103, respectively. In some embodiments, the HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR3 comprising the amino acid sequences of SEQ ID NO:110, SEQ ID NO:111, and SEQ ID NO:112, respectively. In some embodiments, the HC1 comprises a HCDR1, HCDR2, and HCDR3 comprising the amino acid sequences of SEQ ID NO:117, SEQ ID NO:118, and SEQ ID NO:119, respectively, and LC1 comprises a LCDR1, LCDR2, and LCDR3 comprising the amino acid sequences of SEQ ID NO:120, SEQ ID NO:121, and SEQ ID NO:122, respectively. In some embodiments, the HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR3 comprising the amino acid sequences of SEQ ID NO:130, SEQ ID NO:131, and SEQ ID NO:132, respectively. In some embodiments, the HC1 comprises a HCDR1, HCDR2, and HCDR3 comprising the amino acid sequences of SEQ ID NO:160, SEQ ID NO:161, and SEQ ID NO:162, respectively, and LC1 comprises a LCDR1, LCDR2, and LCDR3 comprising the amino acid sequences of SEQ ID NO:163, SEQ ID NO:164, and SEQ ID NO:165, respectively. In some embodiments, the HC1 comprises a HCDR1, HCDR2, and HCDR3 comprising the amino acid sequences of SEQ ID NO:166, SEQ ID NO: 167, and SEQ ID NO:168, respectively, and LC1 comprises a LCDR1, LCDR2, and LCDR3 comprising the amino acid sequences of SEQ ID NO:169, SEQ ID NO:170, and SEQ ID NO:171, respectively. In some embodiments, the HC1 comprises a HCDR1, HCDR2, and HCDR3 comprising the amino acid sequences of SEQ ID NO:172, SEQ ID NO: 173, and SEQ ID NO:174, respectively, and LC1 comprises a LCDR1, LCDR2, and LCDR3 comprising the amino acid sequences of SEQ ID NO:175, SEQ ID NO:176, and SEQ ID NO:177, respectively. In some embodiments, the HC1 comprises a HCDR1, HCDR2, and HCDR3 comprising the amino acid sequences of SEQ ID NO:178, SEQ ID NO: 179, and SEQ ID NO:180, respectively, and LC1 comprises a LCDR1, LCDR2, and LCDR3 comprising the amino acid sequences of SEQ ID NO:181, SEQ ID NO:182, and SEQ ID NO:183, respectively. In some embodiments, the HC1 comprises a HCDR1, HCDR2, and HCDR3 comprising the amino acid sequences of SEQ ID NO:178, SEQ ID NO:700, and SEQ ID NO:701, respectively, and LC1 comprises a LCDR1, LCDR2, and LCDR3 comprising the amino acid sequences of SEQ ID NO:181, SEQ ID NO:182, and SEQ ID NO:183, respectively. In some embodiments, the HC1 comprises a HCDR1, HCDR2, and HCDR3 comprising the amino acid sequences of SEQ ID NO:184, SEQ ID NO: 185, and SEQ ID NO:186, respectively, and LC1 comprises a LCDR1, LCDR2, and LCDR3 comprising the amino acid sequences of SEQ ID NO:187, SEQ ID NO:188, and SEQ ID NO:189, respectively. In some embodiments, the HC1 comprises a HCDR1, HCDR2, and HCDR3 comprising the amino acid sequences of SEQ ID NO:190, SEQ ID NO:191, and SEQ ID NO:192, respectively, and LC1 comprises a LCDR1, LCDR2, and LCDR3 comprising the amino acid sequences of SEQ ID NO:193, SEQ ID NO:194, and SEQ ID NO:195, respectively. In some embodiments, the HC1 comprises a HCDR1, HCDR2, and HCDR3 comprising the amino acid sequences of SEQ ID NO:196, SEQ ID NO: 197, and SEQ ID NO:198, respectively, and LC1 comprises a LCDR1, LCDR2, and LCDR3 comprising the amino acid sequences of SEQ ID NO:199, SEQ ID NO:200, and SEQ ID NO:201, respectively. In some embodiments, the HC1 comprises a HCDR1, HCDR2, and HCDR3 comprising the amino acid sequences of SEQ ID NO:202, SEQ ID NO:203, and SEQ ID NO:204, respectively, and LC1 comprises a LCDR1, LCDR2, and LCDR3 comprising the amino acid sequences of SEQ ID NO:205, SEQ ID NO:206, and SEQ ID NO:207, respectively. In some embodiments, the HC1 comprises a HCDR1, HCDR2, and HCDR3 comprising the amino acid sequences of SEQ ID NO:208, SEQ ID NO:209, and SEQ ID NO:210, respectively, and LC1 comprises a LCDR1, LCDR2, and LCDR3 comprising the amino acid sequences of SEQ ID NO:211, SEQ ID NO:212, and SEQ ID NO:213, respectively. In some embodiments, the HC1 comprises a HCDR1, HCDR2, and HCDR3 comprising the amino acid sequences of SEQ ID NO:214, SEQ ID NO:215, and SEQ ID NO:216, respectively, and LC1 comprises a LCDR1, LCDR2, and LCDR3 comprising the amino acid sequences of SEQ ID NO:217, SEQ ID NO:218, and SEQ ID NO:219, respectively. In some embodiments, the HC1 comprises a HCDR1, HCDR2, and HCDR3 comprising the amino acid sequences of SEQ ID NO:214, SEQ ID NO:702, and SEQ ID NO:703, respectively, and LC1 comprises a LCDR1, LCDR2, and LCDR3 comprising the amino acid sequences of SEQ ID NO:217, SEQ ID NO:218, and SEQ ID NO:219, respectively. In some embodiments, the HC1 comprises a HCDR1, HCDR2, and HCDR3 comprising the amino acid sequences of SEQ ID NO:220, SEQ ID NO:221, and SEQ ID NO:222, respectively, and LC1 comprises a LCDR1, LCDR2, and LCDR3 comprising the amino acid sequences of SEQ ID NO:223, SEQ ID NO:224, and SEQ ID NO:225, respectively. In some embodiments, the HC1 comprises a HCDR1, HCDR2, and HCDR3 comprising the amino acid sequences of SEQ ID NO:226, SEQ ID NO:227, and SEQ ID NO:228, respectively, and LC1 comprises a LCDR1, LCDR2, and LCDR3 comprising the amino acid sequences of SEQ ID NO:229, SEQ ID NO:230, and SEQ ID NO:231, respectively. In some embodiments, the HC1 comprises a HCDR1, HCDR2, and HCDR3 comprising the amino acid sequences of SEQ ID NO:232, SEQ ID NO:233, and SEQ ID NO:234, respectively, and LC1 comprises a LCDR1, LCDR2, and LCDR3 comprising the amino acid sequences of SEQ ID NO:235, SEQ ID NO:236, and SEQ ID NO:237, respectively. In some embodiments, the HC1 comprises a HCDR1, HCDR2, and HCDR3 comprising the amino acid sequences of SEQ ID NO:238, SEQ ID NO:239, and SEQ ID NO:240, respectively, and LC1 comprises a LCDR1, LCDR2, and LCDR3 comprising the amino acid sequences of SEQ ID NO:241, SEQ ID NO:242, and SEQ ID NO:243, respectively. In some embodiments, the HC1 comprises a HCDR1, HCDR2, and HCDR3 comprising the amino acid sequences of SEQ ID NO:244, SEQ ID NO:245, and SEQ ID NO:246, respectively, and LC1 comprises a LCDR1, LCDR2, and LCDR3 comprising the amino acid sequences of SEQ ID NO:247, SEQ ID NO:248, and SEQ ID NO:249, respectively. In some embodiments, the HC1 comprises a HCDR1, HCDR2, and HCDR3 comprising the amino acid sequences of SEQ ID NO:250, SEQ ID NO:251, and SEQ ID NO:252, respectively, and LC1 comprises a LCDR1, LCDR2, and LCDR3 comprising the amino acid sequences of SEQ ID NO:253, SEQ ID NO:254, and SEQ ID NO:255, respectively. In some embodiments, the HC1 comprises a HCDR1, HCDR2, and HCDR3 comprising the amino acid sequences of SEQ ID NO:250, SEQ ID NO:704, and SEQ ID NO:705, respectively, and LC1 comprises a LCDR1, LCDR2, and LCDR3 comprising the amino acid sequences of SEQ ID NO:253, SEQ ID NO:254, and SEQ ID NO:255, respectively. In some embodiments, the HC1 comprises a HCDR1, HCDR2, and HCDR3 comprising the amino acid sequences of SEQ ID NO:256, SEQ ID NO:257, and SEQ ID NO:258, respectively, and LC1 comprises a LCDR1, LCDR2, and LCDR3 comprising the amino acid sequences of SEQ ID NO:259, SEQ ID NO:260, and SEQ ID NO:261, respectively. In some embodiments, the HC1 comprises a HCDR1, HCDR2, and HCDR3 comprising the amino acid sequences of SEQ ID NO:262, SEQ ID NO:263, and SEQ ID NO:264, respectively, and LC1 comprises a LCDR1, LCDR2, and LCDR3 comprising the amino acid sequences of SEQ ID NO:265, SEQ ID NO:266, and SEQ ID NO:267, respectively. In some embodiments, the HC1 comprises a HCDR1, HCDR2, and HCDR3 comprising the amino acid sequences of SEQ ID NO:268, SEQ ID NO:269, and SEQ ID NO:270, respectively, and LC1 comprises a LCDR1, LCDR2, and LCDR3 comprising the amino acid sequences of SEQ ID NO:271, SEQ ID NO:272, and SEQ ID NO:273, respectively. In some embodiments, the HC1 comprises a HCDR1, HCDR2, and HCDR3 comprising the amino acid sequences of SEQ ID NO:274, SEQ ID NO:275, and SEQ ID NO:276, respectively, and LC1 comprises a LCDR1, LCDR2, and LCDR3 comprising the amino acid sequences of SEQ ID NO:277, SEQ ID NO:278, and SEQ ID NO:279, respectively. In some embodiments, the HC1 comprises a HCDR1, HCDR2, and HCDR3 comprising the amino acid sequences of SEQ ID NO:280, SEQ ID NO:281, and SEQ ID NO:282, respectively, and LC1 comprises a LCDR1, LCDR2, and LCDR3 comprising the amino acid sequences of SEQ ID NO:283, SEQ ID NO:284, and SEQ ID NO:285, respectively. In some embodiments, the HC1 comprises a HCDR1, HCDR2, and HCDR3 comprising the amino acid sequences of SEQ ID NO:286, SEQ ID NO:287, and SEQ ID NO:288, respectively, and LC1 comprises a LCDR1, LCDR2, and LCDR3 comprising the amino acid sequences of SEQ ID NO:289, SEQ ID NO:290, and SEQ ID NO:291, respectively. In some embodiments, the HC1 comprises a HCDR1, HCDR2, and HCDR3 comprising the amino acid sequences of SEQ ID NO:286, SEQ ID NO:706, and SEQ ID NO:707, respectively, and LC1 comprises a LCDR1, LCDR2, and LCDR3 comprising the amino acid sequences of SEQ ID NO:289, SEQ ID NO:290, and SEQ ID NO:291, respectively. In some embodiments, the HC1 comprises a HCDR1, HCDR2, and HCDR3 comprising the amino acid sequences of SEQ ID NO:292, SEQ ID NO:293, and SEQ ID NO:294, respectively, and LC1 comprises a LCDR1, LCDR2, and LCDR3 comprising the amino acid sequences of SEQ ID NO:295, SEQ ID NO:296, and SEQ ID NO:297, respectively. In some embodiments, the HC1 comprises a HCDR1, HCDR2, and HCDR3 comprising the amino acid sequences of SEQ ID NO:298, SEQ ID NO:299, and SEQ ID NO:300, respectively, and LC1 comprises a LCDR1, LCDR2, and LCDR3 comprising the amino acid sequences of SEQ ID NO:301, SEQ ID NO:302, and SEQ ID NO:303, respectively. In some embodiments, the HC1 comprises a HCDR1, HCDR2, and HCDR3 comprising the amino acid sequences of SEQ ID NO:304, SEQ ID NO:305, and SEQ ID NO:306, respectively, and LC1 comprises a LCDR1, LCDR2, and LCDR3 comprising the amino acid sequences of SEQ ID NO:307, SEQ ID NO:308, and SEQ ID NO:309, respectively. In some embodiments, the HC1 comprises a HCDR1, HCDR2, and HCDR3 comprising the amino acid sequences of SEQ ID NO:310, SEQ ID NO:311, and SEQ ID NO:312, respectively, and LC1 comprises a LCDR1, LCDR2, and LCDR3 comprising the amino acid sequences of SEQ ID NO:313, SEQ ID NO:314, and SEQ ID NO:315, respectively. In some embodiments, the HC1 comprises a HCDR1, HCDR2, and HCDR3 comprising the amino acid sequences of SEQ ID NO:316, SEQ ID NO:317, and SEQ ID NO:318, respectively, and LC1 comprises a LCDR1, LCDR2, and LCDR3 comprising the amino acid sequences of SEQ ID NO:319, SEQ ID NO:320, and SEQ ID NO:321, respectively. In some embodiments, the HC1 comprises a HCDR1, HCDR2, and HCDR3 comprising the amino acid sequences of SEQ ID NO:322, SEQ ID NO:323, and SEQ ID NO:324, respectively, and LC1 comprises a LCDR1, LCDR2, and LCDR3 comprising the amino acid sequences of SEQ ID NO:325, SEQ ID NO:326, and SEQ ID NO:327, respectively. In some embodiments, the HC1 comprises a HCDR1, HCDR2, and HCDR3 comprising the amino acid sequences of SEQ ID NO:322, SEQ ID NO:708, and SEQ ID NO:709, respectively, and LC1 comprises a LCDR1, LCDR2, and LCDR3 comprising the amino acid sequences of SEQ ID NO:325, SEQ ID NO:326, and SEQ ID NO:327, respectively. In some embodiments, the HC1 comprises a HCDR1, HCDR2, and HCDR3 comprising the amino acid sequences of SEQ ID NO:328, SEQ ID NO:329, and SEQ ID NO:330, respectively, and LC1 comprises a LCDR1, LCDR2, and LCDR3 comprising the amino acid sequences of SEQ ID NO:331, SEQ ID NO:332, and SEQ ID NO:333, respectively. In some embodiments, the HC1 comprises a HCDR1, HCDR2, and HCDR3 comprising the amino acid sequences of SEQ ID NO:334, SEQ ID NO:335, and SEQ ID NO:336, respectively, and LC1 comprises a LCDR1, LCDR2, and LCDR3 comprising the amino acid sequences of SEQ ID NO:337, SEQ ID NO:338, and SEQ ID NO:339, respectively. In some embodiments, the HC1 comprises a HCDR1, HCDR2, and HCDR3 comprising the amino acid sequences of SEQ ID NO:340, SEQ ID NO:341, and SEQ ID NO:342, respectively, and LC1 comprises a LCDR1, LCDR2, and LCDR3 comprising the amino acid sequences of SEQ ID NO:343, SEQ ID NO:344, and SEQ ID NO:345, respectively. In some embodiments, the HC1 comprises a HCDR1, HCDR2, and HCDR3 comprising the amino acid sequences of SEQ ID NO:346, SEQ ID NO:347, and SEQ ID NO:348, respectively, and LC1 comprises a LCDR1, LCDR2, and LCDR3 comprising the amino acid sequences of SEQ ID NO:349, SEQ ID NO:350, and SEQ ID NO:351, respectively. In some embodiments, the HC1 comprises a HCDR1, HCDR2, and HCDR3 comprising the amino acid sequences of SEQ ID NO:352, SEQ ID NO:353, and SEQ ID NO:354, respectively, and LC1 comprises a LCDR1, LCDR2, and LCDR3 comprising the amino acid sequences of SEQ ID NO:355, SEQ ID NO:356, and SEQ ID NO:357, respectively. In some embodiments, the HC1 comprises a HCDR1, HCDR2, and HCDR3 comprising the amino acid sequences of SEQ ID NO:358, SEQ ID NO:359, and SEQ ID NO:360, respectively, and LC1 comprises a LCDR1, LCDR2, and LCDR3 comprising the amino acid sequences of SEQ ID NO:361, SEQ ID NO:362, and SEQ ID NO:363, respectively. In some embodiments, the HC1 comprises a HCDR1, HCDR2, and HCDR3 comprising the amino acid sequences of SEQ ID NO:358, SEQ ID NO:710, and SEQ ID NO:711, respectively, and LC1 comprises a LCDR1, LCDR2, and LCDR3 comprising the amino acid sequences of SEQ ID NO:361, SEQ ID NO:362, and SEQ ID NO:363, respectively. In some embodiments, the HC1 comprises a HCDR1, HCDR2, and HCDR3 comprising the amino acid sequences of SEQ ID NO:364, SEQ ID NO:365, and SEQ ID NO:366, respectively, and LC1 comprises a LCDR1, LCDR2, and LCDR3 comprising the amino acid sequences of SEQ ID NO:367, SEQ ID NO:368, and SEQ ID NO:369, respectively. In some embodiments, the HC1 comprises a HCDR1, HCDR2, and HCDR3 comprising the amino acid sequences of SEQ ID NO:370, SEQ ID NO:371, and SEQ ID NO:372, respectively, and LC1 comprises a LCDR1, LCDR2, and LCDR3 comprising the amino acid sequences of SEQ ID NO:373, SEQ ID NO:374, and SEQ ID NO:375, respectively. In some embodiments, the HC1 comprises a HCDR1, HCDR2, and HCDR3 comprising the amino acid sequences of SEQ ID NO:376, SEQ ID NO:377, and SEQ ID NO:378, respectively, and LC1 comprises a LCDR1, LCDR2, and LCDR3 comprising the amino acid sequences of SEQ ID NO:379, SEQ ID NO:380, and SEQ ID NO:381, respectively. In some embodiments, the HC1 comprises a HCDR1, HCDR2, and HCDR3 comprising the amino acid sequences of SEQ ID NO:382, SEQ ID NO:383, and SEQ ID NO:384, respectively, and LC1 comprises a LCDR1, LCDR2, and LCDR3 comprising the amino acid sequences of SEQ ID NO:385, SEQ ID NO:386, and SEQ ID NO:387, respectively. In some embodiments, the HC1 comprises a HCDR1, HCDR2, and HCDR3 comprising the amino acid sequences of SEQ ID NO:388, SEQ ID NO:389, and SEQ ID NO:390, respectively, and LC1 comprises a LCDR1, LCDR2, and LCDR3 comprising the amino acid sequences of SEQ ID NO:391, SEQ ID NO:392, and SEQ ID NO:393, respectively. In some embodiments, the HC1 comprises a HCDR1, HCDR2, and HCDR3 comprising the amino acid sequences of SEQ ID NO:394, SEQ ID NO:395, and SEQ ID NO:396, respectively, and LC1 comprises a LCDR1, LCDR2, and LCDR3 comprising the amino acid sequences of SEQ ID NO:397, SEQ ID NO:398, and SEQ ID NO:399, respectively. In some embodiments, the HC1 comprises a HCDR1, HCDR2, and HCDR3 comprising the amino acid sequences of SEQ ID NO:394, SEQ ID NO:712, and SEQ ID NO:713, respectively, and LC1 comprises a LCDR1, LCDR2, and LCDR3 comprising the amino acid sequences of SEQ ID NO:397, SEQ ID NO:398, and SEQ ID NO:399, respectively. In some embodiments, the HC1 comprises a HCDR1, HCDR2, and HCDR3 comprising the amino acid sequences of SEQ ID NO:400, SEQ ID NO:401, and SEQ ID NO:402, respectively, and LC1 comprises a LCDR1, LCDR2, and LCDR3 comprising the amino acid sequences of SEQ ID NO:403, SEQ ID NO:404, and SEQ ID NO:405, respectively. In some embodiments, the HC1 comprises a HCDR1, HCDR2, and HCDR3 comprising the amino acid sequences of SEQ ID NO:406, SEQ ID NO:407, and SEQ ID NO:408, respectively, and LC1 comprises a LCDR1, LCDR2, and LCDR3 comprising the amino acid sequences of SEQ ID NO:409, SEQ ID NO:410, and SEQ ID NO:411, respectively. In some embodiments, the HC1 comprises a HCDR1, HCDR2, and HCDR3 comprising the amino acid sequences of SEQ ID NO:412, SEQ ID NO:413, and SEQ ID NO:414, respectively, and LC1 comprises a LCDR1, LCDR2, and LCDR3 comprising the amino acid sequences of SEQ ID NO:415, SEQ ID NO:416, and SEQ ID NO:417, respectively. In some embodiments, the HC1 comprises a HCDR1, HCDR2, and HCDR3 comprising the amino acid sequences of SEQ ID NO:418, SEQ ID NO:419, and SEQ ID NO:420, respectively, and LC1 comprises a LCDR1, LCDR2, and LCDR3 comprising the amino acid sequences of SEQ ID NO:421, SEQ ID NO:422, and SEQ ID NO:423, respectively. In some embodiments, the HC1 comprises a HCDR1, HCDR2, and HCDR3 comprising the amino acid sequences of SEQ ID NO:424, SEQ ID NO:425, and SEQ ID NO:426, respectively, and LC1 comprises a LCDR1, LCDR2, and LCDR3 comprising the amino acid sequences of SEQ ID NO:427, SEQ ID NO:428, and SEQ ID NO:429, respectively. In some embodiments, the HC1 comprises a HCDR1, HCDR2, and HCDR3 comprising the amino acid sequences of SEQ ID NO:430, SEQ ID NO:431, and SEQ ID NO:432, respectively, and LC1 comprises a LCDR1, LCDR2, and LCDR3 comprising the amino acid sequences of SEQ ID NO:433, SEQ ID NO:434, and SEQ ID NO:435, respectively. In some embodiments, the HC1 comprises a HCDR1, HCDR2, and HCDR3 comprising the amino acid sequences of SEQ ID NO:430, SEQ ID NO:714, and SEQ ID NO:715, respectively, and LC1 comprises a LCDR1, LCDR2, and LCDR3 comprising the amino acid sequences of SEQ ID NO:433, SEQ ID NO:434, and SEQ ID NO:435, respectively. In some embodiments, the HC1 comprises a HCDR1, HCDR2, and HCDR3 comprising the amino acid sequences of SEQ ID NO:436, SEQ ID NO:437, and SEQ ID NO:438, respectively, and LC1 comprises a LCDR1, LCDR2, and LCDR3 comprising the amino acid sequences of SEQ ID NO:439, SEQ ID NO:440, and SEQ ID NO:441, respectively. In some embodiments, the HC1 comprises a HCDR1, HCDR2, and HCDR3 comprising the amino acid sequences of SEQ ID NO:442, SEQ ID NO:443, and SEQ ID NO:444, respectively, and LC1 comprises a LCDR1, LCDR2, and LCDR3 comprising the amino acid sequences of SEQ ID NO:445, SEQ ID NO:446, and SEQ ID NO:447, respectively. In some embodiments, the HC1 comprises a HCDR1, HCDR2, and HCDR3 comprising the amino acid sequences of SEQ ID NO:448, SEQ ID NO:449, and SEQ ID NO:450, respectively, and LC1 comprises a LCDR1, LCDR2, and LCDR3 comprising the amino acid sequences of SEQ ID NO:451, SEQ ID NO:452, and SEQ ID NO:453, respectively. In some embodiments, the HC1 comprises a HCDR1, HCDR2, and HCDR3 comprising the amino acid sequences of SEQ ID NO:454, SEQ ID NO:455, and SEQ ID NO:456, respectively, and LC1 comprises a LCDR1, LCDR2, and LCDR3 comprising the amino acid sequences of SEQ ID NO:457, SEQ ID NO:458, and SEQ ID NO:459, respectively. In some embodiments, the HC1 comprises a HCDR1, HCDR2, and HCDR3 comprising the amino acid sequences of SEQ ID NO:460, SEQ ID NO:461, and SEQ ID NO:462, respectively, and LC1 comprises a LCDR1, LCDR2, and LCDR3 comprising the amino acid sequences of SEQ ID NO:463, SEQ ID NO:464, and SEQ ID NO:465, respectively. In some embodiments, the HC1 comprises a HCDR1, HCDR2, and HCDR3 comprising the amino acid sequences of SEQ ID NO:466, SEQ ID NO:467, and SEQ ID NO:468, respectively, and LC1 comprises a LCDR1, LCDR2, and LCDR3 comprising the amino acid sequences of SEQ ID NO:469, SEQ ID NO:470, and SEQ ID NO:471, respectively. In some embodiments, the HC1 comprises a HCDR1, HCDR2, and HCDR3 comprising the amino acid sequences of SEQ ID NO:466, SEQ ID NO:716, and SEQ ID NO:717, respectively, and LC1 comprises a LCDR1, LCDR2, and LCDR3 comprising the amino acid sequences of SEQ ID NO:469, SEQ ID NO:470, and SEQ ID NO:471, respectively. In some embodiments, the HC1 comprises a HCDR1, HCDR2, and HCDR3 comprising the amino acid sequences of SEQ ID NO:472, SEQ ID NO:473, and SEQ ID NO:474, respectively, and LC1 comprises a LCDR1, LCDR2, and LCDR3 comprising the amino acid sequences of SEQ ID NO:475, SEQ ID NO:476, and SEQ ID NO:477, respectively. In some embodiments, the HC1 comprises a HCDR1, HCDR2, and HCDR3 comprising the amino acid sequences of SEQ ID NO:478, SEQ ID NO:479, and SEQ ID NO:480, respectively, and LC1 comprises a LCDR1, LCDR2, and LCDR3 comprising the amino acid sequences of SEQ ID NO:481, SEQ ID NO:482, and SEQ ID NO:483, respectively. In some embodiments, the HC1 comprises a HCDR1, HCDR2, and HCDR3 comprising the amino acid sequences of SEQ ID NO:484, SEQ ID NO:485, and SEQ ID NO:486, respectively, and LC1 comprises a LCDR1, LCDR2, and LCDR3 comprising the amino acid sequences of SEQ ID NO:487, SEQ ID NO:488, and SEQ ID NO:489, respectively. In some embodiments, the HC1 comprises a HCDR1, HCDR2, and HCDR3 comprising the amino acid sequences of SEQ ID NO:490, SEQ ID NO:491, and SEQ ID NO:492, respectively, and LC1 comprises a LCDR1, LCDR2, and LCDR3 comprising the amino acid sequences of SEQ ID NO:493, SEQ ID NO:494, and SEQ ID NO:495, respectively. In some embodiments, the HC1 comprises a HCDR1, HCDR2, and HCDR3 comprising the amino acid sequences of SEQ ID NO:496, SEQ ID NO:497, and SEQ ID NO:498, respectively, and LC1 comprises a LCDR1, LCDR2, and LCDR3 comprising the amino acid sequences of SEQ ID NO:499, SEQ ID NO:500, and SEQ ID NO:501, respectively. In some embodiments, the HC1 comprises a HCDR1, HCDR2, and HCDR3 comprising the amino acid sequences of SEQ ID NO:502, SEQ ID NO:503, and SEQ ID NO:504, respectively, and LC1 comprises a LCDR1, LCDR2, and LCDR3 comprising the amino acid sequences of SEQ ID NO:505, SEQ ID NO:506, and SEQ ID NO:507, respectively. In some embodiments, the HC1 comprises a HCDR1, HCDR2, and HCDR3 comprising the amino acid sequences of SEQ ID NO:502, SEQ ID NO:718, and SEQ ID NO:719, respectively, and LC1 comprises a LCDR1, LCDR2, and LCDR3 comprising the amino acid sequences of SEQ ID NO:505, SEQ ID NO:506, and SEQ ID NO:507, respectively. In some embodiments, the HC1 comprises a HCDR1, HCDR2, and HCDR3 comprising the amino acid sequences of SEQ ID NO:508, SEQ ID NO:509, and SEQ ID NO:510, respectively, and LC1 comprises a LCDR1, LCDR2, and LCDR3 comprising the amino acid sequences of SEQ ID NO:511, SEQ ID NO:512, and SEQ ID NO:513, respectively. In some embodiments, the HC1 comprises a HCDR1, HCDR2, and HCDR3 comprising the amino acid sequences of SEQ ID NO:514, SEQ ID NO:515, and SEQ ID NO:516, respectively, and LC1 comprises a LCDR1, LCDR2, and LCDR3 comprising the amino acid sequences of SEQ ID NO:517, SEQ ID NO:518, and SEQ ID NO:519, respectively. In some embodiments, the HC1 comprises a HCDR1, HCDR2, and HCDR3 comprising the amino acid sequences of SEQ ID NO:520, SEQ ID NO:521, and SEQ ID NO:522, respectively, and LC1 comprises a LCDR1, LCDR2, and LCDR3 comprising the amino acid sequences of SEQ ID NO:523, SEQ ID NO:524, and SEQ ID NO:525, respectively. In some embodiments, the HC1 comprises a HCDR1, HCDR2, and HCDR3 comprising the amino acid sequences of SEQ ID NO:526, SEQ ID NO:527, and SEQ ID NO:528, respectively, and LC1 comprises a LCDR1, LCDR2, and LCDR3 comprising the amino acid sequences of SEQ ID NO:529, SEQ ID NO:530, and SEQ ID NO:531, respectively. In some embodiments, the HC1 comprises a HCDR1, HCDR2, and HCDR3 comprising the amino acid sequences of SEQ ID NO:532, SEQ ID NO:533, and SEQ ID NO:534, respectively, and LC1 comprises a LCDR1, LCDR2, and LCDR3 comprising the amino acid sequences of SEQ ID NO:535, SEQ ID NO:536, and SEQ ID NO:537, respectively. In some embodiments, the HC1 comprises a HCDR1, HCDR2, and HCDR3 comprising the amino acid sequences of SEQ ID NO:538, SEQ ID NO:539, and SEQ ID NO:540, respectively, and LC1 comprises a LCDR1, LCDR2, and LCDR3 comprising the amino acid sequences of SEQ ID NO:541, SEQ ID NO:542, and SEQ ID NO:543, respectively. In some embodiments, the HC1 comprises a HCDR1, HCDR2, and HCDR3 comprising the amino acid sequences of SEQ ID NO:532, SEQ ID NO:533, and SEQ ID NO:534, respectively, and LC1 comprises a LCDR1, LCDR2, and LCDR3 comprising the amino acid sequences of SEQ ID NO:535, SEQ ID NO:536, and SEQ ID NO:537, respectively. In some embodiments, the HC1 comprises a HCDR1, HCDR2, and HCDR3 comprising the amino acid sequences of SEQ ID NO:538, SEQ ID NO:720, and SEQ ID NO:721, respectively, and LC1 comprises a LCDR1, LCDR2, and LCDR3 comprising the amino acid sequences of SEQ ID NO:541, SEQ ID NO:542, and SEQ ID NO:543, respectively. In some embodiments, the HC1 comprises a HCDR1, HCDR2, and HCDR3 comprising the amino acid sequences of SEQ ID NO:544, SEQ ID NO:545, and SEQ ID NO:546, respectively, and LC1 comprises a LCDR1, LCDR2, and LCDR3 comprising the amino acid sequences of SEQ ID NO:547, SEQ ID NO:548, and SEQ ID NO:549, respectively. In some embodiments, the HC1 comprises a HCDR1, HCDR2, and HCDR3 comprising the amino acid sequences of SEQ ID NO:550, SEQ ID NO:551, and SEQ ID NO:552, respectively, and LC1 comprises a LCDR1, LCDR2, and LCDR3 comprising the amino acid sequences of SEQ ID NO:553, SEQ ID NO:554, and SEQ ID NO:555, respectively. In specific embodiments, the HC1 and LC1 form a binding site for a first antigen, and the HC2 and LC2 form a binding site for a second antigen. In some embodiments, HC1 is associated with LC1 and HC2 is associated with LC2. In some embodiments, HC1 and LC1 form a binding site for a first antigen that specifically binds TRGV9. The binding site for a first antigen can, for example, bind a TRGV9 on a γδ T cell. The binding site for a second antigen can, for example, bind a cancer antigen present on the surface of a cancer cell. In some embodiments, the cancer antigen is CD123. The binding of the TRGV9 bispecific antibody to TRGV9 present on the surface of the γδ T cell, and the binding of the tumor associated antigen present on the surface of the cancer cell can, for example, result in the killing of the cancer cell.

In certain embodiments, the first binding domain of the bispecific antibody specifically binds TRGV9. In some embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody.

Also provided herein are anti-TRGV9/anti-CD123 bispecific antibodies or antigen-binding fragments thereof comprising an anti-TRGV9 antibody or an antigen-binding fragment thereof and an anti-CD123 antibody or antigen-binding fragment thereof. In certain embodiments the anti-TRGV9/anti-CD123 bispecific antibody or antigen binding fragment thereof comprises (a) a HC1; (b) a HC2; (c) a LC1; and a LC2. In some embodiments, HC1 is associated with LC1 and HC2 is associated with LC2. HC1 can, for example, comprise a HCDR1, HCDR2, and HCDR3 comprising the amino 5 acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
   and LC1 can, for example, comprise a LCDR1, LCDR2, and LCDR3 comprising the amino acid sequences of SEQ ID NO:4, SEQ ID NO:5, and SEQ ID NO:6, respectively, to form a binding site for a first antigen that specifically binds TRGV9. HC2 can, for example, comprise a HCDR1, HCDR2, and HCDR3 comprising the amino acid sequences of SEQ ID NO:9, SEQ ID NO:10, and SEQ ID NO:11, respectively, and LC2 can, for example, comprise a LCDR1, LCDR2, and LCDR3 comprising the amino acid sequences of SEQ ID NO: 12, SEQ ID NO:13, and SEQ ID NO:14, respectively, to form a binding site for a second antigen that specifically binds CD123. In certain embodiments, the HC1 comprises the amino acid sequence of SEQ ID NO:7, SEQ ID NO:34, SEQ ID NO:35, or SEQ ID NO:36, and LC1 comprises the amino acid sequence of SEQ ID NO:8, and the HC2 comprises the amino acid sequence of SEQ ID NO:15 and LC2 comprises the amino acid sequence of SEQ ID NO:16. In certain embodiments, the TRGV9 is on the surface of a γδ T cell. In certain embodiments, the CD123 is on the surface of a cancer cell (e.g., a leukemia cell).

Also provided herein are anti-TRGV9/anti-CD123 bispecific antibodies or 25 antigen-binding fragments thereof comprising an anti-TRGV9 antibody or an antigen-binding fragment thereof and an anti-CD123 antibody or antigen-binding fragment thereof. In certain embodiments the anti-TRGV9/anti-CD123 bispecific antibody or antigen binding fragment thereof comprises (a) a HC1; (b) a HC2; (c) a LC1; and a LC2. In some embodiments, HC1 is associated with LC1 and HC2 is associated with LC2. In some embodiments, HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds TRGV9. In some embodiments, HC1 comprises a HCDR1, HCDR2, and HCDR3 comprising the amino acid sequences of SEQ ID NO:1, SEQ ID NO:2, and SEQ ID NO:31, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds TRGV9. In some embodiments, HC1 comprises a HCDR1, HCDR2, and HCDR3 comprising the amino acid sequences of SEQ ID NO:1, SEQ ID NO:2, and SEQ ID NO:32, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds TRGV9. In some embodiments, HC1 comprises a HCDR1, HCDR2, and HCDR3 comprising the amino acid sequences of SEQ ID NO:1, SEQ ID NO:2, and SEQ ID NO:33, respectively, and LC1 comprises a LCDR1, LCDR2, and LCDR3 comprising the amino acid sequences of SEQ ID NO:4, SEQ ID NO:5, and SEQ ID NO:6, respectively, to form a binding site for a first antigen that specifically binds TRGV9. In some embodiments, HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR3 comprising the amino acid sequences of SEQ ID NO:77, SEQ ID NO:5, and SEQ ID NO:6, respectively, to form a binding site for a first antigen that specifically binds TRGV9. In some embodiments, HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR3 comprising the amino acid sequences of SEQ ID NO:63, SEQ ID NO:64, and SEQ ID NO:6, respectively, to form a binding site for a first antigen that specifically binds TRGV9. In some embodiments, the HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR3 comprising the amino acid sequences of SEQ ID NO:92, SEQ ID NO:93, and SEQ ID NO:94, respectively, to form a binding site for a first antigen that specifically binds TRGV9. In some embodiments, the HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR3 comprising the amino acid sequences of SEQ ID NO:101, SEQ ID NO:102, and SEQ ID NO:103, respectively, to form a binding site for a first antigen that specifically binds TRGV9. In some embodiments, the HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR3 comprising the amino acid sequences of SEQ ID NO:110, SEQ ID NO:111, and SEQ ID NO:112, respectively, to form a binding site for a first antigen that specifically binds TRGV9. In some embodiments, the HC1 comprises a HCDR1, HCDR2, and HCDR3 comprising the amino acid sequences of SEQ ID NO:117, SEQ ID NO:118, and SEQ ID NO:119, respectively, and LC1 comprises a LCDR1, LCDR2, and LCDR3 comprising the amino acid sequences of SEQ ID NO:120, SEQ ID NO:121, and SEQ ID NO:122, respectively, to form a binding site for a first antigen that specifically binds TRGV9. In some embodiments, the HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR3 comprising the amino acid sequences of SEQ ID NO:130, SEQ ID NO:131, and SEQ ID NO:132, respectively, to form a binding site for a first antigen that specifically binds TRGV9. In some embodiments, the HC1 comprises a HCDR1, HCDR2, and HCDR3 comprising the amino acid sequences of SEQ ID NO:160, SEQ ID NO:161, and SEQ ID NO:162, respectively, and LC1 comprises a LCDR1, LCDR2, and LCDR3 comprising the amino acid sequences of SEQ ID NO:163, SEQ ID NO:164, and SEQ ID NO:165, respectively, to form a binding site for a first antigen that specifically binds TRGV9. In some embodiments, the HC1 comprises a HCDR1, HCDR2, and HCDR3 comprising the amino acid sequences of SEQ ID NO:166, SEQ ID NO:167, and SEQ ID NO:168, respectively, and LC1 comprises a LCDR1, LCDR2, and LCDR3 comprising the amino acid sequences of SEQ ID NO:169, SEQ ID NO:170, and SEQ ID NO:171, respectively, to form a binding site for a first antigen that specifically binds TRGV9. In some embodiments, the HC1 comprises a HCDR1, HCDR2, and HCDR3 comprising the amino acid sequences of SEQ ID NO:172, SEQ ID NO:173, and SEQ ID NO:174, respectively, and LC1 comprises a LCDR1, LCDR2, and LCDR3 comprising the amino acid sequences of SEQ ID NO:175, SEQ ID NO:176, and SEQ ID NO:177, respectively, to form a binding site for a first antigen that specifically binds TRGV9. In some embodiments, the HC1 comprises a HCDR1, HCDR2, and HCDR3 comprising the amino acid sequences of SEQ ID NO:178, SEQ ID NO:179, and SEQ ID NO:180, respectively, and LC1 comprises a LCDR1, LCDR2, and LCDR3 comprising the amino acid sequences of SEQ ID NO:181, SEQ ID NO:182, and SEQ ID NO:183, respectively, to form a binding site for a first antigen that specifically binds TRGV9. In some embodiments, the HC1 comprises a HCDR1, HCDR2, and HCDR3 comprising the amino acid sequences of SEQ ID NO:178, SEQ ID NO:700, and SEQ ID NO:701, respectively, and LC1 comprises a LCDR1, LCDR2, and LCDR3 comprising the amino acid sequences of SEQ ID NO:181, SEQ ID NO:182, and SEQ ID NO:183, respectively, to form a binding site for a first antigen that specifically binds TRGV9. In some embodiments, the HC1 comprises a HCDR1, HCDR2, and HCDR3 comprising the amino acid sequences of SEQ ID NO:184, SEQ ID NO:185, and SEQ ID NO:186, respectively, and LC1 comprises a LCDR1, LCDR2, and LCDR3 comprising the amino acid sequences of SEQ ID NO:187, SEQ ID NO:188, and SEQ ID NO:189, respectively, to form a binding site for a first antigen that specifically binds TRGV9. In some embodiments, the HC1 comprises a HCDR1, HCDR2, and HCDR3 comprising the amino acid sequences of SEQ ID NO:190, SEQ ID NO:191, and SEQ ID NO:192, respectively, and LC1 comprises a LCDR1, LCDR2, and LCDR3 comprising the amino acid sequences of SEQ ID NO:193, SEQ ID NO:194, and SEQ ID NO:195, respectively, to form a binding site for a first antigen that specifically binds TRGV9. In some embodiments, the HC1 comprises a HCDR1, HCDR2, and HCDR3 comprising the amino acid sequences of SEQ ID NO:196, SEQ ID NO:197, and SEQ ID NO:198, respectively, and LC1 comprises a LCDR1, LCDR2, and LCDR3 comprising the amino acid sequences of SEQ ID NO:199, SEQ ID NO:200, and SEQ ID NO:201, respectively, to form a binding site for a first antigen that specifically binds TRGV9. In some embodiments, the HC1 comprises a HCDR1, HCDR2, and HCDR3 comprising the amino acid sequences of SEQ ID NO:202, SEQ ID NO:203, and SEQ ID NO:204, respectively, and LC1 comprises a LCDR1, LCDR2, and LCDR3 comprising the amino acid sequences of SEQ ID NO:205, SEQ ID NO:206, and SEQ ID NO:207, respectively, to form a binding site for a first antigen that specifically binds TRGV9. In some embodiments, the HC1 comprises a HCDR1, HCDR2, and HCDR3 comprising the amino acid sequences of SEQ ID NO:208, SEQ ID NO:209, and SEQ ID NO:210, respectively, and LC1 comprises a LCDR1, LCDR2, and LCDR3 comprising the amino acid sequences of SEQ ID NO:211, SEQ ID NO:212, and SEQ ID NO:213, respectively, to form a binding site for a first antigen that specifically binds TRGV9. In some embodiments, the HC1 comprises a HCDR1, HCDR2, and HCDR3 comprising the amino acid sequences of SEQ ID NO:214, SEQ ID NO:215, and SEQ ID NO:216, respectively, and LC1 comprises a LCDR1, LCDR2, and LCDR3 comprising the amino acid sequences of SEQ ID NO:217, SEQ ID NO:218, and SEQ ID NO:219, respectively, to form a binding site for a first antigen that specifically binds TRGV9. In some embodiments, the HC1 comprises a HCDR1, HCDR2, and HCDR3 comprising the amino acid sequences of SEQ ID NO:214, SEQ ID NO:702, and SEQ ID NO:703, respectively, and LC1 comprises a LCDR1, LCDR2, and LCDR3 comprising the amino acid sequences of SEQ ID NO:217, SEQ ID NO:218, and SEQ ID NO:219, respectively, to form a binding site for a first antigen that specifically binds TRGV9. In some embodiments, the HC1 comprises a HCDR1, HCDR2, and HCDR3 comprising the amino acid sequences of SEQ ID NO:220, SEQ ID NO:221, and SEQ ID NO:222, respectively, and LC1 comprises a LCDR1, LCDR2, and LCDR3 comprising the amino acid sequences of SEQ ID NO:223, SEQ ID NO:224, and SEQ ID NO:225, respectively, to form a binding site for a first antigen that specifically binds TRGV9. In some embodiments, the HC1 comprises a HCDR1, HCDR2, and HCDR3 comprising the amino acid sequences of SEQ ID NO:226, SEQ ID NO:227, and SEQ ID NO:228, respectively, and LC1 comprises a LCDR1, LCDR2, and LCDR3 comprising the amino acid sequences of SEQ ID NO:229, SEQ ID NO:230, and SEQ ID NO:231, respectively, to form a binding site for a first antigen that specifically binds TRGV9. In some embodiments, the HC1 comprises a HCDR1, HCDR2, and HCDR3 comprising the amino acid sequences of SEQ ID NO:232, SEQ ID NO:233, and SEQ ID NO:234, respectively, and LC1 comprises a LCDR1, LCDR2, and LCDR3 comprising the amino acid sequences of SEQ ID NO:235, SEQ ID NO:236, and SEQ ID NO:237, respectively, to form a binding site for a first antigen that specifically binds TRGV9. In some embodiments, the HC1 comprises a HCDR1, HCDR2, and HCDR3 comprising the amino acid sequences of SEQ ID NO:238, SEQ ID NO:239, and SEQ ID NO:240, respectively, and LC1 comprises a LCDR1, LCDR2, and LCDR3 comprising the amino acid sequences of SEQ ID NO:241, SEQ ID NO:242, and SEQ ID NO:243, respectively, to form a binding site for a first antigen that specifically binds TRGV9. In some embodiments, the HC1 comprises a HCDR1, HCDR2, and HCDR3 comprising the amino acid sequences of SEQ ID NO:244, SEQ ID NO:245, and SEQ ID NO:246, respectively, and LC1 comprises a LCDR1, LCDR2, and LCDR3 comprising the amino acid sequences of SEQ ID NO:247, SEQ ID NO:248, and SEQ ID NO:249, respectively, to form a binding site for a first antigen that specifically binds TRGV9. In some embodiments, the HC1 comprises a HCDR1, HCDR2, and HCDR3 comprising the amino acid sequences of SEQ ID NO:250, SEQ ID NO:251, and SEQ ID NO:252, respectively, and LC1 comprises a LCDR1, LCDR2, and LCDR3 comprising the amino acid sequences of SEQ ID NO:253, SEQ ID NO:254, and SEQ ID NO:255, respectively, to form a binding site for a first antigen that specifically binds TRGV9. In some embodiments, the HC1 comprises a HCDR1, HCDR2, and HCDR3 comprising the amino acid sequences of SEQ ID NO:250, SEQ ID NO:704, and SEQ ID NO:705, respectively, and LC1 comprises a LCDR1, LCDR2, and LCDR3 comprising the amino acid sequences of SEQ ID NO:253, SEQ ID NO:254, and SEQ ID NO:255, respectively, to form a binding site for a first antigen that specifically binds TRGV9. In some embodiments, the HC1 comprises a HCDR1, HCDR2, and HCDR3 comprising the amino acid sequences of SEQ ID NO:256, SEQ ID NO:257, and SEQ ID NO:258, respectively, and LC1 comprises a LCDR1, LCDR2, and LCDR3 comprising the amino acid sequences of SEQ ID NO:259, SEQ ID NO:260, and SEQ ID NO:261, respectively, to form a binding site for a first antigen that specifically binds TRGV9. In some embodiments, the HC1 comprises a HCDR1, HCDR2, and HCDR3 comprising the amino acid sequences of SEQ ID NO:262, SEQ ID NO:263, and SEQ ID NO:264, respectively, and LC1 comprises a LCDR1, LCDR2, and LCDR3 comprising the amino acid sequences of SEQ ID NO:265, SEQ ID NO:266, and SEQ ID NO:267, respectively, to form a binding site for a first antigen that specifically binds TRGV9. In some embodiments, the HC1 comprises a HCDR1, HCDR2, and HCDR3 comprising the amino acid sequences of SEQ ID NO:268, SEQ ID NO:269, and SEQ ID NO:270, respectively, and LC1 comprises a LCDR1, LCDR2, and LCDR3 comprising the amino acid sequences of SEQ ID NO:271, SEQ ID NO:272, and SEQ ID NO:273, respectively, to form a binding site for a first antigen that specifically binds TRGV9. In some embodiments, the HC1 comprises a HCDR1, HCDR2, and HCDR3 comprising the amino acid sequences of SEQ ID NO:274, SEQ ID NO:275, and SEQ ID NO:276, respectively, and LC1 comprises a LCDR1, LCDR2, and LCDR3 comprising the amino acid sequences of SEQ ID NO:277, SEQ ID NO:278, and SEQ ID NO:279, respectively, to form a binding site for a first antigen that specifically binds TRGV9. In some embodiments, the HC1 comprises a HCDR1, HCDR2, and HCDR3 comprising the amino acid sequences of SEQ ID NO:280, SEQ ID NO:281, and SEQ ID NO:282, respectively, and LC1 comprises a LCDR1, LCDR2, and LCDR3 comprising the amino acid sequences of SEQ ID NO:283, SEQ ID NO:284, and SEQ ID NO:285, respectively, to form a binding site for a first antigen that specifically binds TRGV9. In some embodiments, the HC1 comprises a HCDR1, HCDR2, and HCDR3 comprising the amino acid sequences of SEQ ID NO:286, SEQ ID NO:287, and SEQ ID NO:288, respectively, and LC1 comprises a LCDR1, LCDR2, and LCDR3 comprising the amino acid sequences of SEQ ID NO:289, SEQ ID NO:290, and SEQ ID NO:291, respectively, to form a binding site for a first antigen that specifically binds TRGV9. In some embodiments, the HC1 comprises a HCDR1, HCDR2, and HCDR3 comprising the amino acid sequences of SEQ ID NO:286, SEQ ID NO:706, and SEQ ID NO:707, respectively, and LC1 comprises a LCDR1, LCDR2, and LCDR3 comprising the amino acid sequences of SEQ ID NO:289, SEQ ID NO:290, and SEQ ID NO:291, respectively, to form a binding site for a first antigen that specifically binds TRGV9. In some embodiments, the HC1 comprises a HCDR1, HCDR2, and HCDR3 comprising the amino acid sequences of SEQ ID NO:292, SEQ ID NO:293, and SEQ ID NO:294, respectively, and LC1 comprises a LCDR1, LCDR2, and LCDR3 comprising the amino acid sequences of SEQ ID NO:295, SEQ ID NO:296, and SEQ ID NO:297, respectively, to form a binding site for a first antigen that specifically binds TRGV9. In some embodiments, the HC1 comprises a HCDR1, HCDR2, and HCDR3 comprising the amino acid sequences of SEQ ID NO:298, SEQ ID NO:299, and SEQ ID NO:300, respectively, and LC1 comprises a LCDR1, LCDR2, and LCDR3 comprising the amino acid sequences of SEQ ID NO:301, SEQ ID NO:302, and SEQ ID NO:303, respectively, to form a binding site for a first antigen that specifically binds TRGV9. In some embodiments, the HC1 comprises a HCDR1, HCDR2, and HCDR3 comprising the amino acid sequences of SEQ ID NO:304, SEQ ID NO:305, and SEQ ID NO:306, respectively, and LC1 comprises a LCDR1, LCDR2, and LCDR3 comprising the amino acid sequences of SEQ ID NO:307, SEQ ID NO:308, and SEQ ID NO:309, respectively, to form a binding site for a first antigen that specifically binds TRGV9. In some embodiments, the HC1 comprises a HCDR1, HCDR2, and HCDR3 comprising the amino acid sequences of SEQ ID NO:310, SEQ ID NO:311, and SEQ ID NO:312, respectively, and LC1 comprises a LCDR1, LCDR2, and LCDR3 comprising the amino acid sequences of SEQ ID NO:313, SEQ ID NO:314, and SEQ ID NO:315, respectively, to form a binding site for a first antigen that specifically binds TRGV9. In some embodiments, the HC1 comprises a HCDR1, HCDR2, and HCDR3 comprising the amino acid sequences of SEQ ID NO:316, SEQ ID NO:317, and SEQ ID NO:318, respectively, and LC1 comprises a LCDR1, LCDR2, and LCDR3 comprising the amino acid sequences of SEQ ID NO:319, SEQ ID NO:320, and SEQ ID NO:321, respectively, to form a binding site for a first antigen that specifically binds TRGV9. In some embodiments, the HC1 comprises a HCDR1, HCDR2, and HCDR3 comprising the amino acid sequences of SEQ ID NO:322, SEQ ID NO:323, and SEQ ID NO:324, respectively, and LC1 comprises a LCDR1, LCDR2, and LCDR3 comprising the amino acid sequences of SEQ ID NO:325, SEQ ID NO:326, and SEQ ID NO:327, respectively, to form a binding site for a first antigen that specifically binds TRGV9. In some embodiments, the HC1 comprises a HCDR1, HCDR2, and HCDR3 comprising the amino acid sequences of SEQ ID NO:322, SEQ ID NO:708, and SEQ ID NO:709, respectively, and LC1 comprises a LCDR1, LCDR2, and LCDR3 comprising the amino acid sequences of SEQ ID NO:325, SEQ ID NO:326, and SEQ ID NO:327, respectively, to form a binding site for a first antigen that specifically binds TRGV9. In some embodiments, the HC1 comprises a HCDR1, HCDR2, and HCDR3 comprising the amino acid sequences of SEQ ID NO:328, SEQ ID NO:329, and SEQ ID NO:330, respectively, and LC1 comprises a LCDR1, LCDR2, and LCDR3 comprising the amino acid sequences of SEQ ID NO:331, SEQ ID NO:332, and SEQ ID NO:333, respectively, to form a binding site for a first antigen that specifically binds TRGV9. In some embodiments, the HC1 comprises a HCDR1, HCDR2, and HCDR3 comprising the amino acid sequences of SEQ ID NO:334, SEQ ID NO:335, and SEQ ID NO:336, respectively, and LC1 comprises a LCDR1, LCDR2, and LCDR3 comprising the amino acid sequences of SEQ ID NO:337, SEQ ID NO:338, and SEQ ID NO:339, respectively, to form a binding site for a first antigen that specifically binds TRGV9. In some embodiments, the HC1 comprises a HCDR1, HCDR2, and HCDR3 comprising the amino acid sequences of SEQ ID NO:340, SEQ ID NO:341, and SEQ ID NO:342, respectively, and LC1 comprises a LCDR1, LCDR2, and LCDR3 comprising the amino acid sequences of SEQ ID NO:343, SEQ ID NO:344, and SEQ ID NO:345, respectively, to form a binding site for a first antigen that specifically binds TRGV9. In some embodiments, the HC1 comprises a HCDR1, HCDR2, and HCDR3 comprising the amino acid sequences of SEQ ID NO:346, SEQ ID NO:347, and SEQ ID NO:348, respectively, and LC1 comprises a LCDR1, LCDR2, and LCDR3 comprising the amino acid sequences of SEQ ID NO:349, SEQ ID NO:350, and SEQ ID NO:351, respectively, to form a binding site for a first antigen that specifically binds TRGV9. In some embodiments, the HC1 comprises a HCDR1, HCDR2, and HCDR3 comprising the amino acid sequences of SEQ ID NO:352, SEQ ID NO:353, and SEQ ID NO:354, respectively, and LC1 comprises a LCDR1, LCDR2, and LCDR3 comprising the amino acid sequences of SEQ ID NO:355, SEQ ID NO:356, and SEQ ID NO:357, respectively, to form a binding site for a first antigen that specifically binds TRGV9. In some embodiments, the HC1 comprises a HCDR1, HCDR2, and HCDR3 comprising the amino acid sequences of SEQ ID NO:358, SEQ ID NO:359, and SEQ ID NO:360, respectively, and LC1 comprises a LCDR1, LCDR2, and LCDR3 comprising the amino acid sequences of SEQ ID NO:361, SEQ ID NO:362, and SEQ ID NO:363, respectively, to form a binding site for a first antigen that specifically binds TRGV9. In some embodiments, the HC1 comprises a HCDR1, HCDR2, and HCDR3 comprising the amino acid sequences of SEQ ID NO:358, SEQ ID NO:710, and SEQ ID NO:711, respectively, and LC1 comprises a LCDR1, LCDR2, and LCDR3 comprising the amino acid sequences of SEQ ID NO:361, SEQ ID NO:362, and SEQ ID NO:363, respectively, to form a binding site for a first antigen that specifically binds TRGV9. In some embodiments, the HC1 comprises a HCDR1, HCDR2, and HCDR3 comprising the amino acid sequences of SEQ ID NO:364, SEQ ID NO:365, and SEQ ID NO:366, respectively, and LC1 comprises a LCDR1, LCDR2, and LCDR3 comprising the amino acid sequences of SEQ ID NO:367, SEQ ID NO:368, and SEQ ID NO:369, respectively, to form a binding site for a first antigen that specifically binds TRGV9. In some embodiments, the HC1 comprises a HCDR1, HCDR2, and HCDR3 comprising the amino acid sequences of SEQ ID NO:370, SEQ ID NO:371, and SEQ ID NO:372, respectively, and LC1 comprises a LCDR1, LCDR2, and LCDR3 comprising the amino acid sequences of SEQ ID NO:373, SEQ ID NO:374, and SEQ ID NO:375, respectively, to form a binding site for a first antigen that specifically binds TRGV9. In some embodiments, the HC1 comprises a HCDR1, HCDR2, and HCDR3 comprising the amino acid sequences of SEQ ID NO:376, SEQ ID NO:377, and SEQ ID NO:378, respectively, and LC1 comprises a LCDR1, LCDR2, and LCDR3 comprising the amino acid sequences of SEQ ID NO:379, SEQ ID NO:380, and SEQ ID NO:381, respectively, to form a binding site for a first antigen that specifically binds TRGV9. In some embodiments, the HC1 comprises a HCDR1, HCDR2, and HCDR3 comprising the amino acid sequences of SEQ ID NO:382, SEQ ID NO:383, and SEQ ID NO:384, respectively, and LC1 comprises a LCDR1, LCDR2, and LCDR3 comprising the amino acid sequences of SEQ ID NO:385, SEQ ID NO:386, and SEQ ID NO:387, respectively, to form a binding site for a first antigen that specifically binds TRGV9. In some embodiments, the HC1 comprises a HCDR1, HCDR2, and HCDR3 comprising the amino acid sequences of SEQ ID NO:388, SEQ ID NO:389, and SEQ ID NO:390, respectively, and LC1 comprises a LCDR1, LCDR2, and LCDR3 comprising the amino acid sequences of SEQ ID NO:391, SEQ ID NO:392, and SEQ ID NO:393, respectively, to form a binding site for a first antigen that specifically binds TRGV9. In some embodiments, the HC1 comprises a HCDR1, HCDR2, and HCDR3 comprising the amino acid sequences of SEQ ID NO:394, SEQ ID NO:395, and SEQ ID NO:396, respectively, and LC1 comprises a LCDR1, LCDR2, and LCDR3 comprising the amino acid sequences of SEQ ID NO:397, SEQ ID NO:398, and SEQ ID NO:399, respectively, to form a binding site for a first antigen that specifically binds TRGV9. In some embodiments, the HC1 comprises a HCDR1, HCDR2, and HCDR3 comprising the amino acid sequences of SEQ ID NO:394, SEQ ID NO:712, and SEQ ID NO:713, respectively, and LC1 comprises a LCDR1, LCDR2, and LCDR3 comprising the amino acid sequences of SEQ ID NO:397, SEQ ID NO:398, and SEQ ID NO:399, respectively, to form a binding site for a first antigen that specifically binds TRGV9. In some embodiments, the HC1 comprises a HCDR1, HCDR2, and HCDR3 comprising the amino acid sequences of SEQ ID NO:400, SEQ ID NO:401, and SEQ ID NO:402, respectively, and LC1 comprises a LCDR1, LCDR2, and LCDR3 comprising the amino acid sequences of SEQ ID NO:403, SEQ ID NO:404, and SEQ ID NO:405, respectively, to form a binding site for a first antigen that specifically binds TRGV9. In some embodiments, the HC1 comprises a HCDR1, HCDR2, and HCDR3 comprising the amino acid sequences of SEQ ID NO:406, SEQ ID NO:407, and SEQ ID NO:408, respectively, and LC1 comprises a LCDR1, LCDR2, and LCDR3 comprising the amino acid sequences of SEQ ID NO:409, SEQ ID NO:410, and SEQ ID NO:411, respectively, to form a binding site for a first antigen that specifically binds TRGV9. In some embodiments, the HC1 comprises a HCDR1, HCDR2, and HCDR3 comprising the amino acid sequences of SEQ ID NO:412, SEQ ID NO:413, and SEQ ID NO:414, respectively, and LC1 comprises a LCDR1, LCDR2, and LCDR3 comprising the amino acid sequences of SEQ ID NO:415, SEQ ID NO:416, and SEQ ID NO:417, respectively, to form a binding site for a first antigen that specifically binds TRGV9. In some embodiments, the HC1 comprises a HCDR1, HCDR2, and HCDR3 comprising the amino acid sequences of SEQ ID NO:418, SEQ ID NO:419, and SEQ ID NO:420, respectively, and LC1 comprises a LCDR1, LCDR2, and LCDR3 comprising the amino acid sequences of SEQ ID NO:421, SEQ ID NO:422, and SEQ ID NO:423, respectively, to form a binding site for a first antigen that specifically binds TRGV9. In some embodiments, the HC1 comprises a HCDR1, HCDR2, and HCDR3 comprising the amino acid sequences of SEQ ID NO:424, SEQ ID NO:425, and SEQ ID NO:426, respectively, and LC1 comprises a LCDR1, LCDR2, and LCDR3 comprising the amino acid sequences of SEQ ID NO:427, SEQ ID NO:428, and SEQ ID NO:429, respectively, to form a binding site for a first antigen that specifically binds TRGV9. In some embodiments, the HC1 comprises a HCDR1, HCDR2, and HCDR3 comprising the amino acid sequences of SEQ ID NO:430, SEQ ID NO:431, and SEQ ID NO:432, respectively, and LC1 comprises a LCDR1, LCDR2, and LCDR3 comprising the amino acid sequences of SEQ ID NO:433, SEQ ID NO:434, and SEQ ID NO:435, respectively, to form a binding site for a first antigen that specifically binds TRGV9. In some embodiments, the HC1 comprises a HCDR1, HCDR2, and HCDR3 comprising the amino acid sequences of SEQ ID NO:430, SEQ ID NO:714, and SEQ ID NO:715, respectively, and LC1 comprises a LCDR1, LCDR2, and LCDR3 comprising the amino acid sequences of SEQ ID NO:433, SEQ ID NO:434, and SEQ ID NO:435, respectively, to form a binding site for a first antigen that specifically binds TRGV9. In some embodiments, the HC1 comprises a HCDR1, HCDR2, and HCDR3 comprising the amino acid sequences of SEQ ID NO:436, SEQ ID NO:437, and SEQ ID NO:438, respectively, and LC1 comprises a LCDR1, LCDR2, and LCDR3 comprising the amino acid sequences of SEQ ID NO:439, SEQ ID NO:440, and SEQ ID NO:441, respectively, to form a binding site for a first antigen that specifically binds TRGV9. In some embodiments, the HC1 comprises a HCDR1, HCDR2, and HCDR3 comprising the amino acid sequences of SEQ ID NO:442, SEQ ID NO:443, and SEQ ID NO:444, respectively, and LC1 comprises a LCDR1, LCDR2, and LCDR3 comprising the amino acid sequences of SEQ ID NO:445, SEQ ID NO:446, and SEQ ID NO:447, respectively, to form a binding site for a first antigen that specifically binds TRGV9. In some embodiments, the HC1 comprises a HCDR1, HCDR2, and HCDR3 comprising the amino acid sequences of SEQ ID NO:448, SEQ ID NO:449, and SEQ ID NO:450, respectively, and LC1 comprises a LCDR1, LCDR2, and LCDR3 comprising the amino acid sequences of SEQ ID NO:451, SEQ ID NO:452, and SEQ ID NO:453, respectively, to form a binding site for a first antigen that specifically binds TRGV9. In some embodiments, the HC1 comprises a HCDR1, HCDR2, and HCDR3 comprising the amino acid sequences of SEQ ID NO:454, SEQ ID NO:455, and SEQ ID NO:456, respectively, and LC1 comprises a LCDR1, LCDR2, and LCDR3 comprising the amino acid sequences of SEQ ID NO:457, SEQ ID NO:458, and SEQ ID NO:459, respectively, to form a binding site for a first antigen that specifically binds TRGV9. In some embodiments, the HC1 comprises a HCDR1, HCDR2, and HCDR3 comprising the amino acid sequences of SEQ ID NO:460, SEQ ID NO:461, and SEQ ID NO:462, respectively, and LC1 comprises a LCDR1, LCDR2, and LCDR3 comprising the amino acid sequences of SEQ ID NO:463, SEQ ID NO:464, and SEQ ID NO:465, respectively, to form a binding site for a first antigen that specifically binds TRGV9. In some embodiments, the HC1 comprises a HCDR1, HCDR2, and HCDR3 comprising the amino acid sequences of SEQ ID NO:466, SEQ ID NO:467, and SEQ ID NO:468, respectively, and LC1 comprises a LCDR1, LCDR2, and LCDR3 comprising the amino acid sequences of SEQ ID NO:469, SEQ ID NO:470, and SEQ ID NO:471, respectively, to form a binding site for a first antigen that specifically binds TRGV9. In some embodiments, the HC1 comprises a HCDR1, HCDR2, and HCDR3 comprising the amino acid sequences of SEQ ID NO:466, SEQ ID NO:716, and SEQ ID NO:717, respectively, and LC1 comprises a LCDR1, LCDR2, and LCDR3 comprising the amino acid sequences of SEQ ID NO:469, SEQ ID NO:470, and SEQ ID NO:471, respectively, to form a binding site for a first antigen that specifically binds TRGV9. In some embodiments, the HC1 comprises a HCDR1, HCDR2, and HCDR3 comprising the amino acid sequences of SEQ ID NO:472, SEQ ID NO:473, and SEQ ID NO:474, respectively, and LC1 comprises a LCDR1, LCDR2, and LCDR3 comprising the amino acid sequences of SEQ ID NO:475, SEQ ID NO:476, and SEQ ID NO:477, respectively, to form a binding site for a first antigen that specifically binds TRGV9. In some embodiments, the HC1 comprises a HCDR1, HCDR2, and HCDR3 comprising the amino acid sequences of SEQ ID NO:478, SEQ ID NO:479, and SEQ ID NO:480, respectively, and LC1 comprises a LCDR1, LCDR2, and LCDR3 comprising the amino acid sequences of SEQ ID NO:481, SEQ ID NO:482, and SEQ ID NO:483, respectively, to form a binding site for a first antigen that specifically binds TRGV9. In some embodiments, the HC1 comprises a HCDR1, HCDR2, and HCDR3 comprising the amino acid sequences of SEQ ID NO:484, SEQ ID NO:485, and SEQ ID NO:486, respectively, and LC1 comprises a LCDR1, LCDR2, and LCDR3 comprising the amino acid sequences of SEQ ID NO:487, SEQ ID NO:488, and SEQ ID NO:489, respectively, to form a binding site for a first antigen that specifically binds TRGV9. In some embodiments, the HC1 comprises a HCDR1, HCDR2, and HCDR3 comprising the amino acid sequences of SEQ ID NO:490, SEQ ID NO:491, and SEQ ID NO:492, respectively, and LC1 comprises a LCDR1, LCDR2, and LCDR3 comprising the amino acid sequences of SEQ ID NO:493, SEQ ID NO:494, and SEQ ID NO:495, respectively, to form a binding site for a first antigen that specifically binds TRGV9. In some embodiments, the HC1 comprises a HCDR1, HCDR2, and HCDR3 comprising the amino acid sequences of SEQ ID NO:496, SEQ ID NO:497, and SEQ ID NO:498, respectively, and LC1 comprises a LCDR1, LCDR2, and LCDR3 comprising the amino acid sequences of SEQ ID NO:499, SEQ ID NO:500, and SEQ ID NO:501, respectively, to form a binding site for a first antigen that specifically binds TRGV9. In some embodiments, the HC1 comprises a HCDR1, HCDR2, and HCDR3 comprising the amino acid sequences of SEQ ID NO:502, SEQ ID NO:503, and SEQ ID NO:504, respectively, and LC1 comprises a LCDR1, LCDR2, and LCDR3 comprising the amino acid sequences of SEQ ID NO:505, SEQ ID NO:506, and SEQ ID NO:507, respectively, to form a binding site for a first antigen that specifically binds TRGV9. In some embodiments, the HC1 comprises a HCDR1, HCDR2, and HCDR3 comprising the amino acid sequences of SEQ ID NO:502, SEQ ID NO:718, and SEQ ID NO:719, respectively, and LC1 comprises a LCDR1, LCDR2, and LCDR3 comprising the amino acid sequences of SEQ ID NO:505, SEQ ID NO:506, and SEQ ID NO:507, respectively, to form a binding site for a first antigen that specifically binds TRGV9. In some embodiments, the HC1 comprises a HCDR1, HCDR2, and HCDR3 comprising the amino acid sequences of SEQ ID NO:508, SEQ ID NO:509, and SEQ ID NO:510, respectively, and LC1 comprises a LCDR1, LCDR2, and LCDR3 comprising the amino acid sequences of SEQ ID NO:511, SEQ ID NO:512, and SEQ ID NO:513, respectively, to form a binding site for a first antigen that specifically binds TRGV9. In some embodiments, the HC1 comprises a HCDR1, HCDR2, and HCDR3 comprising the amino acid sequences of SEQ ID NO:514, SEQ ID NO:515, and SEQ ID NO:516, respectively, and LC1 comprises a LCDR1, LCDR2, and LCDR3 comprising the amino acid sequences of SEQ ID NO:517, SEQ ID NO:518, and SEQ ID NO:519, respectively, to form a binding site for a first antigen that specifically binds TRGV9. In some embodiments, the HC1 comprises a HCDR1, HCDR2, and HCDR3 comprising the amino acid sequences of SEQ ID NO: 520, SEQ ID NO:521, and SEQ ID NO:522, respectively, and LC1 comprises a LCDR1, LCDR2, and LCDR3 comprising the amino acid sequences of SEQ ID NO:523, SEQ ID NO:524, and SEQ ID NO:525, respectively, to form a binding site for a first antigen that specifically binds TRGV9. In some embodiments, the HC1 comprises a HCDR1, HCDR2, and HCDR3 comprising the amino acid sequences of SEQ ID NO:526, SEQ ID NO:527, and SEQ ID NO:528, respectively, and LC1 comprises a LCDR1, LCDR2, and LCDR3 comprising the amino acid sequences of SEQ ID NO:529, SEQ ID NO:530, and SEQ ID NO:531, respectively, to form a binding site for a first antigen that specifically binds TRGV9. In some embodiments, the HC1 comprises a HCDR1, HCDR2, and HCDR3 comprising the amino acid sequences of SEQ ID NO:532, SEQ ID NO:533, and SEQ ID NO:534, respectively, and LC1 comprises a LCDR1, LCDR2, and LCDR3 comprising the amino acid sequences of SEQ ID NO:535, SEQ ID NO:536, and SEQ ID NO:537, respectively, to form a binding site for a first antigen that specifically binds TRGV9. In some embodiments, the HC1 comprises a HCDR1, HCDR2, and HCDR3 comprising the amino acid sequences of SEQ ID NO:538, SEQ ID NO:539, and SEQ ID NO:540, respectively, and LC1 comprises a LCDR1, LCDR2, and LCDR3 comprising the amino acid sequences of SEQ ID NO:541, SEQ ID NO:542, and SEQ ID NO:543, respectively, to form a binding site for a first antigen that specifically binds TRGV9. In some embodiments, the HC1 comprises a HCDR1, HCDR2, and HCDR3 comprising the amino acid sequences of SEQ ID NO:538, SEQ ID NO:720, and SEQ ID NO:721, respectively, and LC1 comprises a LCDR1, LCDR2, and LCDR3 comprising the amino acid sequences of SEQ ID NO:541, SEQ ID NO:542, and SEQ ID NO:543, respectively, to form a binding site for a first antigen that specifically binds TRGV9. In some embodiments, the HC1 comprises a HCDR1, HCDR2, and HCDR3 comprising the amino acid sequences of SEQ ID NO:544, SEQ ID NO:545, and SEQ ID NO:546, respectively, and LC1 comprises a LCDR1, LCDR2, and LCDR3 comprising the amino acid sequences of SEQ ID NO:547, SEQ ID NO:548, and SEQ ID NO:549, respectively, to form a binding site for a first antigen that specifically binds TRGV9. In some embodiments, the HC1 comprises a HCDR1, HCDR2, and HCDR3 comprising the amino acid sequences of SEQ ID NO:550, SEQ ID NO:551, and SEQ ID NO:552, respectively, and LC1 comprises a LCDR1, LCDR2, and LCDR3 comprising the amino acid sequences of SEQ ID NO:553, SEQ ID NO:554, and SEQ ID NO:555, respectively, to form a binding site for a first antigen that specifically binds TRGV9. The HC2 can, for example, comprise a HCDR1, HCDR2, and HCDR3 comprising the amino acid sequences of SEQ ID NO:9, SEQ ID NO:10, and SEQ ID NO:11, respectively, and LC2 can, for example, comprise a LCDR1, LCDR2, and LCDR3 comprising the amino acid sequences of SEQ ID NO:12, SEQ ID NO:13, and SEQ ID NO:14, respectively, to form a binding site for a second antigen that specifically binds CD123. In some embodiments, the HC2 comprises a HCDR1, HCDR2, and HCDR3 comprising the amino acid sequence of SEQ ID NO:556, SEQ ID NO:557, and SEQ ID NO:558, respectively, and a LCDR1, LCDR2, and LCDR3 comprising the amino acid sequence of SEQ ID NO:559, SEQ ID NO:560, and SEQ ID NO:561, respectively, to form a binding site for a second antigen that specifically binds CD123. In some embodiments, the HC2 comprises a HCDR1, HCDR2, and HCDR3 comprising the amino acid sequence of SEQ ID NO:562, SEQ ID NO:563, and SEQ ID NO:564, respectively, and a LCDR1, LCDR2, and LCDR3 comprising the amino acid sequence of SEQ ID NO:565, SEQ ID NO:566, and SEQ ID NO:567, respectively, to form a binding site for a second antigen that specifically binds CD123. In some embodiments, the HC2 comprises a HCDR1, HCDR2, and HCDR3 comprising the amino acid sequence of SEQ ID NO:568, SEQ ID NO:569, and SEQ ID NO:570, respectively, and a LCDR1, LCDR2, and LCDR3 comprising the amino acid sequence of SEQ ID NO:571, SEQ ID NO:572, and SEQ ID NO:573, respectively, to form a binding site for a second antigen that specifically binds CD123. In some embodiments, the HC2 comprises a HCDR1, HCDR2, and HCDR3 comprising the amino acid sequence of SEQ ID NO:574, SEQ ID NO:575, and SEQ ID NO:576, respectively, and a LCDR1, LCDR2, and LCDR3 comprising the amino acid sequence of SEQ ID NO:577, SEQ ID NO:578, and SEQ ID NO:579, respectively, to form a binding site for a second antigen that specifically binds CD123. In some embodiments, the HC2 comprises a HCDR1, HCDR2, and HCDR3 comprising the amino acid sequence of SEQ ID NO:574, SEQ ID NO:722, and SEQ ID NO:723, respectively, and a LCDR1, LCDR2, and LCDR3 comprising the amino acid sequence of SEQ ID NO:577, SEQ ID NO:578, and SEQ ID NO:579, respectively, to form a binding site for a second antigen that specifically binds CD123. In some embodiments, the HC2 comprises a HCDR1, HCDR2, and HCDR3 comprising the amino acid sequence of SEQ ID NO:580, SEQ ID NO:581, and SEQ ID NO:582, respectively, and a LCDR1, LCDR2, and LCDR3 comprising the amino acid sequence of SEQ ID NO:583, SEQ ID NO:584, and SEQ ID NO:585, respectively, to form a binding site for a second antigen that specifically binds CD123. In some embodiments, the HC2 comprises a HCDR1, HCDR2, and HCDR3 comprising the amino acid sequence of SEQ ID NO:586, SEQ ID NO:587, and SEQ ID NO:588; and (ii) a VL comprising a VL CDR1 having an amino acid sequence of SEQ ID NO:589, SEQ ID NO:590, and SEQ ID NO:591, respectively, to form a binding site for a second antigen that specifically binds CD123. In certain embodiments, the HC1 comprises the amino acid sequence of SEQ ID NO:7, SEQ ID NO:34, SEQ ID NO:35, or SEQ ID NO:36, and LC1 comprises the amino acid sequence of SEQ ID NO:8, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:7, and LC1 comprises the amino acid sequence of SEQ ID NO:8, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:34, and LC1 comprises the amino acid sequence of SEQ ID NO:8, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:35, and LC1 comprises the amino acid sequence of SEQ ID NO:8, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:36, and LC1 comprises the amino acid sequence of SEQ ID NO:8, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:65, and LC1 comprises the amino acid sequence of SEQ ID NO:66, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:67, and LC1 comprises the amino acid sequence of SEQ ID NO:68, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:95, and LC1 comprises the amino acid sequence of SEQ ID NO:96, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:104, and LC1 comprises the amino acid sequence of SEQ ID NO:105, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:113, and LC1 comprises the amino acid sequence of SEQ ID NO:114, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:123, and LC1 comprises the amino acid sequence of SEQ ID NO:124, and the HC2 comprises the amino acid sequence of SEQ ID NO:15 and LC2 comprises the amino acid sequence of SEQ ID NO:16. In certain embodiments, the HC1 comprises the amino acid sequence of SEQ ID NO:133, and LC1 comprises the amino acid sequence of SEQ ID NO:134, and the HC2 comprises the amino acid sequence of SEQ ID NO:15 and LC2 comprises the amino acid sequence of SEQ ID NO:16. In certain embodiments, the TRGV9 is on the surface of a γδ T cell. In certain embodiments, the CD123 is on the surface of a cancer cell (e.g., a leukemia cell).

In some embodiments, the antibody specifically binds TRGV9. In other embodiments, the TRGV9 is present on the surface of a γδ T cell. In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In some embodiments, the bispecific antibody provided herein is a diabody, a cross-body, or a bispecific antibody obtained via a controlled Fab arm exchange as those described herein.

In some embodiments, the bispecific antibodies include IgG-like molecules with complementary CH3 domains to force heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) In some embodiments, different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine-China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies provided herein can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope, i.e. an epitope on TRGV9 and an epitope on a tumor antigen.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T3661K392M_T394W/F405A_Y407V, T366L K392M T394W/F405A Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

In addition to methods described above, bispecific antibodies provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody (e.g., anti-CD33 antibody) and the second monospecific bivalent antibody (e.g., anti-CD3 antibody) are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, or a reducing agent selected from the group consisting of. 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In some embodiments, the anti-TRGV9 antibody is a multispecific antibody. In other embodiments, the anti-TRGV9 antibody is a bispecific antibody. In other embodiments, the bispecific antibody comprises an antigen binding fragment of an anti-TRGV9 antibody provided herein.

In some embodiments, the anti-TRGV9 bispecific antibody does not comprise a single chain antibody. In some embodiments, the anti-TRGV9 bispecific antibody does not comprise a single domain antibody. In certain embodiments, the anti-TRGV9 bispecific antibody does not comprise a nanobody. In certain embodiments, the anti-TRGV9 bispecific antibody does not comprise a VHH antibody. In certain embodiments, the anti-TRGV9 bispecific antibody does not comprise a llama antibody.

Also provided herein is a bispecific antibody comprising an anti-TRGV9 antibody or antigen-binding fragment thereof, and an anti-CD123 antibody or antigen-binding fragment thereof. In certain embodiments, the bispecific antibody comprises an anti-TRGV9 antibody, and an anti-CD123 antibody. In certain embodiments, the bispecific antibody comprises an antigen binding fragment of an anti-TRGV9 antibody, and an anti-CD123 antibody. In certain embodiments, the bispecific antibody comprises an anti-TRGV9 antibody, and an antigen-binding fragment of an anti-CD123 antibody. In certain embodiments, the bispecific antibody comprises an antigen-binding fragment of an anti-TRGV9 antibody, and an antigen binding fragment of an anti-CD123 antibody.

In some embodiments, an anti-TRGV9 bispecific antibody provided herein does not comprise a VH CDR1, VH CDR2, and VH CDR3 having the amino acid sequence of SEQ ID NOs:730, 731, and 732, respectively. In some embodiments, an anti-TRGV9 bispecific antibody provided herein does not comprise a VH CDR1, VH CDR2, and VH CDR3 having the amino acid sequence of SEQ ID NOs:733, 734, and 735, respectively. In some embodiments, an anti-TRGV9 bispecific antibody provided herein does not comprise a VH CDR1, VH CDR2, and VH CDR3 having the amino acid sequence of SEQ ID NOs:736, 737, and 738, respectively. In some embodiments, an anti-TRGV9 bispecific antibody provided herein does not comprise a VH CDR1, VH CDR2, and VH CDR3 having the amino acid sequence of SEQ ID NOs:739, 740, and 741, respectively. In some embodiments, an anti-TRGV9 bispecific antibody provided herein does not comprise a VH CDR1, VH CDR2, and VH CDR3 having the amino acid sequence of SEQ ID NOs:742, 743, and 744, respectively. In some embodiments, an anti-TRGV9 bispecific antibody provided herein does not comprise a VH CDR1, VH CDR2, and VH CDR3 having the amino acid sequence of SEQ ID NOs:745, 746, and 747, respectively. In some embodiments, an anti-TRGV9 bispecific antibody provided herein does not comprise a VH CDR1, VH CDR2, and VH CDR3 having the amino acid sequence of SEQ ID NOs:748, 749, and 750, respectively. In some embodiments, an anti-TRGV9 bispecific antibody provided herein does not comprise a VH domain having the amino acid sequence of SEQ ID NO:751. In some embodiments, an anti-TRGV9 bispecific antibody provided herein does not comprise a VH domain having the amino acid sequence of SEQ ID NO:752. In some embodiments, an anti-TRGV9 bispecific antibody provided herein does not comprise a VH domain having the amino acid sequence of SEQ ID NO:753. In some embodiments, an anti-TRGV9 bispecific antibody provided herein does not comprise a VH domain having the amino acid sequence of SEQ ID NO:754. In some embodiments, an anti-TRGV9 bispecific antibody provided herein does not comprise a VH domain having the amino acid sequence of SEQ ID NO:755. In some embodiments, an anti-TRGV9 bispecific antibody provided herein does not comprise a VH domain having the amino acid sequence of SEQ ID NO:756. In some embodiments, an anti-TRGV9 bispecific antibody provided herein does not comprise a VH domain having the amino acid sequence of SEQ ID NO:757.

In certain embodiments, the anti-TRGV9 antibody or antigen-binding fragment thereof comprises a HCDR1, HCDR2, HCDR3, a LCDR1, LCDR2, and LCDR3, having the polypeptide sequence of:
i. SEQ ID NOs:1, 2, 3, 4, 5, and 6, respectively;
ii. SEQ ID NOs:1, 2, 31, 4, 5, and 6, respectively;
iii. SEQ ID NOs:1, 2, 32, 4, 5, and 6, respectively; or
iv. SEQ ID NOs:1, 2, 33, 4, 5, and 6, respectively;
   and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively.

In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:1, 2, 3, 4, 5, and 6, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:1, 2, 31, 4, 5, and 6, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:1, 2, 32, 4, 5, and 6, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:1, 2, 33, 4, 5, and 6, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:1, 76, 3, 77, 5, and 6, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:60, 61, 62, 63, 64, and 6, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:89, 90, 91, 92, 93, and 94, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:98, 99, 100, 101, 102, and 103, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:107, 108, 109, 110, 111, and 112, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:117, 118, 119, 120, 121, and 122, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In certain embodiments, the anti-TRGV9 antibody or antigen-binding fragment thereof comprises a HCDR1, HCDR2, HCDR3, a LCDR1, LCDR2, and LCDR3, having the polypeptide sequence of SEQ ID NOs:127, 128, 129, 130, 131, and 132, respectively; and the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively.

In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:160, 161, 162, 163, 164, and 165, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:166, 167, 168, 169, 170, and 171, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:172, 173, 174, 175, 176, and 177, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:178, 179, 180, 181, 182, and 183, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:178, 700, 701, 181, 182, and 183, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:184, 185, 186, 187, 188, and 189, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:190, 191, 192, 193, 194, and 195, respectively. In certain embodiments, the anti-TRGV9 antibody or antigen-binding 15 fragment thereof comprises a HCDR1, HCDR2, HCDR3, LCDR1, LCDR2, and LCDR3, having the polypeptide sequence of SEQ ID NOs:196, 197, 198, 199, 200, and 201, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:202, 203, 204, 205, 206, and 207, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:208, 209, 210, 211, 212, and 213, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:214, 215, 216, 217, 218, and 219, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:214, 702, 703, 217, 218, and 219, respectively. In certain embodiments, the anti-TRGV9 antibody or antigen-binding 30 fragment thereof comprises a HCDR1, HCDR2, HCDR3, LCDR1, LCDR2, and LCDR3, having the polypeptide sequence of SEQ ID NOs:220, 221, 222, 223, 224, and 225, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:226, 227, 228, 229, 230, and 231, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:232, 233, 234, 235, 236, and 237, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:238, 239, 240, 241, 242, and 243, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:244, 245, 246, 247, 248, and 249, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:250, 251, 252, 253, 254, and 255, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:250, 704, 705, 253, 254, and 255, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:256, 257, 258, 259, 260, and 261, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:262, 263, 264, 265, 266, and 267, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:268, 269, 270, 271, 272, and 273, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:274, 275, 276, 277, 278, and 279, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:280, 281, 282, 283, 284, and 285, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:286, 287, 288, 289, 290, and 291, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:286, 706, 707, 289, 290, and 291, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:292, 293, 294, 295, 296, and 297, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:298, 299, 300, 301, 302, and 303, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:304, 305, 306, 307, 308, and 309, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:310, 311, 312, 313, 314, and 315, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:316, 317, 318, 319, 320, and 321, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:322, 323, 324, 325, 326, and 327, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:322, 708, 709, 325, 326, and 327, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:328, 329, 330, 331, 332, and 333, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:334, 335, 336, 337, 338, and 339, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:340, 341, 342, 343, 344, and 345, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:346, 347, 348, 349, 350, and 351, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:352, 353, 354, 355, 356, and 357, respectively. In certain embodiments, the anti-TRGV9 antibody or antigen-binding 15 fragment thereof comprises a HCDR1, HCDR2, HCDR3, LCDR1, LCDR2, and LCDR3, having the polypeptide sequence of SEQ ID NOs:358, 359, 360, 361, 362, and 363, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:358, 710, 711, 361, 362, and 363, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:364, 365, 366, 367, 368, and 369, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:370, 371, 372, 373, 374, and 375, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:376, 377, 378, 379, 380, and 381, respectively. In certain embodiments, the anti-TRGV9 antibody or antigen-binding 30 fragment thereof comprises a HCDR1, HCDR2, HCDR3, LCDR1, LCDR2, and LCDR3, having the polypeptide sequence of SEQ ID NOs:382, 383, 384, 385, 386, and 387, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:388, 389, 390, 391, 392, and 393, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:394, 395, 396, 397, 398, and 399, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:394, 712, 713, 397, 398, and 399, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:400, 401, 402, 403, 404, and 405, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:406, 407, 408, 409, 410, and 411, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:412, 413, 414, 415, 416, and 417, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:418, 419, 420, 421, 422, and 423, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:424, 425, 426, 427, 428, and 429, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:430, 431, 432, 433, 434, and 435, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:430, 714, 715, 433, 434, and 435, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:436, 437, 438, 439, 440, and 441, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:442, 443, 444, 445, 446, and 447, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:448, 449, 450, 451, 452, and 453, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:454, 455, 456, 457, 458, and 459, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:460, 461, 462, 463, 464, and 465, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:466, 467, 468, 469, 470, and 471, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:466, 716, 717, 469, 470, and 471, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:472, 473, 474, 475, 476, and 477, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:478, 479, 480, 481, 482, and 483, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:484, 485, 486, 487, 488, and 489, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:490, 491, 492, 493, 494, and 495, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:496, 497, 498, 499, 500, and 501, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:502, 503, 504, 505, 506, and 507, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:502, 718, 719, 505, 506, and 507, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:508, 509, 510, 511, 512, and 513, respectively. In certain embodiments, the anti-TRGV9 antibody or antigen-binding 15 fragment thereof comprises a HCDR1, HCDR2, HCDR3, LCDR1, LCDR2, and LCDR3, having the polypeptide sequence of SEQ ID NOs:514, 515, 516, 517, 518, and 519, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:520, 521, 522, 523, 524, and 525, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:526, 527, 528, 529, 530, and 531, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:532, 533, 534, 535, 536, and 537, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:538, 539, 540, 541, 542, and 543, respectively. In certain embodiments, the anti-TRGV9 antibody or antigen-binding 30 fragment thereof comprises a HCDR1, HCDR2, HCDR3, LCDR1, LCDR2, and LCDR3, having the polypeptide sequence of SEQ ID NOs:538, 720, 721, 541, 542, and 543, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:544, 545, 546, 547, 548, and 549, respectively. In certain embodiments, the anti-TRGV9 antibody or antigen-binding fragment thereof comprises a HCDR1, HCDR2, HCDR3, LCDR1, LCDR2, and LCDR3, having the polypeptide sequence of SEQ ID NOs:550, 551, 552, 553, 554, and 555, respectively.

In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:9, 10, 11, 12, 13, and 14, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:556, 557, 558, 559, 560, and 561, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:562, 563, 564, 565, 566, and 567, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:568, 569, 570, 571, 572, and 573, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:574, 575, 576, 577, 578, and 579, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:574, 722, 723, 577, 578, and 579, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:580, 581, 582, 583, 584, and 585, respectively. In some embodiments, the anti-CD123 antibody or antigen-binding fragment thereof comprises a HCDR1, HCDR2, HCDR3, LCDR1, LCDR2, and LCDR3, having the polypeptide sequence of SEQ ID NOs:586, 587, 588, 589, 590, and 591, respectively.

In certain embodiments, the bispecific antibody comprises any of the anti-TRGV9 antibodies provided herein. In some embodiments, the bispecific antibody comprises an antigen binding fragment of any of the anti-TRGV9 antibodies provided herein. In certain embodiments, the bispecific antibody comprises any of the anti-CD123 antibodies provided herein. In some embodiments, the bispecific antibody comprises an antigen binding fragment of any of the anti-CD123 antibodies provided herein. In other embodiments, the bispecific antibody comprises any of the anti-TRGV9 antibodies provided herein, and any of the CD123 antibodies provided herein. In some embodiments, the bispecific antibody comprises any of the anti-TRGV9 antibodies provided herein, and an antigen binding fragment of any of the CD123 antibodies provided herein. In other embodiments, the bispecific antibody comprises an antigen binding fragment any of the anti-TRGV9 antibodies provided herein, and any of the CD123 antibodies provided herein. In yet other embodiments, the bispecific antibody comprises an antigen binding fragment of any of the anti-TRGV9 antibodies provided herein, and an antigen binding fragment of any of the CD123 antibodies provided herein.

According to another particular aspect, provided herein is an isolated anti-TRGV9/anti-CD123 bispecific antibody or antigen-binding fragment thereof that induces antibody-dependent cell-mediated cytotoxicity (ADCC). The bispecific antibody or antigen-binding fragment thereof can, for example, induce ADCC in vitro.

In certain embodiments, the bispecific antibody or antigen-binding fragment thereof induces γδ T cell dependent cytotoxicity of a cancer cell in vitro with an EC₅₀ of less than about 160 pM, when assessed in vitro at an effector to target cell ratio of 1:1. In one such embodiment, the bispecific antibody is an isolated anti-TRGV9/anti-CD123 bispecific antibody or antigen-binding fragment thereof that exhibits an EC₅₀ of less than about 160 pM, when assessed in vitro with a mixture of γδ T effector cells and Kasumi3 AML target cells, where such cells are present in an effector to target cell ratio of about 1:1 and the bispecific antibody or antigen-binding fragment thereof is present at a concentration of about 30 ng/mL.

In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein, HC1 comprises a HCDR1, HCDR2, and HCDR3 comprising the amino acid sequences of (i) SEQ ID NO:1, SEQ ID NO:2, and SEQ ID NO:3, respectively, (ii) SEQ ID NO:1, SEQ ID NO:2, and SEQ ID NO:31, respectively, (iii) SEQ ID NO:1, SEQ ID NO:2, and SEQ ID NO:32, respectively, or (iv) SEQ ID NO:1, SEQ ID NO:2, and SEQ ID NO:33, respectively, and LC1 comprises a LCDR1, LCDR2, and LCDR3 comprising the amino acid sequences of SEQ ID NO:4, SEQ ID NO:5, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein, HC1 comprises a HCDR1, HCDR2, and HCDR3 comprising the amino acid sequences of SEQ ID NO:1, SEQ ID NO:2, and SEQ ID NO:3, respectively, and LC1 comprises a LCDR1, LCDR2, and LCDR3 comprising the amino acid sequences of SEQ ID NO:4, SEQ ID NO:5, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:31, respectively, and LC1 comprises a LCDR1, LCDR2, and LCDR3 comprising the amino acid sequences of SEQ ID NO:4, SEQ ID NO:5, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:32, respectively, and LC1 comprises a LCDR1, LCDR2, and LCDR3 comprising the amino acid sequences of SEQ ID NO:4, SEQ ID NO:5, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:2, and SEQ ID NO:33, respectively, and LC1 comprises a LCDR1, LCDR2, and LCDR3 comprising the amino acid sequences of SEQ ID NO:4, SEQ ID NO:5, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:1, SEQ ID NO:76, and SEQ ID NO:3, respectively, and LC1 comprises a LCDR1, LCDR2, and LCDR3 comprising the amino acid sequences of SEQ ID NO:77, SEQ ID NO:5, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:60, SEQ ID NO:61, and SEQ ID NO:62, respectively, and LC1 comprises a LCDR1, LCDR2, and LCDR3 comprising the amino acid sequences of SEQ ID NO:63, SEQ ID NO:64, and SEQ ID NO:6, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:89, SEQ ID NO:90, and SEQ ID NO:91, respectively, and LC1 comprises a LCDR1, LCDR2, and LCDR3 comprising the amino acid sequences of SEQ ID NO:92, SEQ ID NO:93, and SEQ ID NO:94, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:98, SEQ ID NO:99, and SEQ ID NO:100, respectively, and LC1 comprises a LCDR1, LCDR2, and LCDR3 comprising the amino acid sequences of SEQ ID NO:101, SEQ ID NO: 102, and SEQ ID NO:103, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:107, SEQ ID NO:108, and SEQ ID NO:109, respectively, and LC1 comprises a LCDR1, LCDR2, and LCDR3 comprising the amino acid sequences of SEQ ID NO:110, SEQ ID NO:111, and SEQ ID NO:112, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:117, SEQ ID NO:118, and SEQ ID NO:119, respectively, and LC1 comprises a LCDR1, LCDR2, and LCDR3 comprising the amino acid sequences of SEQ ID NO:120, SEQ ID NO:121, and SEQ ID NO:122, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:127, SEQ ID NO:128, and SEQ ID NO:129, respectively, and LC1 comprises a LCDR1, LCDR2, and LCDR3 comprising the amino acid sequences of SEQ ID NO:130, SEQ ID NO:131, and SEQ ID NO:132, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:160, SEQ ID NO:161, and SEQ ID NO:162, respectively, and LC1 comprises a LCDR1, LCDR2, and LCDR3 comprising the amino acid sequences of SEQ ID NO:163, SEQ ID NO: 164, and SEQ ID NO:165, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:166, SEQ ID NO:167, and SEQ ID NO:168, respectively, and LC1 comprises a LCDR1, LCDR2, and LCDR3 comprising the amino acid sequences of SEQ ID NO:169, SEQ ID NO: 170, and SEQ ID NO:171, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:172, SEQ ID NO:173, and SEQ ID NO:174, respectively, and LC1 comprises a LCDR1, LCDR2, and LCDR3 comprising the amino acid sequences of SEQ ID NO:175, SEQ ID NO: 176, and SEQ ID NO:177, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:178, SEQ ID NO:179, and SEQ ID NO:180, respectively, and LC1 comprises a LCDR1, LCDR2, and LCDR3 comprising the amino acid sequences of SEQ ID NO:181, SEQ ID NO: 182, and SEQ ID NO:183, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:178, SEQ ID NO:700, and SEQ ID NO:701, respectively, and LC1 comprises a LCDR1, LCDR2, and LCDR3 comprising the amino acid sequences of SEQ ID NO:181, SEQ ID NO: 182, and SEQ ID NO:183, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:184, SEQ ID NO:185, and SEQ ID NO:186, respectively, and LC1 comprises a LCDR1, LCDR2, and LCDR3 comprising the amino acid sequences of SEQ ID NO:187, SEQ ID NO: 188, and SEQ ID NO:189, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:190, SEQ ID NO:191, and SEQ ID NO:192, respectively, and LC1 comprises a LCDR1, LCDR2, and LCDR3 comprising the amino acid sequences of SEQ ID NO:193, SEQ ID NO: 194, and SEQ ID NO:195, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:196, SEQ ID NO:197, and SEQ ID NO:198, respectively, and LC1 comprises a LCDR1, LCDR2, and LCDR3 comprising the amino acid sequences of SEQ ID NO:199, SEQ ID NO:200, and SEQ ID NO:201, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:202, SEQ ID NO:203, and SEQ ID NO:204, respectively, and LC1 comprises a LCDR1, LCDR2, and LCDR3 comprising the amino acid sequences of SEQ ID NO:205, SEQ ID NO:206, and SEQ ID NO:207, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:208, SEQ ID NO:209, and SEQ ID NO:210, respectively, and LC1 comprises a LCDR1, LCDR2, and LCDR3 comprising the amino acid sequences of SEQ ID NO:211, SEQ ID NO:212, and SEQ ID NO:213, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:214, SEQ ID NO:215, and SEQ ID NO:216, respectively, and LC1 comprises a LCDR1, LCDR2, and LCDR3 comprising the amino acid sequences of SEQ ID NO:217, SEQ ID NO:218, and SEQ ID NO:219, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:214, SEQ ID NO:702, and SEQ ID NO:703, respectively, and LC1 comprises a LCDR1, LCDR2, and LCDR3 comprising the amino acid sequences of SEQ ID NO:217, SEQ ID NO:218, and SEQ ID NO:219, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:220, SEQ ID NO:221, and SEQ ID NO:222, respectively, and LC1 comprises a LCDR1, LCDR2, and LCDR3 comprising the amino acid sequences of SEQ ID NO:223, SEQ ID NO:224, and SEQ ID NO:225, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:226, SEQ ID NO:227, and SEQ ID NO:228, respectively, and LC1 comprises a LCDR1, LCDR2, and LCDR3 comprising the amino acid sequences of SEQ ID NO:229, SEQ ID NO:230, and SEQ ID NO:231, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:232, SEQ ID NO:233, and SEQ ID NO:234, respectively, and LC1 comprises a LCDR1, LCDR2, and LCDR3 comprising the amino acid sequences of SEQ ID NO:235, SEQ ID NO:236, and SEQ ID NO:237, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:238, SEQ ID NO:239, and SEQ ID NO:240, respectively, and LC1 comprises a LCDR1, LCDR2, and LCDR3 comprising the amino acid sequences of SEQ ID NO:241, SEQ ID NO:242, and SEQ ID NO:243, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:244, SEQ ID NO:245, and SEQ ID NO:246, respectively, and LC1 comprises a LCDR1, LCDR2, and LCDR3 comprising the amino acid sequences of SEQ ID NO:247, SEQ ID NO:248, and SEQ ID NO:249, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:250, SEQ ID NO:251, and SEQ ID NO:252, respectively, and LC1 comprises a LCDR1, LCDR2, and LCDR3 comprising the amino acid sequences of SEQ ID NO:253, SEQ ID NO:254, and SEQ ID NO:255, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:250, SEQ ID NO:704, and SEQ ID NO:705, respectively, and LC1 comprises a LCDR1, LCDR2, and LCDR3 comprising the amino acid sequences of SEQ ID NO:253, SEQ ID NO:254, and SEQ ID NO:255, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:256, SEQ ID NO:257, and SEQ ID NO:258, respectively, and LC1 comprises a LCDR1, LCDR2, and LCDR3 comprising the amino acid sequences of SEQ ID NO:259, SEQ ID NO:260, and SEQ ID NO:261, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:262, SEQ ID NO:263, and SEQ ID NO:264, respectively, and LC1 comprises a LCDR1, LCDR2, and LCDR3 comprising the amino acid sequences of SEQ ID NO:265, SEQ ID NO:266, and SEQ ID NO:267, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:268, SEQ ID NO:269, and SEQ ID NO:270, respectively, and LC1 comprises a LCDR1, LCDR2, and LCDR3 comprising the amino acid sequences of SEQ ID NO:271, SEQ ID NO:272, and SEQ ID NO:273, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:274, SEQ ID NO:275, and SEQ ID NO:276, respectively, and LC1 comprises a LCDR1, LCDR2, and LCDR3 comprising the amino acid sequences of SEQ ID NO:277, SEQ ID NO:278, and SEQ ID NO:279, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:280, SEQ ID NO:281, and SEQ ID NO:282, respectively, and LC1 comprises a LCDR1, LCDR2, and LCDR3 comprising the amino acid sequences of SEQ ID NO:283, SEQ ID NO:284, and SEQ ID NO:285, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:286, SEQ ID NO:287, and SEQ ID NO:288, respectively, and LC1 comprises a LCDR1, LCDR2, and LCDR3 comprising the amino acid sequences of SEQ ID NO:289, SEQ ID NO:290, and SEQ ID NO:291, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:286, SEQ ID NO:706, and SEQ ID NO:707, respectively, and LC1 comprises a LCDR1, LCDR2, and LCDR3 comprising the amino acid sequences of SEQ ID NO:289, SEQ ID NO:290, and SEQ ID NO:291, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:292, SEQ ID NO:293, and SEQ ID NO:294, respectively, and LC1 comprises a LCDR1, LCDR2, and LCDR3 comprising the amino acid sequences of SEQ ID NO:295, SEQ ID NO:296, and SEQ ID NO:297, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:298, SEQ ID NO:299, and SEQ ID NO:300, respectively, and LC1 comprises a LCDR1, LCDR2, and LCDR3 comprising the amino acid sequences of SEQ ID NO:301, SEQ ID NO:302, and SEQ ID NO:303, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:304, SEQ ID NO:305, and SEQ ID NO:306, respectively, and LC1 comprises a LCDR1, LCDR2, and LCDR3 comprising the amino acid sequences of SEQ ID NO:307, SEQ ID NO:308, and SEQ ID NO:309, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:310, SEQ ID NO:311, and SEQ ID NO:312, respectively, and LC1 comprises a LCDR1, LCDR2, and LCDR3 comprising the amino acid sequences of SEQ ID NO:313, SEQ ID NO:314, and SEQ ID NO:315, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:316, SEQ ID NO:317, and SEQ ID NO:318, respectively, and LC1 comprises a LCDR1, LCDR2, and LCDR3 comprising the amino acid sequences of SEQ ID NO:319, SEQ ID NO:320, and SEQ ID NO:321, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:322, SEQ ID NO:323, and SEQ ID NO:324, respectively, and LC1 comprises a LCDR1, LCDR2, and LCDR3 comprising the amino acid sequences of SEQ ID NO:325, SEQ ID NO:326, and SEQ ID NO:327, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:322, SEQ ID NO:708, and SEQ ID NO:709, respectively, and LC1 comprises a LCDR1, LCDR2, and LCDR3 comprising the amino acid sequences of SEQ ID NO:325, SEQ ID NO:326, and SEQ ID NO:327, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:328, SEQ ID NO:329, and SEQ ID NO:330, respectively, and LC1 comprises a LCDR1, LCDR2, and LCDR3 comprising the amino acid sequences of SEQ ID NO:331, SEQ ID NO:332, and SEQ ID NO:333, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:334, SEQ ID NO:335, and SEQ ID NO:336, respectively, and LC1 comprises a LCDR1, LCDR2, and LCDR3 comprising the amino acid sequences of SEQ ID NO:337, SEQ ID NO:338, and SEQ ID NO:339, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:340, SEQ ID NO:341, and SEQ ID NO:342, respectively, and LC1 comprises a LCDR1, LCDR2, and LCDR3 comprising the amino acid sequences of SEQ ID NO:343, SEQ ID NO:344, and SEQ ID NO:345, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:346, SEQ ID NO:347, and SEQ ID NO:348, respectively, and LC1 comprises a LCDR1, LCDR2, and LCDR3 comprising the amino acid sequences of SEQ ID NO:349, SEQ ID NO:350, and SEQ ID NO:351, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:352, SEQ ID NO:353, and SEQ ID NO:354, respectively, and LC1 comprises a LCDR1, LCDR2, and LCDR3 comprising the amino acid sequences of SEQ ID NO:355, SEQ ID NO:356, and SEQ ID NO:357, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:358, SEQ ID NO:359, and SEQ ID NO:360, respectively, and LC1 comprises a LCDR1, LCDR2, and LCDR3 comprising the amino acid sequences of SEQ ID NO:361, SEQ ID NO:362, and SEQ ID NO:363, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:358, SEQ ID NO:710, and SEQ ID NO:711, respectively, and LC1 comprises a LCDR1, LCDR2, and LCDR3 comprising the amino acid sequences of SEQ ID NO:361, SEQ ID NO:362, and SEQ ID NO:363, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:364, SEQ ID NO:365, and SEQ ID NO:366, respectively, and LC1 comprises a LCDR1, LCDR2, and LCDR3 comprising the amino acid sequences of SEQ ID NO:367, SEQ ID NO:368, and SEQ ID NO:369, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:370, SEQ ID NO:371, and SEQ ID NO:372, respectively, and LC1 comprises a LCDR1, LCDR2, and LCDR3 comprising the amino acid sequences of SEQ ID NO:373, SEQ ID NO:374, and SEQ ID NO:375, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:376, SEQ ID NO:377, and SEQ ID NO:378, respectively, and LC1 comprises a LCDR1, LCDR2, and LCDR3 comprising the amino acid sequences of SEQ ID NO:379, SEQ ID NO:380, and SEQ ID NO:381, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:382, SEQ ID NO:383, and SEQ ID NO:384, respectively, and LC1 comprises a LCDR1, LCDR2, and LCDR3 comprising the amino acid sequences of SEQ ID NO:385, SEQ ID NO:386, and SEQ ID NO:387, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:388, SEQ ID NO:389, and SEQ ID NO:390, respectively, and LC1 comprises a LCDR1, LCDR2, and LCDR3 comprising the amino acid sequences of SEQ ID NO:391, SEQ ID NO:392, and SEQ ID NO:393, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:394, SEQ ID NO:395, and SEQ ID NO:396, respectively, and LC1 comprises a LCDR1, LCDR2, and LCDR3 comprising the amino acid sequences of SEQ ID NO:397, SEQ ID NO:398, and SEQ ID NO:399, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:394, SEQ ID NO:712, and SEQ ID NO:713, respectively, and LC1 comprises a LCDR1, LCDR2, and LCDR3 comprising the amino acid sequences of SEQ ID NO:397, SEQ ID NO:398, and SEQ ID NO:399, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:400, SEQ ID NO:401, and SEQ ID NO:402, respectively, and LC1 comprises a LCDR1, LCDR2, and LCDR3 comprising the amino acid sequences of SEQ ID NO:403, SEQ ID NO:404, and SEQ ID NO:405, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:406, SEQ ID NO:407, and SEQ ID NO:408, respectively, and LC1 comprises a LCDR1, LCDR2, and LCDR3 comprising the amino acid sequences of SEQ ID NO:409, SEQ ID NO:410, and SEQ ID NO:411, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:412, SEQ ID NO:413, and SEQ ID NO:414, respectively, and LC1 comprises a LCDR1, LCDR2, and LCDR3 comprising the amino acid sequences of SEQ ID NO:415, SEQ ID NO:416, and SEQ ID NO:417, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:418, SEQ ID NO:419, and SEQ ID NO:420, respectively, and LC1 comprises a LCDR1, LCDR2, and LCDR3 comprising the amino acid sequences of SEQ ID NO:421, SEQ ID NO:422, and SEQ ID NO:423, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:424, SEQ ID NO:425, and SEQ ID NO:426, respectively, and LC1 comprises a LCDR1, LCDR2, and LCDR3 comprising the amino acid sequences of SEQ ID NO:427, SEQ ID NO:428, and SEQ ID NO:429, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:430, SEQ ID NO:431, and SEQ ID NO:432, respectively, and LC1 comprises a LCDR1, LCDR2, and LCDR3 comprising the amino acid sequences of SEQ ID NO:433, SEQ ID NO:434, and SEQ ID NO:435, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:430, SEQ ID NO:714, and SEQ ID NO:715, respectively, and LC1 comprises a LCDR1, LCDR2, and LCDR3 comprising the amino acid sequences of SEQ ID NO:433, SEQ ID NO:434, and SEQ ID NO:435, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:436, SEQ ID NO:437, and SEQ ID NO:438, respectively, and LC1 comprises a LCDR1, LCDR2, and LCDR3 comprising the amino acid sequences of SEQ ID NO:439, SEQ ID NO:440, and SEQ ID NO:441, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:442, SEQ ID NO:443, and SEQ ID NO:444, respectively, and LC1 comprises a LCDR1, LCDR2, and LCDR3 comprising the amino acid sequences of SEQ ID NO:445, SEQ ID NO:446, and SEQ ID NO:447, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:448, SEQ ID NO:449, and SEQ ID NO:450, respectively, and LC1 comprises a LCDR1, LCDR2, and LCDR3 comprising the amino acid sequences of SEQ ID NO:451, SEQ ID NO:452, and SEQ ID NO:453, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:454, SEQ ID NO:455, and SEQ ID NO:456, respectively, and LC1 comprises a LCDR1, LCDR2, and LCDR3 comprising the amino acid sequences of SEQ ID NO:457, SEQ ID NO:458, and SEQ ID NO:459, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:460, SEQ ID NO:461, and SEQ ID NO:462, respectively, and LC1 comprises a LCDR1, LCDR2, and LCDR3 comprising the amino acid sequences of SEQ ID NO:463, SEQ ID NO:464, and SEQ ID NO:465, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:466, SEQ ID NO:467, and SEQ ID NO:468, respectively, and LC1 comprises a LCDR1, LCDR2, and LCDR3 comprising the amino acid sequences of SEQ ID NO:469, SEQ ID NO:470, and SEQ ID NO:471, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:466, SEQ ID NO:716, and SEQ ID NO:717, respectively, and LC1 comprises a LCDR1, LCDR2, and LCDR3 comprising the amino acid sequences of SEQ ID NO:469, SEQ ID NO:470, and SEQ ID NO:471, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:472, SEQ ID NO:473, and SEQ ID NO:474, respectively, and LC1 comprises a LCDR1, LCDR2, and LCDR3 comprising the amino acid sequences of SEQ ID NO:475, SEQ ID NO:476, and SEQ ID NO:477, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:478, SEQ ID NO:479, and SEQ ID NO:480, respectively, and LC1 comprises a LCDR1, LCDR2, and LCDR3 comprising the amino acid sequences of SEQ ID NO:481, SEQ ID NO:482, and SEQ ID NO:483, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:484, SEQ ID NO:485, and SEQ ID NO:486, respectively, and LC1 comprises a LCDR1, LCDR2, and LCDR3 comprising the amino acid sequences of SEQ ID NO:487, SEQ ID NO:488, and SEQ ID NO:489, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:490, SEQ ID NO:491, and SEQ ID NO:492, respectively, and LC1 comprises a LCDR1, LCDR2, and LCDR3 comprising the amino acid sequences of SEQ ID NO:493, SEQ ID NO:494, and SEQ ID NO:495, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:496, SEQ ID NO:497, and SEQ ID NO:498, respectively, and LC1 comprises a LCDR1, LCDR2, and LCDR3 comprising the amino acid sequences of SEQ ID NO:499, SEQ ID NO:500, and SEQ ID NO:501, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:502, SEQ ID NO:503, and SEQ ID NO:504, respectively, and LC1 comprises a LCDR1, LCDR2, and LCDR3 comprising the amino acid sequences of SEQ ID NO:505, SEQ ID NO:506, and SEQ ID NO:507, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:502, SEQ ID NO:503, and SEQ ID NO:719, respectively, and LC1 comprises a LCDR1, LCDR2, and LCDR3 comprising the amino acid sequences of SEQ ID NO:505, SEQ ID NO:506, and SEQ ID NO:507, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:508, SEQ ID NO:509, and SEQ ID NO:510, respectively, and LC1 comprises a LCDR1, LCDR2, and LCDR3 comprising the amino acid sequences of SEQ ID NO:511, SEQ ID NO:512, and SEQ ID NO:513, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:514, SEQ ID NO:515, and SEQ ID NO:516, respectively, and LC1 comprises a LCDR1, LCDR2, and LCDR3 comprising the amino acid sequences of SEQ ID NO:517, SEQ ID NO:518, and SEQ ID NO:519, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:520, SEQ ID NO:521, and SEQ ID NO:522, respectively, and LC1 comprises a LCDR1, LCDR2, and LCDR3 comprising the amino acid sequences of SEQ ID NO:523, SEQ ID NO:524, and SEQ ID NO:525, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:526, SEQ ID NO:527, and SEQ ID NO:528, respectively, and LC1 comprises a LCDR1, LCDR2, and LCDR3 comprising the amino acid sequences of SEQ ID NO:529, SEQ ID NO:530, and SEQ ID NO:531, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:532, SEQ ID NO:533, and SEQ ID NO:534, respectively, and LC1 comprises a LCDR1, LCDR2, and LCDR3 comprising the amino acid sequences of SEQ ID NO:535, SEQ ID NO:536, and SEQ ID NO:537, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:538, SEQ ID NO:539, and SEQ ID NO:540, respectively, and LC1 comprises a LCDR1, LCDR2, and LCDR3 comprising the amino acid sequences of SEQ ID NO:541, SEQ ID NO:542, and SEQ ID NO:543, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:538, SEQ ID NO:720, and SEQ ID NO:721, respectively, and LC1 comprises a LCDR1, LCDR2, and LCDR3 comprising the amino acid sequences of SEQ ID NO:541, SEQ ID NO:542, and SEQ ID NO:543, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:544, SEQ ID NO:545, and SEQ ID NO:546, respectively, and LC1 comprises a LCDR1, LCDR2, and LCDR3 comprising the amino acid sequences of SEQ ID NO:547, SEQ ID NO:548, and SEQ ID NO:549, respectively, to form a first antigen-binding site that specifically binds TRGV9. In another embodiment, the bispecific antibody is an isolated anti-TRGV9/anti-CD123 bispecific antibody or antigen-binding fragment thereof comprising (a) a HC1; (b) a HC2; (c) a LC1; and (d) a LC2, wherein HC1 is associated with LC1 and HC2 is associated with LC2, and wherein HC1 comprises a HCDR1, HCDR2, and HCDR3 comprising the amino acid sequences of SEQ ID NO:550, SEQ ID NO:551, and SEQ ID NO:552, respectively, and LC1 comprises a LCDR1, LCDR2, and LCDR3 comprising the amino acid sequences of SEQ ID NO:553, SEQ ID NO:554, and SEQ ID NO:555, respectively, to form a first antigen-binding site that specifically binds TRGV9. In some embodiments, HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14 respectively, to form a second antigen-binding site that specifically binds CD123, wherein the anti-TRGV9/anti-CD123 bispecific antibody or antigen-binding fragment thereof exhibits an EC₅₀ of less than about 160 pM, when assessed in vitro with a mixture of γδ T effector cells and Kasumi3 AML target cells, where such cells are present in an effector to target cell ratio of about 1:1 and the bispecific antibody or antigen-binding fragment thereof is present at a concentration of about 30 ng/mL. In some embodiments, HC2 comprises a HCDR1, HCDR2, and HCDR3 comprising the amino acid sequences of SEQ ID NO:9, SEQ ID NO:10, and SEQ ID NO:11, respectively, and LC2 comprises a LCDR1, LCDR2, and LCDR3 comprising the amino acid sequences of SEQ ID NO:12, SEQ ID NO:13, and SEQ ID NO:14 respectively, to form a second antigen-binding site that specifically binds CD123. In some embodiments, the HC2 comprises a HCDR1, HCDR2, and HCDR3 comprising the amino acid sequence of SEQ ID NO:556, SEQ ID NO:557, and SEQ ID NO:558, respectively, and a LCDR1, LCDR2, and LCDR3 comprising the amino acid sequence of SEQ ID NO:559, SEQ ID NO:560, and SEQ ID NO:561, respectively, to form a second antigen-binding site that specifically binds CD123. In some embodiments, the HC2 comprises a HCDR1, HCDR2, and HCDR3 comprising the amino acid sequence of SEQ ID NO:562, SEQ ID NO:563, and SEQ ID NO:564, respectively, and a LCDR1, LCDR2, and LCDR3 comprising the amino acid sequence of SEQ ID NO:565, SEQ ID NO:566, and SEQ ID NO:567, respectively, to form a second antigen-binding site that specifically binds CD123. In some embodiments, the HC2 comprises a HCDR1, HCDR2, and HCDR3 comprising the amino acid sequence of SEQ ID NO:568, SEQ ID NO:569, and SEQ ID NO:570, respectively, and a LCDR1, LCDR2, and LCDR3 comprising the amino acid sequence of SEQ ID NO:571, SEQ ID NO:572, and SEQ ID NO:573, respectively, to form a second antigen-binding site that specifically binds CD123. In some embodiments, the HC2 comprises a HCDR1, HCDR2, and HCDR3 comprising the amino acid sequence of SEQ ID NO:574, SEQ ID NO:575, and SEQ ID NO:576, respectively, and a LCDR1, LCDR2, and LCDR3 comprising the amino acid sequence of SEQ ID NO:577, SEQ ID NO:578, and SEQ ID NO:579, respectively, to form a second antigen-binding site that specifically binds CD123. In some embodiments, the HC2 comprises a HCDR1, HCDR2, and HCDR3 comprising the amino acid sequence of SEQ ID NO:574, SEQ ID NO:722, and SEQ ID NO:723, respectively, and a LCDR1, LCDR2, and LCDR3 comprising the amino acid sequence of SEQ ID NO:577, SEQ ID NO:578, and SEQ ID NO:579, respectively, to form a second antigen-binding site that specifically binds CD123. In some embodiments, the HC2 comprises a HCDR1, HCDR2, and HCDR3 comprising the amino acid sequence of SEQ ID NO:580, SEQ ID NO:581, and SEQ ID NO:582, respectively, and a LCDR1, LCDR2, and LCDR3 comprising the amino acid sequence of SEQ ID NO:583, SEQ ID NO:584, and SEQ ID NO:585, respectively, to form a second antigen-binding site that specifically binds CD123. In some embodiments, the HC2 comprises a HCDR1, HCDR2, and HCDR3 comprising the amino acid sequence of SEQ ID NO:586, SEQ ID NO:587, and SEQ ID NO:588; and (ii) a VL comprising a VL CDR1 having an amino acid sequence of SEQ ID NO:589, SEQ ID NO:590, and SEQ ID NO:591, respectively, to form a second antigen-binding site that specifically binds CD123. In certain embodiments, the anti-TRGV9/anti-CD123 bispecific antibody or antigen-binding fragment thereof exhibits an EC₅₀ of less than about 160 pM, when assessed in vitro with a mixture of γδ T effector cells and Kasumi3 AML target cells, where such cells are present in an effector to target cell ratio of about 1:1 and the bispecific antibody or antigen-binding fragment thereof is present at a concentration of about 30 ng/mL.

In certain embodiments, the EC₅₀ is less than about 1000 pM, less than about 900 pM, less than about 800 pM, less than about 700 pM, less than about 600 pM, less than about 500 pM, less than about 400 pM, less than about 300 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about 30 pM, less than about 20 pM, or less than about 10 pM.

In certain embodiments, the effector to target cell ratio can, for example, be 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1,7:1, 8:1, 9:1, or 10:1.

In certain embodiments, the concentration of the bispecific antibody or antigen-binding fragment thereof is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL.

In some embodiments described herein, immune effector properties of the anti-TRGV9/anti-CD123 bispecific antibodies can be enhanced or silenced through Fc modifications by techniques known to those skilled in the art. For example, Fc effector functions such as Clq binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. can be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of 0-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the anti-TRGV9/anti-CD123 bispecific antibodies can also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

According to another particular aspect, provided herein is an isolated anti-TRGV9/anti-CD123 bispecific antibody or antigen-binding fragment thereof. In some embodiments, the anti-TRGV9/anti-CD123 bispecific antibody or antigen-binding fragment thereof is chimeric.

According to another particular aspect, provided herein is an isolated anti-TRGV9/anti-CD123 bispecific antibody or antigen-binding fragment thereof. In some embodiments, the anti-TRGV9/anti-CD123 bispecific antibody or antigen-binding fragment thereof is human or humanized.

Also provided is a nucleic acid encoding an antibody that binds to a TRGV9 provided herein. Also provided is a vector comprising a nucleic acid encoding an antibody that binds to a TRGV9 provided herein. Also provided is a host cell comprising a vector comprising a nucleic acid encoding an antibody that binds to a TRGV9 provided herein. Also provided is a kit comprising the vector comprising a nucleic acid encoding an antibody that binds to a TRGV9 provided herein, and packaging for the same.

Also provided is a nucleic acid encoding a bispecific antibody comprising: (a) a first binding domain that binds to TRGV9, and (b) a second binding domain that binds to a second target that is not TRGV9, as provided herein. Also provided is a vector comprising a nucleic acid encoding a bispecific antibody that binds to a TRGV9 provided herein. Also provided is a host cell comprising a vector comprising a nucleic acid encoding a bispecific antibody that binds to a TRGV9 provided herein. Also provided is a kit comprising the vector comprising a nucleic acid encoding a bispecific antibody that binds to a TRGV9 provided herein, and packaging for the same.

In another general aspect, provided is an isolated nucleic acid encoding an antibody or antigen-binding fragment thereof provided herein. In some embodiments, the antibody is a TRGV9 antibody provided herein. In certain embodiments, the antibody is a monoclonal antibody. In another general aspect, provided is an isolated nucleic acid encoding a bispecific antibody or antigen-binding fragment thereof provided herein. In some embodiments, the antibody is a TRGV bispecific antibody provided herein. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies and/or bispecific antibodies provided herein can be altered without changing the amino acid sequences of the proteins.

In another general aspect, provided is a vector comprising an isolated nucleic acid encoding an antibody or antigen-binding fragment thereof provided herein. In some embodiments, the antibody is a TRGV antibody provided herein. In certain embodiments, the antibody is a monoclonal antibody. In some embodiments, the antibody is a TRGV bispecific antibody provided herein. In another general aspect, provided is a vector comprising an isolated nucleic acid encoding a bispecific antibody or antigen-binding fragment thereof provided herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to certain embodiments. Such techniques are well known to those skilled in the art in view of the present disclosure.

In another general aspect, provided is a host cell comprising an isolated nucleic acid encoding an antibody or antigen-binding fragment thereof provided herein. In some embodiments, the antibody is a TRGV antibody provided herein. In certain embodiments, the antibody is a monoclonal antibody. In some embodiments, the antibody is a TRGV bispecific antibody provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, provided is a method of producing an antibody or antigen-binding fragment thereof disclosed herein. The methods comprise culturing a cell comprising a nucleic acid encoding the antibody or antigen-binding fragment thereof under conditions to produce an antibody or antigen-binding fragment thereof disclosed herein and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

In another general aspect, provided is a method of producing a bispecific antibody or antigen-binding fragment thereof disclosed herein. The methods comprise culturing a cell comprising a nucleic acid encoding the bispecific antibody or antigen-binding fragment thereof under conditions to produce a bispecific antibody or antigen-binding fragment thereof disclosed herein and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

Pharmaceutical Compositions

In another general aspect, provided is a pharmaceutical composition comprising an isolated bispecific antibody or antigen-binding fragment thereof provided herein and a pharmaceutically acceptable carrier. Also provided is a pharmaceutical composition comprising an antibody that binds to a TRGV9 provided herein, and a pharmaceutically acceptable carrier. Also provided is a method of producing the pharmaceutical composition, comprising combining the antibody with a pharmaceutically acceptable carrier to obtain the pharmaceutical composition. In another aspect, provided herein is a pharmaceutical composition comprising a comprising: (a) a first binding domain that binds to TRGV9, and (b) a second binding domain that binds to a second target that is not TRGV9, and a pharmaceutically acceptable carrier. Any of the bispecific antibodies provided herein are contemplated in the pharmaceutical compositions. In certain embodiments, the second binding domain binds to CD123. The term “pharmaceutical composition” as used herein means a product comprising an antibody provided herein together with a pharmaceutically acceptable carrier. Antibodies provided herein and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness or biological activity of a composition provided herein. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment, the pharmaceutical composition is a liquid formulation. One example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment, the pH of the formulation is from about 7.0 to about 9.5. In another embodiment, the pH of the formulation is from about 3.0 to about 7.0.

In another embodiment, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments.

In another embodiment, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments.

In another embodiment, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol. In some embodiments, the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments.

In another embodiment, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments.

In another embodiment, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment, the pharmaceutical composition comprises a stabilizer. In some embodiments, said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments.

In further embodiments, the pharmaceutical composition comprises one or more surfactants, such as a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments.

In a further embodiment, the pharmaceutical composition comprises one or more protease inhibitors, such as, e.g., EDTA, and/or benzamidine hydrochloric acid (HC1). The protease inhibitor can be present individually or in the aggregate, in a concentration from about 0.1 mg/ml to about 20 mg/ml. Pharmaceutical compositions comprising each one of these specific protease inhibitors constitute alternative embodiments.

In another general aspect, provided is a method of producing a pharmaceutical composition comprising a bispecific antibody or antigen-binding fragment thereof provided herein, comprising combining a bispecific antibody or antigen-binding fragment thereof with a pharmaceutically acceptable carrier to obtain the pharmaceutical composition.

Methods of Use

In one general aspect, provided is a method of activating a γδ T cell expressing TRGV9, comprising contacting the γδ T cell with an antibody that binds to a TRGV9 provided herein. In another aspect, provided herein is a method of activating a γδ T cell expressing TRGV9, comprising contacting the γδ T cell with the bispecific 20 antibody, as provided herein. In some embodiments, the contacting results in an increase in CD69, CD25, and/or Granzyme B expression, as compared to a control γδ T cell expressing TRGV9.

In another general aspect, provided is a method of inactivating a γδ T cell expressing TRGV9, comprising contacting the γδ T cell with an antibody that binds to a TRGV9 provided herein. In another aspect, provided herein is a method of inactivating a γδ T cell expressing TRGV9, comprising contacting the γδ T cell with the bispecific antibody, as provided herein.

In another general aspect, provided is a method of blocking activation of a γδ T cell expressing TRGV9, comprising contacting the γδ T cell with an antibody that binds to a TRGV9 provided herein. In another aspect, provided herein is a method of blocking activation of a γδ T cell expressing TRGV9, comprising contacting the γδ T cell with the bispecific antibody, as provided herein.

In another general aspect, provided is a method of modulating the activation of a γδ T cell expressing TRGV9, comprising contacting the γδ T cell with an antibody that binds to a TRGV9 provided herein. In another aspect, provided herein is a method of modulating the activation of a γδ T cell expressing TRGV9, comprising contacting the γδ T cell with the bispecific antibody, as provided herein.

In another aspect, provided herein is a method of directing a γδ T cell expressing TRGV9 to a cancer cell, the method comprising contacting the γδ T cell with a bispecific antibody provided herein. In some embodiments, the contacting directs the γδ T cell to the cancer cell.

In another general aspect, provided is a method of targeting CD123 on the surface of a cancer cell, the method comprising exposing the cancer cell to an anti-TRGV9/anti-CD123 bispecific antibody or antigen binding fragment thereof or a pharmaceutical composition provided herein.

The functional activity of bispecific antibodies and antigen-binding fragments thereof that bind TRGV9 and/or CD123 can be characterized by methods known in the art and as described herein. Methods for characterizing antibodies and antigen-binding fragments thereof that bind TRGV9 and/or CD123 include, but are not limited to, affinity and specificity assays including Biacore, ELISA, and OctetRed analysis; binding assays to detect the binding of antibodies to CD123 on cancer cells by FACS; binding assays to detect the binding of antibodies to TRGV9 on γδ T cells. According to particular embodiments, the methods for characterizing antibodies and antigen-binding fragments thereof that bind TRGV9 and/or CD123 include those described below.

In another general aspect, provided herein is a method of directing Vγ9-expressing γδ T cells to a cancer cell. The methods comprise contacting the Vγ9-expressing γδ T cell with an anti-TRGV9/anti-CD123 bispecific antibody or antigen binding fragment thereof. In some embodiments, the anti-TRGV9/anti-CD123 bispecific antibody or antigen binding fragment thereof directs the Vγ9-expressing γδ T cell to a cancer cell having CD123 on its surface.

In another general aspect, provided herein is a method for inhibiting growth or proliferation of cancer cells. The methods comprise contacting the Vγ9-expressing γδ T cells with an anti-TRGV9/anti-CD123 bispecific antibody or antigen binding fragment thereof. In some embodiments, contacting the cancer cells with the anti-TRGV9/anti-CD123 bispecific antibody or antigen binding fragment thereof composition inhibits the growth or proliferation of the cancer cells.

In another aspect, provided herein is a method of inhibiting growth or proliferation of cancer cells expressing a cancer antigen on the cell surface, the method comprising contacting the cancer cells with a bispecific antibody provided herein. In some embodiments, contacting the cancer cells with the pharmaceutical composition inhibits growth or proliferation of the cancer cells. In some embodiments, the cancer cells are in the presence of a γδ T cell expressing TRGV9 while in contact with the bispecific antibody.

In another aspect, provided herein is a method for eliminating cancer cells in a subject, comprising administering an effective amount of a bispecific antibody, as provided herein, to the subject. In another general aspect, provided is a method of treating a cancer in a subject in need thereof, comprising administering to the subject an isolated bispecific antibody or antigen binding fragment thereof that specifically binds TRGV9 and a tumor-associated antigen presented on the surface of a tumor cell (e.g., CD123) or a pharmaceutical composition disclosed herein. The cancer can, for example, be a CD123-expressing cancer. The cancer can, for example, be a CD123-expressing cancer. The cancer can, for example, be a hematologic cancer. The hematologic cancer can, for example, be a leukemia, a lymphoma, and a myeloma. The leukemia can be an acute myeloid leukemia (AML) or an acute lymphocytic leukemia (ALL). In some embodiments, the subject is a human. In some embodiments, the subject is a subject in need thereof.

According to certain embodiments, the pharmaceutical composition comprises an effective amount of an anti-TRGV9/anti-CD123 bispecific antibody or antigen-binding fragment thereof. As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the compositions can be used in combination with another treatment including, but not limited to, a chemotherapy, an anti-CD20 mAb, an anti-TIM-3 mAb, an anti-CTLA-4 antibody, an anti-PD-L1 antibody, an anti-PD-1 antibody, a PD-1/PD-L1 therapy, IDO, an anti-OX40 antibody, an anti-GITR antibody, an anti-CD40 antibody, an anti-CD38 antibody, cytokines, oncolytic viruses, TLR agonists, STING agonist, other immuno-oncology drugs, an antiangiogenic agent, a radiation therapy, an antibody-drug conjugate (ADC), a targeted therapy, or other anticancer drugs.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Anti-TRGV9 antibodies provided herein may also be used as agents to detect Vγ9-expressing cells. Thus, in another methods, provided is a method of detecting a cell expressing Vγ9, comprising contacting a cell with a TRGV9 antibody provided herein. In certain embodiments, the detecting is by ELISA. In some embodiments, the detecting is by FACS analysis. Also provided are kits comprising a TRGV9 antibody provided herein, and instructions for use.

### Embodiments

This invention provides the following non-limiting embodiments.

In one set of embodiments, provided are:

1. An antibody that binds to T Cell Receptor Gamma Variable 9 (TRGV9).
2. The antibody of embodiment 1, wherein the antibody comprises:
(i) a VH comprising a VH complementarity determining region (CDR) 1 having an amino acid sequence of SEQ ID NO:1, a VH CDR2 having an amino acid sequence of SEQ ID NO:2, and a VH CDR3 having an amino acid sequence of SEQ ID NO:31; and
(ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
   3. The antibody of embodiment 2, wherein the antibody comprises a VH having an amino acid sequence of SEQ ID NO:34.
   4. The antibody of embodiment 2, wherein the antibody comprises a VL having an amino acid sequence of SEQ ID NO:8.
   5. The antibody of embodiment 2, wherein the antibody comprises a VH having an amino acid sequence of SEQ ID NO:34, and a VL having an amino acid sequence of SEQ ID NO:8.
   6. The antibody of embodiment 1, wherein the antibody comprises:
(i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and
(ii) a VL comprising a VL CDR1 having an amino acid sequence of SEQ ID NO:4, a VL CDR2 having an amino acid sequence of SEQ ID NO: 5, and a VL CDR3 having an amino acid sequence of SEQ ID NO:6.
   7. The antibody of embodiment 6, wherein the antibody comprises a VH having an amino acid sequence of SEQ ID NO:35.
   8. The antibody of embodiment 6, wherein the antibody comprises a VL having an amino acid sequence of SEQ ID NO:8.
   9. The antibody of embodiment 6, wherein the antibody comprises a VH having an amino acid sequence of SEQ ID NO:35, and a VL having an amino acid sequence of SEQ ID NO:8.
   10. The antibody of embodiment 1, wherein the antibody comprises:
(i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and
(ii) a VL comprising a VL CDR1 having an amino acid sequence of SEQ ID NO:4, a VL CDR2 having an amino acid sequence of SEQ ID NO: 5, and a VL CDR3 having an amino acid sequence of SEQ ID NO:6.
   11. The antibody of embodiment 10, wherein the antibody comprises a VH having an amino acid sequence of SEQ ID NO:36.
   12. The antibody of embodiment 10, wherein the antibody comprises a VL having an amino acid sequence of SEQ ID NO:8.
   13. The antibody of embodiment 10, wherein the antibody comprises a VH having an amino acid sequence of SEQ ID NO:36, and a VL having an amino acid sequence of SEQ ID NO:8.
   14. The antibody of embodiment 1, wherein the antibody comprises:
(i) a VH comprising a VH CDR1 having an amino acid sequence of SEQ ID NO:1, a VH CDR2 having an amino acid sequence of SEQ ID NO: 76, and a VH CDR3 having an amino acid sequence of SEQ ID NO:3; and
(ii) a VL comprising a VL CDR1 having an amino acid sequence of SEQ ID NO:77, a VL CDR2 having an amino acid sequence of SEQ ID NO: 5, and a VL CDR3 having an amino acid sequence of SEQ ID NO:6.
   15. The antibody of embodiment 1, wherein the antibody comprises:
(i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and
(ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6.
   16. The antibody of embodiment 14 or 15, wherein the antibody comprises a VH having an amino acid sequence of SEQ ID NO:65.
   17. The antibody of embodiment 14 or 15, wherein the antibody comprises a VL having an amino acid sequence of SEQ ID NO:66.
   18. The antibody of embodiment 14 or 15, wherein the antibody comprises a VH having an amino acid sequence of SEQ ID NO:65, and a VL having an amino acid sequence of SEQ ID NO:66.
   19. The antibody of embodiment 14 or 15, wherein the antibody comprises a VH having an amino acid sequence of SEQ ID NO:67.
   20. The antibody of embodiment 14 or 15, wherein the antibody comprises a VL having an amino acid sequence of SEQ ID NO:68.
   21. The antibody of embodiment 14 or 15, wherein the antibody comprises a VH having an amino acid sequence of SEQ ID NO:67, and a VL having an amino acid sequence of SEQ ID NO:68.
   22. The antibody of embodiment 1, wherein the antibody comprises:
(i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100; and
(ii) a VL comprising a VL CDR1 having an amino acid sequence of SEQ ID NO:101, a VL CDR2 having an amino acid sequence of SEQ ID NO: 102, and a VL CDR3 having an amino acid sequence of SEQ ID NO: 103.
   23. The antibody of embodiment 22, wherein the antibody comprises a VH having an amino acid sequence of SEQ ID NO:104.
   24. The antibody of embodiment 22, wherein the antibody comprises a VL having an amino acid sequence of SEQ ID NO:105.
   25. The antibody of embodiment 22, wherein the antibody comprises a VH having an amino acid sequence of SEQ ID NO:104, and a VL having an amino acid sequence of SEQ ID NO:105.
   26. The antibody of embodiment 1, wherein the antibody comprises:
(i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109; and
(ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO: 112.
   27. The antibody of embodiment 26, wherein the antibody comprises a VH having an amino acid sequence of SEQ ID NO:113.
   28. The antibody of embodiment 26, wherein the antibody comprises a VL having an amino acid sequence of SEQ ID NO:114.
   29. The antibody of embodiment 26, wherein the antibody comprises a VH having an amino acid sequence of SEQ ID NO:113, and a VL having an amino acid sequence of SEQ ID NO:114.
   30. The antibody of embodiment 1, wherein the antibody comprises:
(i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119; and
(ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO: 122.
   31. The antibody of embodiment 30, wherein the antibody comprises a VH having an amino acid sequence of SEQ ID NO:123.
   32. The antibody of embodiment 30, wherein the antibody comprises a VL having an amino acid sequence of SEQ ID NO:124.
   33. The antibody of embodiment 30, wherein the antibody comprises a VH having an amino acid sequence of SEQ ID NO:123, and a VL having an amino acid sequence of SEQ ID NO:124.
   34. The antibody of embodiment 1, wherein the antibody comprises:
(i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129; and
(ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO: 132.
   35. The antibody of embodiment 34, wherein the antibody comprises a VH having an amino acid sequence of SEQ ID NO:133.
   36. The antibody of embodiment 34, wherein the antibody comprises a VL having an amino acid sequence of SEQ ID NO:134.
   37. The antibody of embodiment 34, wherein the antibody comprises a VH having an amino acid sequence of SEQ ID NO:133, and a VL having an amino acid sequence of SEQ ID NO:134.
   38. The antibody of any one of embodiments 1 to 37, wherein the TRGV9 is present on the surface of a γδ T cell.
   39. The antibody of any one of embodiments 1 to 38, wherein the antibody is a humanized antibody.
   40. The antibody of any one of embodiments 1 to 39, wherein the antibody is an IgG antibody.
   41. The antibody of embodiment 40, wherein the IgG antibody is an IgG1, IgG2, IgG3, or IgG4 antibody.
   42. The antibody of any one of embodiments 1 to 41, wherein the antibody is multivalent.
   43. The antibody of embodiment 42, wherein the antibody is capable of binding at least three antigens.
   44. The antibody of embodiment 42, wherein the antibody is capable of binding at least five antigens.
   45. A nucleic acid encoding the antibody of any one of embodiments 1 to 44.
   46. A vector comprising the nucleic acid of embodiment 45.
   47. A host cell comprising the vector of embodiment 45.
   48. A kit comprising the vector of embodiment 45 and packaging for the same.
   49. A pharmaceutical composition comprising the antibody of any one of embodiments 1 to 44, and a pharmaceutically acceptable carrier.
   50. A method of producing the pharmaceutical composition of embodiment 49, comprising combining the antibody with a pharmaceutically acceptable carrier to obtain the pharmaceutical composition.
   51. A method of activating a γδ T cell expressing TRGV9, comprising contacting the γδ T cell with the antibody of any one of embodiments 1 to 44.
   52. The method of embodiment 51, wherein the contacting results in an increase in CD69, CD25, and/or Granzyme B expression, as compared to a control γδ T cell expressing TRGV9.
   53. A bispecific antibody comprising:
(a) a first binding domain that binds to TRGV9, and
(b) a second binding domain that binds to a second target that is not TRGV9.
   54. The bispecific antibody of embodiment 53, wherein the first binding domain comprises:
(i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:3; and
(ii) a VL comprising a VL CDR1 having an amino acid sequence of SEQ ID NO:4, a VL CDR2 having an amino acid sequence of SEQ ID NO: 5, and a VL CDR3 having an amino acid sequence of SEQ ID NO:6.
   55. The bispecific antibody of embodiment 54, wherein the first binding domain comprises a VH having an amino acid sequence of SEQ ID NO:7.
   56. The bispecific antibody of embodiment 54, wherein the first binding domain comprises a VL having an amino acid sequence of SEQ ID NO:8.
   57. The bispecific antibody of embodiment 54, wherein the first binding domain comprises a VH having an amino acid sequence of SEQ ID NO:7, and a VL having an amino acid sequence of SEQ ID NO:8.
   58. The bispecific antibody of embodiment 53, wherein the first binding domain comprises:
(i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:31; and
(ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
   59. The bispecific antibody of embodiment 58, wherein the first binding domain comprises a VH having an amino acid sequence of SEQ ID NO:34.
   60. The bispecific antibody of embodiment 58, wherein the first binding domain comprises a VL having an amino acid sequence of SEQ ID NO:8.
   61. The bispecific antibody of embodiment 58, wherein the first binding domain comprises a VH having an amino acid sequence of SEQ ID NO:34, and a VL having an amino acid sequence of SEQ ID NO:8.
   62. The bispecific antibody of embodiment 53, wherein the first binding domain comprises:
(i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:32; and
(ii) a VL comprising a VL CDR1 having an amino acid sequence of SEQ ID NO:4, a VL CDR2 having an amino acid sequence of SEQ ID NO:5, and a VL CDR3 having an amino acid sequence of SEQ ID NO:6.
   63. The bispecific antibody of embodiment 62, wherein the first binding domain comprises a VH having an amino acid sequence of SEQ ID NO:35.
   64. The bispecific antibody of embodiment 62, wherein the first binding domain comprises a VL having an amino acid sequence of SEQ ID NO:8.
   65. The bispecific antibody of embodiment 62, wherein the first binding domain comprises a VH having an amino acid sequence of SEQ ID NO:35, and a VL having an amino acid sequence of SEQ ID NO:8.
   66. The bispecific antibody of embodiment 53, wherein the first binding domain comprises:
(i) a VH comprising a VH CDR1 having an amino acid sequence of SEQ ID NO:1, a VH CDR2 having an amino acid sequence of SEQ ID NO:2, and a VH CDR3 having an amino acid sequence of SEQ ID NO:33; and
(ii) a VL comprising a VL CDR1 having an amino acid sequence of SEQ ID NO:4, a VL CDR2 having an amino acid sequence of SEQ ID NO: 5, and a VL CDR3 having an amino acid sequence of SEQ ID NO:6.
   67. The bispecific antibody of embodiment 66, wherein the first binding domain comprises a VH having an amino acid sequence of SEQ ID NO:36.
   68. The bispecific antibody of embodiment 66, wherein the first binding domain comprises a VL having an amino acid sequence of SEQ ID NO:8.
   69. The bispecific antibody of embodiment 66, wherein the first binding domain comprises a VH having an amino acid sequence of SEQ ID NO:36, and a VL having an amino acid sequence of SEQ ID NO:8.
   70. The bispecific antibody of embodiment 53, wherein the first binding domain comprises:
(i) a VH comprising a VH CDR1 having an amino acid sequence of SEQ ID NO:1, a VH CDR2 having an amino acid sequence of SEQ ID NO: 76, and a VH CDR3 having an amino acid sequence of SEQ ID NO:3; and
(ii) a VL comprising a VL CDR1 having an amino acid sequence of SEQ ID NO:77, a VL CDR2 having an amino acid sequence of SEQ ID NO: 5, and a VL CDR3 having an amino acid sequence of SEQ ID NO:6.
   71. The bispecific antibody of embodiment 53, wherein the first binding domain comprises:
(i) a VH comprising a VH CDR1 having an amino acid sequence of SEQ ID NO:60, a VH CDR2 having an amino acid sequence of SEQ ID NO:61, and a VH CDR3 having an amino acid sequence of SEQ ID NO:62; and
(ii) a VL comprising a VL CDR1 having an amino acid sequence of SEQ ID NO:63, a VL CDR2 having an amino acid sequence of SEQ ID NO:64, and a VL CDR3 having an amino acid sequence of SEQ ID NO:6.
   72. The bispecific antibody of embodiment 70 or 71, wherein the first binding domain comprises a VH having an amino acid sequence of SEQ ID NO:65.
   73. The bispecific antibody of embodiment 70 or 71, wherein the first binding domain comprises a VL having an amino acid sequence of SEQ ID NO:66.
   74. The bispecific antibody of embodiment 70 or 71, wherein the first binding domain comprises a VH having an amino acid sequence of SEQ ID NO:65, and a VL having an amino acid sequence of SEQ ID NO:66.
   75. The bispecific antibody of embodiment 70 or 71, wherein the first binding domain comprises a VH having an amino acid sequence of SEQ ID NO:67.
   76. The bispecific antibody of embodiment 70 or 71, wherein the first binding domain comprises a VL having an amino acid sequence of SEQ ID NO:68.
   77. The bispecific antibody of embodiment 70 or 71, wherein the first binding domain a VH having an amino acid sequence of SEQ ID NO:67, and a VL having an amino acid sequence of SEQ ID NO:68.
   78. The bispecific antibody of embodiment 53, wherein the first binding domain comprises:
(i) a VH comprising a VH CDR1 having an amino acid sequence of SEQ ID NO:89, a VH CDR2 having an amino acid sequence of SEQ ID NO:90, and a VH CDR3 having an amino acid sequence of SEQ ID NO:91; and
(ii) a VL comprising a VL CDR1 having an amino acid sequence of SEQ ID NO:92, a VL CDR2 having an amino acid sequence of SEQ ID NO:93, and a VL CDR3 having an amino acid sequence of SEQ ID NO:94.
   79. The bispecific antibody of embodiment 78, wherein the first binding domain comprises a VH having an amino acid sequence of SEQ ID NO:95.
   80. The bispecific antibody of embodiment 78, wherein the first binding domain comprises a VL having an amino acid sequence of SEQ ID NO:96.
   81. The bispecific antibody of embodiment 78, wherein the first binding domain comprises a VH having an amino acid sequence of SEQ ID NO:95, and a VL having an amino acid sequence of SEQ ID NO:96.
   82. The bispecific antibody of embodiment 53, wherein the first binding domain comprises:
(i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100, and
(ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO: 103.
   83. The bispecific antibody of embodiment 82, wherein the first binding domain comprises a VH having an amino acid sequence of SEQ ID NO:104.
   84. The bispecific antibody of embodiment 82, wherein the first binding domain comprises a VL having an amino acid sequence of SEQ ID NO:105.
   85. The bispecific antibody of embodiment 82, wherein the first binding domain comprises a VH having an amino acid sequence of SEQ ID NO:104, and a VL having an amino acid sequence of SEQ ID NO:105.
   86. The bispecific antibody of embodiment 53, wherein the first binding domain comprises:
(i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109, and
(ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO: 112.
   87. The bispecific antibody of embodiment 86, wherein the first binding domain comprises a VH having an amino acid sequence of SEQ ID NO:113.
   88. The bispecific antibody of embodiment 86, wherein the first binding domain comprises a VL having an amino acid sequence of SEQ ID NO: 114.
   89. The bispecific antibody of embodiment 86, wherein the first binding domain comprises a VH having an amino acid sequence of SEQ ID NO:113, and a VL having an amino acid sequence of SEQ ID NO:114.
   90. The bispecific antibody of embodiment 53, wherein the first binding domain comprises:
(i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119, and
(ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO: 122.
   91. The bispecific antibody of embodiment 90, wherein the first binding domain comprises a VH having an amino acid sequence of SEQ ID NO:123.
   92. The bispecific antibody of embodiment 90, wherein the first binding domain comprises a VL having an amino acid sequence of SEQ ID NO:124.
   93. The bispecific antibody of embodiment 90, wherein the first binding domain comprises a VH having an amino acid sequence of SEQ ID NO:123, and a VL having an amino acid sequence of SEQ ID NO:124.
   94. The bispecific antibody of embodiment 53, wherein the first binding domain comprises:
(i) a VH comprising a VH CDR1 having an amino acid sequence of SEQ ID NO:127, a VH CDR2 having an amino acid sequence of SEQ ID NO:128, and a VH CDR3 having an amino acid sequence of SEQ ID NO:129, and
(ii) a VL comprising a VL CDR1 having an amino acid sequence of SEQ ID NO:130, a VL CDR2 having an amino acid sequence of SEQ ID NO:131, and a VL CDR3 having an amino acid sequence of SEQ ID NO: 132.
   95. The bispecific antibody of embodiment 94, wherein the first binding domain comprises a VH having an amino acid sequence of SEQ ID NO:133.
   96. The bispecific antibody of embodiment 94, wherein the first binding domain comprises a VL having an amino acid sequence of SEQ ID NO:134.
   97. The bispecific antibody of embodiment 94, wherein the first binding domain comprises a VH having an amino acid sequence of SEQ ID NO:133, and a VL having an amino acid sequence of SEQ ID NO:134.
   98. The bispecific antibody of any one of embodiments 53 to 97, wherein the second target is a cancer antigen present on the surface of a cancer cell.
   99. The bispecific antibody of embodiment 98, wherein the antigen on the surface of the cancer cell is a tumor-specific antigen, a tumor associated antigen, or a neoantigen.
   100. The bispecific antibody of any one of embodiments 53 to 99, wherein the second target is CD123.
   101. The bispecific antibody of embodiment 100, wherein the second binding domain comprises:
(i) a VH comprising a VH CDR1 having an amino acid sequence of SEQ ID NO:9, a VH CDR2 having an amino acid sequence of SEQ ID NO:10, and a VH CDR3 having an amino acid sequence of SEQ ID NO:11, and
(ii) a VL comprising a VL CDR1 having an amino acid sequence of SEQ ID NO:12, a VL CDR2 having an amino acid sequence of SEQ ID NO:13, and a VL CDR3 having an amino acid sequence of SEQ ID NO: 14.
   102. The bispecific antibody of embodiment 101, wherein the second binding domain comprises a VH having an amino acid sequence of SEQ ID NO:15.
   103. The bispecific antibody of embodiment 101, wherein the second binding domain comprises a VL having an amino acid sequence of SEQ ID NO:16.
   104. The bispecific antibody of embodiment 101, wherein the second binding domain comprises a VH having an amino acid sequence of SEQ ID NO:15, and a VL having an amino acid sequence of SEQ ID NO:16.
   105. The bispecific antibody of any one of embodiments 53 to 104, wherein the TRGV9 is present on the surface of a γδ T cell.
   106. The bispecific antibody of any one of embodiments 53 to 104, wherein the TRGV9 is present on the surface of a γδ T cell, and the second target is a cancer antigen expressed on the surface of the cancer cell.
   107. The bispecific antibody of embodiment 106, wherein the cancer cell is killed when the bispecific antibody binds to the TRGV9 on the surface of the γδ T cell and the antigen on the surface of the cancer cell.
   108. The bispecific antibody of any one of embodiments 53 to 107, wherein the first binding domain is humanized, the second binding domain is humanized, or both the first binding domain and the second binding domain are humanized.
   109. The bispecific antibody of any one of embodiments 53 to 108, wherein the bispecific antibody is an IgG antibody.
   110. The bispecific antibody of embodiment 109, wherein the IgG antibody is an IgG1, IgG2, IgG3, or IgG4 antibody.
   111. A pharmaceutical composition comprising the bispecific antibody of any one of embodiments 53 to 110, and a pharmaceutically acceptable carrier.
   112. A method of directing a γδ T cell expressing TRGV9 to a cancer cell, the method comprising contacting the γδ T cell with the bispecific antibody of any one of embodiments 53 to 110, wherein the contacting directs the γδ T cell to the cancer cell.
   113. A method of inhibiting growth or proliferation of cancer cells expressing a cancer antigen on the cell surface, the method comprising contacting the cancer cells with the bispecific antibody of any one of embodiments 53 to 110, wherein contacting the cancer cells with the pharmaceutical composition inhibits growth or proliferation of the cancer cells.
   114. The method of embodiment 113, wherein the cancer cells are in the presence of a γδ T cell expressing TRGV9 while in contact with the bispecific antibody.
   115. A method for eliminating cancer cells in a subject, comprising administering an effective amount of the bispecific antibody of any one of embodiments 53 to 110 to the subject.
   116. The method of embodiment 115, wherein the subject is a subject in need thereof.
   117. The method of embodiments 115 or 116, wherein the subject is a human.
   118. A method of activating a γδ T cell expressing TRGV9, comprising contacting the γδ T cell with the bispecific antibody of any one of embodiments 53 to 110.

In another set of embodiments, provided are:

1. An isolated TRGV9 bispecific antibody or antigen binding fragment thereof, the isolated TRGV9 bispecific antibody or antigen binding fragment thereof comprising:
a. a first heavy chain (HC1);
b. a second heavy chain (HC2);
c. a first light chain (LC1); and
d. a second light chain (LC2),
wherein HC1 is associated with LC1 and HC2 is associated with LC2, and
wherein HC1 comprises a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
and LC1 comprises a light chain complementarity determining region 1 (LCDR1), LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen.
   2. The isolated TRGV9 bispecific antibody or antigen binding fragment thereof of embodiment 1, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
   3. The isolated TRGV9 bispecific antibody or antigen binding fragment thereof of embodiment 2, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
   4. The isolated TRGV9 bispecific antibody or antigen binding fragment thereof any one of embodiments 1 to 3, wherein the binding site for a first antigen binds to TRGV9 on a γδ T cell.
   5. The isolated TRGV9 bispecific antibody or antigen binding fragment thereof any one of embodiments 1 to 4, wherein the binding site for a second antigen binds to a cancer antigen present on the surface of a cancer cell.
   6. The isolated TRGV9 bispecific antibody or antigen binding fragment of embodiment 5, wherein the binding of the bispecific antibody to TRGV9 present on the surface of the γδ T cell and the binding of the cancer antigen present on the surface of the cancer cell results in the killing of the cancer cell.
   7. The isolated TRGV9 bispecific antibody or antigen binding fragment of any one of embodiments 1 to 6, wherein HC1 and LC1 are humanized.
   8. The isolated TRGV9 bispecific antibody or antigen binding fragment thereof any one of embodiments 1 to 7, wherein HC2 and LC2 bind to CD123.
   9. The isolated TRGV9 bispecific antibody or antigen binding fragment thereof any one of embodiments 1 to 8, wherein the bispecific antibody or antigen binding fragment thereof is an IgG1, an IgG2, an IgG3, or an IgG4 isotype.
   10. The isolated TRGV9 bispecific antibody or antigen binding fragment thereof any one of embodiments 1 to 9, wherein the bispecific antibody or antigen binding fragment thereof is an IgG4 isotype.
   11. The isolated TRGV9 bispecific antibody or antigen binding fragment thereof any one of embodiments 1 to 10, wherein the bispecific antibody or antigen binding fragment thereof induces γδ T cell dependent cytotoxicity of a cancer cell in vitro with an EC₅₀ of less than about 500 pM.
   12. The isolated TRGV9 bispecific antibody or antigen binding fragment thereof of embodiment 11, wherein the bispecific antibody or antigen binding fragment thereof induces γδ T cell dependent cytotoxicity of a cancer cell in vitro with an EC₅₀ of less than about 300 pM.
   13. The isolated TRGV9 bispecific antibody or antigen binding fragment thereof of embodiment 11, wherein the bispecific antibody or antigen binding fragment thereof induces γδ T cell dependent cytotoxicity of a cancer cell in vitro with an EC₅₀ of less than about 160 pM.
   14. The isolated TRGV9 bispecific antibody or antigen binding fragment thereof any one of embodiments 11 to 13, wherein the EC₅₀ is assessed with a mixture of γδ T effector cells and Kasumi3 AML target cells.
   15. The isolated TRGV9 bispecific antibody or antigen binding fragment thereof of embodiment 14, wherein the effector cell to target cell ratio is about 0.01 to 1 to about 5 to 1.
   16. The isolated TRGV9 bispecific antibody or antigen binding fragment thereof of embodiment 15, wherein the effector cell to target cell ratio is about 0.1 to 1 to about 2 to 1.
   17. The isolated TRGV9 bispecific antibody or antigen binding fragment thereof of embodiment 16, wherein the effector cell to target cell ratio is about 1:1.
   18. The isolated TRGV9 bispecific antibody or antigen binding fragment thereof any one of embodiments 1 to 17, wherein the bispecific antibody or antigen binding fragment thereof is multivalent.
   19. The isolated TRGV9 bispecific antibody or antigen binding fragment thereof of embodiment 18, wherein the bispecific antibody or antigen binding fragment thereof is capable of binding at least three antigens.
   20. The isolated TRGV9 bispecific antibody or antigen binding fragment thereof of embodiment 18, wherein the bispecific antibody or antigen binding fragment thereof is capable of binding at least five antigens.
   21. An isolated γδ T cell bispecific antibody or antigen binding fragment thereof, the isolated γδ T cell bispecific antibody or antigen binding fragment thereof comprising:
a. a first heavy chain (HC1);
b. a second heavy chain (HC2);
c. a first light chain (LC1); and
d. a second light chain (LC2),
wherein HC1 is associated with LC1 and HC2 is associated with LC2,
wherein HC1 and LC1 form a binding site for a first antigen on a γδ T cell, and
wherein HC2 and LC2 form a binding site for a second antigen.
   22. A bispecific antibody comprising: a first means capable of specifically binding a T cell receptor gamma chain; and a second means capable of specifically binding a target molecule that is not a T cell receptor gamma chain.
   23. A process for making a molecule capable of specifically binding to more than one target molecule, the molecule comprising: a step for performing a function of obtaining an oligopeptide or polypeptide capable of binding to a T cell receptor gamma chain; a step for performing a function of obtaining an oligopeptide or polypeptide capable of binding to a target; and a step for performing a function of providing a molecule capable of specifically binding to a T cell receptor gamma chain and a target molecule.
   24. The process of embodiment 23, wherein the step for performing a function of obtaining an oligopeptide or polypeptide capable of binding to a target is repeated n times and further comprising n steps for performing a function of providing a molecule capable of specifically binding to a T cell receptor gamma chain and n number of target molecules, wherein n is at least 2.

In another set of embodiments, provided are:

1. An isolated anti-TRGV9/anti-CD123 bispecific antibody or antigen binding fragment thereof comprising:
a. a first heavy chain (HC1);
b. a second heavy chain (HC2)
c. a first light chain (LC1); and
d. a second light chain (LC2),
wherein HC1 is associated with LC1 and HC2 is associated with LC2, and
wherein HC1 comprises a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
and LC1 comprises a light chain complementarity determining region 1 (LCDR1), LCDR2, and LCDR3 comprising the amino acid sequences of SEQ ID NO:4, SEQ ID NO:5, and SEQ ID NO:6, respectively, to form a binding site for a first antigen that specifically binds Vγ9, and wherein HC2 comprises a heavy chain complementarity determining region 1 (HCDR1), HCDR2, and HCDR3 comprising the amino acid sequences of SEQ ID NO:9, SEQ ID NO:10, and SEQ ID NO:11, respectively, and LC2 comprises a light chain complementarity determining region 1 (LCDR1), LCDR2, and LCDR3 comprising the amino acid sequences of SEQ ID NO: 12, SEQ ID NO:13, and SEQ ID NO:14, respectively, to form a binding site for a second antigen that specifically binds CD123.
   2. The isolated anti-TRGV9/anti-CD123 bispecific antibody or antigen binding fragment thereof of embodiment 1, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
   3. The isolated anti-TRGV9/anti-CD123 bispecific antibody or antigen binding fragment thereof of embodiment 2, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
   4. The isolated anti-TRGV9/anti-CD123 bispecific antibody or antigen binding fragment thereof any one of embodiments 1 to 3, wherein HC2 comprises an amino acid sequence having at least 95% identity to the amino acid sequence of SEQ ID NO:15 and LC2 comprises an amino acid sequence having at least 95% identity to the amino acid sequence of SEQ ID NO:16.
   5. The isolated anti-TRGV9/anti-CD123 bispecific antibody or antigen binding fragment thereof of embodiment 4, wherein HC2 comprises the amino acid sequence of SEQ ID NO:15 and LC2 comprises the amino acid sequence of SEQ ID NO:16.
   6. The isolated anti-TRGV9/anti-CD123 bispecific antibody or antigen binding fragment thereof any one of embodiments 1 to 5, wherein the TRGV9 is on the surface of a γδ T cell.
   7. The isolated anti-TRGV9/anti-CD123 bispecific antibody or antigen binding fragment thereof any one of embodiments 1 to 6, wherein the CD123 is on the surface of a tumor cell or a CD34+ stem cell.
   8. The isolated anti-TRGV9/anti-CD123 bispecific antibody or antigen binding fragment thereof any one of embodiments 1 to 7, wherein the binding of the bispecific antibody to TRGV9 present on the surface of the γδ T cell and the binding of the CD123 on the surface of the cancer cell results in the killing of the cancer cell.
   9. The isolated anti-TRGV9/anti-CD123 bispecific antibody or antigen binding fragment thereof any one of embodiments 1 to 8, wherein HC1 and LC1 are humanized.
   10. The isolated anti-TRGV9/anti-CD123 bispecific antibody or antigen binding fragment thereof any one of embodiments 1 to 9, wherein HC2 and LC2 are humanized.
   11. The isolated anti-TRGV9/anti-CD123 bispecific antibody or antigen binding fragment thereof any one of embodiments 1 to 10, wherein the bispecific antibody or antigen binding fragment thereof is an IgG1, an IgG2, an IgG3, or an IgG4 isotype.
   12. The isolated anti-TRGV9/anti-CD123 bispecific antibody or antigen binding fragment thereof any one of embodiments 1 to 11, wherein the bispecific antibody or antigen binding fragment thereof is an IgG4 isotype.
   13. The isolated anti-TRGV9/anti-CD123 bispecific antibody or antigen binding fragment thereof any one of embodiments 1 to 12, wherein the bispecific antibody or antigen binding fragment thereof induces γδ T cell dependent cytotoxicity of a cancer cell in vitro with an EC₅₀ of less than about 500 pM.
   14. The isolated anti-TRGV9/anti-CD123 bispecific antibody or antigen binding fragment thereof of embodiment 13, wherein the bispecific antibody or antigen binding fragment thereof induces γδ T cell dependent cytotoxicity of a cancer cell in vitro with an EC₅₀ of less than about 300 pM.
   15. The isolated anti-TRGV9/anti-CD123 bispecific antibody or antigen binding fragment thereof of embodiment 13, wherein the bispecific antibody or antigen binding fragment thereof induces γδ T cell dependent cytotoxicity of a cancer cell in vitro with an EC₅₀ of less than about 160 pM.
   16. The isolated anti-TRGV9/anti-CD123 bispecific antibody or antigen binding fragment thereof any one of embodiments 13 to 15, wherein the EC₅₀ is assessed with a mixture of γδ T effector cells and Kasumi3 AML target cells.
   17. The isolated anti-TRGV9/anti-CD123 bispecific antibody or antigen binding fragment thereof of embodiment 16, wherein the effector cell to target cell ratio is about 0.01 to 1 to about 5 to 1.
   18. The isolated anti-TRGV9/anti-CD123 bispecific antibody or antigen binding fragment thereof of embodiment 17, wherein the effector cell to target cell ratio is about 0.1 to 1 to about 2 to 1.
   19. The isolated anti-TRGV9/anti-CD123 bispecific antibody or antigen binding fragment thereof of embodiment 18, wherein the effector cell to target cell ratio is about 1:1.
   20. A method of making the isolated anti-TRGV9/anti-CD123 bispecific antibody or antigen binding fragment thereof any one of embodiments 1 to 19, the method comprising culturing a cell comprising a nucleic acid encoding the anti-TRGV9/anti-CD123 bispecific antibody or antigen binding fragment thereof under conditions to produce the bispecific antibody or antigen binding fragment thereof and recovering the bispecific antibody or antigen binding fragment thereof.

In another set of embodiments, provided are:

1. An isolated TRGV9 bispecific antibody or antigen epitope binding fragment thereof, wherein the isolated TRGV9 bispecific antibody or antigen epitope binding fragment thereof comprises a binding site for a first antigen and a binding site for a second antigen, wherein the binding site for the first antigen binds a TRGV9 epitope on a γδ T cell and the binding site for the second antigen binds an epitope of the second antigen on a surface of a target cell, and the binding of the TRGV9 epitope on the γδ T cell and the binding of the second antigen epitope on the target cell results in the killing of the target cell.
2. The isolated TRGV9 bispecific antibody or antigen binding fragment thereof, wherein the isolated TRGV9 bispecific antibody or antigen binding fragment thereof comprises:
a. a first heavy chain (HC1);
b. a second heavy chain (HC2);
c. a first light chain (LC1); and
d. a second light chain (LC2),
wherein HC1 is associated with LC1 and HC2 is associated with LC2, and
wherein HC1 comprises a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
and LC1 comprises a light chain complementarity determining region 1 (LCDR1), LCDR2, and LCDR2 comprising the amino acid sequences of SEQ ID NO:4, SEQ ID NO:5, and SEQ ID NO:6, respectively, to form the binding site for the first antigen, and wherein HC2 and LC2 form the binding site for the second antigen epitope.
   3. The isolated TRGV9 bispecific antibody or antigen binding fragment thereof of embodiment 2, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
   4. The isolated TRGV9 bispecific antibody or antigen binding fragment thereof of embodiment 3, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
   5. The isolated TRGV9 bispecific antibody or antigen binding fragment of any one of embodiments 2 to 4, wherein HC1 and LC1 are humanized.
   6. The isolated TRGV9 bispecific antibody or antigen binding fragment thereof any one of embodiments 2 to 5, wherein HC2 and LC2 bind to a CD123 epitope.
   7. The isolated TRGV9 bispecific antibody or antigen binding fragment thereof of embodiment 6, wherein HC2 comprises an amino acid sequence having at least 95% identity to the amino acid sequence of SEQ ID NO:15 and LC2 comprises an amino acid sequence having at least 95% identity to the amino acid sequence of SEQ ID NO:16.
   8. The isolated TRGV9 bispecific antibody or antigen binding fragment thereof of embodiment 7, wherein HC2 comprises the amino acid sequence of SEQ ID NO:15 and LC2 comprises the amino acid sequence of SEQ ID NO:16.
   9. The isolated TRGV9 bispecific antibody or antigen binding fragment thereof any one of embodiments 1 to 8, wherein the bispecific antibody or antigen binding fragment thereof is an IgG1, an IgG2, an IgG3, or an IgG4 isotype.
   10. The isolated TRGV9 bispecific antibody or antigen binding fragment thereof any one of embodiments 1 to 9, wherein the bispecific antibody or antigen binding fragment thereof is an IgG4 isotype.
   11. The isolated TRGV9 bispecific antibody or antigen binding fragment thereof any one of embodiments 1 to 10, wherein the bispecific antibody or antigen binding fragment thereof induces γδ T cell dependent cytotoxicity of a cancer cell in vitro with an EC₅₀ of less than about 500 pM.
   12. The isolated TRGV9 bispecific antibody or antigen binding fragment thereof of embodiment 11, wherein the bispecific antibody or antigen binding fragment thereof induces γδ T cell dependent cytotoxicity of a cancer cell in vitro with an EC₅₀ of less than about 300 pM.
   13. The isolated TRGV9 bispecific antibody or antigen binding fragment thereof of embodiment 12, wherein the bispecific antibody or antigen binding fragment thereof induces γδ T cell dependent cytotoxicity of a cancer cell in vitro with an EC₅₀ of less than about 160 pM.
   14. The isolated TRGV9 bispecific antibody or antigen binding fragment thereof any one of embodiments 11 to 13, wherein the EC₅₀ is assessed with a mixture of γδ T effector cells and Kasumi3 AML target cells.
   15. The isolated TRGV9 bispecific antibody or antigen binding fragment thereof of embodiment 14, wherein the effector cell to target cell ratio is about 0.01 to 1 to about 5 to 1.
   16. The isolated TRGV9 bispecific antibody or antigen binding fragment thereof of embodiment 15, wherein the effector cell to target cell ratio is about 0.1 to 1 to about 2 to 1.
   17. The isolated TRGV9 bispecific antibody or antigen binding fragment thereof of embodiment 16, wherein the effector cell to target cell ratio is about 1:1.
   18. An isolated γδ T cell bispecific antibody or antigen binding fragment thereof, wherein the isolated γδ T cell bispecific antibody or antigen binding fragment thereof comprises a binding site for a first antigen epitope and a binding site for a second antigen epitope, wherein the binding site for the first antigen epitope binds a first antigen on a γδ T cell and the binding site for the second antigen epitope binds the second antigen epitope on a surface of a target cell, and the binding of the first antigen epitope on the γδ T cell and the binding of the second antigen epitope on the target cell results in the killing of the target cell.

In another set of embodiments, provided are:

1. An isolated nucleic acid encoding a TRGV9 bispecific antibody or antigen binding fragment thereof, the isolated TRGV9 bispecific antibody or antigen binding fragment thereof comprising:
a. a first heavy chain (HC1);
b. a second heavy chain (HC2);
c. a first light chain (LC1); and
d. a second light chain (LC2),
wherein HC1 is associated with LC1 and HC2 is associated with LC2, and
wherein HC1 comprises a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
and LC1 comprises a light chain complementarity determining region 1 (LCDR1), LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen.
   2. The isolated nucleic acid of embodiment 1, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
   3. The isolated nucleic acid of embodiment 2, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO:8.
   4. The isolated nucleic acid of any one of embodiments 1 to 3, wherein the binding site for a first antigen binds to TRGV9 on a γδ T cell.
   5. The isolated nucleic acid of any one of embodiments 1 to 4, wherein the binding site for a second antigen binds to a cancer antigen present on the surface of a cancer cell.
   6. The isolated nucleic acid of embodiment 5, wherein the binding of the bispecific antibody to TRGV9 present on the surface of the γδ T cell and the binding of the cancer antigen present on the surface of the cancer cell results in the killing of the cancer cell.
   7. The isolated nucleic acid of any one of embodiments 1 to 6, wherein HC1 and LC1 are humanized.
   8. The isolated nucleic acid of any one of embodiments 1 to 7, wherein HC2 and LC2 bind to CD123.
   9. The isolated nucleic acid of any one of embodiments 1 to 8, wherein the bispecific antibody or antigen binding fragment thereof is an IgG1, an IgG2, an IgG3, or an IgG4 isotype.
   10. The isolated nucleic acid of any one of embodiments 1 to 9, wherein the bispecific antibody or antigen binding fragment thereof is an IgG4 isotype.
   11. The isolated nucleic acid of any one of embodiments 1 to 10, wherein the bispecific antibody or antigen binding fragment thereof induces γδ T cell dependent cytotoxicity of a cancer cell in vitro with an EC₅₀ of less than about 500 pM.
   12. The isolated nucleic acid of embodiment 11, wherein the bispecific antibody or antigen binding fragment thereof induces γδ T cell dependent cytotoxicity of a cancer cell in vitro with an EC₅₀ of less than about 300 pM.
   13. The isolated nucleic acid of embodiment 11, wherein the bispecific antibody or antigen binding fragment thereof induces γδ T cell dependent cytotoxicity of a cancer cell in vitro with an EC₅₀ of less than about 160 pM.
   14. The isolated nucleic acid of any one of embodiments 11 to 13, wherein the EC₅₀ is assessed with a mixture of γδ T effector cells and Kasumi3 AML target cells.
   15. The isolated nucleic acid of embodiment 14, wherein the effector cell to target cell ratio is about 0.01 to 1 to about 5 to 1.
   16. The isolated nucleic acid of embodiment 15, wherein the effector cell to target cell ratio is about 0.1 to 1 to about 2 to 1.
   17. The isolated nucleic acid of embodiment 16, wherein the effector cell to target cell ratio is about 1:1.
   18. The isolated nucleic acid of any one of embodiments 1 to 17, wherein the bispecific antibody or antigen binding fragment thereof is multivalent.
   19. The isolated nucleic acid of embodiment 18, wherein the bispecific antibody or antigen binding fragment thereof is capable of binding at least three antigens.
   20. The isolated nucleic acid of embodiment 18, wherein the bispecific antibody or antigen binding fragment thereof is capable of binding at least five antigens.
   21. A vector comprising the isolated nucleic acid of any one of embodiments 1 to 20.
   22. A host cell comprising the vector of embodiment 21.
   23. A kit comprising the vector of embodiment 21 and packaging for the same.

In another set of embodiments, provided are:

1. A pharmaceutical composition comprising an isolated TRGV9 bispecific antibody or antigen binding fragment thereof, the isolated TRGV9 bispecific antibody or antigen binding fragment thereof comprising:
a. a first heavy chain (HC1);
b. a second heavy chain (HC2);
c. a first light chain (LC1); and
d. a second light chain (LC2),
wherein HC1 is associated with LC1 and HC2 is associated with LC2, and
wherein HC1 comprises a heavy chain complementarity determining region 1 (HCDR1), HCDR2, and HCDR3 comprising the amino acid sequences of:
i. SEQ ID NO:1, SEQ ID NO:2, and SEQ ID NO:3, respectively,
ii. SEQ ID NO:1, SEQ ID NO:2, and SEQ ID NO:31, respectively,
iii. SEQ ID NO:1, SEQ ID NO:2, and SEQ ID NO:32, respectively, or
iv. SEQ ID NO:1, SEQ ID NO:2, and SEQ ID NO:33, respectively,
and LC1 comprises a light chain complementarity determining region 1 (LCDR1), LCDR2, and LCDR2 comprising the amino acid sequences of SEQ ID NO:4, SEQ ID NO:5, and SEQ ID NO:6, respectively, to form a binding site for a first antigen, and wherein HC2 and LC2 form a binding site for a second antigen, and a pharmaceutically acceptable carrier.
   2. The pharmaceutical composition of embodiment 1, wherein HC1 comprises an amino acid sequence having at least 95% identity to an amino acid sequence selected from SEQ ID NO:7, SEQ ID NO:34, SEQ ID NO:35, or SEQ ID NO:36, and LC1 comprises an amino acid sequence having at least 95% identity to the amino acid sequence of SEQ ID NO:8.
   3. The pharmaceutical composition of embodiment 2, wherein HC1 comprises the amino acid sequence selected from SEQ ID NO:7, SEQ ID NO:34, SEQ ID NO:35, or SEQ ID NO:36, and LC1 comprises the amino acid sequence of SEQ ID NO: 8.
   4. The pharmaceutical composition of any one of embodiments 1 to 3, wherein the binding site for a first antigen binds to TRGV9 on a γδ T cell.
   5. The pharmaceutical composition of any one of embodiments 1 to 4, wherein the binding site for a second antigen binds to a cancer antigen present on the surface of a cancer cell.
   6. The pharmaceutical composition of embodiment 5, wherein the binding of the bispecific antibody to TRGV9 present on the surface of the γδ T cell and the binding of the cancer antigen present on the surface of the cancer cell results in the killing of the cancer cell.
   7. The pharmaceutical composition of any one of embodiments 1 to 6, wherein HC1 and LC1 are humanized.
   8. The pharmaceutical composition of any one of embodiments 1 to 7, wherein HC2 and LC2 bind to CD123.
   9. The pharmaceutical composition of any one of embodiments 1 to 8, wherein the bispecific antibody or antigen binding fragment thereof is an IgG1, an IgG2, an IgG3, or an IgG4 isotype.
   10. A method of directing a Vγ9-expressing γδ T cell to a cancer cell, the method comprising contacting a Vγ9-expressing γδ T cell with the pharmaceutical composition of any one of embodiments 1 to 9, wherein contacting the Vγ9-expressing γδ T cell with the pharmaceutical composition directs the Vγ9-expressing γδ T cell to a cancer cell.
   11. A method of inhibiting growth or proliferation of cancer cells expressing a cancer antigen on the cell surface, the method comprising contacting the cancer cells with the pharmaceutical composition of any one of embodiments 1 to 9, wherein contacting the cancer cells with the pharmaceutical composition inhibits growth or proliferation of the cancer cells.
   12. The method of embodiment 11, wherein the cancer cell is in the presence of a Vγ9-expressing γδ T cell while in contact with anti-TRGV9 bispecific antibody or antigen binding fragment thereof.
   13. A method for treating a cancer in a subject in need thereof, the method comprising:
a. identifying a subject in need of cancer treatment; and
b. administering to the subject in need thereof the pharmaceutical composition of any one of embodiments 1 to 9,
wherein administering the pharmaceutical composition to the subject in need thereof treats the cancer in the subject.
   14. A method of activating a Vγ9-expressing γδ T cell, the method comprising contacting the Vγ9-expressing γδ T cell with the pharmaceutical composition of any one of embodiments 1 to 9, wherein contacting the Vγ9-expressing γδ T cell with the pharmaceutical composition results in an increase in CD69, CD25, and/or Granzyme B expression as compared to a control Vγ9-expressing γδ T cell.
   15. A method of producing the pharmaceutical composition of any one of embodiments 1 to 9, the method comprising combining the bispecific antibody or antigen binding fragment thereof with a pharmaceutically acceptable carrier to obtain the pharmaceutical composition.

Provided in the Examples herein are exemplary multi-specific (bispecific) antibodies that bind to TRGV9 and CD123 (also known as IL3RA). CD123 is expressed on a variety of cell types in various tissues, including adipose tissue, adrenal gland, appendix, bone marrow, breast, bronchus, caudate, cerebellum, cerebral cortex, cervix, uterine, colon, duodenum, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine, smooth muscle, soft tissue, spleen, stomach, testis, thyroid gland, tonsil, urinary bladder, and vagina (see, e.g., proteinatlas.org). Thus, these Examples are illustrative of exemplary bispecific antibodies that can effectively target a wide variety of cells and tissues in a subject.

In some embodiments, provided herein is a bispecific antibody comprising: (a) a first binding domain that binds to a TRGV9 antigen, and (b) a second binding domain that binds to a second target antigen. In some embodiments, provided herein is a bispecific antibody comprising: (a) a first binding domain that specifically binds to a TRGV9 antigen, and (b) a second binding domain that specifically binds to a second target antigen. In some embodiments, provided herein is a bispecific antibody comprising: (a) a first binding domain that binds to a first epitope on a TRGV9 antigen, and (b) a second binding domain that binds to a second epitope on a second target antigen. In some embodiments, provided herein is a bispecific antibody comprising: (a) a first binding domain that specifically binds to a first epitope on a TRGV9 antigen, and (b) a second binding domain that specifically binds to a second epitope on a second target antigen. In certain embodiments, the second target antigen is a cancer antigen on the surface of a cancer cell. In certain embodiments, the second target antigen is CD123.

Exemplary binding agents that bind to TRGV9, as well as exemplary binding agents that bind to CD123 are provided elsewhere herein, for example in the Examples, as well as in Tables 1-31.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

### Example 1: Production of Multispecific Antibodies that Bind γδ T Cells

1.1: Production of Mabs that Bind γδ T Cell Antigens

Antigens or portions of antigens specific for γδ T cells are used to immunize an animal (e.g., a mouse or a rabbit). To generate the γδ T cell monoclonal antibodies, peripheral blood mononuclear cells are isolated from the whole blood of the immunized animal, and antigen specific B cells are grown. B cells secreting reactive antibodies for the γδ T cell antigens are identified by an antigen-binding ELISA screening of the B cell culture supernatants. High binding ELISA plates are coated with the γδ T cell antigen overnight. The ELISA plates are blocked, and diluted B cell culture supernatants are added to the plates. The plates are incubated at room temperature and following incubation, a secondary antibody specific for recognizing the γδ T cell antigen antibody is added to the plate to determine if the γδ T cell antigen antibody bound the γδ T cell antigen. Binding of the antibody is determined by reaction of a substrate on the secondary antibody.

After the identification of monoclonal antibodies that are capable of binding γδ T cell antigens, the variable regions of the heavy and light chains of the γδ T cell antibody are sequenced. Constructs are created for the expression of the heavy and light chain of the γδ T cell antibody. The constructs are transfected into a host cell to express the heavy and light chains, and the γδ T cell antibody is isolated from the supernatant.

### 1.2: Production of γδ T Cell Bispecific Antibodies

The variable region sequence of the γδ T cell monoclonal antibody and a second monoclonal antibody capable of binding a target antigen on a target cell of interest are used to generate a bispecific antibody to be tested for γδ T cell re-directed killing of the target cells. Target antigens of interest can be selected from, but not limited, antigens described in Zhang et al., Nucleic Acids Research 47(D1):D721-D728 (2019). γδ T cell bispecific antibodies are produced as full-length antibodies in the knob-into-hole format as human IgG4, as previously described (Atwell et al., J. Mol. Biol. 270:26-35 (1997)). Nucleic acid sequences encoding variable regions are sub-cloned into custom mammalian expression vectors containing the constant region of IgG4 expression cassettes using standard PCR restriction enzyme based cloning techniques. The bispecific antibodies are expressed by transient transfection in Chinese hamster ovary cell line. The antibodies are initially purified by MAB SELECT SURE Protein A column (GE Healthcare, Piscataway, New Jersey) (Brown, Bottomley et al. Biochem Soc Trans. 1998 August; 26(3):S249). The column is equilibrated with Phosphate Buffer Saline (PBS), pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### 1.3: Production of Anti-Trgv9 Bispecific Antibodies

Variable region sequences of exemplary anti-TRGV9 monoclonal antibodies are provided below in Table 1 and Table 2.

TABLE 1
Anti-TRGV9 mAb CDR Sequences

| Antibody | VH CDR1 | VH CDR2 | VH CDR3 | VL CDR1 | VL CDR2 | VL CDR3 |
|---|---|---|---|---|---|---|
| TRGV9Ab_1 | DHYIN | QIYPGDGNT | NYGDYTIDF | KSSQSLLYS | WASTRES | QQYYRYHT |
| (LP7A5_1) | (SEQ ID | YYNQKFKG | (SEQ ID | SNQKNYLA | (SEQ ID | (SEQ ID |
| | NO: 1) | (SEQ ID | NO: 3) | (SEQ ID | NO: 5) | NO: 6) |
| | | NO: 2) | | NO: 4) | | |
| TRGV9Ab_2 | DHYIN | QIYPGDGNT | NMGMYTIDF | KSSQSLLYS | WASTRES | QQYYRYHT |
| (LP7A5_2) | (SEQ ID | YYNQKFKG | (SEQ ID | SNQKNYLA | (SEQ ID | (SEQ ID |
| | NO: 1) | (SEQ ID | NO: 31) | (SEQ ID | NO: 5) | NO: 6) |
| | | NO: 2) | | NO: 4) | | |
| TRGV9Ab_3 | DHYIN | QIYPGDGNT | NMGMYTLDF | KSSQSLLYS | WASTRES | QQYYRYHT |
| (LP7A5_3) | (SEQ ID | YYNQKFKG | (SEQ ID | SNQKNYLA | (SEQ ID | (SEQ ID |
| | NO: 1) | (SEQ ID | NO: 32) | (SEQ ID | NO: 5) | NO: 6) |
| | | NO: 2) | | NO: 4) | | |
| TRGV9Ab_4 | DHYIN | QIYPGDGNT | NYGDYTLDF | KSSQSLLYS | WASTRES | QQYYRYHT |
| (LP7A5_4) | (SEQ ID | YYNQKFKG | (SEQ ID | SNQKNYLA | (SEQ ID | (SEQ ID |
| | NO: 1) | (SEQ ID | NO: 33) | (SEQ ID | NO: 5) | NO: 6) |
| | | NO: 2) | | NO: 4) | | |

TABLE 2
Anti-TRGV9 mAb VH and VL Domain Sequences

| Antibody | VH | VL |
|---|---|---|
| TRGV9Ab_1 | EVQLQQSGAELARPGASVKLSCKASGFTFTDHY | DIVMSQSPSSLAVSVGEKVTMSCKSSQSLLYSSN |
| (LP7A5_1) | INWVKQRTGQGLEWIGQIYPGDGNTYYNQKFKG | QKNYLAWYQQKPGQSPKLLIYWASTRESGVPDRF |
| | KATLTADKSSSTAYMQLSSLTSEDSAVYFCAP | TGSGSGTDFTLTISSVKAEDLAVYYCQQYYRY |
| | NYGDYTIDFWGQGTSVTVSS | HTFGTGTKLEIK |
| | (SEQ ID NO: 7) | (SEQ ID NO: 8) |
| TRGV9Ab_2 | EVQLQQSGAELARPGASVKLSCKASGFTFTDHY | DIVMSQSPSSLAVSVGEKVTMSCKSSQSLLYSSN |
| (LP7A5_2) | INWVKQRTGQGLEWIGQIYPGDGNTYYNQKFKG | QKNYLAWYQQKPGQSPKLLIYWASTRESGVPDRF |
| | KATLTADKSSSTAYMQLSSLTSEDSAVYFCAP | TGSGSGTDFTLTISSVKAEDLAVYYCQQYYRY |
| | NMGMYTIDFWGQGTSVTVSS | HTFGTGTKLEIK |
| | (SEQ ID NO: 34) | (SEQ ID NO: 8) |
| TRGV9Ab_3 | EVQLQQSGAELARPGASVKLSCKASGFTFTDHY | DIVMSQSPSSLAVSVGEKVTMSCKSSQSLLYSSN |
| (LP7A5_3) | INWVKQRTGQGLEWIGQIYPGDGNTYYNQKFKG | QKNYLAWYQQKPGQSPKLLIYWASTRESGVPDRF |
| | KATLTADKSSSTAYMQLSSLTSEDSAVYFCAP | TGSGSGTDFTLTISSVKAEDLAVYYCQQYYRY |
| | NMGMYTLDFWGQGTSVTVSS | HTFGTGTKLEIK |
| | (SEQ ID NO: 35) | (SEQ ID NO: 8) |
| TRGV9Ab_4 | EVQLQQSGAELARPGASVKLSCKASGFTFTDHY | DIVMSQSPSSLAVSVGEKVTMSCKSSQSLLYSSN |
| (LP7A5_4) | INWVKQRTGQGLEWIGQIYPGDGNTYYNQKFKG | QKNYLAWYQQKPGQSPKLLIYWASTRESGVPDRF |
| | KATLTADKSSSTAYMQLSSLTSEDSAVYFCAP | TGSGSGTDFTLTISSVKAEDLAVYYCQQYYRY |
| | NYGDYTLDFWGQGTSVTVSS | HTFGTGTKLEIK |
| | (SEQ ID NO: 36) | (SEQ ID NO: 8) |

Variable region sequences of an anti-TRGV9 monoclonal antibody and a second monoclonal antibody capable of binding a target antigen on a target cell of interest are used to generate a bispecific antibody to be tested for γδ T cell re-directed killing of the target cells. Target antigens of interest can be selected from, but not limited to, antigens described in Zhang et al., Nucleic Acids Research 47(D1):D721-D728 (2019). Anti-TRGV9 bispecific antibodies are produced as full-length antibodies in the knob-into-hole format as human IgG4, as previously described (Atwell et al., J. Mol. Biol. 270:26-35 (1997)). Nucleic acid sequences encoding variable regions are sub-cloned into custom mammalian expression vectors containing the constant region of IgG4 expression cassettes using standard PCR restriction enzyme based cloning techniques. The bispecific antibodies are expressed by transient transfection in Chinese hamster ovary cell line. The antibodies are initially purified by MAB SELECT SURE Protein A column (GE Healthcare, Piscataway, New Jersey) (Brown, Bottomley et al. Biochem Soc Trans. 1998 August; 26(3):S249). The column is equilibrated with Phosphate Buffer Saline (PBS), pH 7.2 and is loaded with fermentation supernatant at a flow rate of 2 mL/min. After loading, the column is washed with PBS (4 column volumes (CV)) followed by elution in 30 mM sodium acetate, pH 3.5. Fractions containing protein peaks as monitored by Absorbance at 280 nm in Akta Explorer (GE healthcare) are pooled together and are neutralized to pH 5.0 by adding 1% of 3M sodium acetate, pH 9.0. As a polishing step, the antibodies are purified on a preparative size exclusion chromatography (SEC) using a SUPERDEX 200 column (GE healthcare). The integrity of sample is assessed by endotoxin measurement and SDS polyacrylamide gel electrophoresis under reducing and non-reducing conditions. The final protein concentrations are determined.

### Example 2—Bispecific Antibodies that Bind Trgv9 and a Cancer Antigen

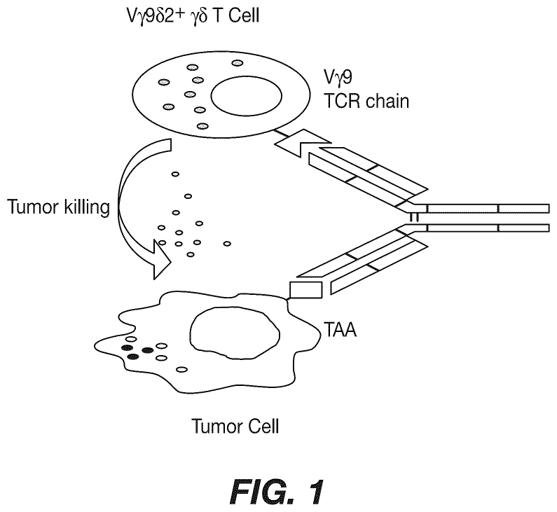

Examples 2.1-2.4 are based on the premise that γδ T cells, which mainly express heterodimers of TRGV9 and Vδ2 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a tumor-associated antigen expressed by the tumor cells. Thus, the bispecific antibody bridges the effector and target cells together-resulting in tumor cell killing. This mechanism of action is described in the schematic outlined in FIG. 1.

The subsequent examples can be divided into the following categories: (1) Generation and characterization of bispecific antibodies capable of binding to the TRGV9 arm expressed on γδ T cells and a cancer antigen (e.g., CD123) on cancer cells (Examples 2.1, 2.2, and 2.3); and (2) Evidence for bispecific antibody-enabled target cell killing by γδ T cells expanded in vitro (Example 2.4).

### Example 2.1: Production of Anti-Trgv9 Mab

The mouse IgG1 anti-human T cell receptor TRGV9 clone 7A5 was sourced commercially. Sample preparation and LC/MSMS analysis were performed by Lake Pharma. (San Carlos, CA). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZIPTIP C18 Pipette Tips and separated on-line using reverse phase chromatography. Mass spectrometry was performed on THERMO Q-EXACTIVE spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra

The sequences obtained are shown in Tables 3-7.

TABLE 3
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_1 | DHYIN | 1 | QIYPGDGNT | 2 | NYGDYTIDF | 3 |
| | | | YYNQKFKG | | | |
| | | SEQ | | SEQ | | SEQ |
| | | ID | | ID | | ID |
| Antibody | LCDR1 | NO: | LCDR2 | NO: | LCDR3 | NO: |
| LP7A5_1 | KSSQSLLYS | 4 | WASTRES | 5 | QQYYRYHT | 6 |
| | SNQKNYLA | | | | | |

TABLE 4
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_2 | DHYIN | 1 | QIYPGDGNT | 2 | NMGMYTIDF | 31 |
| | | | YYNQKFKG | | | |
| | | SEQ | | SEQ | | SEQ |
| | | ID | | ID | | ID |
| Antibody | LCDR1 | NO: | LCDR2 | NO: | LCDR3 | NO: |
| LP7A5_2 | KSSQSLLYS | 4 | WASTRES | 5 | QQYYRYHT | 6 |
| | SNQKNYLA | | | | | |

TABLE 5
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_3 | DHYIN | 1 | QIYPGDGNT | 2 | NMGMYTLDF | 32 |
| | | | YYNQKFKG | | | |
| | | SEQ | | SEQ | | SEQ |
| | | ID | | ID | | ID |
| Antibody | LCDR1 | NO: | LCDR2 | NO: | LCDR3 | NO: |
| LP7A5_3 | KSSQSLLYS | 4 | WASTRES | 5 | QQYYRYHT | 6 |
| | SNQKNYLA | | | | | |

TABLE 6
CDR Sequences of anti-TRGV9 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| LP7A5_4 | DHYIN | 1 | QIYPGDGNT | 2 | NYGDYTLDF | 33 |
| | | | YYNQKFKG | | | |
| | | SEQ | | SEQ | | SEQ |
| | | ID | | ID | | ID |
| Antibody | LCDR1 | NO: | LCDR2 | NO: | LCDR3 | NO: |
| LP7A5_4 | KSSQSLLYS | 4 | WASTRES | 5 | QQYYRYHT | 6 |
| | SNQKNYLA | | | | | |

TABLE 7
Heavy chain and light chain sequences
of anti-TRGV9 mAb.

| | | SEQ |
|---|---|---|
| | | ID |
| mAb | Heavy Chain Amino Acid Sequence | NO: |
| LP7A5_1 | EVQLQQSGAELARPGASVKLSCKASGFTFTDHY | 7 |
| | INWVKQRTGQGLEWIGQIYPGDGNTYYNQKFKG | |
| | KATLTADKSSSTAYMQLSSLTSEDSAVYFCAPN | |
| | YGDYTIDFWGQGTSVTVSS | |
| LP7A5_2 | EVQLQQSGAELARPGASVKLSCKASGFTFTDHY | 34 |
| | INWVKQRTGQGLEWIGQIYPGDGNTYYNQKFKG | |
| | KATLTADKSSSTAYMQLSSLTSEDSAVYFCAPN | |
| | MGMYTIDFWGQGTSVTVSS | |
| LP7A5_3 | EVQLQQSGAELARPGASVKLSCKASGFTFTDHY | 35 |
| | INWVKQRTGQGLEWIGQIYPGDGNTYYNQKFKG | |
| | KATLTADKSSSTAYMQLSSLTSEDSAVYFCAPN | |
| | MGMYTLDFWGQGTSVTVSS | |
| LP7A5_4 | EVQLQQSGAELARPGASVKLSCKASGFTFTDHY | 36 |
| | INWVKQRTGQGLEWIGQIYPGDGNTYYNQKFKG | |
| | KATLTADKSSSTAYMQLSSLTSEDSAVYFCAPN | |
| | YGDYTLDFWGQGTSVTVSS | |
| | | SEQ |
| | | ID |
| | Light Chain Amino Acid Sequence | NO: |
| LP7A5_1 | DIVMSQSPSSLAVSVGEKVTMSCKSSQSLLYSS | 8 |
| | NQKNYLAWYQQKPGQSPKLLIYWASTRESGVPD | |
| | RFTGSGSGTDFTLTISSVKAEDLAVYYCQQYYR | |
| | YHTFGTGTKLEIK | |

### Example 2.2: Preparation of Anti-Trgv9/Anti-CD123 Bispecific Antibodies

The variable region sequence of LP7A5 (anti-TRGV9) and I3RB217 (anti-CD123 antibody) (HCDRs and LCDRs in Table 8, HC and LC in Table 9) were used to generate a bispecific antibody to be tested for T cell re-directed killing of acute myeloid leukemia (AML) cells. VG1 (anti-TRGV9 x CD123) and VG3 (anti-TRGV9 x Null) bispecific antibodies were produced as full-length antibodies in the knob-into-hole format as human IgG4, as previously described (Atwell et al. J. Mol. Biol. 270: 26-35, 1997). Nucleic acid sequences encoding variable regions were sub-cloned into a custom mammalian expression vectors containing constant region of IgG4 expression cassettes using standard PCR restriction enzyme based cloning techniques. The bispecific antibodies were expressed by transient transfection in Chinese hamster ovary cell line. The antibodies were initially purified by MAB SELECT SURE Protein A column (GE Healthcare, Piscataway, New Jersey) (Brown, Bottomley et al. Biochem Soc Trans. 1998 August; 26(3):S249.). The column was equilibrated with Phosphate Buffer Saline (PBS), pH 7.2 and loaded with fermentation supernatant at a flow rate of 2 mL/min. After loading, the column was washed with PBS (4 column volumes (CV)) followed by elution in 30 mM sodium acetate, pH 3.5. Fractions containing protein peaks as monitored by Absorbance at 280 nm in AKTA EXPLORER (GE healthcare) were pooled together and were neutralized to pH 5.0 by adding 1% of 3M sodium acetate, pH 9.0. As a polishing step, the antibodies were purified on a preparative size exclusion chromatography (SEC) using a SUPERDEX 200 column (GE healthcare). The integrity of sample was assessed by endotoxin measurement and SDS polyacrylamide gel electrophoresis under reducing and non-reducing conditions. The final protein concentrations were 1.0 mg/ml for anti-TRGV9/anti-CD123 and 1.0 mg/mL for anti-TRGV9/Null. The final EU levels of anti-TRGV9/anti-CD123 and anti-TRGV9/Null based on these were <3.0 EU/mg.

TABLE 8
CDR Sequences of anti-CD123 mAb.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| I3RB217 | SYWIS | 9 | IIDPSDSDT | 10 | GDGSTDLDY | 11 |
| | | | RYSPSFQG | | | |
| | | SEQ | | SEQ | | SEQ |
| | | ID | | ID | | ID |
| Antibody | LCDR1 | NO: | LCDR2 | NO: | LCDR3 | NO: |
| I3RB217 | RASQSV | 12 | GASSRAT | 13 | QQDYGFPWT | 14 |
| | SSSYL | | | | | |

TABLE 9
Heavy chain and light chain sequences
of anti-CD123 mAb.

| | | SEQ |
|---|---|---|
| | | ID |
| mAb ID | Heavy Chain Amino Acid Sequence | NO: |
| I3RB217 | EVQLVQSGAEVKKPGESLKISCKGSGYSFTS | 15 |
| | YWISWVRQMPGKGLEWMGIIDPSDSDTRYSP | |
| | SFQGQVTISADKSISTAYLQWSSLKASDTAM | |
| | YYCARGDGSTDLDYWGQGTLVTVSS | |
| | | SEQ |
| | | ID |
| | Light Chain Amino Acid Sequence | NO: |
| I3RB217 | EIVLTQSPGTLSLSPGERATLSCRASQSVSS | 16 |
| | SYLAWYQQKPGQAPRLLIYGASSRATGIPDR | |
| | FSGSGSGTDFTLTISRLEPEDFAVYYCQQDY | |
| | GFPWTFGQGTKVEIK | |

TABLE 10
Sequences of half antibodies expressed in CHO cells.

| | | SEQ |
|---|---|---|
| | | ID |
| mAb ID | ‘Knob’ arm and ‘hole’ arm amino acid sequence | NO: |
| VG1 | MAWVWTLLFLMAAAQSIQADIVMSQSPSSLAVSVGEKVTMSCKSSQSLLYS | 17 |
| (ANTI- | SNQKNYLAWYQQKPGQSPKLLIYWASTRESGVPDRFTGSGSGTDFTLTISSV | |
| TRGV9 | KAEDLAVYYCQQYYRYHTFGTGTKLEIKRTVAAPSVFIFPPSDEQLKSGTAS | |
| half | VVCLLNNFYPREAKVQWKVDNALQSGNSQESVTEQDSKDSTYSLSSTLTLS | |
| antibody) | KADYEKHKVYACEVTHQGLSSPVTKSFNRGECGGSEGKSSGSGSESKSTEG | |
| | KSSGSGSESKSTGGSEVQLQQSGAELARPGASVKLSCKASGFTFTDHYINWV | |
| | KQRTGQGLEWIGQIYPGDGNTYYNQKFKGKATLTADKSSSTAYMQLSSLTS | |
| | EDSAVYFCAPNYGDYTIDFWGQGTSVTVSSASTKGPSVFPLAPCSRSTSEST | |
| | AALGCLVKDYFPEPVTVSWNSGALTSGVHTFPAVLQSSGLYSLSSVVTVPSS | |
| | SLGTKTYTCNVDHKPSNTKVDKRVESKYGPPCPPCPAPEAAGGPSVFLFPPK | |
| | PKDTLMISRTPEVTCVVVDVSQEDPEVQFNWYVDGVEVHNAKTKPREEQF | |
| | NSTYRVVSVLTVLHQDWLNGKEYKCKVSNKGLPSSIEKTISKAKGQPREPQ | |
| | VYTLPPSQEEMTKNQVSLSCAVKGFYPSDIAVEWESNGQPENNYKTTPPVL | |
| | DSDGSFFLVSRLTVDKSRWQEGNVFSCSVMHEALHNRFTQKSLSLSLGK | |
| VG1 | MAWVWTLLFLMAAAQSIQAEIVLTQSPGTLSLSPGERATLSCRASQSVSSSY | 18 |
| (anti- | LAWYQQKPGQAPRLLIYGASSRATGIPDRFSGSGSGTDFTLTISRLEPEDFAV | |
| CD123 | YYCQQDYGFPWTFGQGTKVEIKRTVAAPSVFIFPPSDEQLKSGTASVVCLLN | |
| half Ab) | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSTEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSD GSFFL | |
| | YSRLTVDKSRWQEGNVFSCSVMHEALHNHYTQKSLSLSLGK | |
| VG3 | MAWVWTLLFLMAAAQSIQAEIVLTQSPGTLSLSPGERATLSCRASQSVSSSY | 19 |
| (B23B49 | LAWYQQKPGQAPRLLIYGASSRATGIPDRFSGSGSGTDFTLTISRLEPEDFAV | |
| Null half | YYCQQDYGFPWTFGQGTKVEIKRTVAAPSVFIFPPSDEQLKSGTASVVCLLN | |
| Ab) | NFYPREAKVQWKVDNALQSGNSQESVTEQDSKDSTYSLSSTLTLSKADYEK | |
| | HKVYACEVTHQGLSSPVTKSFNRGECGGSEGKSSGSGSESKSTEGKSSGSGS | |
| | ESKSTGGSEVQLVQSGAEVKKPGESLKISCKGSGYSFTSYWISWVRQMPGK | |
| | GLEWMGIIDPSDSDTRYSPSFQGQVTISADKSISTAYLQWSSLKASDTAMYY | |
| | CARGDGSTDLDYWGQGTLVTVSSASTKGPSVFPLAPCSRSTSESTAALGCLV | |
| | KDYFPEPVTVSWNSGALTSGVHTFPAVLQSSGLYSLSSVVTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPPSQ | |
| | EEMTKNQVSLWCLVKGFYPSDIAVEWESNGQPENNYKTTPPVLDSDGSFFL | |
| | YSRLTVDKSRWQEGNVFSCSVMHEALHNHYTQKSLSLSLGK | |
| | Half Antibody DNA sequence | |
| VG1 | ATGGCCTGGGTGTGGACCCTGCTGTTCCTGATGGCCGCCGCCCAGAGCAT | 20 |
| (ANTI- | CCAGGCCGACATCGTGATGAGCCAGAGCCCAAGCAGCCTGGCCGTGAGC | |
| TRGV9 | GTGGGCGAGAAGGTGACCATGAGCTGCAAGAGCAGCCAGAGCCTGCTGT | |
| half Ab) | ACAGCAGCAACCAGAAGAACTACCTGGCCTGGTACCAGCAGAAGCCAG | |
| | GCCAGAGCCCAAAGCTGCTGATCTACTGGGCCAGCACCCGCGAGAGCGG | |
| | CGTGCCAGACCGCTTCACCGGCAGCGGCAGCGGCACCGACTTCACCCTG | |
| | ACCATCAGCAGCGTGAAGGCCGAGGACCTGGCCGTGTACTACTGCCAGC | |
| | AGTACTACCGCTACCACACCTTCGGCACCGGCACCAAGCTGGAGATCAA | |
| | GCGCACCGTGGCCGCCCCAAGCGTGTTCATCTTCCCACCAAGCGACGAG | |
| | CAGCTGAAGAGCGGCACCGCCAGCGTGGTGTGCCTGCTGAACAACTTCT | |
| | ACCCACGCGAGGCCAAGGTGCAGTGGAAGGTGGACAACGCCCTGCAGA | |
| | GCGGCAACAGCCAGGAGAGCGTGACCGAGCAGGACAGCAAGGACAGCA | |
| | CCTACAGCCTGAGCAGCACCCTGACCCTGAGCAAGGCCGACTACGAGAA | |
| | GCACAAGGTGTACGCCTGCGAGGTGACCCACCAGGGCCTGAGCAGCCCA | |
| | GTGACCAAGAGCTTCAACCGCGGCGAGTGCggcggcagcgagggcaaga | |
| | gcagcggcagcggcagcgagagcaagagcaccgagggcaagagcagcgg | |
| | cagcggcagcgagagcaagagcaccggcggca | |
| | gcGAGGTGCAGCTGCAGCAGAGCGGCGCCGAGCTGGCCCGCCCAGGCGC | |
| | CAGCGTGAAGCTGAGCTGCAAGGCCAGCGGCTTCACCTTCACCGACCAC | |
| | TACATCAACTGGGTGAAGCAGCGCACCGGCCAGGGCCTGGAGTGGATCG | |
| | GCCAGATCTACCCAGGCGACGGCAACACCTACTACAACCAGAAGTTCAA | |
| | GGGCAAGGCCACCCTGACCGCCGACAAGAGCAGCAGCACCGCCTACATG | |
| | CAGCTGAGCAGCCTGACCAGCGAGGACAGCGCCGTGTACTTCTGCGCCC | |
| | CAAACTACGGCGACTACACCATCGACTTCTGGGGCCAGGGCACCAGCGT | |
| | GACCGTGAGCAGCGCCAGCACCAAGGGCCCAAGCGTGTTCCCACTGGCC | |
| | CCATGCAGCCGCAGCACCAGCGAGAGCACCGCCGCCCTGGGCTGCCTGG | |
| | TGAAGGACTACTTCCCAGAGCCAGTGACCGTGAGCTGGAACAGCGGCGC | |
| | CCTGACCAGCGGCGTGCACACCTTCCCAGCCGTGCTGCAGAGCAGCGGC | |
| | CTGTACAGCCTGAGCAGCGTGGTGACCGTGCCAAGCAGCAGCCTGGGCA | |
| | CCAAGACCTACACCTGCAACGTGGACCACAAGCCAAGCAACACCAAGGT | |
| | GGACAAGCGCGTGGAGAGCAAGTACGGCCCACCATGCCCACCATGCCCA | |
| | GCCCCAGAGGCCGCCGGCGGCCCAAGCGTGTTCCTGTTCCCACCAAAGC | |
| | CAAAGGACACCCTGATGATCAGCCGCACCCCAGAGGTGACCTGCGTGGT | |
| | GGTGGACGTGAGCCAGGAGGACCCAGAGGTGCAGTTCAACTGGTACGTG | |
| | GACGGCGTGGAGGTGCACAACGCCAAGACCAAGCCACGCGAGGAGCAG | |
| | TTCAACAGCACCTACCGCGTGGTGAGCGTGCTGACCGTGCTGCACCAGG | |
| | ACTGGCTGAACGGCAAGGAGTACAAGTGCAAGGTGAGCAACAAGGGCC | |
| | TGCCAAGCAGCATCGAGAAGACCATCAGCAAGGCCAAGGGCCAGCCAC | |
| | GCGAGCCACAGGTGTACACCCTGCCACCAAGCCAGGAGGAGATGACCA | |
| | AGAACCAGGTGAGCCTGTGGTGCCTGGTGAAGGGCTTCTACCCAAGCGA | |
| | CATCGCCGTGGAGTGGGAGAGCAACGGCCAGCCAGAGAACAACTACAA | |
| | GACCACCCCACCAGTGCTGGACAGCGACGGCAGCTTCTTCCTGTACAGC | |
| | CGCCTGACCGTGGACAAGAGCCGCTGGCAGGAGGGCAACGTGTTCAGCT | |
| | GCAGCGTGATGCACGAGGCCCTGCACAACCACTACACCCAGAAGAGCCT | |
| | GAGCCTGAGCCTGGGCAAG | |
| VG1 | ATGGCCTGGGTGTGGACCCTGCTGTTCCTGATGGCCGCCGCCCAGAGCAT | 21 |
| (anti- | CCAGGCCGAGATCGTGCTGACCCAGAGCCCAGGCACCCTGAGCCTGAGC | |
| CD123 | CCAGGCGAGCGCGCCACCCTGAGCTGCCGCGCCAGCCAGAGCGTGAGCA | |
| half Ab) | GCAGCTACCTGGCCTGGTACCAGCAGAAGCCAGGCCAGGCCCCACGCCT | |
| | GCTGATCTACGGCGCCAGCAGCCGCGCCACCGGCATCCCAGACCGCTTC | |
| | AGCGGCAGCGGCAGCGGCACCGACTTCACCCTGACCATCAGCCGCCTGG | |
| | AGCCAGAGGACTTCGCCGTGTACTACTGCCAGCAGGACTACGGCTTCCC | |
| | ATGGACCTTCGGCCAGGGCACCAAGGTGGAGATCAAGCGCACCGTGGCC | |
| | GCCCCAAGCGTGTTCATCTTCCCACCAAGCGACGAGCAGCTGAAGAGCG | |
| | GCACCGCCAGCGTGGTGTGCCTGCTGAACAACTTCTACCCACGCGAGGC | |
| | CAAGGTGCAGTGGAAGGTGGACAACGCCCTGCAGAGCGGCAACAGCCA | |
| | GGAGAGCGTGACCGAGCAGGACAGCAAGGACAGCACCTACAGCCTGAG | |
| | CAGCACCCTGACCCTGAGCAAGGCCGACTACGAGAAGCACAAGGTGTAC | |
| | GCCTGCGAGGTGACCCACCAGGGCCTGAGCAGCCCAGTGACCAAGAGCT | |
| | TCAACCGCGGCGAGTGCggcggcagcgagggcaagagcagcggcagcgg | |
| | cagcgagagcaagagcaccgagggcaagagcagcggcagcggcagcgag | |
| | agcaagagcaccggcggcagcGAGGTGCAGCT | |
| | GGTGCAGAGCGGCGCCGAGGTGAAGAAGCCAGGCGAGAGCCTGAAGAT | |
| | CAGCTGCAAGGGCAGCGGCTACAGCTTCACCAGCTACTGGATCAGCTGG | |
| | GTGCGCCAGATGCCAGGCAAGGGCCTGGAGTGGATGGGCATCATCGACC | |
| | CAAGCGACAGCGACACCCGCTACAGCCCAAGCTTCCAGGGCCAGGTGAC | |
| | CATCAGCGCCGACAAGAGCATCAGCACCGCCTACCTGCAGTGGAGCAGC | |
| | CTGAAGGCCAGCGACACCGCCATGTACTACTGCGCCCGCGGCGACGGCA | |
| | GCACCGACCTGGACTACTGGGGCCAGGGCACCCTGGTGACCGTGAGCAG | |
| | CGCCAGCACCAAGGGCCCAAGCGTGTTCCCACTGGCCCCATGCAGCCGC | |
| | AGCACCAGCGAGAGCACCGCCGCCCTGGGCTGCCTGGTGAAGGACTACT | |
| | TCCCAGAGCCAGTGACCGTGAGCTGGAACAGCGGCGCCCTGACCAGCGG | |
| | CGTGCACACCTTCCCAGCCGTGCTGCAGAGCAGCGGCCTGTACAGCCTG | |
| | AGCAGCGTGGTGACCGTGCCAAGCAGCAGCCTGGGCACCAAGACCTACA | |
| | CCTGCAACGTGGACCACAAGCCAAGCAACACCAAGGTGGACAAGCGCG | |
| | TGGAGAGCAAGTACGGCCCACCATGCCCACCATGCCCAGCCCCAGAGGC | |
| | CGCCGGCGGCCCAAGCGTGTTCCTGTTCCCACCAAAGCCAAAGGACACC | |
| | CTGATGATCAGCCGCACCCCAGAGGTGACCTGCGTGGTGGTGGACGTGA | |
| | GCCAGGAGGACCCAGAGGTGCAGTTCAACTGGTACGTGGACGGCGTGGA | |
| | GGTGCACAACGCCAAGACCAAGCCACGCGAGGAGCAGTTCAACAGCAC | |
| | CTACCGCGTGGTGAGCGTGCTGACCGTGCTGCACCAGGACTGGCTGAAC | |
| | GGCAAGGAGTACAAGTGCAAGGTGAGCAACAAGGGCCTGCCAAGCAGC | |
| | ATCGAGAAGACCATCAGCAAGGCCAAGGGCCAGCCACGCGAGCCACAG | |
| | GTGTACACCCTGCCACCAAGCCAGGAGGAGATGACCAAGAACCAGGTG | |
| | AGCCTGTGGTGCCTGGTGAAGGGCTTCTACCCAAGCGACATCGCCGTGG | |
| | AGTGGGAGAGCAACGGCCAGCCAGAGAACAACTACAAGACCACCCCAC | |
| | CAGTGCTGGACAGCGACGGCAGCTTCTTCCTGTACAGCCGCCTGACCGT | |
| | GGACAAGAGCCGCTGGCAGGAGGGCAACGTGTTCAGCTGCAGCGTGATG | |
| | CACGAGGCCCTGCACAACCACTACACCCAGAAGAGCCTGAGCCTGAGCC | |
| | TGGGCAAG | |
| VG3 | ATGGCCTGGGTGTGGACCCTGCTGTTCCTGATGGCCGCCGCCCAGAGCAT | 22 |
| (Null half | CCAGGCCGACATCGTGATGACCCAGAGCCCAGACAGCCTGGCCGTGAGC | |
| Ab) | CTGGGCGAGCGCGCCACCATCAACTGCCGCGCCAGCCAGAGCGTGGACT | |
| | ACAACGGCATCAGCTACATGCACTGGTACCAGCAGAAGCCAGGCCAGCC | |
| | ACCAAAGCTGCTGATCTACGCCGCCAGCAACCCAGAGAGCGGCGTGCCA | |
| | GACCGCTTCAGCGGCAGCGGCAGCGGCACCGACTTCACCCTGACCATCA | |
| | GCAGCCTGCAGGCCGAGGACGTGGCCGTGTACTACTGCCAGCAGATCAT | |
| | CGAGGACCCATGGACCTTCGGCCAGGGCACCAAGGTGGAGATCAAGCGC | |
| | ACCGTGGCCGCCCCAAGCGTGTTCATCTTCCCACCAAGCGACGAGCAGC | |
| | TGAAGAGCGGCACCGCCAGCGTGGTGTGCCTGCTGAACAACTTCTACCC | |
| | ACGCGAGGCCAAGGTGCAGTGGAAGGTGGACAACGCCCTGCAGAGCGG | |
| | CAACAGCCAGGAGAGCGTGACCGAGCAGGACAGCAAGGACAGCACCTA | |
| | CAGCCTGAGCAGCACCCTGACCCTGAGCAAGGCCGACTACGAGAAGCAC | |
| | AAGGTGTACGCCTGCGAGGTGACCCACCAGGGCCTGAGCAGCCCAGTGA | |
| | CCAAGAGCTTCAACCGCGGCGAGTGCGGCGGCAGCGAGGGCAAGAGCA | |
| | GCGGCAGCGGCAGCGAGAGCAAGAGCACCGAGGGCAAGAGCAGCGGCA | |
| | GCGGCAGCGAGAGCAAGAGCACCGGCGGCAGCCAGATCACCCTGAAGG | |
| | AGAGCGGCCCAACCCTGGTGAAGCCAACCCAGACCCTGACCCTGACCTG | |
| | CACCTTCAGCGGCTTCAGCCTGAGCACCAGCGGCATGGGCGTGAGCTGG | |
| | ATCCGCCAGCCACCAGGCAAGGCCCTGGAGTGGCTGGCCCACATCTACT | |
| | GGGACGACGACAAGCGCTACAACCCAAGCCTGAAGAGCCGCCTGACCAT | |
| | CACCAAGGACACCAGCAAGAACCAGGTGGTGCTGACCATGACCAACATG | |
| | GACCCAGTGGACACCGCCACCTACTACTGCGCCCGCCTGTACGGCTTCAC | |
| | CTACGGCTTCGCCTACTGGGGCCAGGGCACCCTGGTGACCGTGAGCAGC | |
| | GCCAGCACCAAGGGCCCAAGCGTGTTCCCACTGGCCCCATGCAGCCGCA | |
| | GCACCAGCGAGAGCACCGCCGCCCTGGGCTGCCTGGTGAAGGACTACTT | |
| | CCCAGAGCCAGTGACCGTGAGCTGGAACAGCGGCGCCCTGACCAGCGGC | |
| | GTGCACACCTTCCCAGCCGTGCTGCAGAGCAGCGGCCTGTACAGCCTGA | |
| | GCAGCGTGGTGACCGTGCCAAGCAGCAGCCTGGGCACCAAGACCTACAC | |
| | CTGCAACGTGGACCACAAGCCAAGCAACACCAAGGTGGACAAGCGCGT | |
| | GGAGAGCAAGTACGGCCCACCATGCCCACCATGCCCAGCCCCAGAGGCC | |
| | GCCGGCGGCCCAAGCGTGTTCCTGTTCCCACCAAAGCCAAAGGACACCC | |
| | TGATGATCAGCCGCACCCCAGAGGTGACCTGCGTGGTGGTGGACGTGAG | |
| | CCAGGAGGACCCAGAGGTGCAGTTCAACTGGTACGTGGACGGCGTGGAG | |
| | GTGCACAACGCCAAGACCAAGCCACGCGAGGAGCAGTTCAACAGCACCT | |
| | ACCGCGTGGTGAGCGTGCTGACCGTGCTGCACCAGGACTGGCTGAACGG | |
| | CAAGGAGTACAAGTGCAAGGTGAGCAACAAGGGCCTGCCAAGCAGCAT | |
| | CGAGAAGACCATCAGCAAGGCCAAGGGCCAGCCACGCGAGCCACAGGT | |
| | GTACACCCTGCCACCAAGCCAGGAGGAGATGACCAAGAACCAGGTGAG | |
| | CCTGTGGTGCCTGGTGAAGGGCTTCTACCCAAGCGACATCGCCGTGGAG | |
| | TGGGAGAGCAACGGCCAGCCAGAGAACAACTACAAGACCACCCCACCA | |
| | GTGCTGGACAGCGACGGCAGCTTCTTCCTGTACAGCCGCCTGACCGTGG | |
| | ACAAGAGCCGCTGGCAGGAGGGCAACGTGTTCAGCTGCAGCGTGATGCA | |
| | CGAGGCCCTGCACAACCACTACACCCAGAAGAGCCTGAGCCTGAGCCTG | |
| | GGCAAG | |

TABLE 11
Heavy and Light Chain Sequences for TRGV9 bispecific antibodies

| Bispecific | | |
|---|---|---|
| Antibody | | Amino Acid Sequence |
| VG1 | Heavy chain 1 | EVQLQQSGAELARPGASVKLSCKASGFTFTDHYINWVKQRT |
| (ANTI- | VG1 (SEQ ID | GQGLEWIGQIYPGDGNTYYNQKFKGKATLTADKSSSTAYM |
| TRGV9/anti- | NO: 23) | QLSSLTSEDSAVYFCAPNYGDYTIDFWGQGTSVTVSSASTK |
| CD123) | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEAAGGPSVFLFPPKPKDT |
| | | LMISRTPEVTCVVVDVSQEDPEVQFNWYVDGVEVHNAKTK |
| | | PREEQFNSTYRVVSVLTVLHQDWLNGKEYKCKVSNKGLPS |
| | | SIEKTISKAKGQPREPQVYTLPPSQEEMTKNQVSLSCAVKGF |
| | | YPSDIAVEWESNGQPENNYKTTPPVLDSDGSFFLVSRLTVD |
| | | KSRWQEGNVFSCSVMHEALHNRFTQKSLSLSLGK |
| | Light Chain 1 | DIVMSQSPSSLAVSVGEKVTMSCKSSQSLLYSSNQKNYLAW |
| | VG1 (SEQ ID | YQQKPGQSPKLLIYWASTRESGVPDRFTGSGSGTDFTLTISS |
| | NO: 24) | VKAEDLAVYYCQQYYRYHTFGTGTKLEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| | Heavy chain 2 | EVQLVQSGAEVKKPGESLKISCKGSGYSFTSYWISWVRQMP |
| | VG1 (SEQ ID | GKGLEWMGIIDPSDSDTRYSPSFQGQVTISADKSISTAYLQW |
| | NO: 25) | SSLKASDTAMYYCARGDGSTDLDYWGQGTLVTVSSASTKG |
| | | PSVFPLAPCSRSTSESTAALGCLVKDYFPEPVTVSWNSGALT |
| | | SGVHTFPAVLQSSGLYSLSSVVTVPSSSLGTKTYTCNVDHKP |
| | | SNTKVDKRVESKYGPPCPPCPAPEAAGGPSVFLFPPKPKDTL |
| | | MISRTPEVTCVVVDVSQEDPEVQFNWYVDGVEVHNAKTKP |
| | | REEQFNSTYRVVSVLTVLHQDWLNGKEYKCKVSNKGLPSSI |
| | | EKTISKAKGQPREPQVYTLPPSQEEMTKNQVSLWCLVKGFY |
| | | PSDIAVEWESNGQPENNYKTTPPVLDSDGSFFLYSRLTVDKS |
| | | RWQEGNVFSCSVMHEALHNHYTQKSLSLSLGK |
| | Light Chain 2 | EIVLTQSPGTLSLSPGERATLSCRASQSVSSSYLAWYQQKPG |
| | VG1 (SEQ ID | QAPRLLIYGASSRATGIPDRFSGSGSGTDFTLTISRLEPEDFA |
| | NO: 26) | VYYCQQDYGFPWTFGQGTKVEIKRTVAAPSVFIFPPSDEQL |
| | | KSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| ANTI- | Heavy chain 1 | QVQLQESGPGLVKPSETLSLTCTVSGYSITSGYFWNWIRQPP |
| TRGV9 x | VG3 (SEQ ID | GKGLEWIGYISYDGSNNYNPSLKSRVTISRDTSKNQFSLKLS |
| Null | NO: 27) | SVTAADTAVYYCASPSPGTGYAVDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEAAGGPSVFLFPPKPKDT |
| | | LMISRTPEVTCVVVDVSQEDPEVQFNWYVDGVEVHNAKTK |
| | | PREEQFNSTYRVVSVLTVLHQDWLNGKEYKCKVSNKGLPS |
| | | SIEKTISKAKGQPREPQVYTLPPSQEEMTKNQVSLSCAVKGF |
| | | YPSDIAVEWESNGQPENNYKTTPPVLDSDGSFFLVSRLTVD |
| | | KSRWQEGNVFSCSVMHEALHNRFTQKSLSLSLGK |
| | Light Chain 1 | DIQMTQSPSSLSASVGDRVTITCRSSQSLVHSNGNTYLHWY |
| | VG3 (SEQ ID | QQKPGKAPKFLIYKVSNRFSGVPSRFSGSGSGTDFTLTISSLQ |
| | NO: 28) | PEDFATYYCSQSTHVPFTFGQGTKLEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| | Heavy chain 2 | QITLKESGPTLVKPTQTLTLTCTFSGFSLSTSGMGVSWIRQPP |
| | VG3 (SEQ ID | GKALEWLAHIYWDDDKRYNPSLKSRLTITKDTSKNQVVLT |
| | NO: 29) | MTNMDPVDTATYYCARLYGFTYGFAYWGQGTLVTVSSAS |
| | | TKGPSVFPLAPCSRSTSESTAALGCLVKDYFPEPVTVSWNSG |
| | | ALTSGVHTFPAVLQSSGLYSLSSVVTVPSSSLGTKTYTCNVD |
| | | HKPSNTKVDKRVESKYGPPCPPCPAPEAAGGPSVFLFPPKPK |
| | | DTLMISRTPEVTCVVVDVSQEDPEVQFNWYVDGVEVHNAK |
| | | TKPREEQFNSTYRVVSVLTVLHQDWLNGKEYKCKVSNKGL |
| | | PSSIEKTISKAKGQPREPQVYTLPPSQEEMTKNQVSLWCLVK |
| | | GFYPSDIAVEWESNGQPENNYKTTPPVLDSDGSFFLYSRLTV |
| | | DKSRWQEGNVFSCSVMHEALHNHYTQKSLSLSLGK |
| | Light Chain 2 | DIVMTQSPDSLAVSLGERATINCRASQSVDYNGISYMHWYQ |
| | VG3 (SEQ ID | QKPGQPPKLLIYAASNPESGVPDRFSGSGSGTDFTLTISSLQA |
| | NO: 30) | EDVAVYYCQQIIEDPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |

### Example 2.3: Characterization of Vγ9+(γδ) T Cells and Pan T Cells

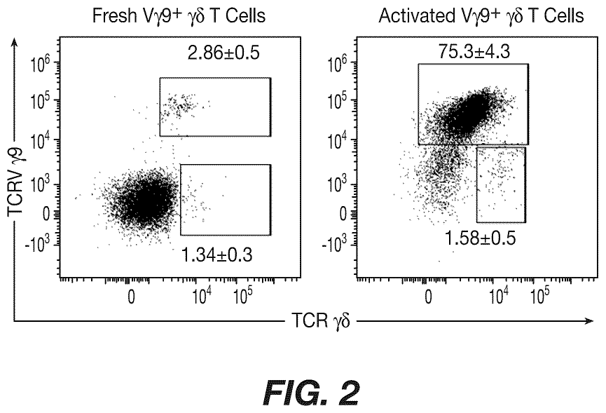

Zoledronic acid selectively expands Vγ9⁺γδ T cells from whole PBMCs. PBMCs were isolated from whole fresh PBMCs using EASYSEP Human γδ T cell isolation kit (Stem cell Technologies; Vancouver, CA) according to manufacturer instructions. Isolated PBMCs were cultured in RPMI-10 (RPMI supplemented with 10% FBS, 1× Pen/Strep) medium with recombinant human IL-2 (rhIL-2) to a final concentration of 1000 IU/mL and recombinant human IL-15 (rhIL-15) to a final concentration of 10 ng/mL and Zoledronic acid to a final concentration of 5 pM. for 14 days. Numbers in representative dot plots show the frequency (mean±SEM) of Vγ9⁺ and Vγ9⁻ TCR γδ T cells among total PBMCs on day 0 (left) and day 14 of PBMCs cultured with Zoledronic acid+IL-2+IL-15 (right). Represented data is mean (±SEM) of five donors (n=5) from a single experiment (FIG. 2).

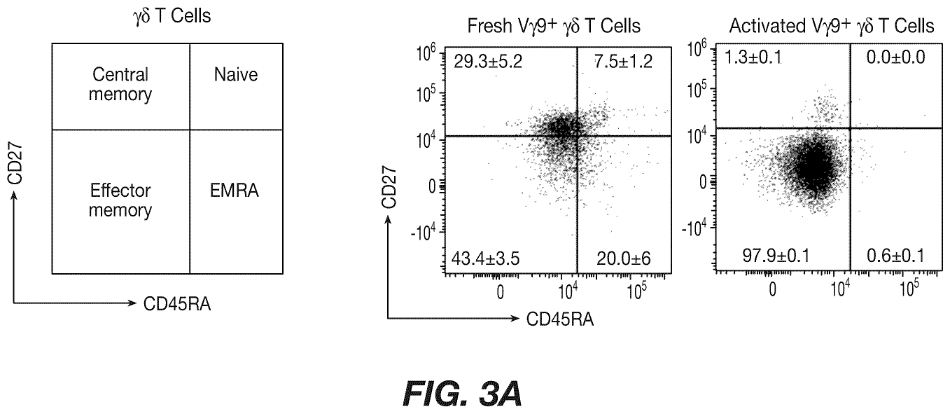

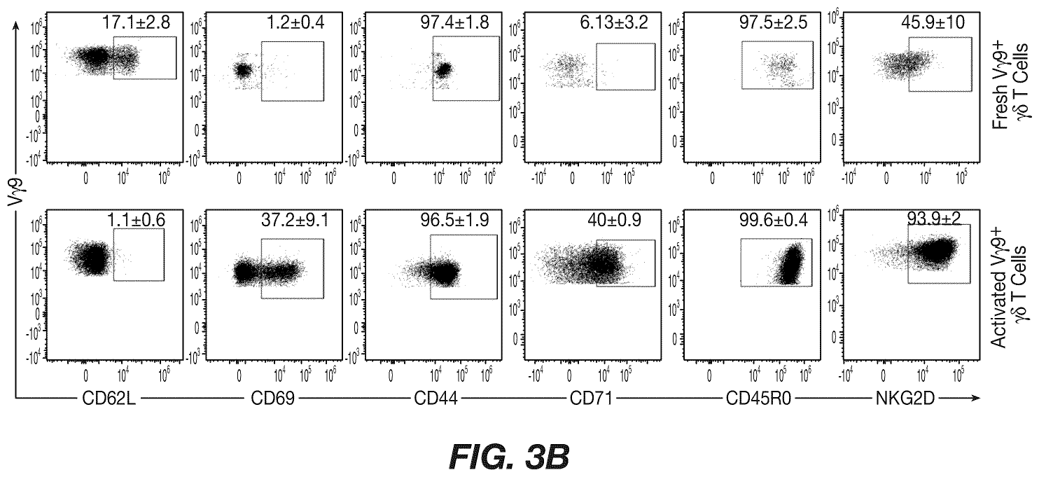

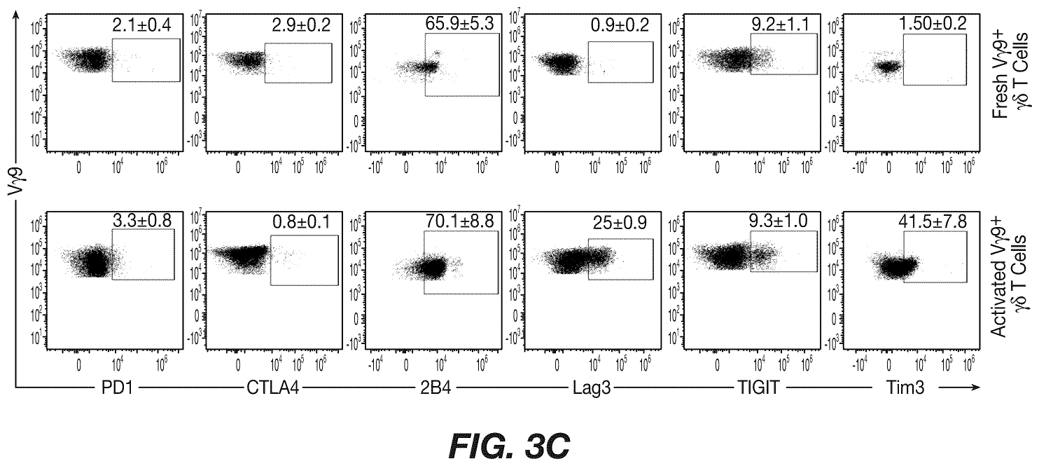

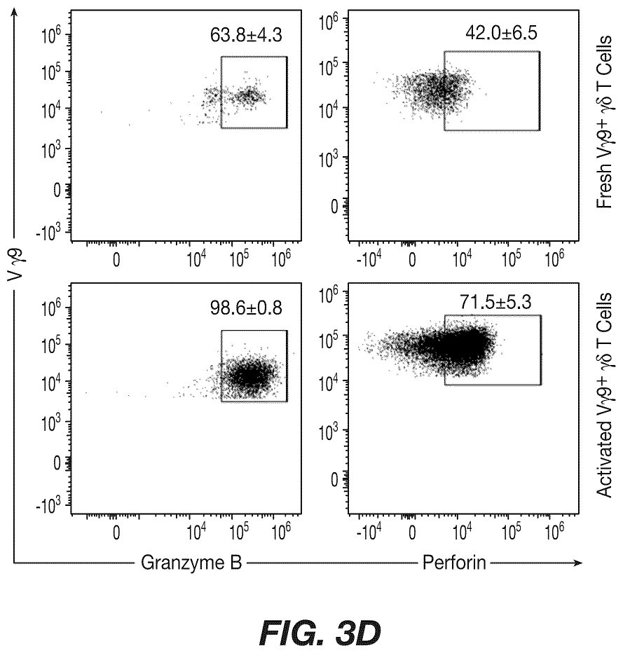

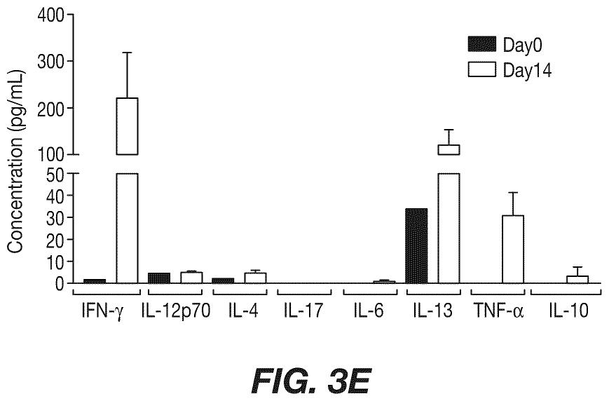

FIGS. 3A to 3E demonstrate the phenotypic characterization of Vγ9⁺ γδ T cells. FIG. 3A shows a schematic depiction of gates used to describe the differentiation of γδ T cells (left). Representative FACS-dot plots show the differentiation profile of Vγ9⁺ γδ T cells from fresh PBMCs (left) and PBMCs cultured ex vivo with Zoledronic acid+IL-2+IL-15 for 14 days (right). Numbers in quadrants mirror the frequency (mean±SEM) of the respective population among fresh and activated Vγ9⁺γδ T cells. Represented data is mean (±SEM) of five donors (n=5) from a single experiment. FIG. 3B shows numbers in representative dot plots mirroring the frequency (mean±SEM) of Vγ9⁺γδ T cells positive for respective activation marker either from fresh PBMCs (upper row) or PBMCs cultured with Zoledronic acid+IL-2+IL-15 for 14 days (lower row). Represented data is mean (±SEM) of seven donors (n=7) for CD62L, CD69, CD44 expression data from two independent experiments. n=5 donors for NKG2D and 2 donors for CD45RO and CD71 expression data respectively from a single experiment. FIG. 3C shows numbers above gates in dot plots depicting the frequency (mean±SEM) of Vγ9⁺γδ T cells positive for respective inhibitory receptor surface expression either from fresh PBMCs (upper row) or PBMCs cultured with Zoledronic acid+IL-2+IL-15 for day 14 days (lower row). Data shown here is mean (±SEM) of five donors (n=5) for PD1, CTLA4, TIGIT and LAG3 surface expression and seven donors (n=7) for 2B4 and TIM3 surface expression data from two independent experiments. FIG. 3D shows representative FACS dot plots demonstrating the frequency (mean±SEM) of Vγ9⁺γδ T cells expressing intracellular Granzyme B (left column) and Perforin (right column) from fresh PBMCs (upper row) and PBMCs cultured ex vivo with Zoledronic acid+IL-2+IL-15 for 14 days (lower row). Depicted data is mean (±SEM) of four (n=4) and seven (n=7) donors for Granzyme B and Perforin data respectively from two independent experiments. FIG. 3E shows bars representing the mean (±SEM) concentration (pg/mL) of cytokine in the cell culture supernatant on day 0 and day 14 of PBMCs culture with Zoledronic acid+IL-2+IL-15. Represented data is mean (±SEM) of four wells (n=4) from a single donor.

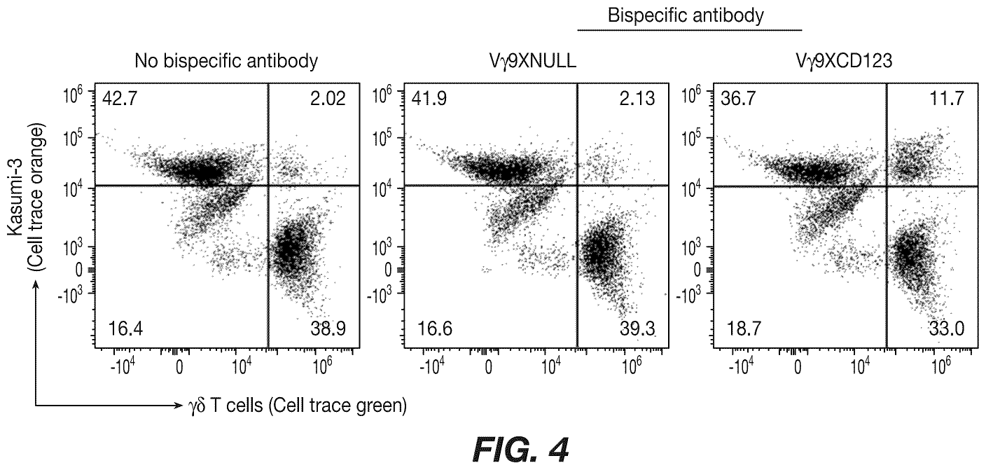

FIG. 4 shows that the anti-TRGV9/anti-CD123 bispecific antibody recruits γδ T cells into biphasic cell-cell conjugate. γδ T cells (effector cells) were isolated from whole fresh PBMCs using EASYSEP Human γδ T cell isolation kit (Stem cell Technologies) according to manufacturer instructions. γδ T cells were labelled with 0.25 μM CELLTRACKER™ Green CMFDA Dye for 30 min. and Kasumi-3 (Targets) cells were labelled with 1 μM CELLTRACKER™ Orange CMRA Dye in RPMI medium for 30 minutes at 37° C. Both labeled γδ T cells and Kasumi-3 were co-cultured. Labeled Effector (E) and Target (T) cells at an E:T ratio of 1:1 (50,000 cells of each cell type/well) with 1 microgram per ML of bispecific antibody (anti-TRGV9/anti-CD123, anti-TRGV9/anti-NULL) and incubated at 37° C. for 1 hour. At the end of the incubation, cells were spun down at 1200 rpm for 5 minutes and resuspended in FACS buffer. Cells were acquired utilizing the flow cytometer and analysis was performed using FLOWJO analysis software. Numbers in quadrants of representative FACS plots show the frequency of recruited or non-recruited cells to the cell-cell conjugate either in the absence (left dot plot) or presence of anti-TRGV9/anti-NULL (middle dot plot) and anti-TRGV9/anti-CD123 (right dot plot) bispecific antibody. Data shown here is from a single experiment.

### Example 2.4: Evaluation of Binding and Cytotoxic Properties of the Anti-Trgv9/Anti-Cd123 Bispecific Antibody Using Kasumi-3 Cells and Human γδ T Cells

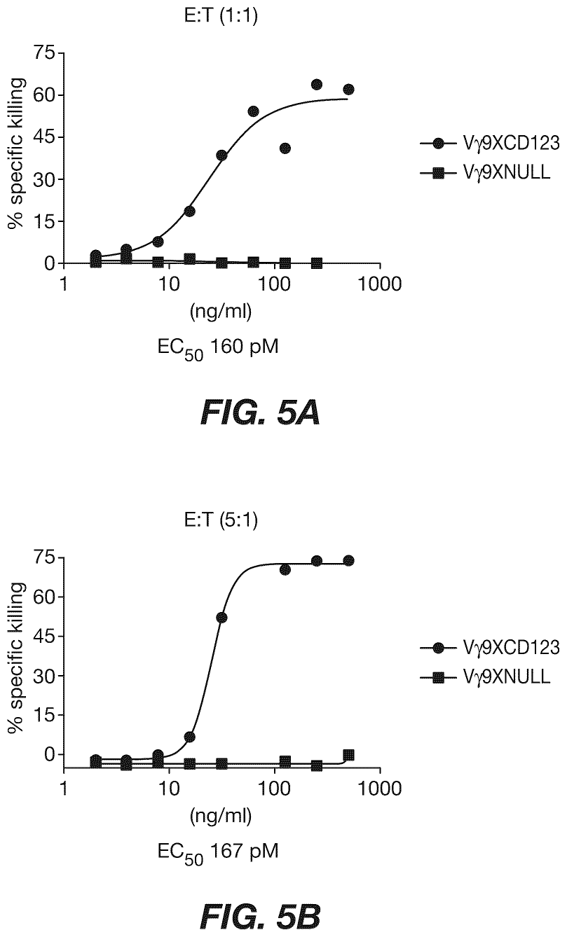

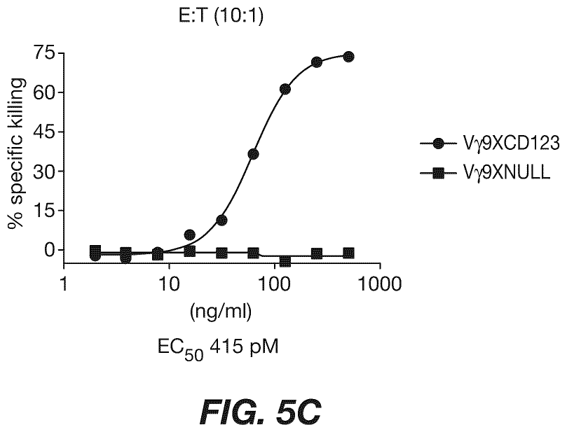

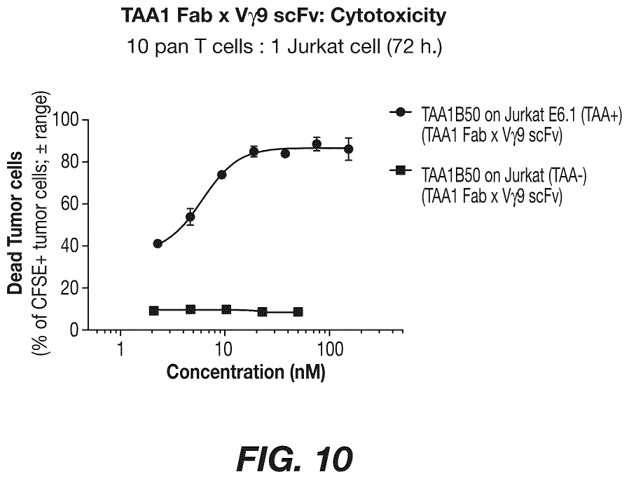

FIG. 5 shows that the anti-TRGV9/anti-CD123 bispecific antibody mediates γδ T cell cytotoxicity against CD123 expressing Kasumi-3 cells in vitro. Enriched γδ T cells (Effectors), isolated from PBMCs cultured with Zoledronic acid+IL-2+IL-15 for 12 days, were co-cultured with CFSE labelled Kasumi-3 cells (Targets) at 1:1, 5:1 and 10:1 E:T ratios in the presence of various concentrations of the bispecific antibody for 24 hours. Dose response curves show anti-TRGV9/anti-CD123 and anti-TRGV9/anti-NULL bispecific mediated γδ T cell cytotoxicity against CD123 expressing Kasumi-3 cells in a dose dependent manner at 1:1 (FIG. 5A) 5:1 (FIG. 5B) and 10:1 (FIG. 5C) E:T ratios. Cytotoxicity values represented here were subtracted of basal cytotoxicity value observed in the absence of bispecific antibody. EC₅₀ values were calculated as described in methods. Representative data shown here are from a single experiment.

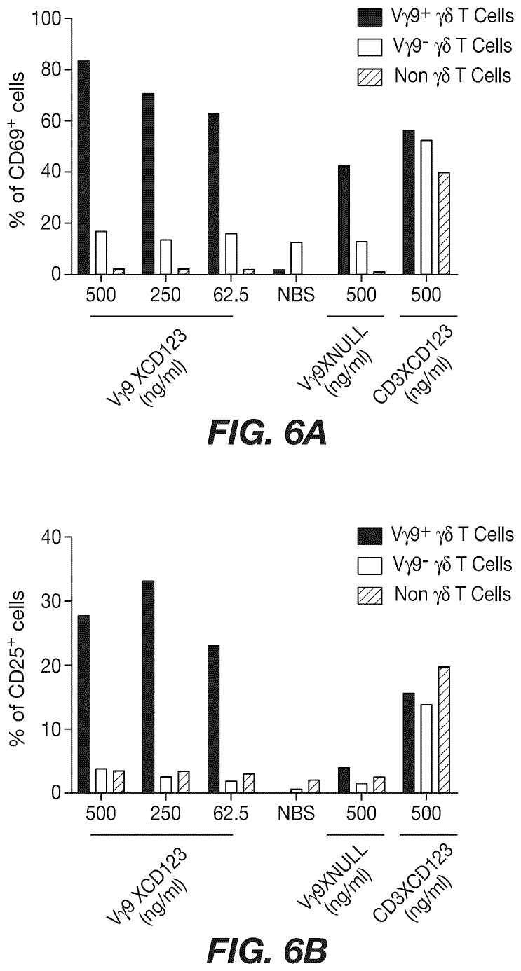

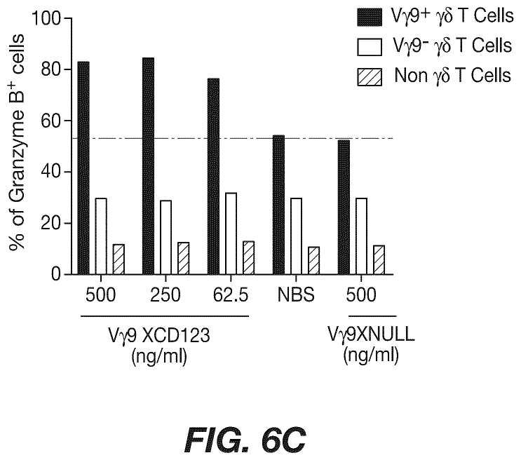

FIG. 6 shows that the anti-TRGV9/anti-CD123 bispecific selectively activates Vγ9⁺γδ T cells. Whole fresh PBMCs were co-cultured with Kasumi-3 cells in the presence of various concentrations of the anti-TRGV9/anti-CD123 bispecific antibody for 72 hours at 37° C. As a positive and negative control, co-cultured cells were stimulated with anti-CD3/anti-CD123 and anti-TRGV9/anti-NULL bispecifics for 72 hours at 37° C. Bars represent the frequency of Vγ9⁺, Vγ9⁻ γδ T cells and non-γδ T cells positive for CD69 (FIG. 6A, left), CD25 (FIG. 6A, right) surface expression, and intracellular Granzyme B (FIG. 6B) expression. The dotted line in FIG. 6B indicates the basal levels of Granzyme B expression in Vγ9⁺γδ T cells. NBS denotes no bispecific antibody added to the co-cultured cells. Data shown here are from a single experiment.

### Example 3: Humanization of Anti-Trgv9 Clone 7A5

Mouse anti-human Vγ9 clone LP7A5 (7A5) binds to the antigen (Vγ9-V62 fused to human Fc) with a K_{D} of 1.9 nM. Humanization of murine 7A5 was performed following the approach outlined by Singh et al., mAbs J, 2015. Based on sequence conservation, the heavy chain germline IGHV1-8*01 was chosen for framework adaption. Three somatic hypermutation sites in the heavy chain were chosen for binary library design. A potential Iso-Asp isomerization site (DG motif) in the CDR-H2 was also included in the design to mitigate this potential liability. For light chain frame adaption, IGKV4-1*01 was chosen as the closest homologous human germline. Owing to high sequence homology, only one position (Asn22) was included in the library design. The variants were cloned and expressed in E. coli. The supernatants were screened in single point ELISA and the periplasmic preparation was used for dose response. A mouse/human chimeric 7A5 Fab was used as parental control. Clone 7A5_17 (7A5_var17) maintained the binding activity similar to murine 7A5 and was converted to IgG for additional profiling. The EC₅₀ for primary cell binding for clone 7A5_17 and 7A5 were 200 pM and 159 pM.

The sequences obtained are shown in Tables 12-15. The three VH CDR and three VL CDR sequences of the humanized anti-human TRGV9 clone 7A5_var17 are shown in Table 12 (two versions, depending on CDR type, are provided); and the VH and VL sequences of the humanized anti-human TRGV9 clone 7A5_var17 are shown in Table 14 (SEQ ID NOs:65 and 66, respectively). The three VH CDR and three VL CDR sequences of the humanized anti-human TRGV9 clone 7A5_var29 are shown in Table 13 (two versions, depending on CDR type, are provided); and the VH and VL sequences of the humanized anti-human TRGV9 clone 7A5_var29 are shown in Table 15 (SEQ ID NOs:67 and 68, respectively).

TABLE 12
CDR sequences of humanized anti-human
TRGV9 clone 7A5_var 17.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| 7A5_var17 | GFTFTDHY | 60 | IYPGSGNT | 61 | ARNYGD | 62 |
| (CDR v.1) | | | | | YTIDF | |
| 7A5_var17 | DHYIN | 1 | QIYPGSGNT | 76 | NYGDYT | 3 |
| (CDR v.2) | | | YYNQKFKG | | IDF | |
| | | SEQ | | SEQ | | SEQ |
| | | ID | | ID | | ID |
| mAb ID | LCDR1 | NO: | LCDR2 | NO: | LCDR3 | NO: |
| 7A5_var17 | QSVLYSSN | 63 | WAS | 64 | QQYYRYHT | 6 |
| (CDR v.1) | NKNY | | | | | |
| 7A5_var17 | KSSQSVLYS | 77 | WASTRES | 5 | QQYYRYHT | 6 |
| (CDR v.2) | SNNKNYLA | | | | | |

TABLE 13
CDR sequences of humanized anti-human
TRGV9 clone 7A5_var 29.

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| 7A5_var29 | GFTFTDHY | 60 | IYPGSGNT | 61 | ARNYGD | 62 |
| (CDR v.1) | | | | | YTIDF | |
| 7A5_var29 | DHYIN | 1 | QIYPGSGNT | 76 | NYGDYT | 3 |
| (CDR v.2) | | | YYNQKFKG | | IDF | |
| | | SEQ | | SEQ | | SEQ |
| | | ID | | ID | | ID |
| mAb ID | LCDR1 | NO: | LCDR2 | NO: | LCDR3 | NO: |
| 7A5_var29 | QSVLYSSN | 63 | WAS | 64 | QQYYRYHT | 6 |
| (CDR v.1) | NKNY | | | | | |
| 7A5 var29 | KSSQSVLYS | 77 | WASTRES | 5 | QQYYRYHT | 6 |
| (CDR v.2) | SNNKNYLA | | | | | |

TABLE 14
Heavy chain and light chain V-region sequences of humanized anti-
human TRGV9 clone 7A5_var17.

| | | SEQ ID |
|---|---|---|
| mAb ID | Heavy Chain V-region Amino Acid Sequence | NO: |
| 7A5_var17 | QVQLVQSGAE VKKPGASVKV SCKASGFTFT DHYINWVRQA | 65 |
| | TGQGLEWMGQ IYPGSGNTYY NQKFKGRVTM TRDTSISTAY | |
| | MELSSLRSED TAVYYCARNY GDYTIDFWGQ GTSVTVSS | |
| | | SEQ ID |
| mAb ID | Light Chain V-region Amino Acid Sequence | NO: |
| 7A5_var17 | DIVMTQSPDS LAVSLGERAT INCKSSQSVL YSSNNKNYLA | 66 |
| | WYQQKPGQPP KLLIYWASTR ESGVPDRFSG SGSGTDFTLT | |
| | ISSLQAEDVA VYYCQQYYRY HTFGTGTKLE IK | |

TABLE 15
Heavy chain and light chain V-region sequences of
humanized anti-human TRGV9 clone 7A5_var29.

| | | SEQ ID |
|---|---|---|
| mAb ID | Heavy Chain V-region Amino Acid Sequence | NO: |
| 7A5_var29 | QVQLVQSGAE VKKPGASVKV SCKASGFTFT DHYINWVRQA | 67 |
| | TGQGLEWMGQ IYPGSGNTYY NQKFKGRVTM TRNTSISTAY | |
| | MELSSLRSED TAVYYCARNY GDYTIDFWGQ GTSVTVSS | |
| | | SEQ ID |
| mAb ID | Light Chain V-region Amino Acid Sequence | NO: |
| 7A5_var29 | DIVMTQSPDS LAVSLGERAT ISCKSSQSVL YSSNNKNYLA | 68 |
| | WYQQKPGQPP KLLIYWASTR ESGVPDRFSG SGSGTDFTLT | |
| | ISSLQAEDVA VYYCQQYYRY HTFGTGTKLE IK | |

### Example 4—Multispecific Antibodies that Bind Trgv9 and Taa1

T cell acute lymphoblastic leukemia (T-ALL) are aggressive neoplasms characterized by the proliferation and accumulation in blood, bone marrow and lymphoid organs of T cell precursors abnormally arrested in differentiation. Current first-line chemotherapy regimens provide overall survival rates of approximately 85-90% in children and about 50% in adults (Pui et al. J. Clin. Oncol. Off J. Am. Soc. Clin. Oncol. 2015; 33(27):2938-2948; Litzow et al. Blood. 2015; 126(7):833-841). T-ALL represent a heterogeneous group of malignancies classified into different molecular subtypes on the basis of aberrant expression of specific driver oncogenic transcription factors and global transcriptomic signatures (Belver et al. Nat. Rev. Cancer. 2016; 16(8):494-507; Nat. Genet. 2017; 49(8):1211-1218). T-cells are the most abundant (˜75% of blood lymphocytes) and potent immune killer cells. The role of effector T-cells in the anti-cancer immune response is strongly supported by in vitro studies and the observation that a high infiltration of CD8⁺ T cells in several types of cancers correlates with a favorable clinical prognostic.

V79V62 T lymphocytes, a major γ/δ T cell subset in humans, recognize phosphoantigens, certain tumor cells, and cells treated with aminobisphosphonates. This cell population displays cytolytic activity against various tumor cells. The γ/δ TCR is a heterodimeric TCR complex composed of covalently bound γ and δ chains involved in antigen recognition and the non-covalently associated monomorphic proteins CD3δ, γ, ε, and (chains. The Vγ9 TCR is a variant of the TCR γ chain expressed on a subset of γ/δ T cells.

Examples 4.1-4.6 are based on the premise that γδ T cells, which mainly express heterodimers of TRGV9 and V62 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a T-cell Tumor-Associated Antigen (TAA1) expressed by the tumor cells (e.g., T cell lymphomas). Thus, the bispecific antibody bridges the effector and target cells together, resulting in tumor cell killing. This mechanism of action is described in the schematic outlined in FIG. 1.

The subsequent examples can be divided into the following categories: (1) Generation and characterization of bispecific antibodies capable of binding to the TRGV9 arm expressed on γδ T cells and TAA1 on αβ T cells (Examples 4.1, 4.2, and 4.3); and (2) Evidence for bispecific antibody binding and bispecific antibody-enabled target cell killing by γδ T cells expanded in vitro (Example 4.4 and 4.5).

γδ T cell stimulation and expansion was performed. Expansion of Vγ9-V62 T cells was carried out by treating PBMCs in complete RPMI media containing rhIL-2 (1000 IU/mL), rhIL-15 (10 ng/mL) and zoledronic acid (5 μM) for 14 days.

### Example 4.1: Production and De Novo Sequencing of Anti-Trgv9 Mab

The mouse IgG1 anti-human T cell receptor anti-TRGV9 clone 7A5 was sourced commercially. Sample preparation and LC-MS/MS analysis were performed by Lake Pharma (San Carlos, CA). The sample was reduced and alkylated, divided into seven aliquots, and proteolytically digested with Trypsin/LysC, Chymotrypsin, LysC, Pepsin, and AspN, Elastase, and Proteinase K enzymes. Resulting peptides were desalted using a ZIPTIP C18 Pipette Tips and separated on-line using reverse phase chromatography. Mass spectrometry was performed on THERMO Q-EXACTIVE spectrometer using HCD fragmentation. MS data sets were analyzed using PEAKS software by matching de novo sequence tags to an IMGT-based antibody sequences database. Gaps in the sequence were assigned using Contig sequence assembly of de novo identified peptides. All CDRs and hyper-mutations were confirmed by inspecting the MS/MS spectra.

The three VH CDR and three VL CDR sequences of the mouse anti-human TRGV9 clone 7A5 (LP7A5_1) are previously shown in Table 3 (SEQ ID NOs:1-6, respectively); and the VH and VL sequences of the mouse anti-human TRGV9 clone 7A5 (LP7A5_1) are previously shown in Table 7 (SEQ ID NOs:7 and 8, respectively).

### Example 4.2: Production and De Novo Sequencing of Anti-Taa1 Mab

The mouse IgG2a monoclonal anti-human TAA1 clone was commercially sourced. The VH CDR1-3, VL CDR1-3, VH and VL sequences of this clone (data not shown) were obtained using a similar procedure as described above for the anti-TRGV9 clone 7A5.

### Example 4.3: Preparation of Anti-Trgv9/Anti-Taa1 Bispecific Antibodies

The variable region sequence of clone 7A5 (anti-TRGV9 antibody) and the TAA1 clone (anti-TAA1 antibody) were used to generate a bispecific antibody to be tested for T cell re-directed killing of acute myeloid leukemia (AML) cells. The bispecific antibodies VG4 (anti-TRGV9 x TAA1) and VG3 (anti-TRGV9 x Null) were produced as full-length antibodies in the knob-into-hole format as human IgG4. Nucleic acid sequences encoding variable regions were sub-cloned into a custom mammalian expression vectors containing constant region of human IgG4 expression cassettes using standard PCR restriction enzyme based standard cloning techniques, and sequenced verified. The bispecific antibodies were expressed by transient transfection in a Chinese hamster ovary (CHO) cell line. Sequences of certain bispecific antibodies expressed in the CHO cells are shown in Table 16 below. Certain individual heavy and light chain antibody sequences are shown in Table 17 below

TABLE 16
Sequences of antibodies expressed in CHO cells

| | | | SEQ |
|---|---|---|---|
| mAb | ‘Knob’ arm and ‘hole’ arm amino | | ID |
| ID | acid sequence | | NO: |
| Anti- | Heavy | MAWVWTLLFLMAAAQSIQAEVQLQQSGAELARPGA | 78 |
| TRGV9_ | Chain | SVKLSCKASGFTFTDHYINWVKQRTGQGLEWIGQI | |
| 7A5_1 | A | YPGDGNTYYNQKFKGKATLTADKSSSTAYMQLSSL | |
| (half- | | TSEDSAVYFCAPNYGDYTIDFWGQGTSVTVSSAST | |
| mAb) | | KGPSVFPLAPSSKSTSGGTAALGCLVKDYFPEPVT | |
| | | VSWNSGALTSGVHTFPAVLQSSGLYSLSSVVTVPS | |
| | | SSLGTQTYICNVNHKPSNTKVDKKVEPKSCDKTHT | |
| | | CPPCPAPEAAGGPSVFLFPPKPKDTLMISRTPEVT | |
| | | CVVVSVSHEDPEVKFNWYVDGVEVHNAKTKPREEQ | |
| | | YNSTYRVVSVLTVLHQDWLNGKEYKCKVSNKALPA | |
| | | PIEKTISKAKGQPREPQVYVYPPSREEMTKNQVSL | |
| | | TCLVKGFYPSDIAVEWESNGQPENNYKTTPPVLDS | |
| | | DGSFALVSKLTVDKSRWQQGNVFSCSVMHEALHNR | |
| | | FTQKSLSLSPGK | |
| | Light | MAWVWTLLFLMAAAQSIQADIVMSQSPSSLAVSVG | 79 |
| | Chain | EKVTMSCKSSQSLLYSSNQKNYLAWYQQKPGQSPK | |
| | | LLIYWASTRESGVPDRFTGSGSGTDFTLTISSVKA | |
| | | EDLAVYYCQQYYRYHTFGTGTKLEIKRTVAAPSVF | |
| | | IFPPSDEQLKSGTASVVCLLNNFYPREAKVQWKVD | |
| | | NALQSGNSQESVTEQDSKDSTYSLSSTLTLSKADY | |
| | | EKHKVYACEVTHQGLSSPVTKSFNRGEC | |
| Anti- | Heavy | MAWVWTLLFLMAAAQSIQAQVQLVQSGAEVKKPGA | 80 |
| TRGV9_ | Chain | SVKVSCKASGFTFTDHYINWVRQATGQGLEWMGQI | |
| 7A5_ | A | YPGSGNTYYNQKFKGRVTMTRDTSISTAYMELSSL | |
| var | | RSEDTAVYYCARNYGDYTIDFWGQGTSVTVSSAST | |
| 17 | | KGPSVFPLAPSSKSTSGGTAALGCLVKDYFPEPVT | |
| | | VSWNSGALTSGVHTFPAVLQSSGLYSLSSVVTVPS | |
| | | SSLGTQTYICNVNHKPSNTKVDKKVEPKSCDKTHT | |
| | | CPPCPAPEAAGGPSVFLFPPKPKDTLMISRTPEVT | |
| | | CVVVSVSHEDPEVKFNWYVDGVEVHNAKTKPREEQ | |
| | | YNSTYRVVSVLTVLHQDWLNGKEYKCKVSNKALPA | |
| | | PIEKTISKAKGQPREPQVYVYPPSREEMTKNQVSL | |
| | | TCLVKGFYPSDIAVEWESNGQPENNYKTTPPVLDS | |
| | | DGSFALVSKLTVDKSRWQQGNVFSCSVMHEALHNR | |
| | | FTQKSLSLSPGK | |
| | Light | MAWVWTLLFLMAAAQSIQADIVMTQSPDSLAVSLG | 81 |
| | Chain | ERATINCKSSQSVLYSSNNKNYLAWYQQKPGQPPK | |
| | | LLIYWASTRESGVPDRFSGSGSGTDFTLTISSLQA | |
| | | EDVAVYYCQQYYRYHTFGTGTKLEIKRTVAAPSVF | |
| | | IFPPSDEQLKSGTASVVCLLNNFYPREAKVQWKVD | |
| | | NALQSGNSQESVTEQDSKDSTYSLSSTLTLSKADY | |
| | | EKHKVYACEVTHQGLSSPVTKSFNRGEC | |
| Anti- | Heavy | MAWVWTLLFLMAAAQSIQAQVQLVQSGAEVKKPGA | 82 |
| TRGV9_ | Chain | SVKVSCKASGFTFTDHYINWVRQATGQGLEWMGQI | |
| 7A5_ | A | YPGSGNTYYNQKFKGRVTMTRNTSISTAYMELSSL | |
| var | | RSEDTAVYYCARNYGDYTIDFWGQGTSVTVSSAST | |
| 29 | | KGPSVFPLAPSSKSTSGGTAALGCLVKDYFPEPVT | |
| | | VSWNSGALTSGVHTFPAVLQSSGLYSLSSVVTVPS | |
| | | SSLGTQTYICNVNHKPSNTKVDKKVEPKSCDKTHT | |
| | | CPPCPAPEAAGGPSVFLFPPKPKDTLMISRTPEVT | |
| | | CVVVSVSHEDPEVKFNWYVDGVEVHNAKTKPREEQ | |
| | | YNSTYRVVSVLTVLHQDWLNGKEYKCKVSNKALPA | |
| | | PIEKTISKAKGQPREPQVYVYPPSREEMTKNQVSL | |
| | | TCLVKGFYPSDIAVEWESNGQPENNYKTTPPVLDS | |
| | | DGSFALVSKLTVDKSRWQQGNVFSCSVMHEALHNR | |
| | | FTQKSLSLSPGK | |
| | Light | MAWVWTLLFLMAAAQSIQADIVMTQSPDSLAVSLG | 83 |
| | Chain | ERATISCKSSQSVLYSSNNKNYLAWYQQKPGQPPK | |
| | | LLIYWASTRESGVPDRFSGSGSGTDFTLTISSLQA | |
| | | EDVAVYYCQQYYRYHTFGTGTKLEIKRTVAAPSVF | |
| | | IFPPSDEQLKSGTASVVCLLNNFYPREAKVQWKVD | |
| | | NALQSGNSQESVTEQDSKDSTYSLSSTLTLSKADY | |
| | | EKHKVYACEVTHQGLSSPVTKSFNRGEC | |
| Anti- | Heavy | SEQUENCE NOT SHOWN | — |
| TAA1 | Chain | | |
| (half- | B | | |
| mAb) | Light | SEQUENCE NOT SHOWN | — |
| | Chain | | |
| Anti- | Heavy | MAWVWTLLFLMAAAQSIQAQITLKESGPTLVKPTQ | 86 |
| RSV | Chain | TLTLTCTFSGESLSTSGMGVSWIRQPPGKALEWLA | |
| (half- | B | HIYWDDDKRYNPSLKSRLTITKDTSKNQVVLTMTN | |
| mAb) | | MDPVDTATYYCARLYGFTYGFAYWGQGTLVTVSSA | |
| | | STKGPSVFPLAPSSKSTSGGTAALGCLVKDYFPEP | |
| | | VTVSWNSGALTSGVHTFPAVLQSSGLYSLSSVVTV | |
| | | PSSSLGTQTYICNVNHKPSNTKVDKKVEPKSCDKT | |
| | | HTCPPCPAPEAAGGPSVFLEPPKPKDTLMISRTPE | |
| | | VTCVVVSVSHEDPEVKFNWYVDGVEVHNAKTKPRE | |
| | | EQYNSTYRVVSVLTVLHQDWLNGKEYKCKVSNKAL | |
| | | PAPIEKTISKAKGQPREPQVYVLPPSREEMTKNQV | |
| | | SLLCLVKGFYPSDIAVEWESNGQPENNYLTWPPVL | |
| | | DSDGSFFLYSKLTVDKSRWQQGNVFSCSVMHEALH | |
| | | NHYTQKSLSLSPGK | |
| | Light | METHSQVFVYMLLWLSGVEGDIVMTQSPDSLAVSL | 87 |
| | Chain | GERATINCRASQSVDYNGISYMHWYQQKPGQPPKL | |
| | | LIYAASNPESGVPDRFSGSGSGTDFTLTISSLQAE | |
| | | DVAVYYCQQIIEDPWTEGQGTKVEIKRTVAAPSVF | |
| | | IFPPSDEQLKSGTASVVCLLNNFYPREAKVQWKVD | |
| | | NALQSGNSQESVTEQDSKDSTYSLSSTLTLSKADY | |
| | | EKHKVYACEVTHQGLSSPVTKSFNRGEC | |

scFv Sequences

| | | | |
|---|---|---|---|
| Anti- | Heavy | MAWVWTLLFLMAAAQSIQADIVMTQSPDSLAVSLG | 70 |
| TRGV9_ | Chain | ERATINCKSSQSVLYSSNNKNYLAWYQQKPGQPPK | |
| 7A5_ | A | LLIYWASTRESGVPDRFSGSGSGTDFTLTISSLQA | |
| var | | EDVAVYYCQQYYRYHTFGTGTKLEIKGGSEGKSSG | |
| 17- | | SGSESKSTGGSQVQLVQSGAEVKKPGASVKVSCKA | |
| scFv | | SGFTFTDHYINWVRQATGQGLEWMGQIYPGSGNTY | |
| | | YNQKFKGRVTMTRDTSISTAYMELSSLRSEDTAVY | |
| | | YCARNYGDYTIDFWGQGTSVTVSSEPKSSDKTHTC | |
| | | PPCPAPEAAGGPSVFLEPPKPKDTLMISRTPEVTC | |
| | | VVVSVSHEDPEVKFNWYVDGVEVHNAKTKPREEQY | |
| | | NSTYRVVSVLTVLHQDWLNGKEYKCKVSNKALPAP | |
| | | IEKTISKAKGQPREPQVYVYPPSREEMTKNQVSLT | |
| | | CLVKGFYPSDIAVEWESNGQPENNYKTTPPVLDSD | |
| | | GSFALVSKLTVDKSRWQQGNVFSCSVMHEALHNHY | |
| | | TQKSLSLSPGK | |
| Anti- | Heavy | MAWVWTLLFLMAAAQSIQADIVMTQSPDSLAVSLG | 73 |
| TRGV9_ | Chain | ERATISCKSSQSVLYSSNNKNYLAWYQQKPGQPPK | |
| 7A5_ | A | LLIYWASTRESGVPDRFSGSGSGTDFTLTISSLQA | |
| var | | EDVAVYYCQQYYRYHTFGTGTKLEIKGGSEGKSSG | |
| 29- | | SGSESKSTGGSQVQLVQSGAEVKKPGASVKVSCKA | |
| scFv | | SGFTFTDHYINWVRQATGQGLEWMGQIYPGSGNTY | |
| | | YNQKFKGRVTMTRNTSISTAYMELSSLRSEDTAVY | |
| | | YCARNYGDYTIDFWGQGTSVTVSSEPKSSDKTHTC | |
| | | PPCPAPEAAGGPSVFLFPPKPKDTLMISRTPEVTC | |
| | | VVVSVSHEDPEVKFNWYVDGVEVHNAKTKPREEQY | |
| | | NSTYRVVSVLTVLHQDWLNGKEYKCKVSNKALPAP | |
| | | IEKTISKAKGQPREPQVYVYPPSREEMTKNQVSLT | |
| | | CLVKGFYPSDIAVEWESNGQPENNYKTTPPVLDSD | |
| | | GSFALVSKLTVDKSRWQQGNVFSCSVMHEALHNHY | |
| | | TQKSLSLSPGK | |
| Anti- | Heavy | SEQUENCE NOT SHOWN | — |
| TAA1- | Chain | | |
| scFv | B | | |
| Anti- | Heavy | MAWVWTLLFLMAAAQSIQADIQMTQSPSSLSASVG | 88 |
| Null- | Chain | DRVTITCRASQSISSYLNWYQQKPGCAPKLLIYAA | |
| scFv | B | SSLQSGVPSRFSGSGSGTDFTLTISSLQPEDFATY | |
| | | YCQQSYSTPLTFGQGTKVEIKGGGSGGSGGCPPCG | |
| | | GSGGEVQLLESGGGLVQPGGSLRLSCAASGFTFSS | |
| | | YAMSWVRQAPGKGLEWVSAISGSGGSTYYADSVKG | |
| | | RFTISRDNSKNTLYLQMNSLRAEDTAVYYCAKYDG | |
| | | IYGELDFWGCGTLVTVSSEPKSSDKTHTCPPCPAP | |
| | | EAAGGPSVFLFPPKPKDTLMISRTPEVTCVVVSVS | |
| | | HEDPEVKFNWYVDGVEVHNAKTKPREEQYNSTYRV | |
| | | VSVLTVLHQDWLNGKEYKCKVSNKALPAPIEKTIS | |
| | | KAKGQPREPQVYVLPPSREEMTKNQVSLLCLVKGF | |
| | | YPSDIAVEWESNGQPENNYLTWPPVLDSDGSFFLY | |
| | | SKLTVDKSRWQQGNVFSCSVMHEALHNHYTQKSLS | |
| | | LSPGK | |

TABLE 17
Anti-TRGV9 Heavy and Light Chain Sequences

| Anti- | | |
|---|---|---|
| body | Heavy Chaim | Light Chain |
| TRGV9_ | EVQLQQSGAELARPGASVKLSCKASGF | DIVMSQSPSSLAVSVG |
| 7A5_1 | TFTDHYINWVKQRTGQGLEWIGQIYPG | EKVTMSCKSSQSLLYS |
| | DGNTYYNQKFKGKATLTADKSSSTAYM | SNQKNYLAWYQQKPGQ |
| | QLSSLTSEDSAVYFCAPNYGDYTIDFW | SPKLLIYWASTRESGV |
| | GQGTSVTVSSASTKGPSVFPLAPSSKS | PDRFTGSGSGTDFTLT |
| | TSGGTAALGCLVKDYFPEPVTVSWNSG | ISSVKAEDLAVYYCQQ |
| | ALTSGVHTFPAVLQSSGLYSLSSVVTV | YYRYHTFGTGTKLEIK |
| | PSSSLGTQTYICNVNHKPSNTKVDKKV | RTVAAPSVFIFPPSDE |
| | EPKSCDKTHTCPPCPAPEAAGGPSVFL | QLKSGTASVVCLLNNF |
| | FPPKPKDTLMISRTPEVTCVVVSVSHE | YPREAKDPEVKFNWYV |
| | NSTYRVVSVLTVLHQDWLNGKEYKCKV | DGVEVHNAKTKPREEQ |
| | SNKALPAPIEKTISKAKGQPREPQVYV | YVQWKVDNALQSGNSQ |
| | YPPSREEMTKNQVSLTCLVKGFYPSDI | ESVTEQDSKDSTYSLS |
| | AVEWESNGQPENNYKTTPPVLDSDGSF | STLTLSKADYEKHKVY |
| | ALVSKLTVDKSRWQQGNVFSCSVMHEA | ACEVTHQGLSSPVTKS |
| | LHNRFTQKSLSLSPGK | FNRGEC |
| | (SEQ ID NO: 69) | (SEQ ID NO: 24) |
| TRGV9_ | QVQLVQSGAEVKKPGASVKVSCKASGF | DIVMTQSPDSLAVSLG |
| var17 | TFTDHYINWVRQATGQGLEWMGQIYPG | ERATINCKSSQSVLYS |
| | SGNTYYNQKFKGRVTMTRDTSISTAYM | SNNKNYLAWYQQKPGQ |
| | ELSSLRSEDTAVYYCARNYGDYTIDFW | PPKLLIYWASTRESGV |
| | GQGTSVTVSSASTKGPSVFPLAPSSKS | PDRFSGSGSGTDFTLT |
| | TSGGTAALGCLVKDYFPEPVTVSWNSG | ISSLQAEDVAVYYCQQ |
| | ALTSGVHTFPAVLQSSGLYSLSSVVTV | YYRYHTFGTGTKLEIK |
| | PSSSLGTQTYICNVNHKPSNTKVDKKV | RTVAAPSVFIFPPSDE |
| | EPKSCDKTHTCPPCPAPEAAGGPSVFL | QLKSGTASVVCLLNNF |
| | FPPKPKDTLMISRTPEVTCVVVSVSHE | YPREAKVQWKVDNALQ |
| | DPEVKFNWYVDGVEVHNAKTKPREEQY | SGNSQESVTEQDSKDS |
| | NSTYRVVSVLTVLHQDWLNGKEYKCKV | TYSLSSTLTLSKADYE |
| | SNKALPAPIEKTISKAKGQPREPQVYV | KHKVYACEVTHQGLSS |
| | YPPSREEMTKNQVSLTCLVKGFYPSDI | PVTKSFNRGEC |
| | AVEWESNGQPENNYKTTPPVLDSDGSF | (SEQ ID NO: 72) |
| | ALVSKLTVDKSRWQQGNVFSCSVMHEA | |
| | LHNRFTQKSLSLSPGK | |
| | (SEQ ID NO: 71) | |
| TRGV9_ | QVQLVQSGAEVKKPGASVKVSCKASGF | DIVMTQSPDSLAVSLG |
| var29 | TFTDHYINWVRQATGQGLEWMGQIYPG | ERATISCKSSQSVLYS |
| | SGNTYYNQKFKGRVTMTRNTSISTAYM | SNNKNYLAWYQQKPGQ |
| | ELSSLRSEDTAVYYCARNYGDYTIDFW | PPKLLIYWASTRESGV |
| | GQGTSVTVSSASTKGPSVFPLAPSSKS | PDRFSGSGSGTDFTLT |
| | TSGGTAALGCLVKDYFPEPVTVSWNSG | ISSLQAEDVAVYYCQQ |
| | ALTSGVHTFPAVLQSSGLYSLSSVVTV | YYRYHTFGTGTKLEIK |
| | PSSSLGTQTYICNVNHKPSNTKVDKKV | RTVAAPSVFIFPPSDE |
| | EPKSCDKTHTCPPCPAPEAAGGPSVFL | QLKSGTASVVCLLNNF |
| | FPPKPKDTLMISRTPEVTCVVVSVSHE | YPREAKVQWKVDNALQ |
| | DPEVKFNWYVDGVEVHNAKTKPREEQY | SGNSQESVTEQDSKDS |
| | NSTYRVVSVLTVLHQDWLNGKEYKCKV | TYSLSSTLTLSKADYE |
| | SNKALPAPIEKTISKAKGQPREPQVYV | KHKVYACEVTHQGLSS |
| | YPPSREEMTKNQVSLTCLVKGFYPSDI | PVTKSFNRGEC |
| | AVEWESNGQPENNYKTTPPVLDSDGSF | (SEQ ID NO: 75) |
| | ALVSKLTVDKSRWQQGNVFSCSVMHEA | |
| | LHNRFTQKSLSLSPGK | |
| | (SEQ ID NO: 74) | |
| TAA1 | SEQUENCE NOT SHOWN | SEQUENCE NOT |
| | | SHOWN |

The antibodies were initially purified by MAB SELECT SURE Protein A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative SUPERDEX 200 10/300 GL (GE healthcare) size exclusion chromatography (SEC) column equilibrated with PBS buffer. The integrity of the sample was assessed by endotoxin measurement and SDS-PAGE under reducing and non-reducing conditions. The final protein concentrations were 1.0 mg/ml for anti-TRGV9/anti-TAA1 and 1.0 mg/mL for ANTI-TRGV9/Null. The final EU levels of ANTI-TRGV9/anti-TAA1 and ANTI-TRGV9/Null based on these were <3.0 EU/mg.

### Example 4.4: Binding Activity of Anti-Taa1 and Anti-Trgv9 Antibodies on Target Cell Lines

Binding of antibodies to TAA1 expressing cell line and γδ T cells was carried out by flow cytometry. Briefly, 50,000 target cells or γδ T cells were incubated at 4° C. for 45 minutes with serial dilutions of various antibodies. After washing with wash buffer (PBS+2% FBS), antibody bound to cell surface was detected by incubating the cells with PE labelled mouse anti human IgG1 secondary antibody (Southern Biotech, Birmingham, AL) for 30 minutes at 4° C. Cells were washed with wash buffer (PBS+2% FBS) and the fluorescence of stained cells was measured on Novocyte flow cytometer. Cells were visualized on forward and sideward scatter and doublets were excluded. No secondary antibody control was used to establish background fluorescence and to gate on specific population. Background value was subtracted from main samples to get specific binding value.

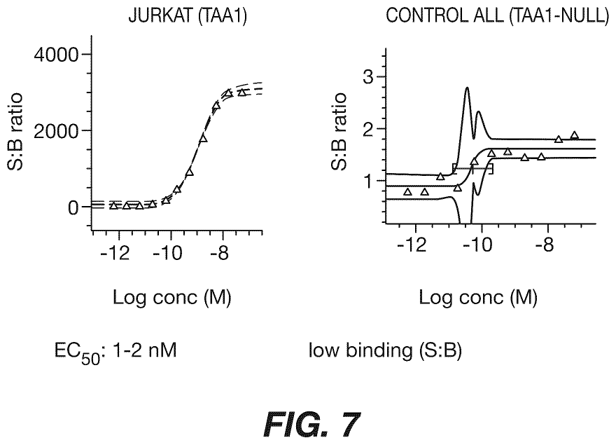

As shown in FIG. 7, the EC50 for binding of anti-TAA1 antibodies (TAA1 mIgG2a, TAA1B1) to TAA1 expressing Jurkat cell lines was ˜1 to 2 nM. TAA1B1 did not show any significant binding to a control ALL cell line that endogenously expresses an unrelated protein (TAA1-NULL), but does not express TAA1.

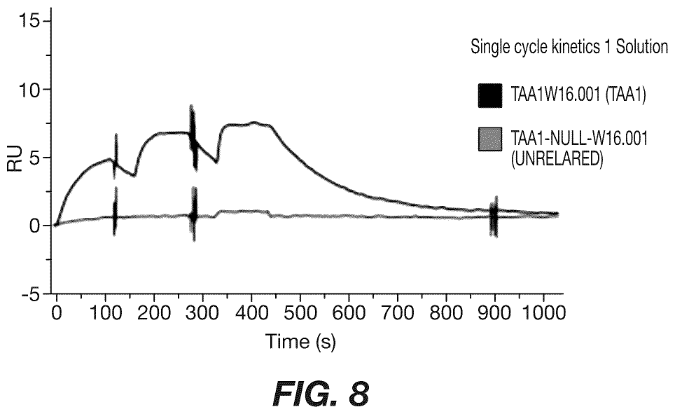

Additional surface plasmon resonance (SPR) experiments were used to determine specific binding of anti-TAA1 mAb to TAA1. Briefly, SPR experiments were carried out in HBSP buffer at 25° C. The experimental set up was following: Goat anti-murine Fc surface was immobilized on a sensor chip, and binding was tested by capturing the mouse anti-human TAA1 clone mAb at different densities. The recombinant TAA1 protein (TAA1W16) and an unrelated protein (TAA1-NULL-W16) were used as analyte to bind in solution in a single cycle kinetics. Raw binding data were processed by double referencing, e.g., interspot on an empty chip surface. As shown in FIG. 8, the anti-TAA1 antibodies (TAA1 mIgG2a, TAA1B1) specifically bound to recombinant TAA1 (TAA1W16). TAA1B1 did not show any significant binding to the unrelated protein (TAA1-NULL-W16).

### Example 4.5: Evaluation of Binding and Cytotoxic Properties of the Anti-Trgv9/Anti-Taa1 Bispecific Antibody Using Jurkat Cells and Human γδ T Cells

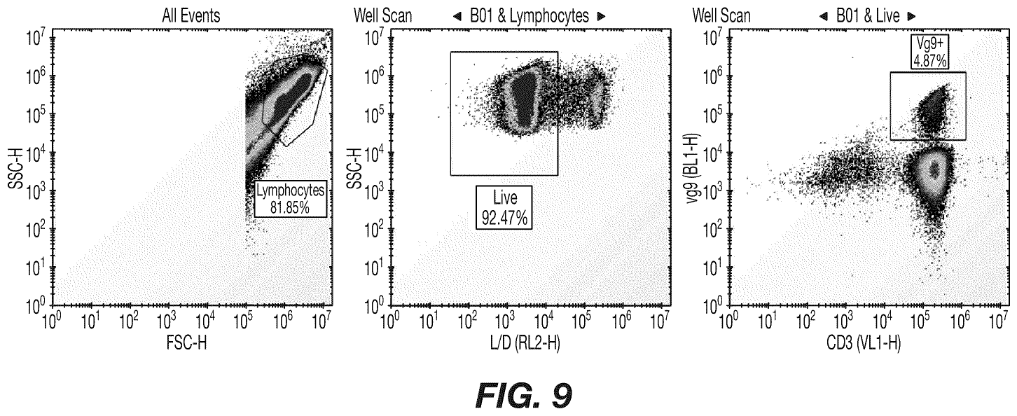

FIG. 9 shows the phenotyping of Vγ9+ cells (TAA1B50) from a healthy donor of used for cytotoxicity studies of an anti-TAA1 x Vγ9 bispecific. FIG. 10 shows that the anti-TRGV9/anti-TAA1 bispecific antibody mediates γδ T cell (TAA1B50) cytotoxicity against TAA1-expressing Jurkat cells in vitro. Enriched γδ T cells (Effectors), isolated from PBMCs cultured with Zoledronic acid+IL-2+IL-15 for 12 days, were co-cultured with CFSE labelled Jurkat cells (Targets) at 0.5:1 to 10:1 E:T ratios in the presence of various concentrations of the bispecific antibody for 24 to 72 hours. Dose response curves show an anti-TRGV9/anti-TAA1 (TAA1 Fab x Vγ9 scFv) bispecific mediated γδ T cell cytotoxicity against TAA1-expressing Jurkat cells in a dose dependent manner, as compared to Jurkat cells that do not endogenously express TAA1. Cytotoxicity values represented here were subtracted of basal cytotoxicity value observed in the absence of bispecific antibody. EC₅₀ values were calculated as described above. Representative data shown are from a single experiment.

To additionally study the ability of a Vγ9xTAA1 bispecific to mediate 76 T cell cytotoxicity against Jurkat cells with γδ T cells from different donors, γδ T cells were enriched. In particular, Vγ9Vδ2 T cells from 5 different donors (328337, 328676, 327587, 328630, 326287) were expanded from total PBMC population for 13 days. Briefly, PBMC were cultured in the presence of zoledronic acid (Sigma, SML0223) (350 nM, days 0 to 13), rhIL-2 (Miltenyi, 130-097-748) (1000 U/mL days 0-2, 800 U/mL days 2-5, 100 U/mL days 5-13) and rhIL-15 (Miltenyi, 130-095-765) (10 ng/mL days 0-13) in complete growth media (RPMI, 10% HI FBS, 1% Pen/strep). At day 13 of expansion, cells were harvested and enriched with EASYSEP™ Human Gamma/Delta T Cell Isolation Kit (Stem Cell Technologies, 19255) according to manufacturer's instructions. Following enrichment procedure, cells were seeded at 1×10⁶/mL in complete growth media with addition of 350 nM zoledronic acid, 100 U/mL IL-2 and 10 ng/mL IL-15 and rested overnight.

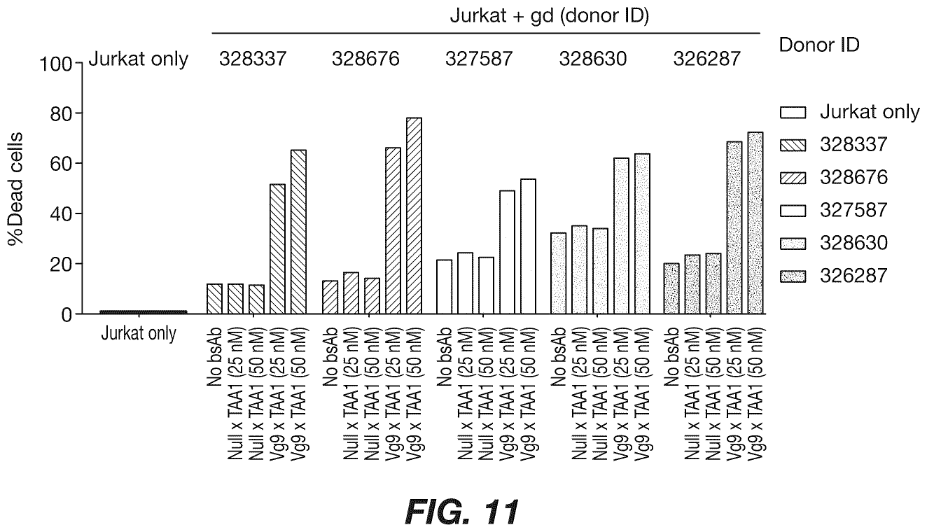

For the killing assay, rested γδ T cells were harvested, and cell number and viability were determined. Target cells (Jurkat cells expressing TAA1) were labelled with 0.25 μM CFSE (Thermo, C34554) for 5 min. at room temperature. Cells were washed 3 times and cell number and viability were determined. Killing assay at an E:T ratio of 1:1 (10⁵ effector cells: 10⁵ target cells) was set up for 16 hours in 96 well plate in complete growth media in the absence on zoledronic acid and cytokines. Vγ9xTAA1 bispecific molecules were adjusted in concentration by limiting dilution to yield final concentration 25 nM and 50 nM in final volume 150 μL/well. After 16 hours, cells were harvested and stained with cocktail containing antibodies against: CD3 (Biolegend, 300424), Vγ9 (Biolegend, 331310), CD25 (Biolegend, 356142), CD69 (Biolegend, 310930), as well as Near-IR (Thermo, L34975) and Fc block (BD, 546219). Cells were washed 3 times, fixed and analysed by flow cytometry. FIG. 11 depicts anti-TRGV9/anti-TAA1 bispecific antibody mediated cytotoxicity. VT9xTAA1 bispecific antibodies at both concentrations tested resulted in greater killing in all five of the donor Vγ9Vδ2 T cell populations as compared to the addition of a NULL/TAA1 bispecific or no bispecific antibody. The additional of Vγ9xTAA1 bispecific antibodies also failed to kill Jurkat cells when donor γδ T cells were not present.

### Example 5—Multispecific Antibodies that Bind Trgv9 and Taa2

Example 5 is based on the premise that γδ T cells, which mainly express heterodimers of TRGV9 and V62 chains demonstrate potent anti-tumor functions. These cells express TCR-TRGV9 and the majority, if not all, of these cells exhibit efficient cytotoxicity of tumor target cells. This ability is then harnessed using bispecific antibodies constructed such that one arm binds to the TRGV9 structure and the other arm binds to a second T cell Tumor-Associated Antigen (TAA2) expressed by the tumor cells (e.g., certain leukemias and lymphomas). Thus, the bispecific antibody bridges the effector and target cells together, resulting in tumor cell killing. This mechanism of action is described in the schematic outlined in FIG. 1.

The subsequent examples can be divided into the following categories: (1) Generation and characterization of bispecific antibodies capable of binding to the TRGV9 arm expressed on γδ T cells and a certain tumor associated antigen (TAA2) (Examples 5.1, 5.2, 5.3 and 5.4); and (2) Evidence for bispecific antibody-enabled target cell killing by γδ T cells expanded in vitro (Example 5.5).

### Example 5.1: Anti-Vg9 Antibody Generation

Immunogen. A recombinant human TCR Vγ9 x Vδ2 fused to a human Fc was used as an immunogen, and the sequence is listed in Table 18.

TABLE 18
Amino acid sequence of recombinant human TCR
Vγ9 × Vδ2 heterodimeric protein fused to
human Fc

| | Pro- | | |
|---|---|---|---|
| | tein | | |
| Name | ID | Sequence | |
| Recom- | Vg9 | MAWVWTLLFLMAAAQSIQAAGHLEQPQISST | SEQ |
| binant | chain | KTLSKTARLECVVSGITISATSVYWYRERPG | ID |
| human | | EVIQFLVSISYDGTVRKESGIPSGKFEVDRI | NO: |
| [TCR | | PETSTSTLTIHNVEKQDIATYYCALWEAQQE | 156 |
| Vg9 × | | LGKKIKVFGPGTKLIITDKQLDADVSPKPTI | |
| Vd2]- | | FLPSIAETKLQKAGTYLCLLEKFFPDVIKIH | |
| hFc | | WEEKKSNTILGSQEGNTMKTNDTYMKFSWLT | |
| | | VPEKSLDKEHRCIVRHENNKNGVDQEIIFPP | |
| | | IKTDVITMDPKDNEPKSCDKTHTCPPCPAPE | |
| | | LLGGPSVFLFPPKPKDTLMISRIPEVTCVVV | |
| | | DVSHEDPEVKFNWYVDGVEVHNAKTKPREEQ | |
| | | YNSTYRVVSVLTVLHQDWLNGKEYKCKVSNK | |
| | | ALPAPIEKTISKAKGQPREPQVYVYPPSREE | |
| | | MTKNQVSLTCLVKGFYPSDIAVEWESNGQPE | |
| | | NNYKTTPPVLDSDGSFALVSKLTVDKSRWQQ | |
| | | GNVFSCSVMHEALHNHYTQKSLSLSPGK | |
| | Vd2 | MAWVWTLLFLMAAAQSIQAAIELVPEHQTVP | SEQ |
| | chain | VSIGVPATLRCSMKGEAIGNYYINWYRKTQG | ID |
| | | NTMTFIYREKDIYGPGFKDNFQGDIDIAKNL | NO: |
| | | AVLKILAPSERDEGSYYCACDTLGMGGEYTD | 157 |
| | | KLIFGKGTRVIVEPRSQPHTKPSVFVMKNGT | |
| | | NVACLVKEFYPKDIRINLVSSKKITEFDPAI | |
| | | VISPSGKYNAVKLGKYEDSNSVTCSVQHDNK | |
| | | TVHSTDFEVKTDSTDHVKPKETENTKQPSKS | |
| | | EPKSCDKTHTCPPCPAPELLGGPSVFLFPPK | |
| | | PKDTLMISRTPEVTCVVVDVSHEDPEVKFNW | |
| | | YVDGVEVHNAKTKPREEQYNSTYRVVSVLTV | |
| | | LHQDWLNGKEYKCKVSNKALPAPIEKTISKA | |
| | | KGQPREPQVYVLPPSREEMTKNQVSLLCLVK | |
| | | GFYPSDIAVEWESNGQPENNYLTWPPVLDSD | |
| | | GSFFLYSKLTVDKSRWQQGNVFSCSVMHEAL | |
| | | HNHYTQKSLSLSPGK | |

Protein Production of the Immunogen. Expression plasmids encoding the immunogen (see Table 18) were transfected into CHO cell at a DNA ratio of 1:1. Total amount of DNA for a 750 mL expression scale was 750 μg. Final expression volume was 1 L after two feedings and enhancer additions. Using an ÄKTAPRIME plus instrument (GE Healthcare Life Sciences), supernatant (1 L) after 7 days was applied with a flow-rate of 5 mL/min to a MAB SELECT SURE (GE Life Sciences) with a column volume (CV) of 10 mL pre-equilibrated with phosphate buffered saline (PBS), pH 6.8. Non-specific proteins binding to the column material was washed off with PBS supplemented with 500 mM NaCl, pH 6.8 (5 CV). The Fc-containing immunogen was eluted stepwise with 40 mM sodium acetate pH 5.0 (5 CV), pH 4.5 (5 CV), pH 4.0 (10 CV), pH 3.5 (5 CV), and pH 3.0 (5 CV). Fractions were pooled, and applied (5 mL) at a flow-rate of 0.2 mL/min on to a HiLoad 16/600 SUPERDEX (GE Healthcare) column pre-equilibrated with PBS (pH 6.8). Target protein was eluted, pooled, and analyzed by SDS-PAGE, analytic SEC, and intact mass by mass spectrometry. Purity was estimated to >99%.

Antibodies were generated using ALIVAMAB transgenic mice technology (Ablexis). ALIVAMAB mice were immunized with recombinant human Vγ9/Vδ2 TCR protein. Lymphocytes were extracted from secondary lymphoid organs and either fused with FO mouse myeloma cell line for hybridoma generation or subjected to single cell sorting via FACS. Hybridoma supernatants were screened by MSD electrochemiluminescence or by FACS for binding to γδ T cells. Confirmed cell binders were light chain isotyped via ELISA. Single cell sorting supernatants were screened by MSD electrochemiluminescence for binding to recombinant human Vγ9/Vδ2 protein. Several hits with the desired binding profile were selected and sequenced, as provided below.

The CDR sequences of certain VG9 antibodies are provided in Tables 19-22, and the respective VH and VL regions are provided in Tables 23-26, respectively.

TABLE 19
CDR Sequences of anti-TRGV9 antibody (Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9B420- | GFTFSNYD | 98 | ISSSSSYI | 99 | ARDVGVTDYYYYGMDV | 100 |
| LH | | | | | | |
| | | SEQ | | SEQ | | SEQ |
| | | ID | | ID | | ID |
| Antibody | LCDR1 | NO: | LCDR2 | NO: | LCDR3 | NO: |
| VG9B420- | QSVASSY | 101 | GAS | 102 | QQYGSSPPYT | 103 |
| LH | | | | | | |

TABLE 20
CDR Sequences of anti-TRGV9 antibody (Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9SB10SC1087_ | GDTFNNYA | 107 | IIPFFGTP | 108 | ARPGSGSPD | 109 |
| P18_D08 | | | | | YYYYDMDV | |
| | | SEQ | | SEQ | | SEQ |
| | | ID | | ID | | ID |
| Antibody | LCDR1 | NO: | LCDR2 | NO: | LCDR3 | NO: |
| VG9SB10SC1087_ | QSLVHSDG | 110 | KIS | 111 | MQATQFPLT | 112 |
| P18_D08 | NTY | | | | | |

TABLE 21
CDR Sequences of anti-TRGV9 antibody (Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9SB10SC1087_ | GGTFS | 117 | NIPIFNTA | 118 | VREGTGYSY | 119 |
| P18_C12 | SYA | | | | GLDY | |
| | | SEQ | | SEQ | | SEQ |
| | | ID | | ID | | ID |
| Antibody | LCDR1 | NO: | LCDR2 | NO: | LCDR3 | NO: |
| VG9SB10SC1087_ | QSLIHS | 120 | KIS | 121 | MQAKQFPIT | 122 |
| P18_C12 | DGNTY | | | | | |

TABLE 22
CDR Sequences of anti-TRGV9 antibody (Vγ9 clone).

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| VG9SB10SC1087_ | GGSISSG | 127 | IYNSGST | 128 | ARDSNYEWF | 129 |
| P19_C03 | GSY | | | | FDL | |
| | | SEQ | | SEQ | | SEQ |
| | | ID | | ID | | ID |
| Antibody | LCDR1 | NO: | LCDR2 | NO: | LCDR3 | NO: |
| VG9SB1-SC1087_ | QSVSSY | 130 | DAS | 131 | QQRSNWPLT | 132 |
| P19_C03 | | | | | | |

TABLE 23
Heavy chain and light chain V-region sequences
of anti-TRGV9 antibody (Vγ9 clone).

| | | SEQ |
|---|---|---|
| | | ID |
| mAb ID | | NO: |
| | Heavy Chain V-region Amino Acid | |
| | Sequence | |
| VG9B420- | EVQLVESGGGLVKPGGSLRLSCSASGFTFSNYDMNW | 104 |
| LH | VRQAPGKGLEWVSSISSSSSYIYYADSVKGRFTISR | |
| | DNAKNSLYLQMNSLRAEDTAVYHCARDVGVTTDYYY | |
| | YGMDVWGQGTMVTVSS | |
| | Light Chain V-region Amino Acid | |
| | Sequence | |
| VG9B420- | EIVMTQSPGTLSLSPGDRATLSCRASQSVASSYLAW | 105 |
| LH | YQQKPGQSPRLLIYGASSRATGIPDRFSGSGSGTDF | |
| | TLTISRLEPEDFAVYYCQQYGSSPPYTFGQGTRLEI | |
| | K | |

TABLE 24
Heavy chain and light chain V-region sequences
of anti-TRGV9 antibody (Vγ9 clone).

| | | SEQ |
|---|---|---|
| | | ID |
| mAb ID | | NO: |
| | Heavy Chain V-region Amino Acid | |
| | Sequence | |
| VG9SB10SC1 | EVQLVQSGAEVKKPGSSVKVSCKASGDTFNNYA | 113 |
| 087_P18_D08 | ISWVRQAPGQGLEWMGGIIPFFGTPDYAQKFQG | |
| | RVTITADKSTSTAYMELSGLRSEDTAVYYCARP | |
| | GSGSPDYYYYDMDVWGQGTTVTVSS | |
| | Light Chain V-region Amino Acid | |
| | Sequence | |
| VG9SB10SC1 | DIVMTQTPLSSPVTLGQPASISCRSSQSLVHSD | 114 |
| 087_P18_D08 | GNTYLSWLQQRPGQPPRLLIYKISNRFSGVPDR | |
| | FSGSGAGTDFTLKINRVEAEDVGVYYCMQATQF | |
| | PLTFGGGTKVEIK | |

TABLE 25
Heavy chain and light chain V-region sequences
of anti-TRGV9 antibody (Vγ9 clone).

| | | SEQ |
|---|---|---|
| | | ID |
| mAb ID | | NO: |
| | Heavy Chain V-region Amino Acid | |
| | Sequence | |
| VG9SB10SC1 | EVQLVQSGAEVKKPGSSVKVSCKASGGTFSSYA | 123 |
| 087_P18_C12 | ISWVRQAPGQGLEWMGGNIPIFNTANYAQKFQD | |
| | RVTITADKSTSTAYMELSSLRSEDTAVYYCVRE | |
| | GTGYSYGLDYWGQGTPVTVSS | |
| | Light Chain V-region Amino Acid | |
| | Sequence | |
| VG9SB10SC1 | EIVMTQSPLSSPVTLGQPASISCRSSQSLIHSD | 124 |
| 087_P18_C12 | GNTYLSWLQQRPGQPPRLLIYKISNRFSGVPDR | |
| | F4SGSGAGTDFTLKISRVEAEDVGIYYCMQAKQ | |
| | FPITFGQGTKVDIK | |

TABLE 26
Heavy chain and light chain V-region sequences
of anti-TRGV9 antibody (Vγ9 clone).

| | | SEQ |
|---|---|---|
| | | ID |
| mAb ID | | NO: |
| | Heavy Chain V-region Amino Acid | |
| | Sequence | |
| VG9SB10SC1 | QVQLQESGPGLVKPSQTLSLTCTVSGGSISSGG | 133 |
| 087_P19_C03 | SYWSWIRQHPGKGLEWIGYIYNSGSTYYNPSLK | |
| | SRVSMSVDTSKNQFSLKLSSVTAADTAVYYCAR | |
| | DSNYEWFFDLWGPGTLVTVSS | |
| | Light Chain V-region Amino Acid | |
| | Sequence | |
| VG9SB10SC1 | EIVMTQSPATLSLSPGERATLSCRASQSVSSYL | 134 |
| 087_P19_C03 | AWYQQKPGQAPRLLIYDASNRATGIPARFSGSG | |
| | SGTDFTLTISSLEPEDFAVYYCQQRSNWPLTFG | |
| | GGTKVEIK | |

Variable Region Cloning. Both RNA purified by QIAGEN kit (RNEASY Plus Mini Kit) and B cells lysate were used for cDNA synthesis using the Smarter cDNA synthesis kit (Clontech, Mount View, CA). To facilitate cDNA synthesis, oligodT was used to prime reverse transcription of all messenger RNAs followed by “5′ capping” with a SMARTER IIA oligonucleotide. Subsequent amplification of the VH and VL fragments was performed using a two-step PCR amplification using 5′ primers targeting the Smarter IIA cap and 3′ primers targeting consensus regions in CH1. Briefly, each 50 μl PCR reaction consists of 20 μM of forward and reverse primer mixes, 25 μl of PRIMESTAR MAX DNA polymerase premix (Clontech), 2 μl of unpurified cDNA, and 21 μl of double-distilled H₂ O. The cycling program started at 94° C. for 3 min, followed by 35 cycles (94° C. for 30 Sec, 55° C. for 1 min, 68° C. for 1 min), and ended at 72° C. for 7 min. The second round PCR was performed with VL and VH 2nd round primers containing 15 bp complementary extensions that “overlap” respective regions in their respective Lonza mother vector (VH and VL). Second round PCR was performed with the following program: 94° C. for 3 min; 35 cycles (94° C. for 30 Sec, 55° C. for 1 min, 68° C. for 1 min), and ended at 72° C. for 7 min. IN-FUSION® HD Cloning Kit (Clonetech, U.S.A.) was used for directional cloning of VL gene into Lonza huIgK or Lambda vector and VH gene into Lonza huIgG1 vector. To facilitate IN-FUSION® HD Cloning, PCR products were treated with Cloning Enhancer before IN-FUSION HD Cloning. Cloning and transformation were performed according to manufacturer's protocol (Clonetech, U.S.A.). Mini-prep DNAs were subjected to Sanger sequencing to confirm that complete V-gene fragments were obtained.

### Example 5.2: Production and De Novo Sequencing of Anti-Trgv9 Mab

The mouse IgG1 anti-human T cell receptor anti-TRGV9 clone B3 was sourced commercially. Sample preparation and LC-MS/MS analysis were performed by RAPID NOVOR (ON, Canada). Twenty-one in-solution and in-gel digestions were prepared using six different enzymes (Pepsin, Trypsin, Chymotrypsin, Asp N, Lys C, Glu C). The in-solution digestions for the sample was processed with disulfide reduction, alkylation, and then enzyme digestion. Each digestion contains peptides from all immunoglobulin chains. The in-gel digestions were prepared for immunoglobulin chains after gel separation. The sample was processed with disulfide reduction, gel separation, deglycosylation, disulfide reduction a second time, alkylation and then digestion. Digestions were analyzed by LC-MS/MS using THERMO-FISHER Q EXACTIVE™, ORBITRAP FUSION™ mass spectrometers. Peptides were characterized from LC-MS/MS data using de novo peptide sequencing and then assembled into antibody sequences.

The three VH CDR and three VL CDR sequences of anti-human T cell receptor Vγ9 clone B3 are shown in Table 27 (SEQ ID NOs:89-94, respectively); and the VH and VL sequences of the anti-human T cell receptor anti-human T cell receptor Vγ9 clone B3 are shown in Table 28 (SEQ ID NOs:95 and 96, respectively).

TABLE 27
CDR sequences of mouse anti-human TCR Vγ9
clone B3.

| | | SEQ | | SEQ | | SEQ |
|---|---|---|---|---|---|---|
| | | ID | | ID | | ID |
| Antibody | HCDR1 | NO: | HCDR2 | NO: | HCDR3 | NO: |
| Vg9_B3_RN | GFTFSSNY | 89 | IHGGTGGI | 90 | ARRGYGAWFAY | 91 |
| | | SEQ | | SEQ | | SEQ |
| | | ID | | ID | | ID |
| Antibody | LCDR1 | NO: | LCDR2 | NO: | LCDR3 | NO: |
| Vg9_B3_RN | ENIHNY | 92 | NAK | 93 | QHFWSYPLT | 94 |

TABLE 28
Heavy chain and light chain sequences of mouse
anti-human TCR Vγ9 clone B3.

| | | SEQ |
|---|---|---|
| | | ID |
| mAb ID | | NO: |
| | Heavy Chain Amino Acid Sequence | |
| | (from VG9B2) | |
| Vg9_B3_RN | QGQMQQSGAELVKPGASVKLSCKTSGFTFSSNY | 95 |
| | ISWLKQKPGQSLEWIAWIHGGTGGIGYNQKFTG | |
| | KAQLTVDTSSTTAYMQFSSLTTEDSAIYYCARR | |
| | GYGAWFAYWGQGTLVTVSA | |
| | Light Chain Amino Acid Sequence | |
| | (from VG9B2) | |
| Vg9_B3_RN | DIQMTQSPASLSASVGETVTITCRASENIHNYL | 96 |
| | AWYQQKQGKSPQLLVYNAKTLADGVPSRFSGSG | |
| | SGTQYSLKINSLQPEDFGNYYCQHFWSYPLTFG | |
| | AGTKLELK | |

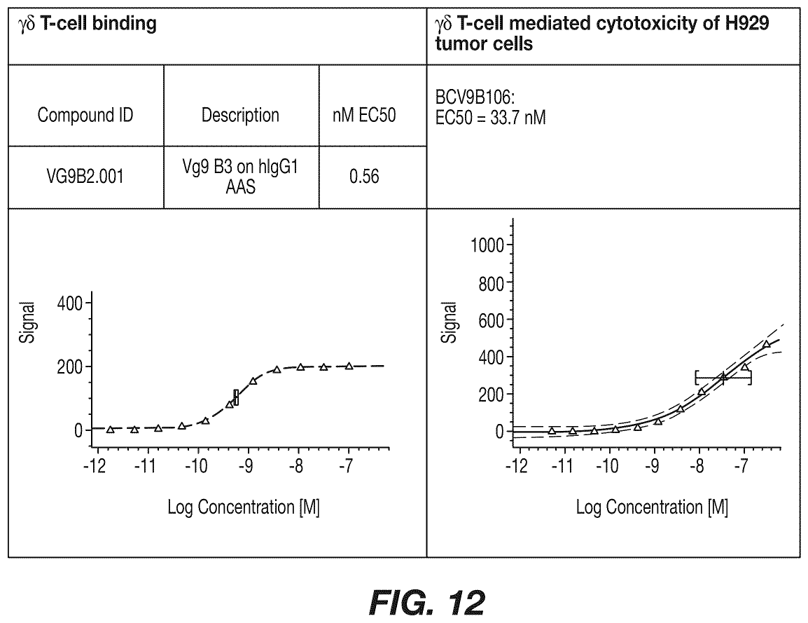

The two antibodies (VG9B2) were expressed in CHO-Expi cells. The purified chimera human IgG1 mAb (silent Fc) demonstrated binding to human γδ T cells showing specificity toward TCR Vγ9, as shown in FIG. 12 (left panel).

### Example 5.3: Production and De Novo Sequencing of Anti-Taa2 Mab

An anti-TAA2 clone was obtained and sequenced (data not shown).

### Example 5.4: Preparation of Anti-Trgv9/Anti-Taa2 Bispecific Antibodies

The variable region sequence of anti-TRGV9 and anti-TAA2 antibodies was used to generate a bispecific human IgG1 antibody to be tested for T cell re-directed killing of H929 cells, which express TAA2. A summary of Vγ9 and TAA2 clones is provided in Table 29.

TABLE 29
Summary of Vγ9 and TAA2 clones

| | | B # |
|---|---|---|
| i. | VG9B420-LH-scFv Half Ab | TAA2V9B101 |
| ii. | VG9SB10SC1087_P18_D08-Fab Half Ab | TAA2V9B100 |
| iii. | VG9SB10SC1087_P18_C12-Fab Half Ab | TAA2V9B101 |
| iv. | TAA2 | Not Shown |
| v. | VG9SB10SC1087_P19_C03-Fab Half Ab | TAA2V9B103 |

The bispecific antibodies were produced as Fab (Vg9) x scFv (TAA2) and scFv (Vg9) x Fab (TAA2) antibodies in the knob-into-hole format as human IgG1 with silent Fe. Nucleic acid sequences encoding variable regions were subcloned into a custom mammalian expression vectors containing constant region of human IgG1 expression cassettes using standard PCR restriction enzyme based standard cloning techniques, and sequenced verified. The bispecific antibodies were expressed by transient transfection in Chinese hamster ovary cell line.

The sequences of the bispecific antibodies expressed in the CHO cells are shown in Table 30 below.

TABLE 30
Sequences of antibodies expressed in CHO cells

| | | | SEQ |
|---|---|---|---|
| | ‘Knob’ arm and ‘hole’ arm amino | | ID |
| mAb ID | acid sequence | | NO: |
| TAA2- | Heavy | SEQUENCE NOT SHOWN | — |
| Fab | Chain | | |
| (half- | B | | |
| mAb) | TAA2V9B7 | | |
| | 1 | | |
| | Light | SEQUENCE NOT SHOWN | — |
| | Chain | | |
| | TAA2V9B7 | | |
| | 1 | | |
| VG9SB10 | Heavy | MAWVWTLLFLMAAAQSIQAEVQLV | 150 |
| SC1087_ | Chain | QSGAEVKKPGSSVKVSCKASGDTF | |
| P | A | NNYAISWVRQAPGQGLEWMGGIIP | |
| 18_D08- | TAA2V9B1 | FFGTPDYAQKFQGRVTITADKSTS | |
| Fab | 00 | TAYMELSGLRSEDTAVYYCARPGS | |
| (half- | | GSPDYYYYDMDVWGQGTTVTVSSA | |
| mAb) | | STKGPSVFPLAPSSKSTSGGTAAL | |
| | | GCLVKDYFPEPVTVSWNSGALTSG | |
| | | VHTFPAVLQSSGLYSLSSVVTVPS | |
| | | SSLGTQTYICNVNHKPSNTKVDKK | |
| | | VEPKSCDKTHTCPPCPAPEAAGGP | |
| | | SVFLFPPKPKDTLMISRTPEVTCV | |
| | | VVSVSHEDPEVKFNWYVDGVEVHN | |
| | | AKTKPREEQYNSTYRVVSVLTVLH | |
| | | QDWLNGKEYKCKVSNKALPAPIEK | |
| | | TISKAKGQPREPQVYVYPPSREEM | |
| | | TKNQVSLTCLVKGFYPSDIAVEWE | |
| | | SNGQPENNYKTTPPVLDSDGSFAL | |
| | | VSKLTVDKSRWQQGNVFSCSVMHE | |
| | | ALHNRFTQKSLSLSPGK | |
| | Light | MAWVWTLLFLMAAAQSIQADIVMT | 151 |
| | Chain | QTPLSSPVTLGQPASISCRSSQSL | |
| | A | VHSDGNTYLSWLQQRPGQPPRLLI | |
| | TAA2V9B1 | YKISNRFSGVPDRFSGSGAGTDFT | |
| | 00 | LKINRVEAEDVGVYYCMQATQFPL | |
| | | TFGGGTKVEIKRTVAAPSVFIFPP | |
| | | SDEQLKSGTASVVCLLNNFYPREA | |
| | | KVQWKVDNALQSGNSQESVTEQDS | |
| | | KDSTYSLSSTLTLSKADYEKHKVY | |
| | | ACEVTHQGLSSPVTKSFNRGEC | |
| VG9SB10 | Heavy | MAWVWTLLFLMAAAQSIQAEVQLV | 152 |
| SC1087_ | Chain | QSGAEVKKPGSSVKVSCKASGGTF | |
| P | A | SSYAISWVRQAPGQGLEWMGGNIP | |
| 18_C12- | TAA2V9B1 | IFNTANYAQKFQDRVTITADKSTS | |
| Fab | 01 | TAYMELSSLRSEDTAVYYCVREGT | |
| (half- | | GYSYGLDYWGQGTPVTVSSASTKG | |
| mAb) | | PSVFPLAPSSKSTSGGTAALGCLV | |
| | | KDYFPEPVTVSWNSGALTSGVHTF | |
| | | PAVLQSSGLYSLSSVVTVPSSSLG | |
| | | TQTYICNVNHKPSNTKVDKKVEPK | |
| | | SCDKTHTCPPCPAPEAAGGPSVFL | |
| | | FPPKPKDTLMISRTPEVTCVVVSV | |
| | | SHEDPEVKFNWYVDGVEVHNAKTK | |
| | | PREEQYNSTYRVVSVLTVLHQDWL | |
| | | NGKEYKCKVSNKALPAPIEKTISK | |
| | | AKGQPREPQVYVYPPSREEMTKNQ | |
| | | VSLTCLVKGFYPSDIAVEWESNGQ | |
| | | PENNYKTTPPVLDSDGSFALVSKL | |
| | | TVDKSRWQQGNVFSCSVMHEALHN | |
| | | RFTQKSLSLSPGK | |
| | Light | MAWVWTLLFLMAAAQSIQAEIVMT | 153 |
| | Chain | QSPLSSPVTLGQPASISCRSSQSL | |
| | A | IHSDGNTYLSWLQQRPGQPPRLLI | |
| | TAA2V9B1 | YKISNRFSGVPDRFSGSGAGTDFT | |
| | 01 | LKISRVEAEDVGIYYCMQAKQFPI | |
| | | TFGQGTKVDIKRTVAAPSVFIFPP | |
| | | SDEQLKSGTASVVCLLNNFYPREA | |
| | | KVQWKVDNALQSGNSQESVTEQDS | |
| | | KDSTYSLSSTLTLSKADYEKHKVY | |
| | | ACEVTHQGLSSPVTKSFNRGEC | |
| VG9SB10 | Heavy | MAWVWTLLFLMAAAQSIQAQVQLQ | 154 |
| SC1087_ | Chain | ESGPGLVKPSQTLSLTCTVSGGSI | |
| P | A | SSGGSYWSWIRQHPGKGLEWIGYI | |
| 19_C03- | TAA2V9B1 | YNSGSTYYNPSLKSRVSMSVDTSK | |
| Fab | 03 | NQFSLKLSSVTAADTAVYYCARDS | |
| (half- | | NYEWFFDLWGPGTLVTVSSASTKG | |
| mAb) | | PSVFPLAPSSKSTSGGTAALGCLV | |
| | | KDYFPEPVTVSWNSGALTSGVHTF | |
| | | PAVLQSSGLYSLSSVVTVPSSSLG | |
| | | TQTYICNVNHKPSNTKVDKKVEPK | |
| | | SCDKTHTCPPCPAPEAAGGPSVFL | |
| | | FPPKPKDTLMISRTPEVTCVVVSV | |
| | | SHEDPEVKFNWYVDGVEVHNAKTK | |
| | | PREEQYNSTYRVVSVLTVLHQDWL | |
| | | NGKEYKCKVSNKALPAPIEKTISK | |
| | | AKGQPREPQVYVYPPSREEMTKNQ | |
| | | VSLTCLVKGFYPSDIAVEWESNGQ | |
| | | PENNYKTTPPVLDSDGSFALVSKL | |
| | | TVDKSRWQQGNVFSCSVMHEALHN | |
| | | RFTQKSLSLSPGK | |
| | Light | MAWVWTLLFLMAAAQSIQAEIVMT | 155 |
| | Chain | QSPATLSLSPGERATLSCRASQSV | |
| | A | SSYLAWYQQKPGQAPRLLIYDASN | |
| | TAA2V9B1 | RATGIPARFSGSGSGTDFTLTISS | |
| | 03 | LEPEDFAVYYCQQRSNWPLTFGGG | |
| | | TKVEIKRTVAAPSVFIFPPSDEQL | |
| | | KSGTASVVCLLNNFYPREAKVQWK | |
| | | VDNALQSGNSQESVTEQDSKDSTY | |
| | | SLSSTLTLSKADYEKHKVYACEVT | |
| | | HQGLSSPVTKSFNRGEC | |

scFv Sequences

| | | | |
|---|---|---|---|
| Vg9-B3- | Heavy | MAWVWTLLFLMAAAQSIQADIQMT | SEQ |
| LH-scFv | Chain | QSPASLSASVGETVTITCRASENI | ID |
| (half- | A | HNYLAWYQQKQGKSPQLLVYNAKT | NO: |
| mAb) | TAA2V9B1 | LADGVPSRFSGSGSGTQYSLKINS | 97 |
| | 06 | LQPEDFGNYYCQHFWSYPLTFGAG | |
| | | TKLELKGGSEGKSSGSGSESKSTG | |
| | | GSQGQMQQSGAELVKPGASVKLSC | |
| | | KTSGFTFSSNYISWLKQKPGQSLE | |
| | | WIAWIHGGTGGIGYNQKFTGKAQL | |
| | | TVDTSSTTAYMQFSSLTTEDSAIY | |
| | | YCARRGYGAWFAYWGQGTLVTVSA | |
| | | EPKSSDKTHTCPPCPAPEAAGGPS | |
| | | VFLFPPKPKDTLMISRTPEVTCVV | |
| | | VSVSHEDPEVKFNWYVDGVEVHNA | |
| | | KTKPREEQYNSTYRVVSVLTVLHQ | |
| | | DWLNGKEYKCKVSNKALPAPIEKT | |
| | | ISKAKGQPREPQVYVYPPSREEMT | |
| | | KNQVSLTCLVKGFYPSDIAVEWES | |
| | | NGQPENNYKTTPPVLDSDGSFALV | |
| | | SKLTVDKSRWQQGNVFSCSVMHEA | |
| | | LHNHYTQKSLSLSPGK | |
| VG9B420- | Heavy | MAWVWTLLFLMAAAQSIQAEIVMT | SEQ |
| LH-scFv | Chain | QSPGTLSLSPGDRATLSCRASQSV | ID |
| (half- | A | ASSYLAWYQQKPGQSPRLLIYGAS | NO: |
| mAb) | TAA2V9B7 | SRATGIPDRFSGSGSGTDFTLTIS | 106 |
| | 1 | RLEPEDFAVYYCQQYGSSPPYTFG | |
| | | QGTRLEIKGGSEGKSSGSGSESKS | |
| | | TGGSEVQLVESGGGLVKPGGSLRL | |
| | | SCSASGFTFSNYDMNWVRQAPGKG | |
| | | LEWVSSISSSSSYIYYADSVKGRF | |
| | | TISRDNAKNSLYLQMNSLRAEDTA | |
| | | VYHCARDVGVTTDYYYYGMDVWGQ | |
| | | GTMVTVSSEPKSSDKTHTCPPCPA | |
| | | PEAAGGPSVFLFPPKPKDTLMISR | |
| | | TPEVTCVVVSVSHEDPEVKFNWYV | |
| | | DGVEVHNAKTKPREEQYNSTYRVV | |
| | | SVLTVLHQDWLNGKEYKCKVSNKA | |
| | | LPAPIEKTISKAKGQPREPQVYVY | |
| | | PPSREEMTKNQVSLTCLVKGFYPS | |
| | | DIAVEWESNGQPENNYKTTPPVLD | |
| | | SDGSFALVSKLTVDKSRWQQGNVF | |
| | | SCSVMHEALHNHYTQKSLSLSPGK | |
| TAA2- | Heavy | SEQUENCE NOT SHOWN | — |
| scFv | Chain | | |
| (half- | B | | |
| mAb) | TAA2V9B1 | | |
| | 01 | | |

TABLE 31
Anti-TRGV9 and Anti-TAA2 Heavy and Light
Chain Sequences

| Antibody | Heavy Chain | Light Chain |
|---|---|---|
| VG9SB10 | EVQLVQSGAEVKKPGSSVKVS | DIVMTQTPLSSPVT |
| SC1087_ | CKASGDTFNNYAISWVRQAPG | LGQPASISCRSSQS |
| P18_D08 | QGLEWMGGIIPFFGTPDYAQK | LVHSDGNTYLSWLQ |
| | FQGRVTITADKSTSTAYMELS | QRPGQPPRLLIYKI |
| | GLRSEDTAVYYCARPGSGSPD | SNRFSGVPDRFSGS |
| | YYYYDMDVWGQGTTVTVSSAS | GAGTDFTLKINRVE |
| | TKGPSVFPLAPSSKSTSGGTA | AEDVGVYYCMQATQ |
| | ALGCLVKDYFPEPVTVSWNSG | FPLTFGGGTKVEIK |
| | ALTSGVHTFPAVLQSSGLYSL | RTVAAPSVFIFPPS |
| | SSVVTVPSSSLGTQTYICNVN | DEQLKSGTASVVCL |
| | HKPSNTKVDKKVEPKSCDKTH | LNNFYPREAKVQWK |
| | TCPPCPAPEAAGGPSVFLFPP | VDNALQSGNSQESV |
| | KPKDTLMISRTPEVTCVVVSV | TEQDSKDSTYSLSS |
| | SHEDPEVKFNWYVDGVEVHNA | TLTLSKADYEKHKV |
| | KTKPREEQYNSTYRVVSVLTV | YACEVTHQGLSSPV |
| | LHQDWLNGKEYKCKVSNKALP | TKSFNRGEC |
| | APIEKTISKAKGQPREPQVYV | (SEQ ID |
| | YPPSREEMTKNQVSLTCLVKG | NO: 116) |
| | FYPSDIAVEWESNGQPENNYK | |
| | TTPPVLDSDGSFALVSKLTVD | |
| | KSRWQQGNVFSCSVMHEALHN | |
| | RFTQKSLSLSPGK | |
| | (SEQ ID NO: 115) | |
| VG9SB1 | EVQLVQSGAEVKKPGSSVKVS | EIVMTQSPLSSPVT |
| 0SC108 | CKASGGTFSSYAISWVRQAPG | LGQPASISCRSSQS |
| 7_P18_ | QGLEWMGGNIPIFNTANYAQK | LIHSDGNTYLSWLQ |
| C12 | FQDRVTITADKSTSTAYMELS | QRPGQPPRLLIYKI |
| | SLRSEDTAVYYCVREGTGYSY | SNRFSGVPDRFSGS |
| | GLDYWGQGTPVTVSSASTKGP | GAGTDFTLKISRVE |
| | SVTSGGTAALGCLVKDYFPEP | AEDVGIYYCMQAKQ |
| | VTVSWNSGALTSGVHTFPAVL | FPITFGQGTKVDIK |
| | QSSGLYSLSSVVTVPSSSLGT | RTVAAPSVFIFPPS |
| | QTYICNVNHKPSNTKVDKKVE | DEQLKSGTASVVCL |
| | PKSCDKTHTCPPCPAPEAAGG | LNNFYPREAKVQWK |
| | PSVFLFPPKPKDTLMISRTPE | VDNALQSGNSQESV |
| | VTCVVVSVSHEDPEVKFNWYV | TEQDSKDSTYSLSS |
| | DGVEVHNAKTKPREEQYNSTY | TLTLSKADYEKHKV |
| | RVVSVLTVLHQDWLNGKEYKC | YACEVTHQGLSSPV |
| | KVSNKALPAPIEKTISKAKGQ | TKSFNRGEC |
| | PREPQVYVYPPSREEMTKNQV | (SEQ ID |
| | SLTCLVKGFYPSDIAVEWESN | NO: 126) |
| | GQPENNYKTTPPVLDSDGSFA | |
| | LVSKLTVDKSRWQQGNVFSCS | |
| | VMHEALHNRFTQKSLSLSPGK | |
| | (SEQ ID NO: 125) | |
| VG9SB1 | QVQLQESGPGLVKPSQTLSLT | EIVMTQSPATLSLS |
| 0SC108 | CTVSGGSISSGGSYWSWIRQH | PGERATLSCRASQS |
| 7_P19_ | PGKGLEWIGYIYNSGSTYYNP | VSSYLAWYQQKPGQ |
| C03 | SLKSRVSMSVDTSKNQFSLKL | APRLLIYDASNRAT |
| | SSVTAADTAVYYCARDSNYEW | GIPARFSGSGSGTD |
| | FFDLWGPGTLVTVSSASTKGP | FTLTISSLEPEDFA |
| | SVFPLAPSSKSTSGGTAALGC | VYYCQQRSNWPLTE |
| | LVKDYFPEPVTVSWNSGALTS | GGGTKVEIKRTVAA |
| | GVHTFPAVLQSSGLYSLSSVV | PSVFIFPPSDEQLK |
| | TVPSSSLGTQTYICNVNHKPS | SGTASVVCLLNNFY |
| | NTKVDKKVEPKSCDKTHTCPP | PREAKVQWKVDNAL |
| | CPAPEAAGGPSVFLFPPKPKD | QSGNSQESVTEQDS |
| | TLMISRTPEVTCVVVSVSHED | KDSTYSLSSTLTLS |
| | PEVKFNWYVDGVEVHNAKTKP | KADYEKHKVYACEV |
| | REEQYNSTYRVVSVLTVLHQD | THQGLSSPVTKSFN |
| | WLNGKEYKCKVSNKALPAPIE | RGEC |
| | KTISKAKGQPREPQVYVYPPS | (SEQ ID |
| | REEMTKNQVSLTCLVKGFYPS | NO: 136) |
| | DIAVEWESNGQPENNYKTTPP | |
| | VLDSDGSFALVSKLTVDKSRW | |
| | QQGNVFSCSVMHEALHNRFTQ | |
| | KSLSLSPGK | |
| | (SEQ ID NO: 135) | |
| TAA2 | SEQUENCE NOT SHOWN | SEQUENCE NOT |
| | | SHOWN |

The antibodies were initially purified by MABSELECT SURE PROTEIN A column (GE Healthcare). The column was equilibrated with PBS pH 7.2 and loaded with fermentation supernatant at a flow rate of 2 mL/min. After loading, the column was washed with 4 column volumes of PBS followed by elution in 30 mM sodium acetate, pH 3.5. Fractions containing protein peaks as monitored by absorbance at 280 nm were pooled and neutralized to pH 5.0 by adding 1% 3 M sodium acetate pH 9.0. The bispecific mAbs were further purified on a preparative SUPERDEX 200 10/300 GL (GE healthcare) size exclusion chromatography (SEC) column equilibrated with PBS buffer. The integrity of sample was assessed by endotoxin measurement (<3.0 EU/mg), SDS-PAGE under reducing and non-reducing conditions, SEC, and intact mass by MS.

### Example 5.5: Evaluation of Binding and Cytotoxic Properties of the Anti-Trgv9/Anti-Taa2 Bispecific Antibody Using H929 Cells and Human γδ T Cells

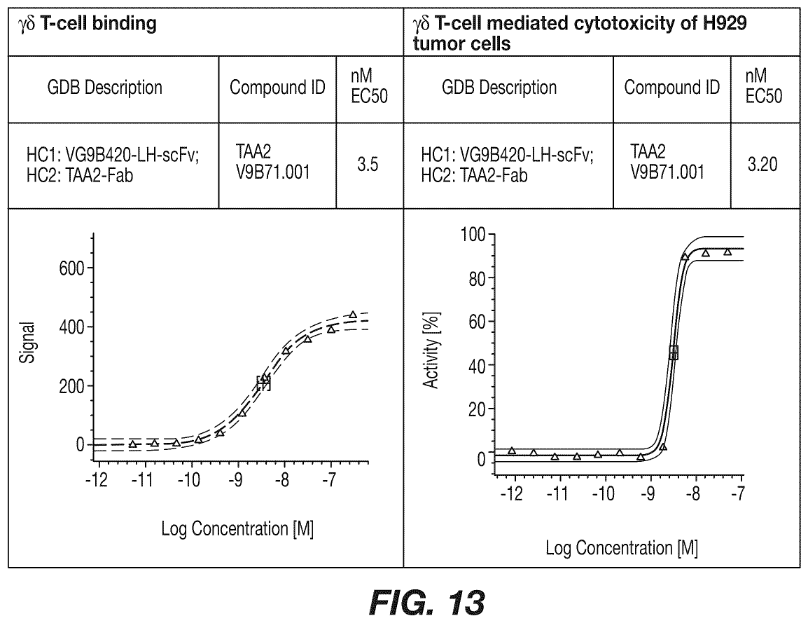

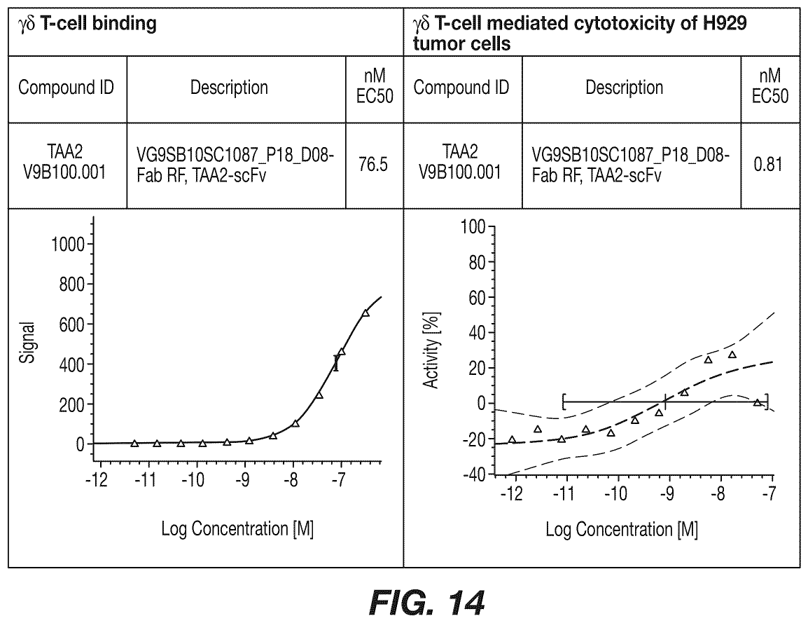

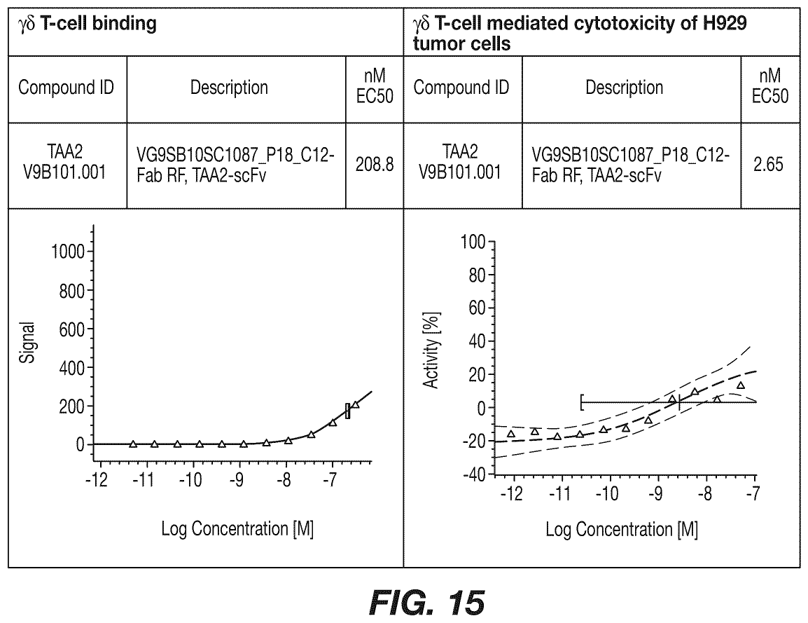

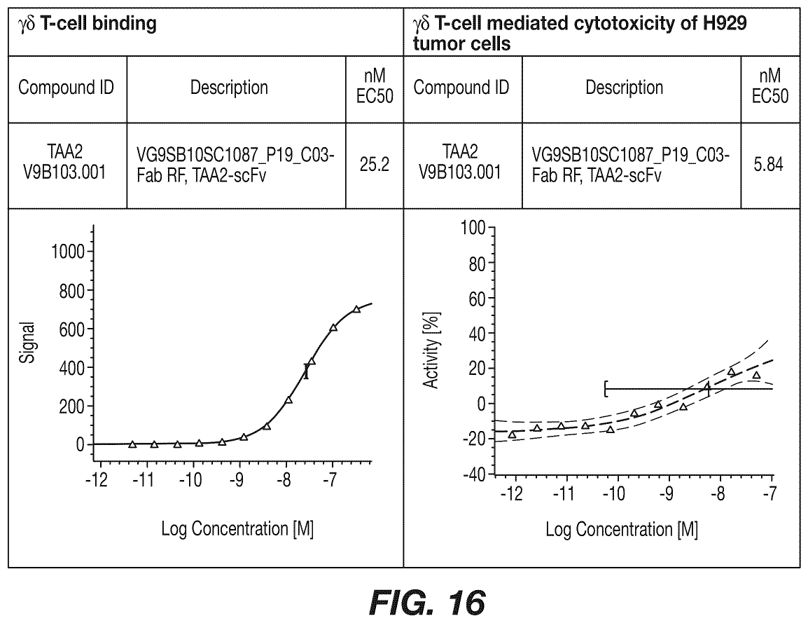

Each of FIGS. 12-16 shows that the anti-TRGV9/anti-TAA2 bispecific antibodies bind γδ T cells (left panels) and mediate γδ T cell cytotoxicity against TAA2 expressing H929 cells in vitro (right panels). For the binding assays, γδ-enriched T cells were used, and samples incubated for 1 hour at 37° C. prior to measurements. For the killing assays, expanded γδ T cells (effectors) were co-cultured with H929 at 5:1 E:T ratios in the presence of various concentrations of the bispecific antibody for 72 hours at 37° C. Bispecific constructs were tested in 11-point titration curve with a 3-fold dilution series starting at 50 nM antibody concentration. Human pan T cells were used as effector cells, as was done previously (see above). H929-WT tumor cell line was used as target cells. Dose response curves show anti-TRGV9/anti-TAA2 bispecific mediated γδ T cell cytotoxicity against TAA2 expressing H929 cells in a dose dependent manner. EC₅₀ values were calculated as described in methods. Representative data shown are from a single experiment.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An antibody that binds to T Cell Receptor Gamma Variable 9 (TRGV9), wherein the antibody comprises a heavy chain variable region (VH) and a light chain variable region (VL), wherein the antibody comprises:
(i) a VH comprising a VH CDR1 having an amino acid sequence of SEQ ID NO:98, a VH CDR2 having an amino acid sequence of SEQ ID NO:99, and a VH CDR3 having an amino acid sequence of SEQ ID NO:100; and
(ii) a VL comprising a VL CDR1 having an amino acid sequence of SEQ ID NO:101, a VL CDR2 having an amino acid sequence of SEQ ID NO:102, and a VL CDR3 having an amino acid sequence of SEQ ID NO:103.

2-22. (canceled)

23. The antibody of claim 22, wherein the antibody comprises a VH having an amino acid sequence of SEQ ID NO:104.

24. The antibody of claim 22, wherein the antibody comprises a VL having an amino acid sequence of SEQ ID NO:105.

25. (canceled)

26. An antibody that binds to T Cell Receptor Gamma Variable 9 (TRGV9), wherein the antibody comprises a heavy chain variable region (VH) and a light chain variable region (VL), wherein the antibody comprises:
(i) a VH comprising a VH CDR1 having an amino acid sequence of SEQ ID NO:107, a VH CDR2 having an amino acid sequence of SEQ ID NO:108, and a VH CDR3 having an amino acid sequence of SEQ ID NO:109; and
(ii) a VL comprising a VL CDR1 having an amino acid sequence of SEQ ID NO:110, a VL CDR2 having an amino acid sequence of SEQ ID NO:111, and a VL CDR3 having an amino acid sequence of SEQ ID NO:112.

27. The antibody of claim 26, wherein the antibody comprises a VH having an amino acid sequence of SEQ ID NO:113.

28. The antibody of claim 26, wherein the antibody comprises a VL having an amino acid sequence of SEQ ID NO:114.

29. (canceled)

30. An antibody that binds to T Cell Receptor Gamma Variable 9 (TRGV9), wherein the antibody comprises a heavy chain variable region (VH) and a light chain variable region (VL), wherein the antibody comprises:
(i) a VH comprising a VH CDR1 having an amino acid sequence of SEQ ID NO:117, a VH CDR2 having an amino acid sequence of SEQ ID NO:118, and a VH CDR3 having an amino acid sequence of SEQ ID NO:119; and
(ii) a VL comprising a VL CDR1 having an amino acid sequence of SEQ ID NO:120, a VL CDR2 having an amino acid sequence of SEQ ID NO:121, and a VL CDR3 having an amino acid sequence of SEQ ID NO:122.

31. The antibody of claim 30, wherein the antibody comprises a VH having an amino acid sequence of SEQ ID NO:123.

32. The antibody of claim 30, wherein the antibody comprises a VL having an amino acid sequence of SEQ ID NO:124.

33-45. (canceled)

46. A vector comprising a nucleic acid encoding the antibody of claim 1.

47. A host cell comprising the vector of claim 45.

48. (canceled)

49. A pharmaceutical composition comprising the antibody of claim 1, and a pharmaceutically acceptable carrier.

50-218. (canceled)

219. A vector comprising a nucleic acid encoding the antibody of claim 26.

220. A host cell comprising the vector of claim 219.

221. A pharmaceutical composition comprising the antibody of claim 26, and a pharmaceutically acceptable carrier.

222. A vector comprising a nucleic acid encoding the antibody of claim 30.

223. A host cell comprising the vector of claim 222.

224. A pharmaceutical composition comprising the antibody of claim 30, and a pharmaceutically acceptable carrier.
